(19) United States
(12) Patent Application Publication
(10) Pub. No.: US 2025/0194413 A1
(43) Pub. Date: Jun. 12, 2025

(54) DEUTERATED COMPOSITION, ORGANIC LIGHT-EMITTING DEVICE AND DISPLAY DEVICE

(71) Applicant: FUYANG SINEVA NEW MATERIAL TECHNOLOGY CO., LTD., Anhui (CN)

(72) Inventors: Zhanqi WANG, Beijing (CN); Zhiqiang LI, Anhui (CN); Jinbo LU, Beijing (CN); Changgang HUANG, Beijing (CN); Jin Woo KIM, Beijing (CN)

(21) Appl. No.: 18/842,256

(22) PCT Filed: Feb. 22, 2023
(86) PCT No.: PCT/CN2023/077721

§ 371 Date: Aug. 28, 2024

(30) Foreign Application Priority Data
Mar. 1, 2022 (CN) 202210193865.9
Nov. 29, 2022 (CN) 202211516618.4
Feb. 17, 2023 (CN) 202310129202.5

(51) Int. Cl. H10K 85/60; C07C 15/28; C07D 307/92; H10K 50/11

(52) U.S. Cl. CPC H10K 85/626; C07C 15/28; C07D 307/92; H10K 50/11; C07B 2200/05

(57) ABSTRACT

A deuterated composition, an organic light-emitting device and a display device. The deuterated composition comprises a deuterated mixture which is prepared from compound A by means of a deuteration reaction, compound A having a structure represented by formula (I). The organic light-emitting device prepared by using as a blue light host material the deuterated composition provided by the present application has a relatively low driving voltage, a relatively high current efficiency and a relatively long service life.

This application claims priority to Chinese Patent Application No. 202211516618.4 filed with the China National Intellectual Property Administration (CNIPA) on Nov. 29, 2022, claims priority to Chinese Patent Application No. 202210193865.9 filed with the CNIPA on Mar. 1, 2022 and claims priority to Chinese Patent Application No. 202310129202.5 filed with the CNIPA on Feb. 17, 2023, the disclosures of which are incorporated herein by reference in their entireties.

### TECHNICAL FIELD

The present application belongs to the technical field of organic electroluminescent materials and, in particular, relates to a deuterated composition, an organic electroluminescent device and a display device.

### BACKGROUND

An organic electroluminescence phenomenon was discovered early in 1963 but did not attract much attention at that time. The technology did not regain attention and a brand-new research field was opened up until a Tang research group of American Eastman Kodak Company invented a high-brightness and high-efficiency thin-film organic electroluminescent device (OLED) made of an organic fluorescent material and a hole material and driven by a low direct current voltage in 1987.

Compared with other display technologies, OLED technology has outstanding advantages such as low power consumption, a fast response speed, easy bending, a wide viewing angle, large-area display and complete emitted colors and can be compatible with multiple existing standards and technologies to prepare low-cost light-emitting devices, thereby showing a wide application prospect in color flat-panel display. Over the past decades, as a new display technology, OLED has achieved great development and is widely used in the fields of flat-panel display, flexible display, solid-state lighting and in-vehicle display.

Currently, organic electroluminescence (OLED) has become a mainstream display technology. Correspondingly, various new OLED materials are developed. At present, a compound obtained after positions 9 and 10 of anthracene are substituted with aryl groups is mainly used as a host material for blue light. The aryl groups mainly include groups such as phenyl, naphthyl, anthryl, dibenzofuranyl, dibenzothienyl, benzodibenzofuranyl and benzodibenzothienyl. However, various types of performance of the host material for blue light still need to be improved, especially efficiency, lifetime and voltage.

Therefore, it is a research focus in the art to develop more types of host materials for blue light with more perfect performance to meet usage requirements of high-performance OLED devices.

### SUMMARY

The following is a summary of the subject matter described in detail herein. This summary is not intended to limit the scope of the claims.

In view of deficiencies of the prior art, an object of the present application is to provide a deuterated composition, an organic electroluminescent device and a display device. In the present application, an anthracene compound substituted with an aryl group is subjected to a deuterated reaction to obtain a deuterated mixture, the obtained deuterated mixture is used as a host material for blue light, and the OLED light-emitting device prepared in this manner has relatively high current efficiency and a relatively long lifetime. In addition, in the present application, a deuterated mixture obtained after a compound having a particular structure is subjected to a deuterated reaction is further used as a host material of a light-emitting layer to obtain the organic electroluminescent device with excellent overall performance.

To achieve the object, the present application adopts the technical solutions below.

In a first aspect, the present application provides a deuterated composition including a deuterated mixture prepared from Compound A by means of a deuterated reaction:
wherein Compound A has a structure represented by Formula I:

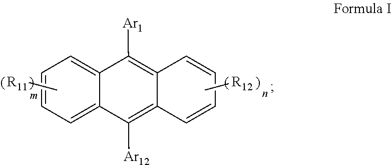

wherein Ar₁₁ and Ar₁₂ are each independently selected from any one of substituted or unsubstituted C6 to C40 aryl, or substituted or unsubstituted C12 to C40 heteroaryl;
R₁₁ and R₁₂ are each independently selected from any one of deuterium, substituted or unsubstituted C1 to C6 linear or branched alkyl, substituted or unsubstituted C6 to C40 aryl, or substituted or unsubstituted C12 to C40 heteroaryl;
substituted substituents in Ar₁₁, Ar₁₂, R₁₁ and R₁₂ are each independently selected from at least one of C1 to C6 linear or branched alkyl, C6 to C20 aryl or C6 to C20 heteroaryl; and
m and n are each independently selected from integers between 0 and 4.

It is to be noted that C6 to C40 aryl and C12 to C40 heteroaryl in substituted or unsubstituted C6 to C40 aryl and substituted or unsubstituted C12 to C40 heteroaryl in the above Ar₁₁, Ar₁₂, R₁₁ and R₁₂ are selected from any one of phenyl, biphenyl, naphthyl, phenanthryl, anthryl, fluorenyl, benzofluorenyl, dibenzofluorenyl, triphenylenyl, fluoranthenyl, pyrenyl, perylenyl, spriofluorenyl, indenofluorenyl or hydrogenated benzanthryl.

In the present application, an anthracene compound substituted with an aryl group is subjected to a deuterated reaction to obtain a deuterated mixture, the obtained deuterated mixture is used as a host material for blue light, and an OLED light-emitting device prepared in this manner has relatively high current efficiency and a relatively long lifetime.

In the field of display technologies, a compound obtained after positions 9 and 10 of anthracene are substituted with aryl groups is generally used as a host material for blue light for preparing an organic electroluminescent device. However, various types of performance of the organic electroluminescent device prepared in this manner still need to be improved, especially efficiency, lifetime and voltage. Therefore, in the prior art, an anthracene compound is generally substituted with a deuterated compound to expect to obtain a deuterated anthracene derivative, thereby improving the various types of performance of the organic electroluminescent device. However, when anthracene is substituted with the deuterated compound, a relatively high requirement is imposed on the purity of the deuterated compound. A purification process of the deuterated compound is relatively cumbersome, a condition for the deuterated compound to perform a substitution reaction on anthracene is relatively strict, and a post-treatment process is complex. As shown in the following, in the prior art, the following steps are generally needed to obtain a deuterated compound

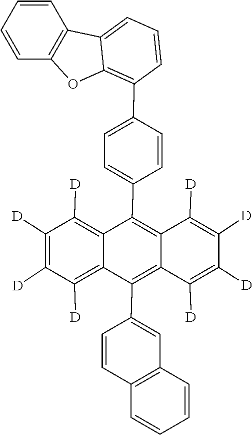 [SMILES: [2H]c1c([2H])c([2H])c2c(-c3ccc4ccccc4c3)c3c([2H])c([2H])c([2H])c([2H])c3c(-c3ccc(-c4cccc5c4oc4ccccc45)cc3)c2c1[2H]]

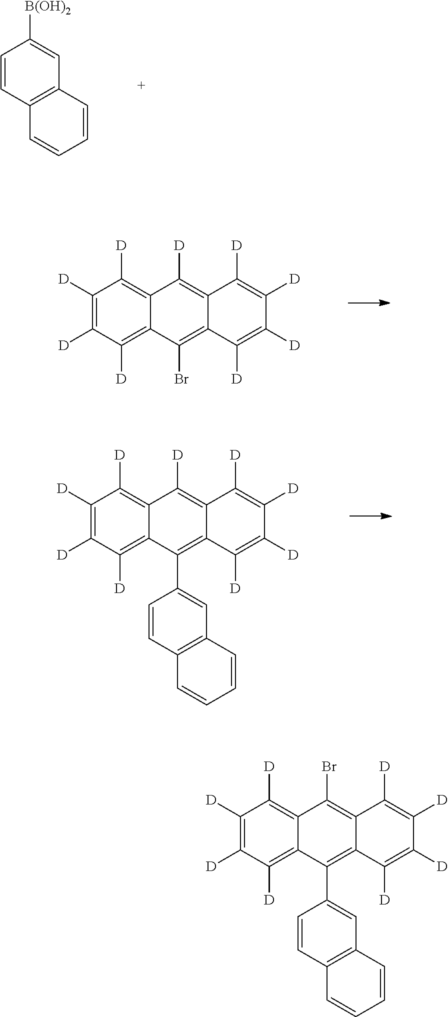 [SMILES: OB(O)c1ccc2ccccc2c1 ; [2H]c1c([2H])c([2H])c2c(Br)c3c([2H])c([2H])c([2H])c([2H])c3c([2H])c2c1[2H] ; [2H]c1c([2H])c([2H])c2c(-c3ccc4ccccc4c3)c3c([2H])c([2H])c([2H])c([2H])c3c([2H])c2c1[2H] ; [2H]c1c([2H])c([2H])c2c(-c3ccc4ccccc4c3)c3c([2H])c([2H])c([2H])c([2H])c3c(Br)c2c1[2H]]

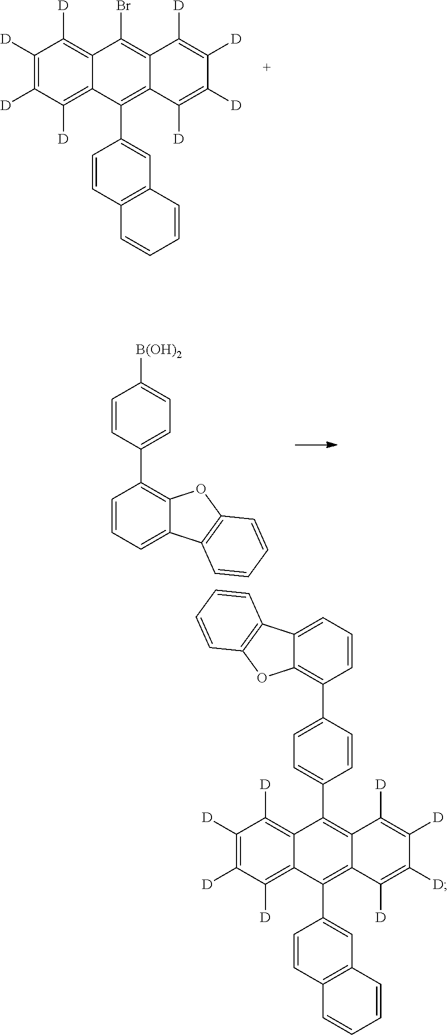

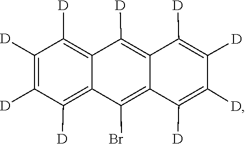

which needs to be used in the above reaction process, needs to be prepared by using fully deuterated anthracene. In a process of preparing

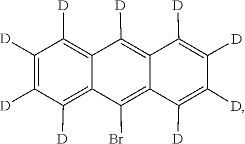

to obtain a high-purity deuterated product, a large number of operations need to be performed in a reaction process and a purification process, which is relatively cumbersome. Moreover, when the prepared

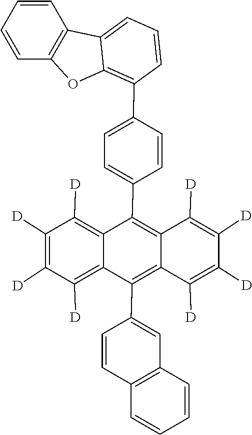 [SMILES: [2H]c1c([2H])c([2H])c2c(-c3ccc4ccccc4c3)c3c([2H])c([2H])c([2H])c([2H])c3c(-c3ccc(-c4cccc5c4oc4ccccc45)cc3)c2c1[2H]]

is used as a host material for blue light, overall performance of a prepared OLED light-emitting device is relatively poor and still needs to be improved.

In the present application, an anthracene compound (Compound A) having a particular structure is subjected to a deuterated reaction, thereby avoiding cumbersome purification work. The preparation method is simple, the reaction condition is mild, and the post treatment is simple. Moreover, after the anthracene compound (Compound A) having the particular structure is subjected to the deuterated reaction, a deuterated mixture can be obtained. When the prepared deuterated mixture is used as a host material for blue light, overall performance of a prepared OLED light-emitting device is relatively good with a relatively low driving voltage, a relatively high current effect and a relatively long service life. For example, in the present application, the following deuterated reaction can be simply conducted to obtain a deuterated mixture

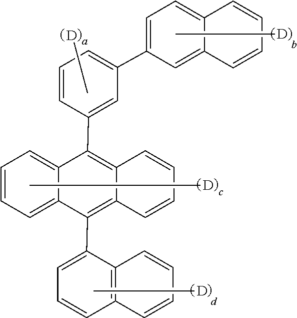

(wherein a is selected from integers between 0 and 4, b is selected from integers between 0 and 7, c is selected from integers between 0 and 8, d is selected from integers between 0 and 7; and a+b+c+d≥1):

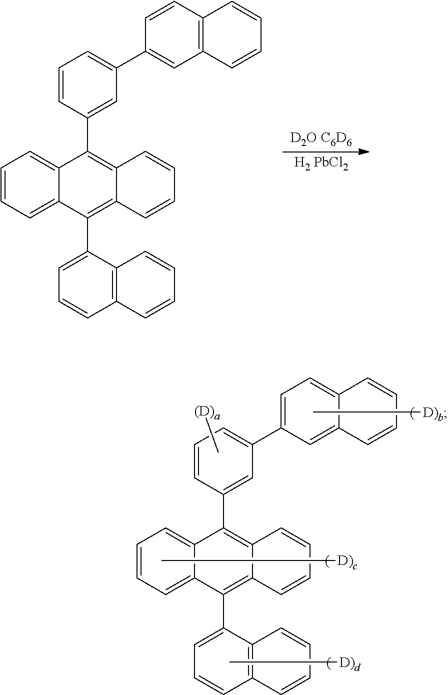

wherein a is selected from integers between 0 and 4, b is selected from integers between 0 and 7, c is selected from integers between 0 and 8, d is selected from integers between 0 and 7, and a+b+c+d≥1.

As can be seen from the above reaction formula, in the present application, the relatively simple deuterated reaction can be conducted to obtain the deuterated mixture, thereby avoiding treatment processes such as cumbersome purification and simplifying a process.

It is to be noted that in an organic reaction process, when reaction active sites on reactive molecules are not significantly different, it is difficult to conduct a substitution reaction on a characteristic reaction site. Therefore, in the present application, after Compound A is subjected to the deuterated reaction, a mixture (the deuterated mixture) is obtained.

In the present application, Ar₁₁ and Ar₁₂ are each independently selected from any one of substituted or unsubstituted C6 to C40 aryl (which may be, for example, C6, C8, C10, C12, C16, C20, C24, C28, C30, C32, C36 or C40), or substituted or unsubstituted C12 to C40 heteroaryl (which may be, for example, C6, C8, C10, C12, C16, C20, C24, C28, C30, C32, C36 or C40);
R₁₁ and R₁₂ are each independently selected from any one of deuterium, substituted or unsubstituted C1 to C6 linear or branched alkyl (which may be, for example, methyl, ethyl, propyl, n-butyl, tert-butyl, n-pentyl, isopentyl or hexyl), substituted or unsubstituted C6 to C40 aryl (which may be, for example, C6, C8, C10, C12, C16, C20, C24, C28, C30, C32, C36 or C40), or substituted or unsubstituted C12 to C40 heteroaryl (which may be, for example, C6, C8, C10, C12, C16, C20, C24, C28, C30, C32, C36 or C40);
the substituted substituents in Ar₁₁, Ar₁₂, R₁₁ and R₁₂ are each independently selected from at least one of C1 to C6 linear or branched alkyl (which may be, for example, methyl, ethyl, propyl, n-butyl, tert-butyl, n-pentyl, isopentyl or hexyl), or C6 to C20 aryl (which may be, for example, C6, C8, C10, C12, C16 or C20), or C6 to C20 (which may be, for example, C6, C8, C10, C12, C16 or C20) heteroaryl; and
m and n are each independently selected from integers between 0 and 4, which may be, for example, 0, 1, 2, 3 or 4.

Optional technical solutions of the present application are set forth below and not intended to limit the technical solutions provided in the present application. Objects and beneficial effects of the present application can be achieved through the optional technical solutions set forth below.

As a preferred technical solution of the present optional technical solution, the deuterated composition includes a deuterated mixture prepared from at least one type of Compound A by means of a deuterated reaction:
wherein the Compound A has a structure represented by Formula I′:

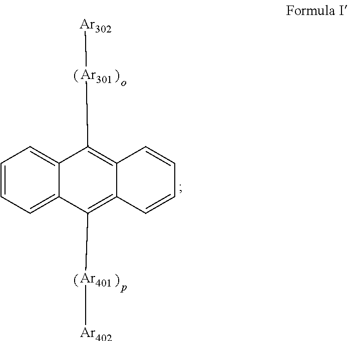

wherein Ar₃₀₁, Ar₃₀₂, Ar₄₀₁ and Ar₄₀₂ are each independently selected from one of phenyl, naphthyl or phenanthryl, and Ar₃₀₁, Ar₃₀₂, Ar₄₀₁ and Ar₄₀₂ are not phenanthryl at the same time; the structure represented by Formula I′ contains only one phenanthryl; and
and p are each independently selected from 0 or 1.

As a preferred technical solution of the present optional technical solution, the Compound A is not

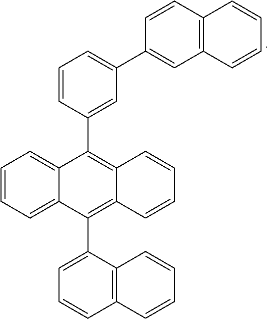 [SMILES: c1cc(-c2ccc3ccccc3c2)cc(-c2c3ccccc3c(-c3cccc4ccccc34)c3ccccc23)c1]

In the present application, an anthracene compound substituted with an aryl group is subjected to a deuterated reaction to obtain a deuterated mixture, the obtained deuterated mixture is used as a host material of a light-emitting layer, and an OLED light-emitting device prepared in this manner has relatively high current efficiency and a relatively long lifetime.

In the present application, an anthracene compound (Compound A) having a particular structure is subjected to a deuterated reaction, thereby avoiding cumbersome purification work. The preparation method is simple, the reaction condition is mild, and the post treatment is simple. Moreover, after the anthracene compound (Compound A) having the particular structure is subjected to the deuterated reaction, a deuterated mixture can be obtained. When the prepared deuterated mixture is used as a host material of a light-emitting layer, overall performance of a prepared OLED light-emitting device is relatively good with a relatively low driving voltage, a relatively high current effect and a relatively long service life.

It is to be noted that in an organic reaction process, when reaction active sites on reactive molecules are not significantly different, it is difficult to conduct a substitution reaction on a characteristic reaction site. Therefore, in the present application, after Compound A is subjected to the deuterated reaction, a mixture (the deuterated mixture) is obtained, even if only one H atom is substituted with a deuterium atom on a reactant molecule. Since activity of H atoms on the reactant is not significantly different, a molecular ion peak of the mixture obtained in this case is increased by 1 compared with that of the reactant if a mass spectrum is made. However, the mixture obtained in this case is a mixture of various isomers obtained after substitution is performed by single deuterium atoms.

An example is as follows:

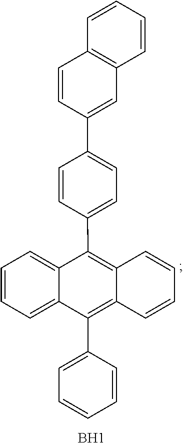

BH1 contains 24 hydrogen atoms, and isomers obtained after one of the H atoms is substituted with a deuterium atom include, but are not limited to, the following:

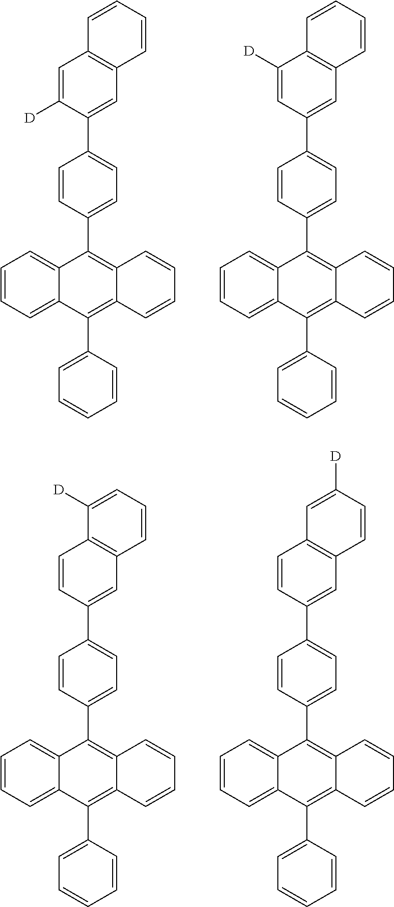

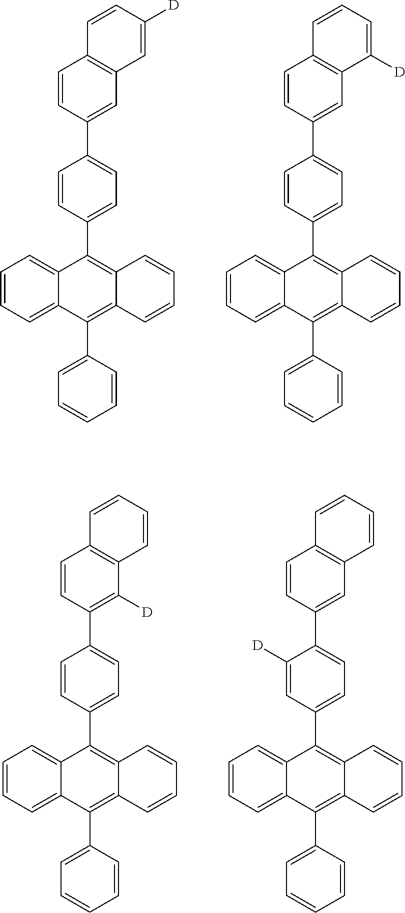 [SMILES: [2H]c1ccc2ccc(-c3ccc(-c4c5ccccc5c(-c5ccccc5)c5ccccc45)cc3)cc2c1 ; [2H]c1cccc2ccc(-c3ccc(-c4c5ccccc5c(-c5ccccc5)c5ccccc45)cc3)cc12 ; [2H]c1c(-c2ccc(-c3c4ccccc4c(-c4ccccc4)c4ccccc34)cc2)ccc2ccccc12 ; [2H]c1cc(-c2c3ccccc3c(-c3ccccc3)c3ccccc23)ccc1-c1ccc2ccccc2c1]

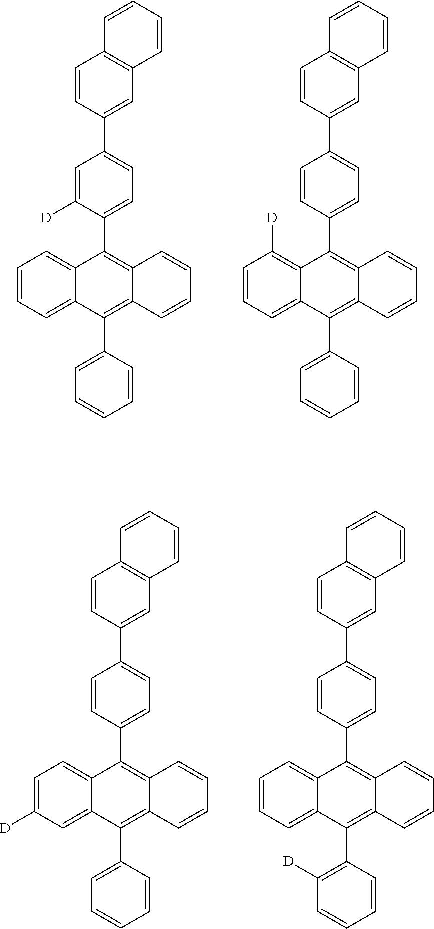 [SMILES: [2H]c1cc(-c2ccc3ccccc3c2)ccc1-c1c2ccccc2c(-c2ccccc2)c2ccccc12 ; [2H]c1cccc2c(-c3ccccc3)c3ccccc3c(-c3ccc(-c4ccc5ccccc5c4)cc3)c12 ; [2H]c1ccc2c(-c3ccc(-c4ccc5ccccc5c4)cc3)c3ccccc3c(-c3ccccc3)c2c1 ; [2H]c1ccccc1-c1c2ccccc2c(-c2ccc(-c3ccc4ccccc4c3)cc2)c2ccccc12]

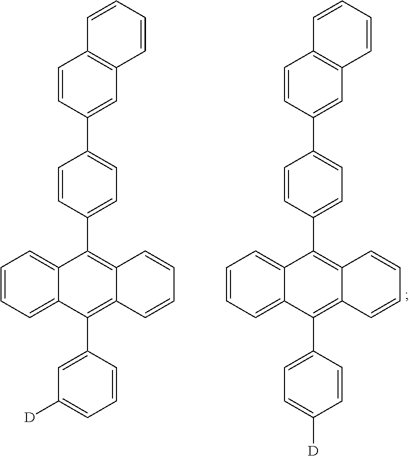 [SMILES: [2H]c1cccc(-c2c3ccccc3c(-c3ccc(-c4ccc5ccccc5c4)cc3)c3ccccc23)c1 ; [2H]c1ccc(-c2c3ccccc3c(-c3ccc(-c4ccc5ccccc5c4)cc3)c3ccccc23)cc1]

where molecular ion peaks of mass spectral peaks have the same m/z when the above compounds exist in the deuterated composition.

BH1 contains 24 hydrogen atoms, and isomers obtained after 23 of the H atoms are substituted with deuterium atoms include, but are not limited to, the following:

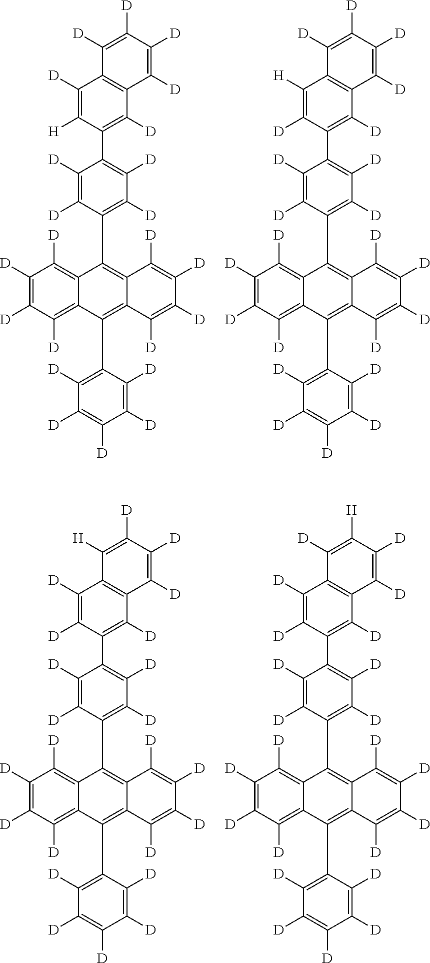

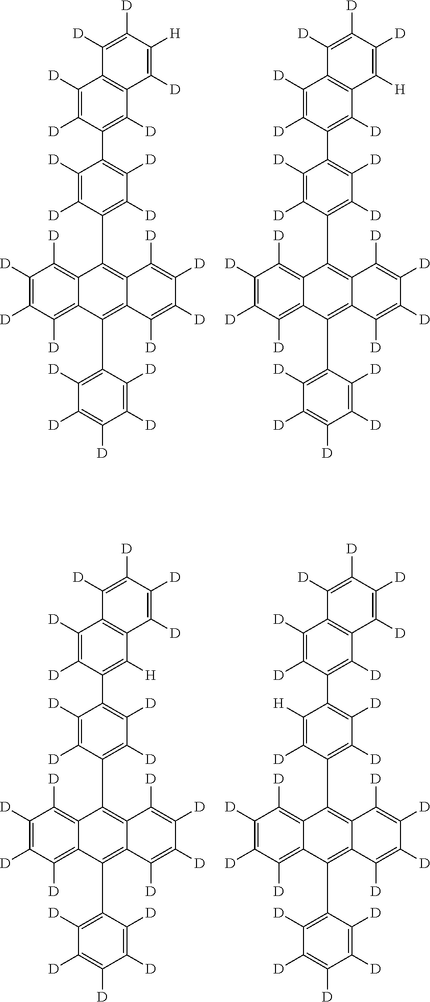

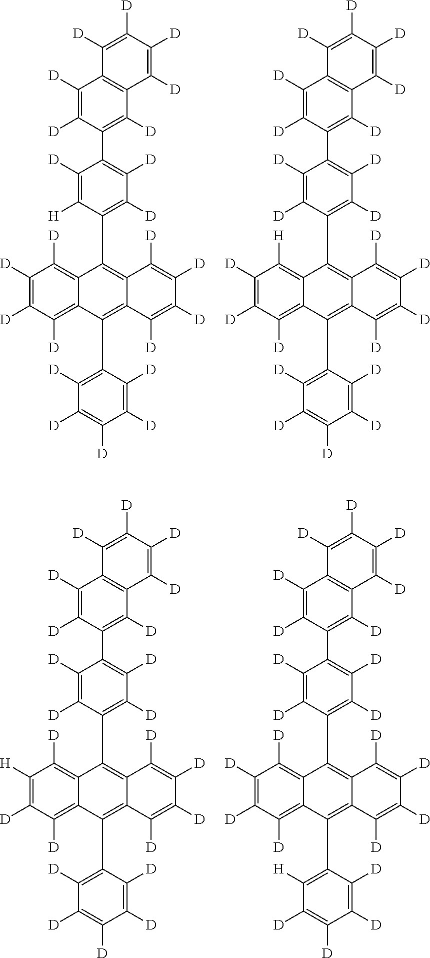

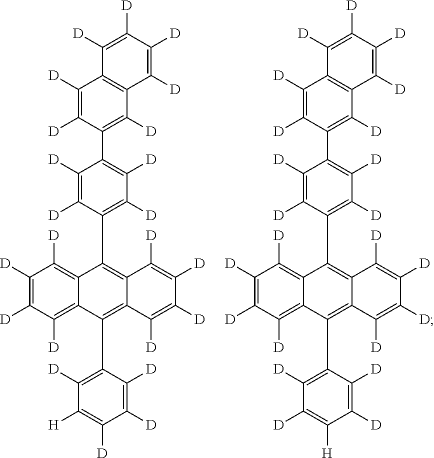

wherein molecular ion peaks of mass spectral peaks have the same m/z when the above compounds exist in the deuterated composition.

Similarly, BH1 contains 24 hydrogen atoms, and isomers obtained after 2 to 22 of the H atoms are substituted with deuterium atoms are numerous in variety and complex. Those skilled in the art may draw specific structures according to common knowledge.

As long as the deuterated reaction is conducted and the deuteration rate is not 0 or 100%, the obtained deuterated composition must contain multiple components.

As long as the deuterated reaction is conducted, the deuterated combination may contain any one or a combination of at least two of a product having one H atom substituted with deuterium, a product having two H atoms substituted with deuterium, a product having three H atoms substituted with deuterium, a product having four H atoms substituted with deuterium, a product having five H atoms substituted with deuterium, a product having six H atoms substituted with deuterium, a product having seven H atoms substituted with deuterium, a product having eight H atoms substituted with deuterium, a product having nine H atoms substituted with deuterium, . . . or a product having 24 H atoms substituted with deuterium.

Moreover, in a condition of the deuterated reaction of the present application, assuming that BH1 has a deuteration rate of 12.5%, it is theoretically calculated that three H atoms are substituted with deuterium atoms. According to a reaction principle and common knowledge, it is inevitable that not all BH1 molecules have three H atoms substituted with deuterium atoms, and products having two H, one H, four H and five H substituted with deuterium atoms must exist. In this manner, if m/z of a mass spectrum of BH1 is represented by M and the deuterated composition has a deuteration rate of 12.5%, a mass spectral peak on a right-most side of a mass spectrum has at least five m/z, which are M+1, M+2, M+3, M+4 and M+5, respectively, and two peaks M+6 and M+7 appear and are isotope peaks. Of course, it may be determined according to common knowledge in the art that whether the two peaks M+6 and M+7 are contributed by isotopes of separate M+4 and M+5 or a product having 6 H substituted with deuterium atoms and a product having 7 H substituted with deuterium atoms are in the deuterated composition.

As a preferred technical solution of the present optional technical solution, the deuterated composition includes a deuterated mixture prepared from two types of Compound A by means of a deuterated reaction.

Preferably, the deuterated mixture includes at least five types of compounds, which may be, for example, five types, six types, seven types, eight types, nine types, ten types, eleven types or twelve types, etc.

It is to be noted that in the present application, each type of compounds refer to compounds obtained after Compound A is subjected to the deuterated reaction and having the same number of deuterium atoms. After a mass spectrometry test is performed on compounds of the same type, m/z of mass spectral peaks on a right side of mass spectrums of the compounds of the same type is the same peak. For example, in the above Compound BH1, only one hydrogen atom is substituted with a deuterium atom, and a compound having one deuterium atom is referred to as a type 1 compound. Moreover, since a position substituted with a deuterium atom is not certain, any one of the 24 hydrogen atoms in Compound BH1 may be substituted with a deuterium atom. Therefore, this type of compounds each having one deuterium atom include 24 types of compounds.

Preferably, the deuterated mixture includes at least six types of compounds, which may be, for example, six types, seven types, eight types, nine types, ten types, eleven types or twelve types, etc.

Preferably, the deuterated mixture includes at least seven types of compounds, which may be, for example, seven types, eight types, nine types, ten types, eleven types or twelve types, etc.

As a preferred technical solution of the present optional technical solution, Compound A is selected from any one of the following compounds:

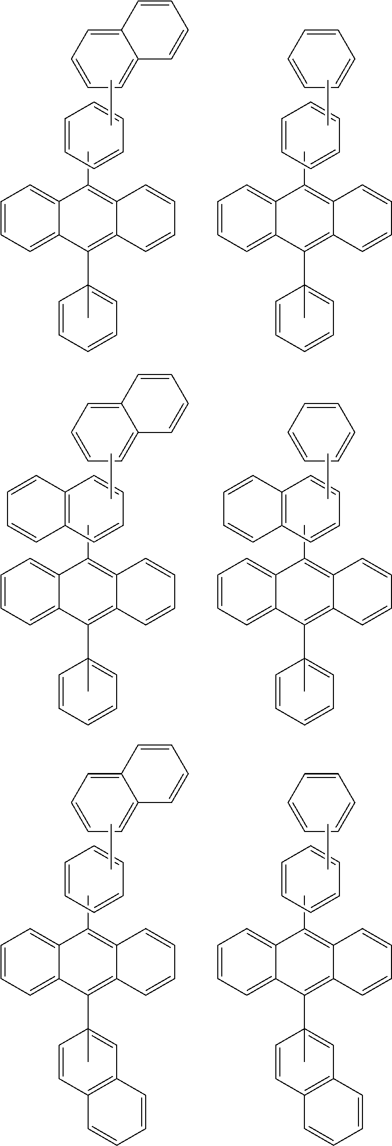

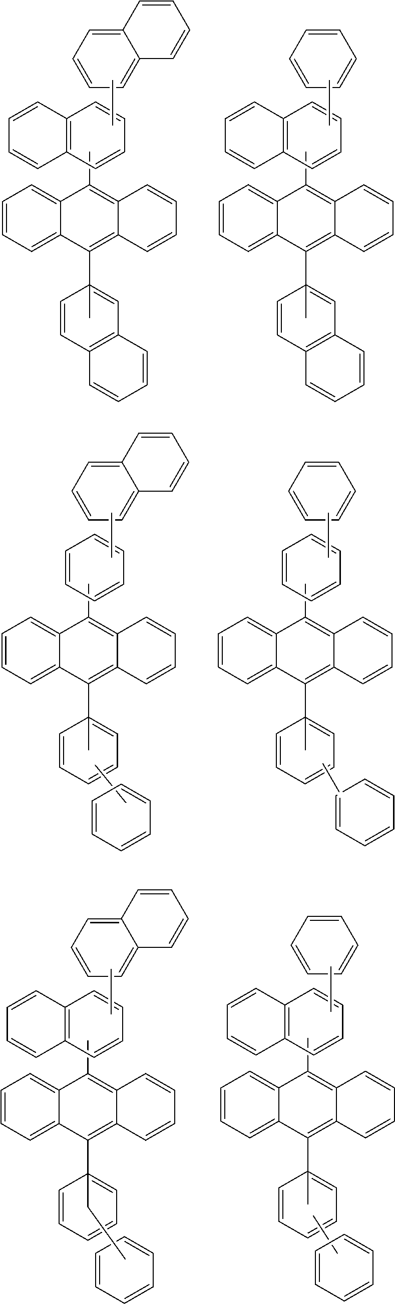

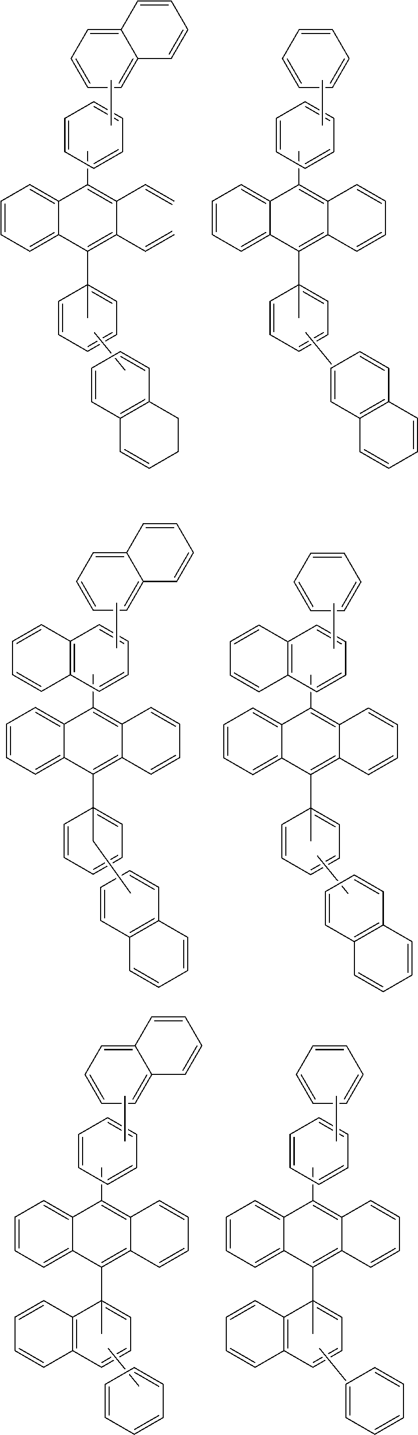

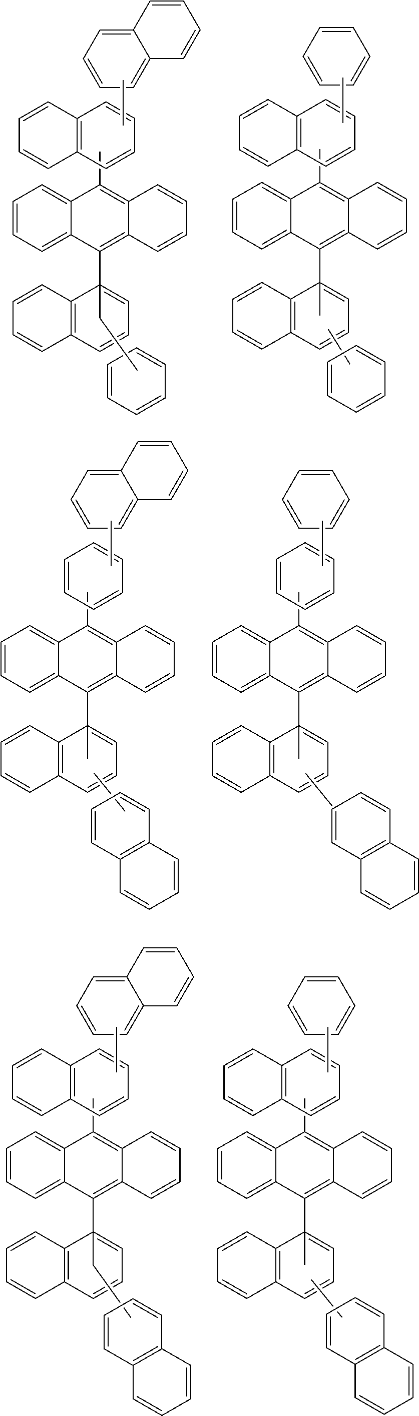

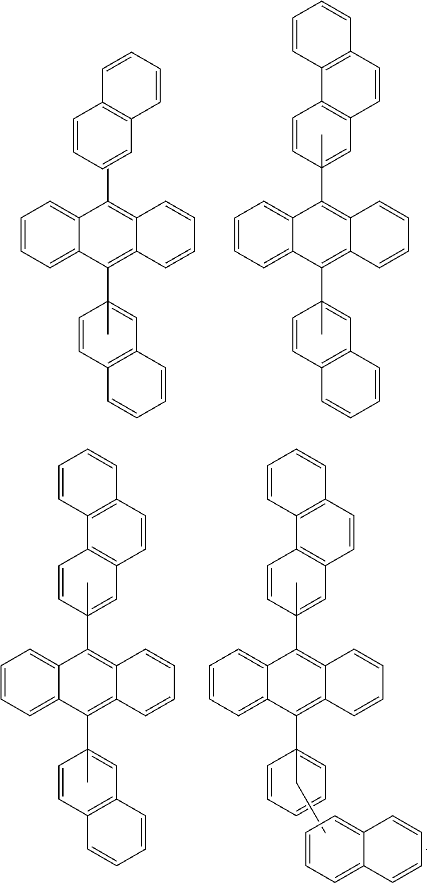

As a preferred technical solution of the present application, Ar₁₁, Ar₁₂, R₁₁ and R₁₂ are each independently selected from any one of

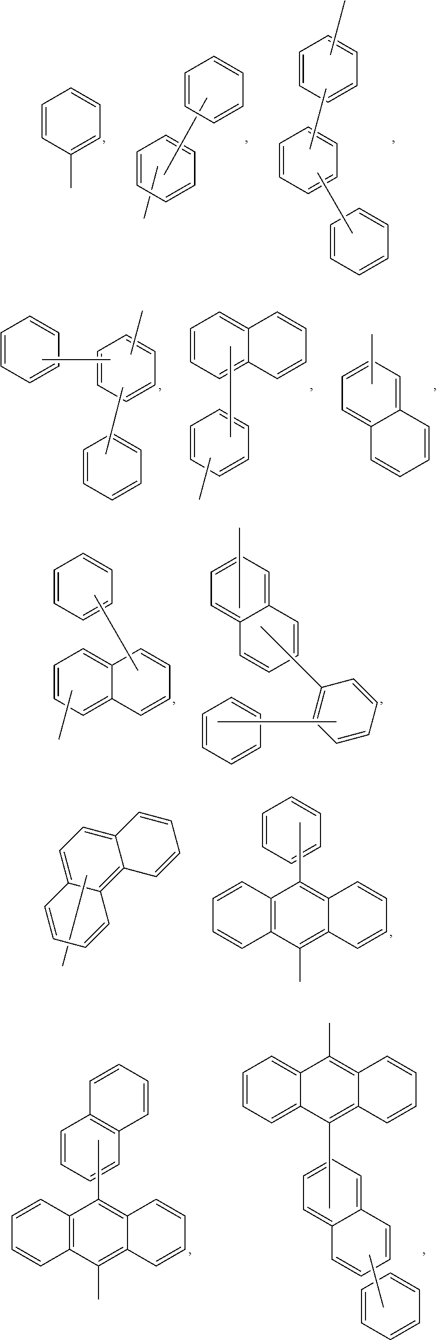

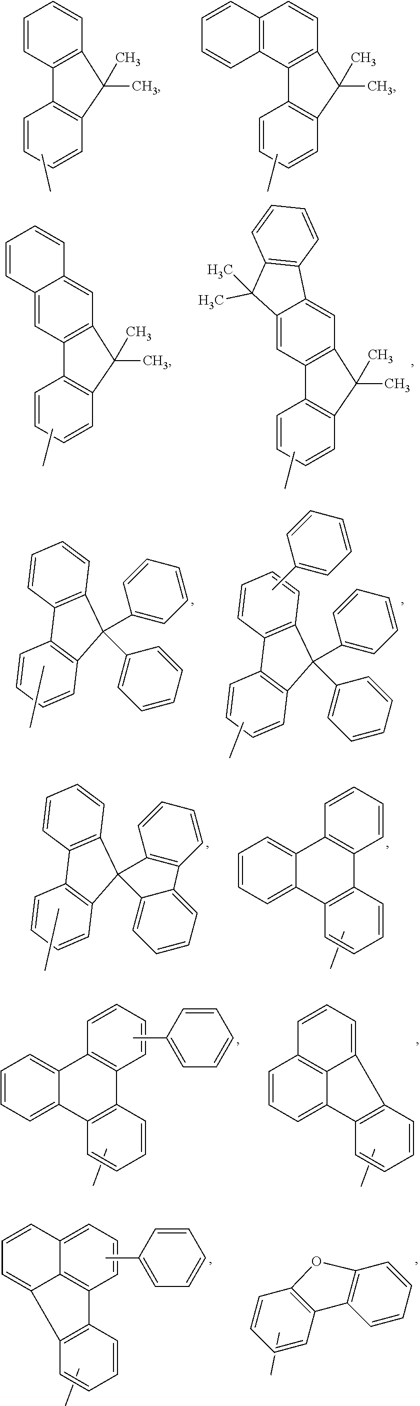

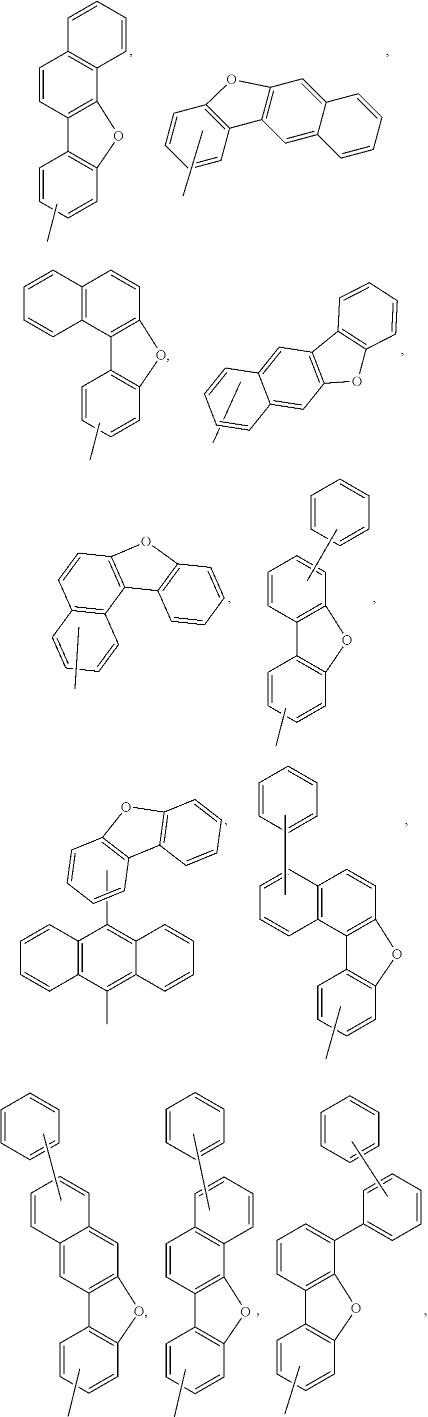

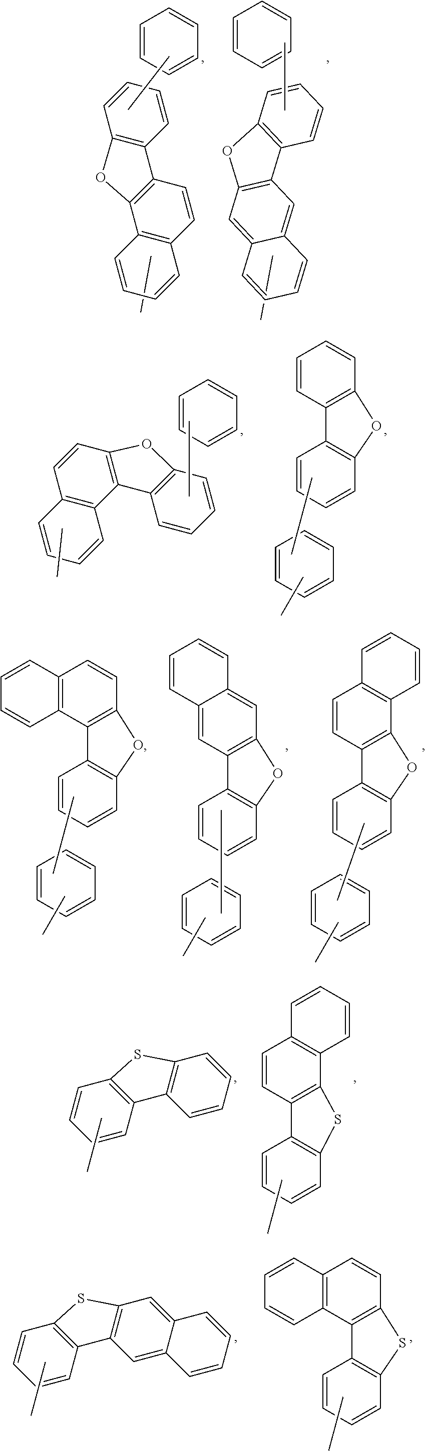

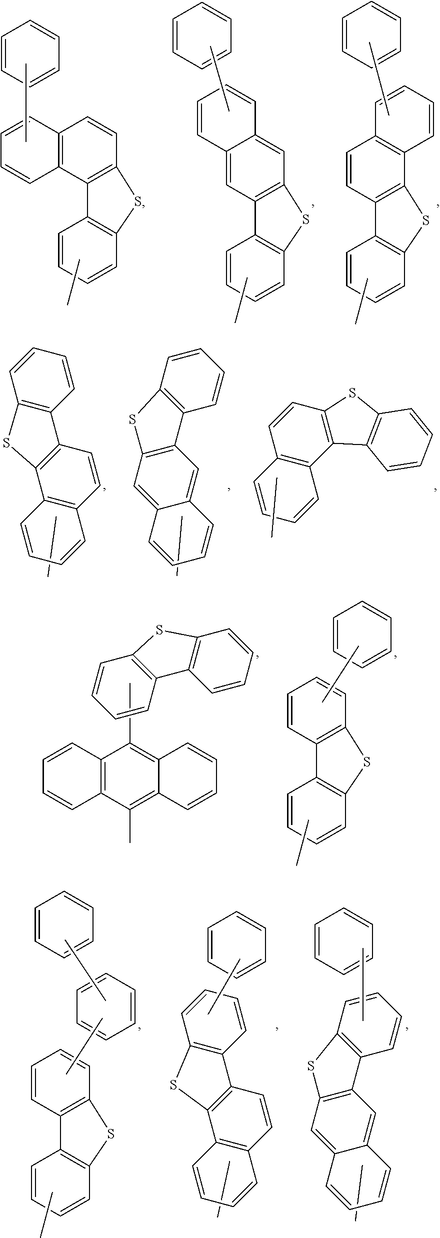

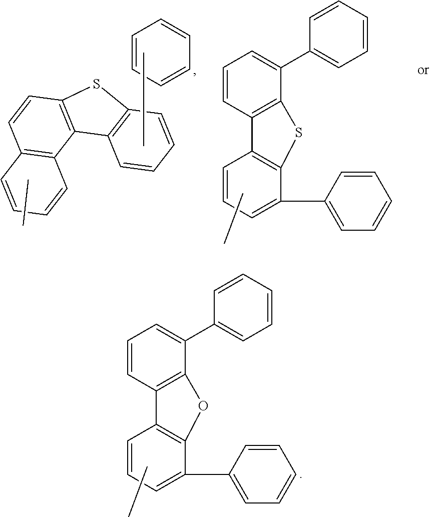

Preferably, Ar₁₂ is selected from any one of

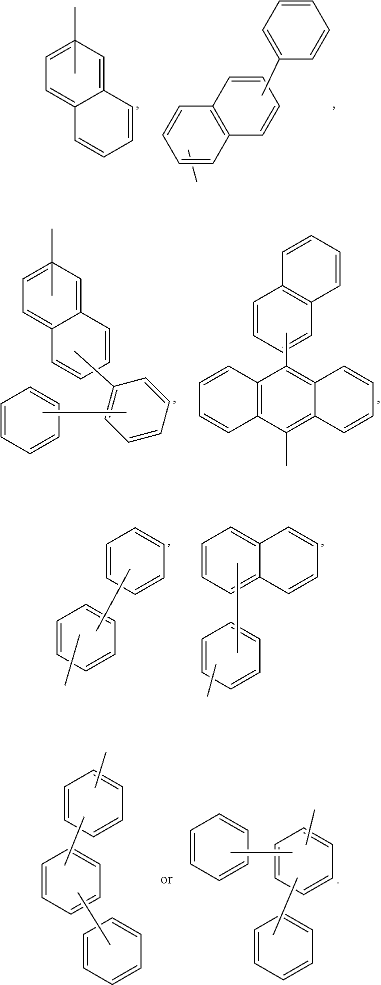

Preferably, R₁₁ and R₁₂ are each independently selected from any one of methyl, ethyl, propyl, butyl,

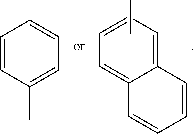

As a preferred technical solution of the present optional technical solution, Compound A is selected from any one of the following compounds:

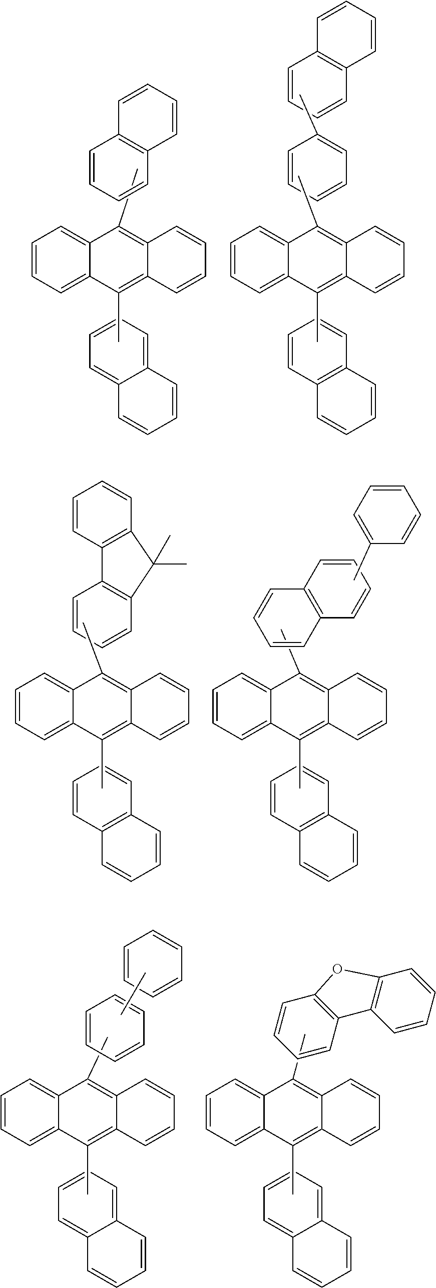

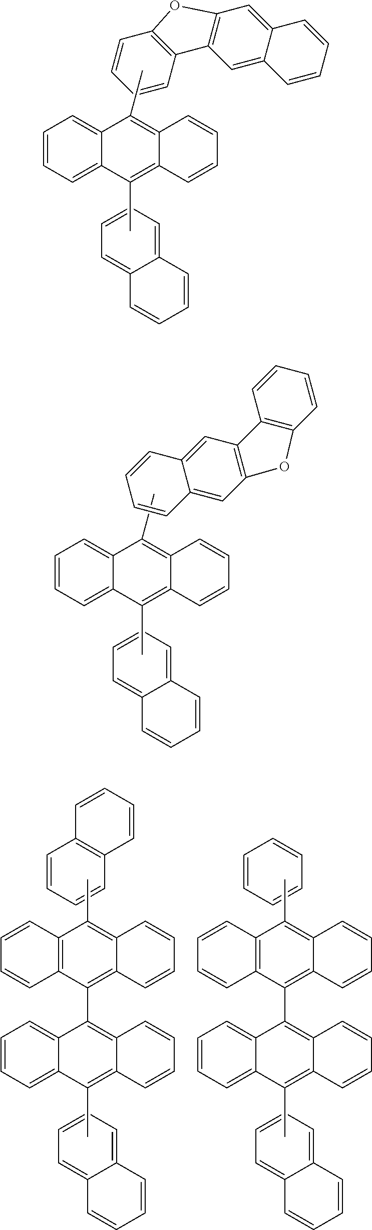

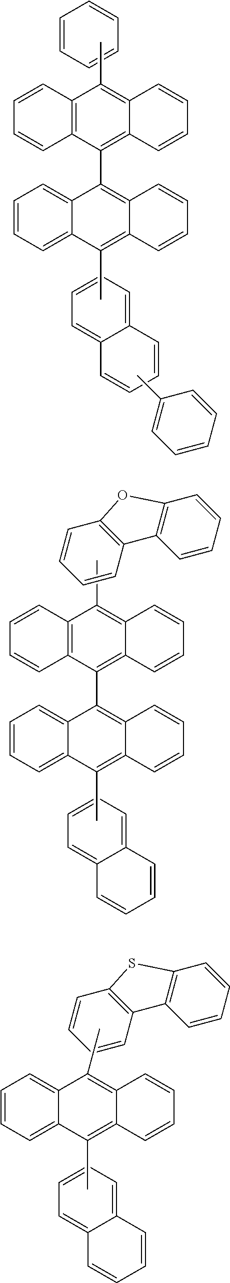

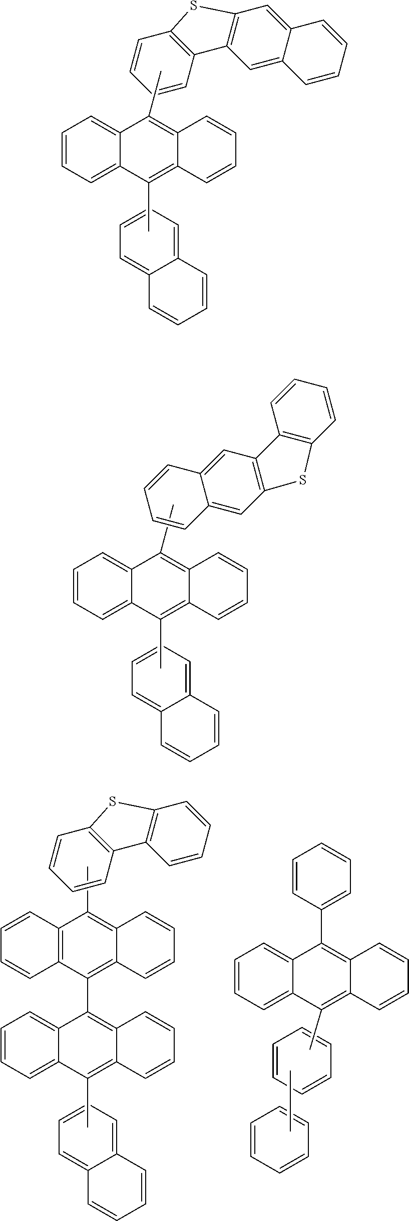

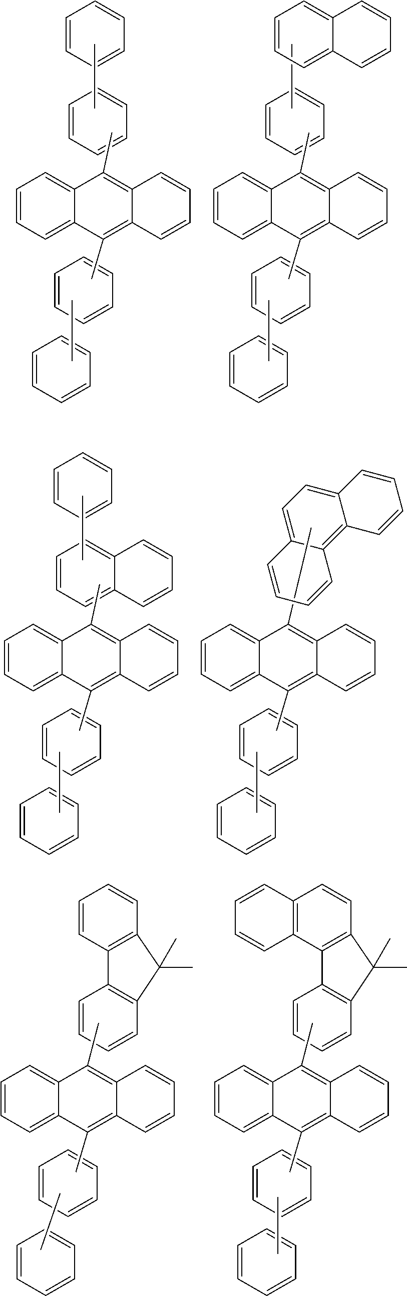

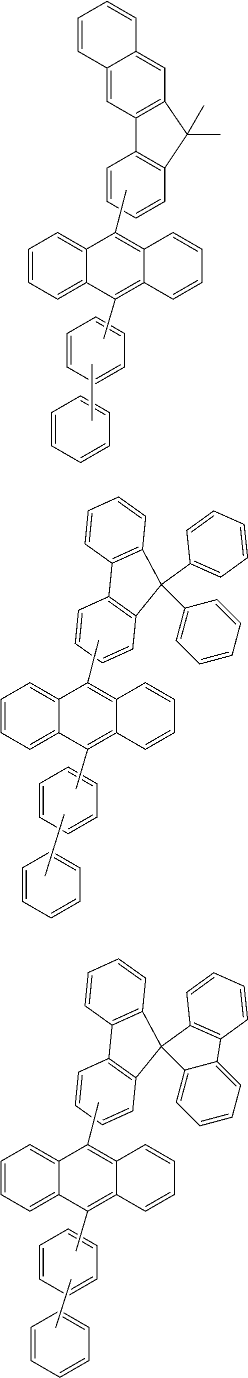

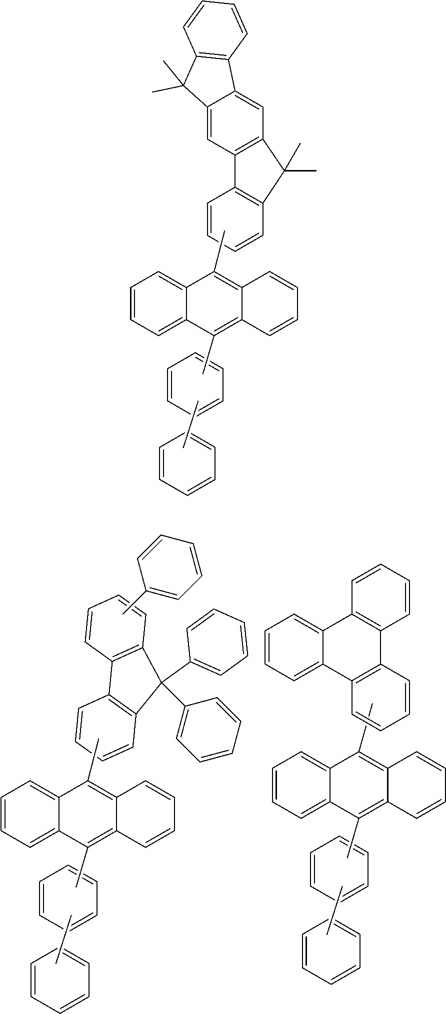

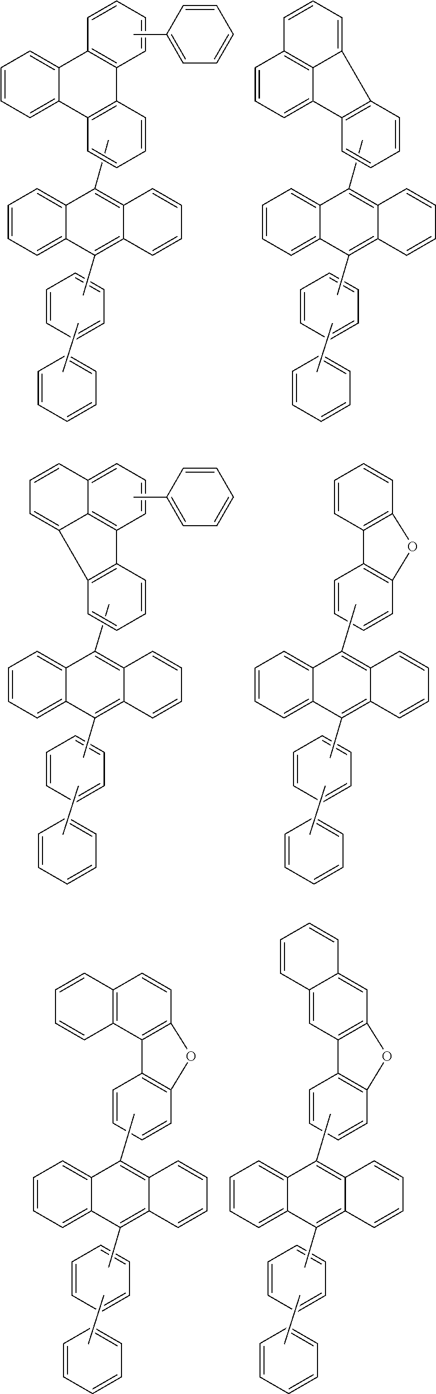

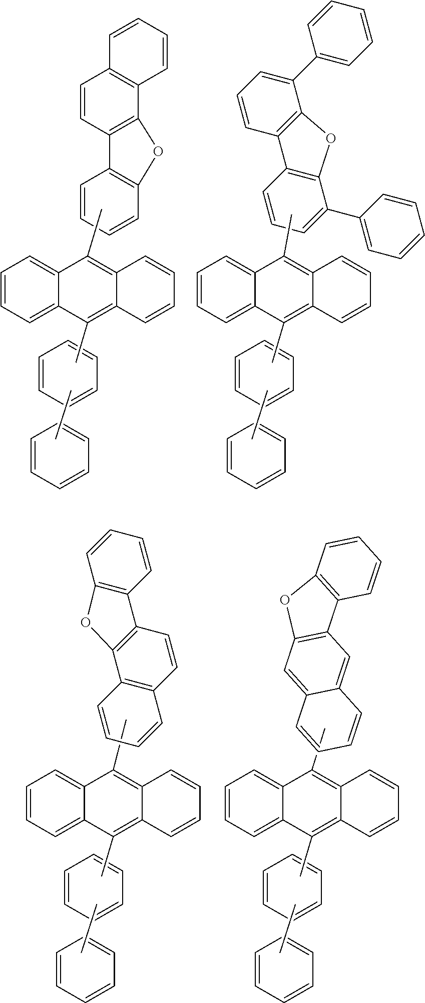

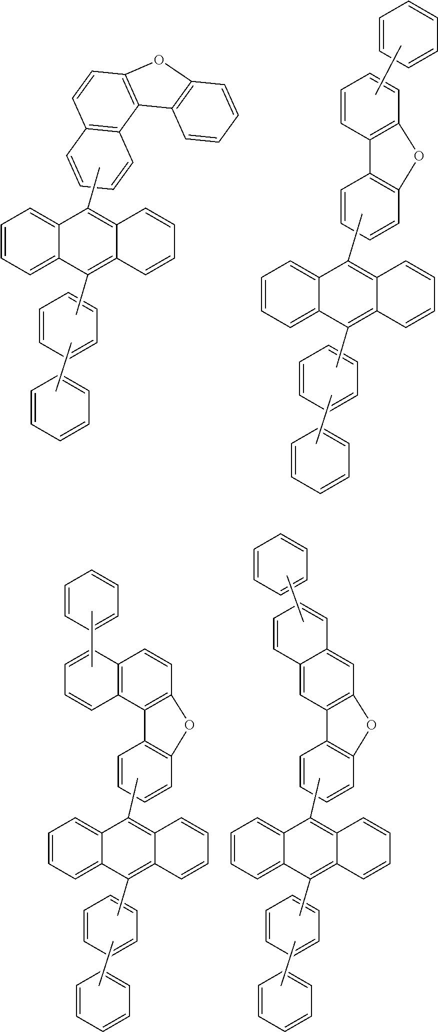

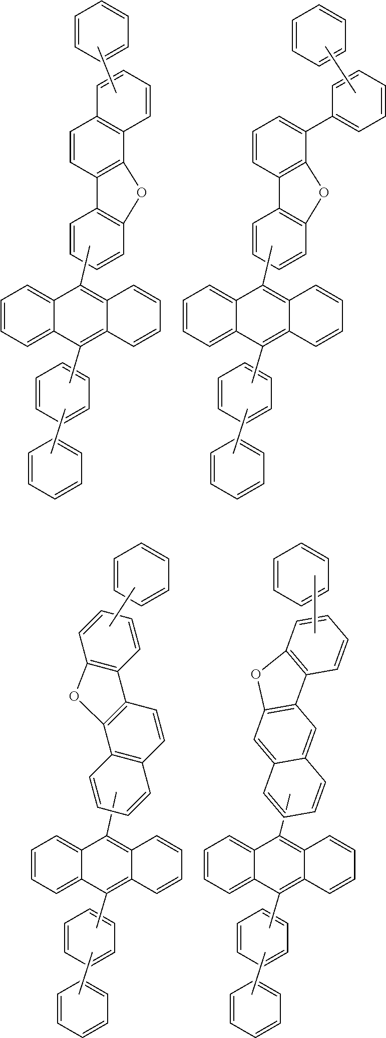

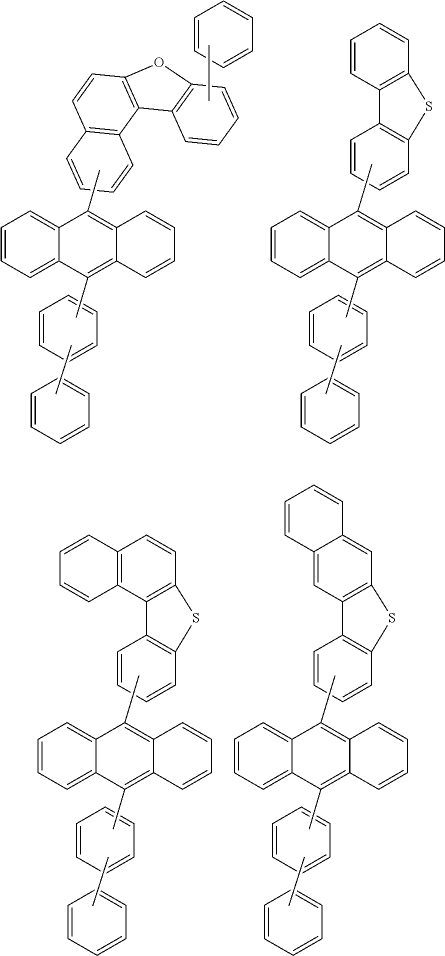

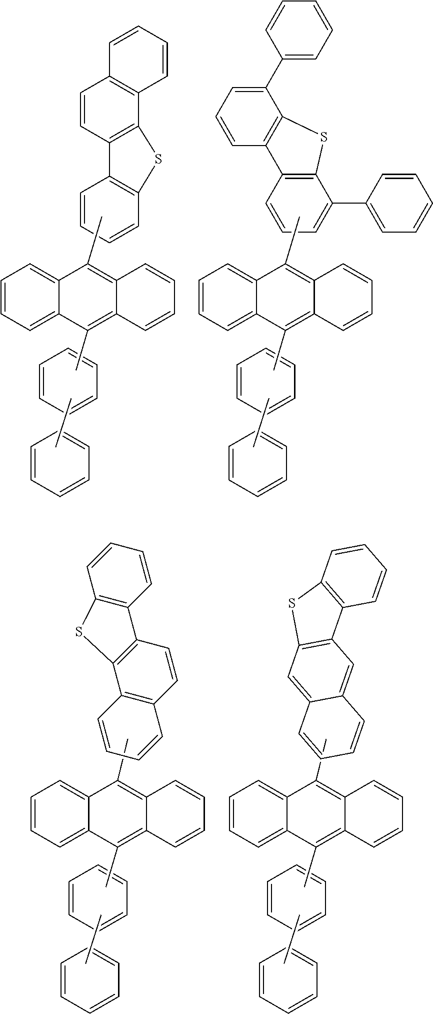

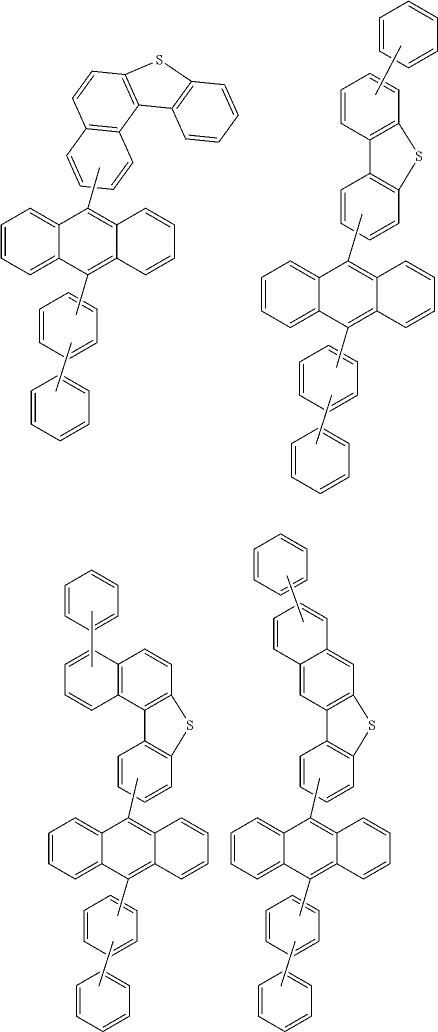

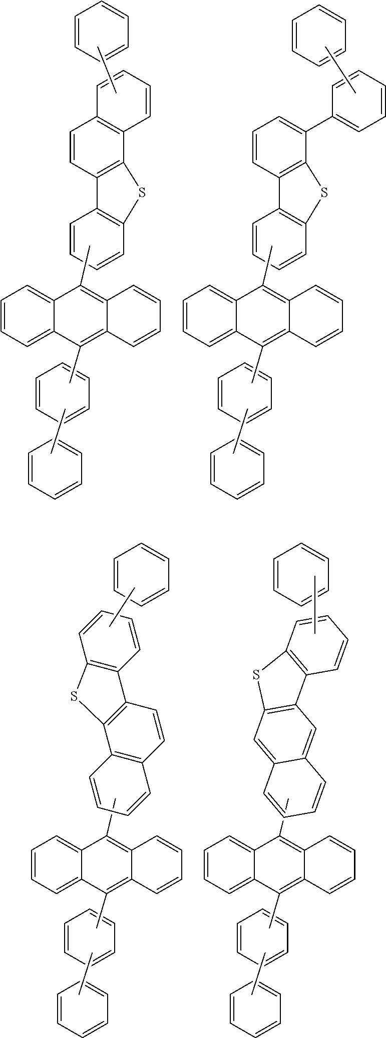

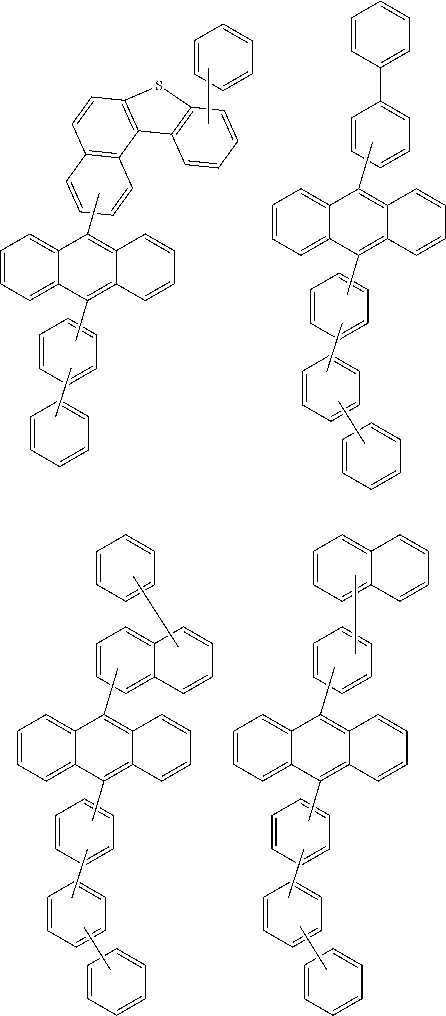

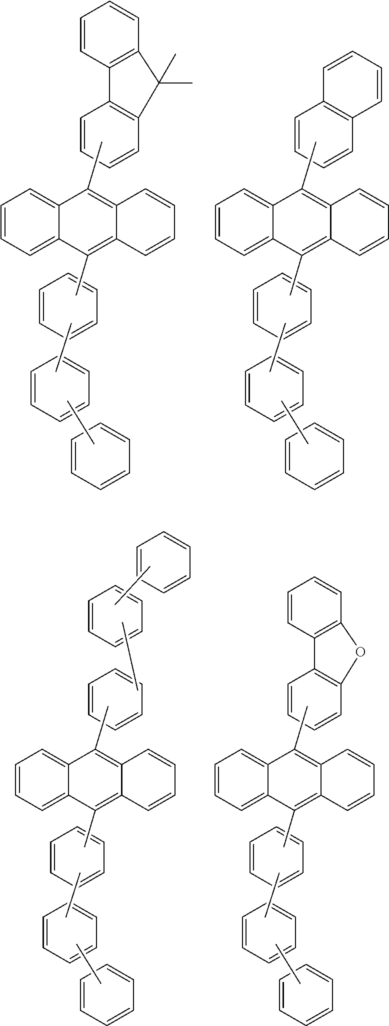

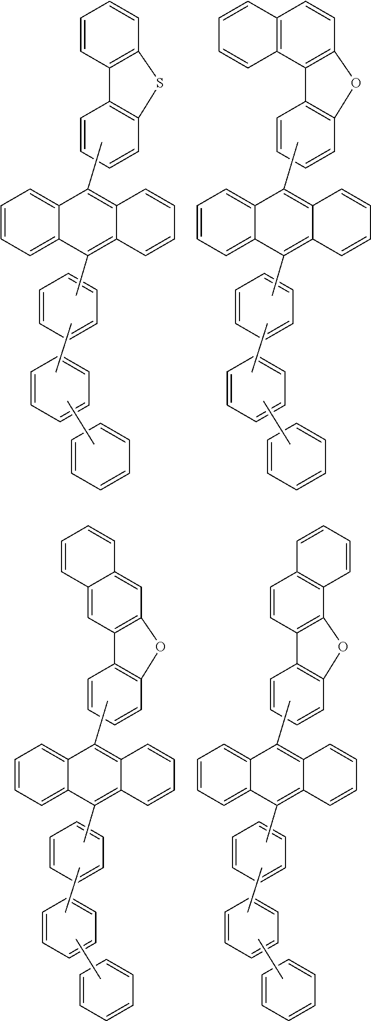

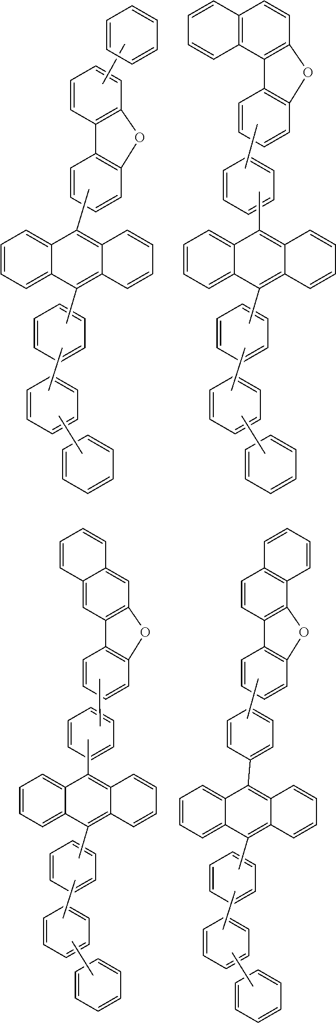

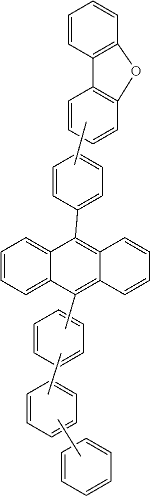

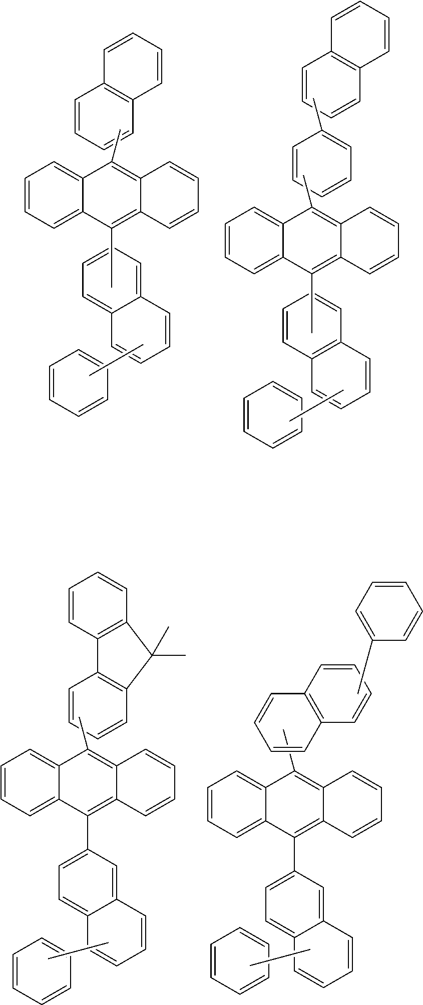

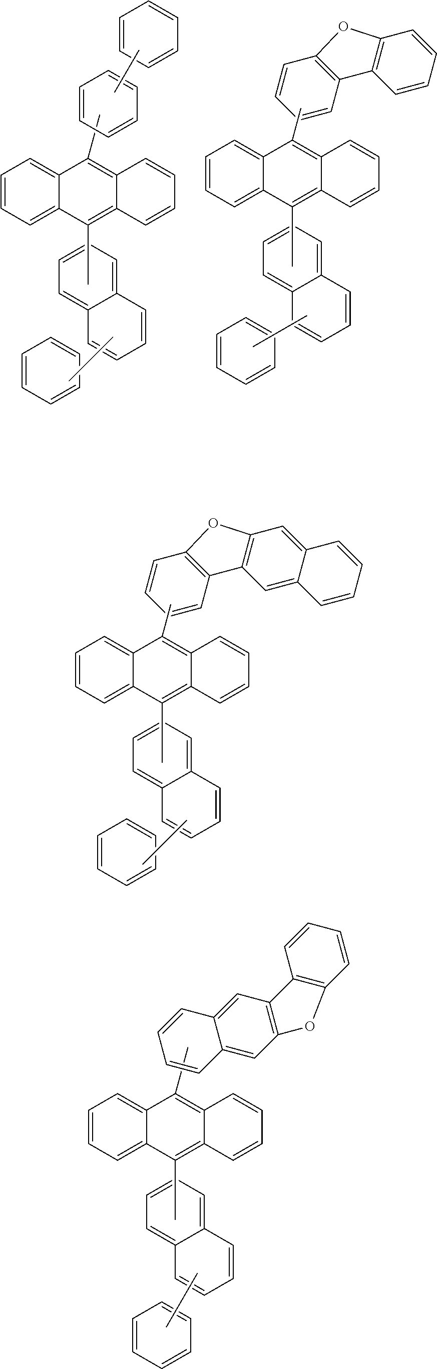

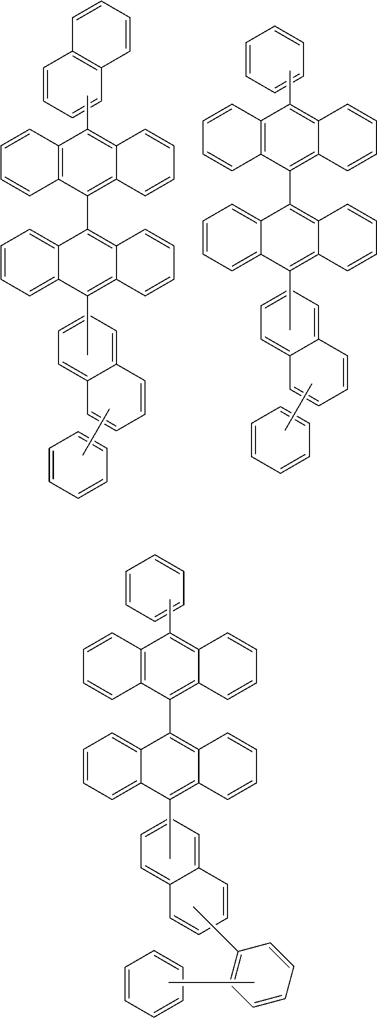

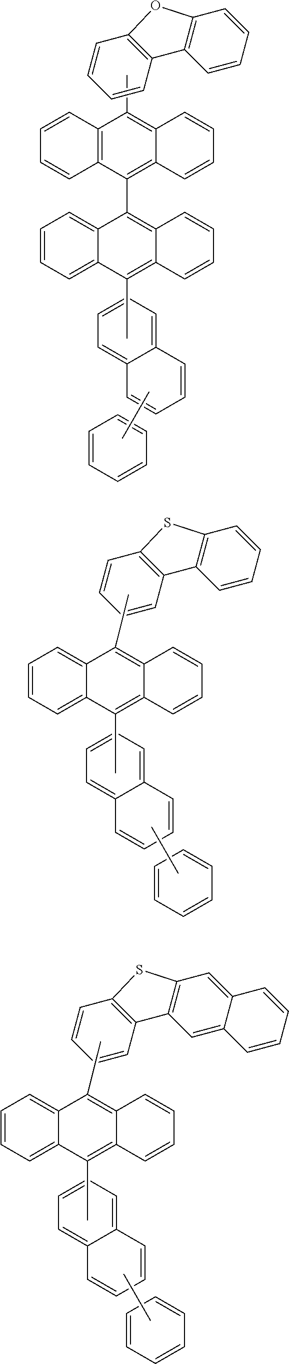

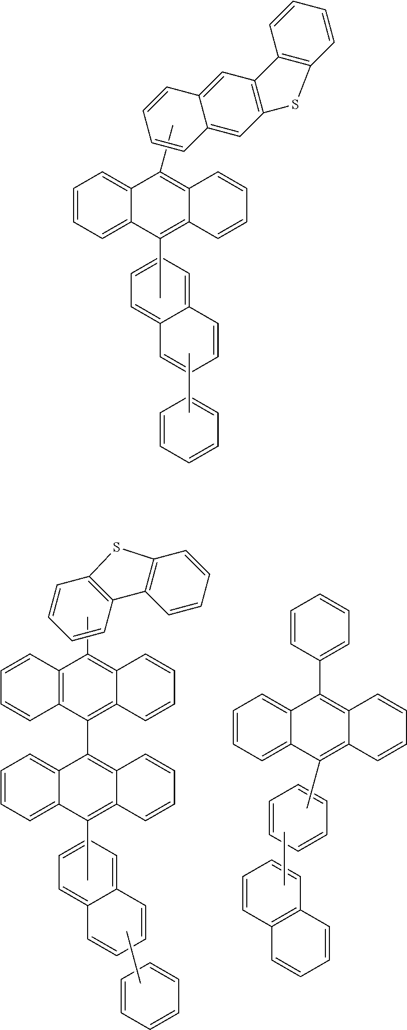

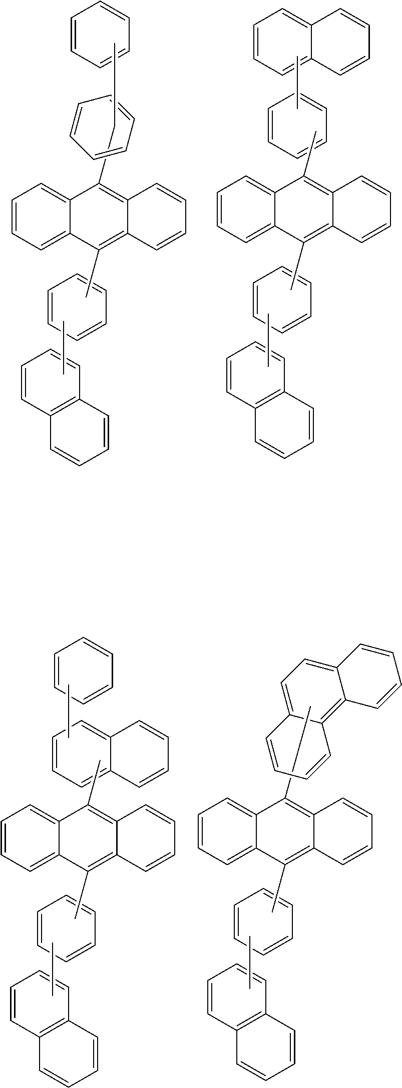

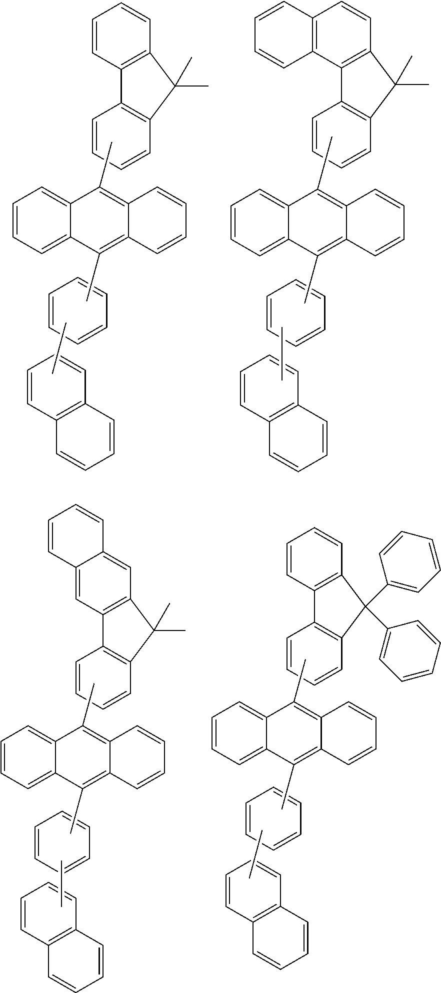

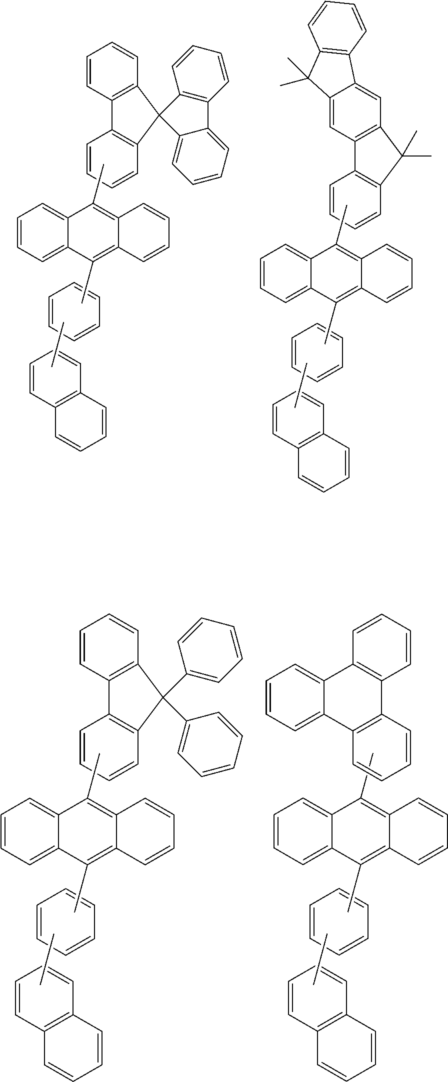

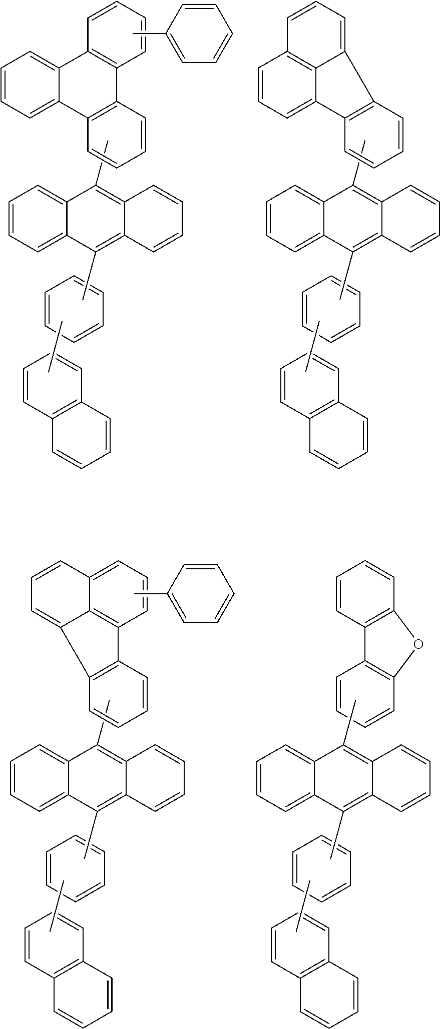

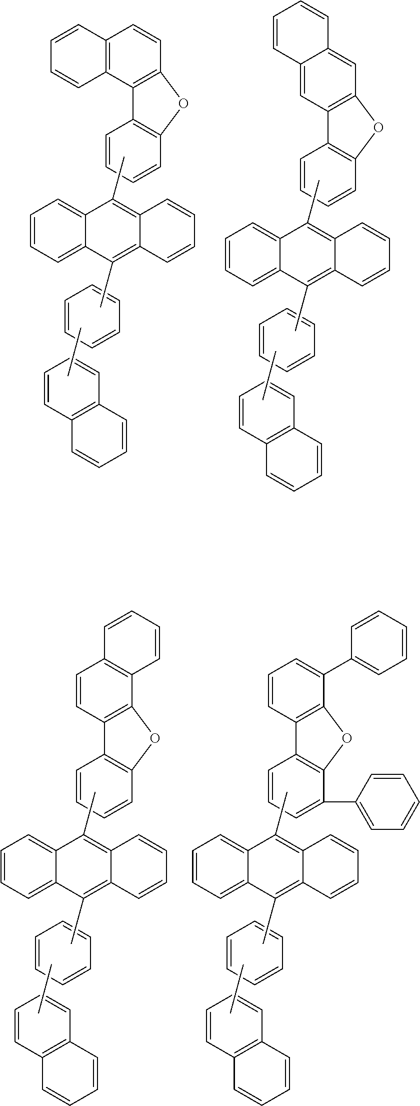

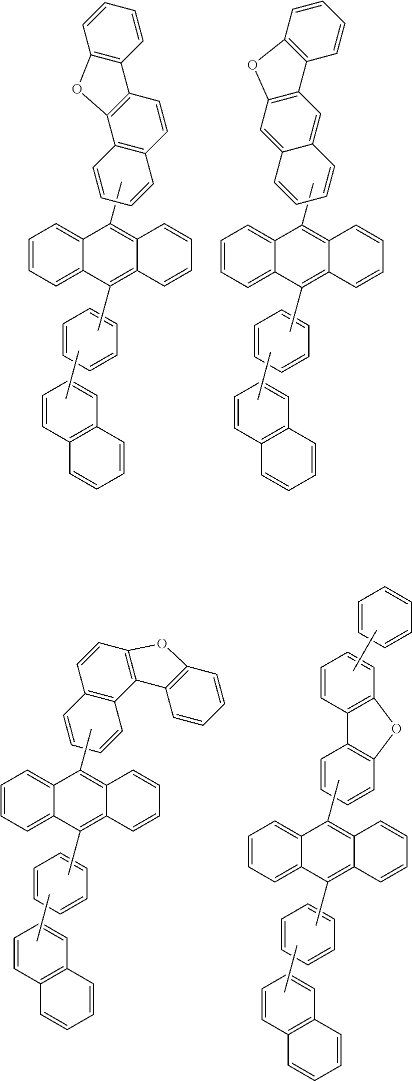

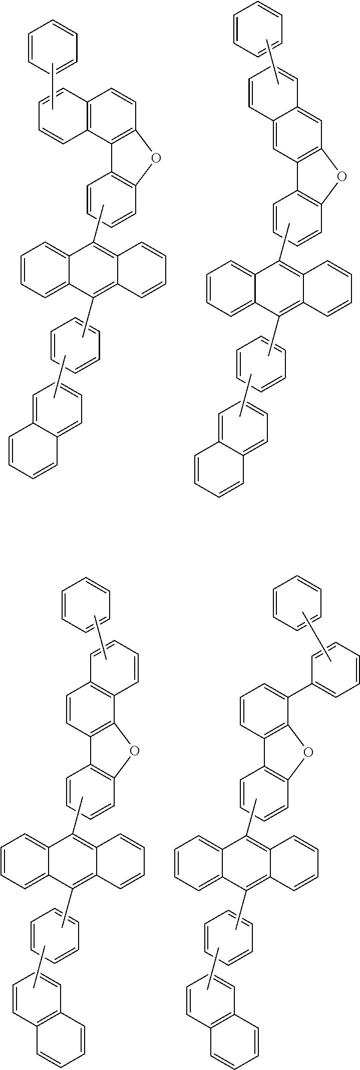

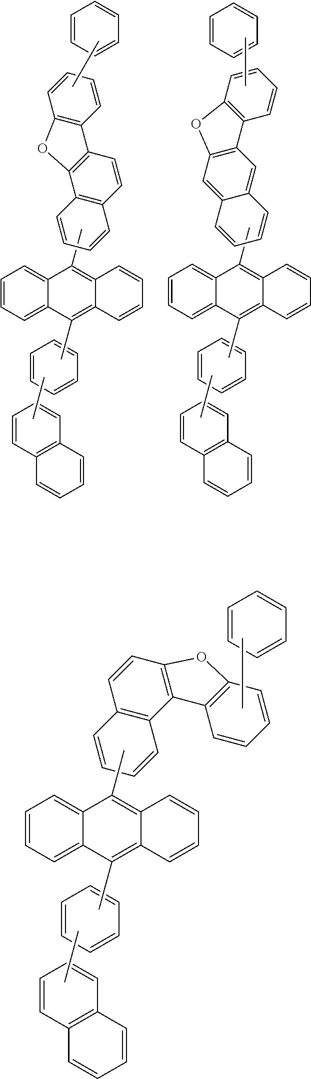

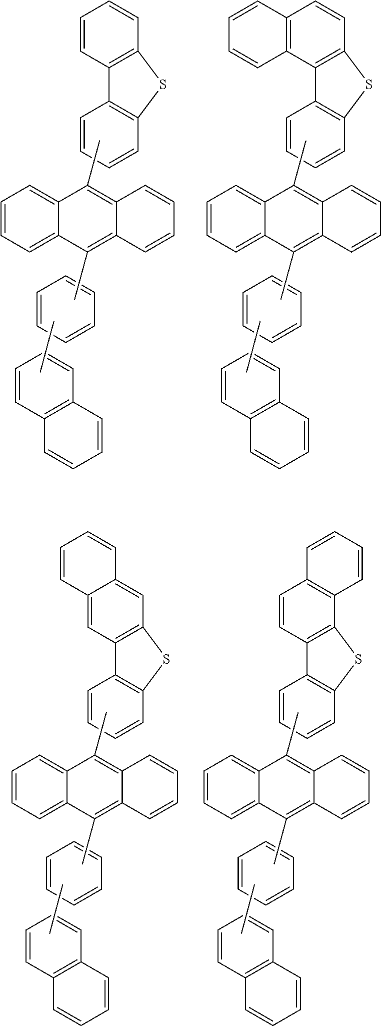

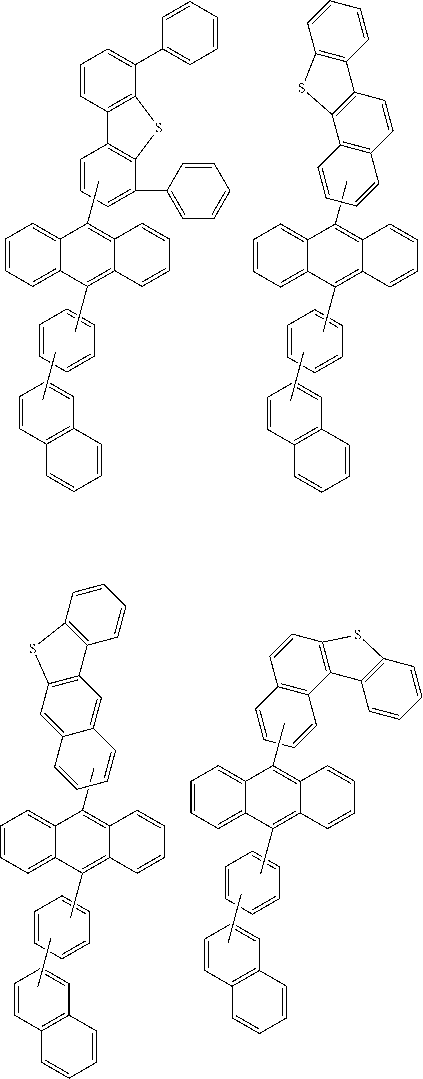

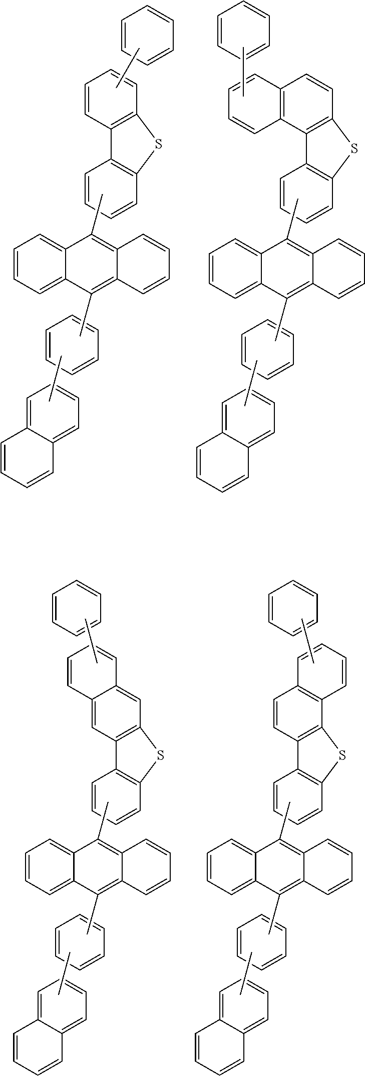

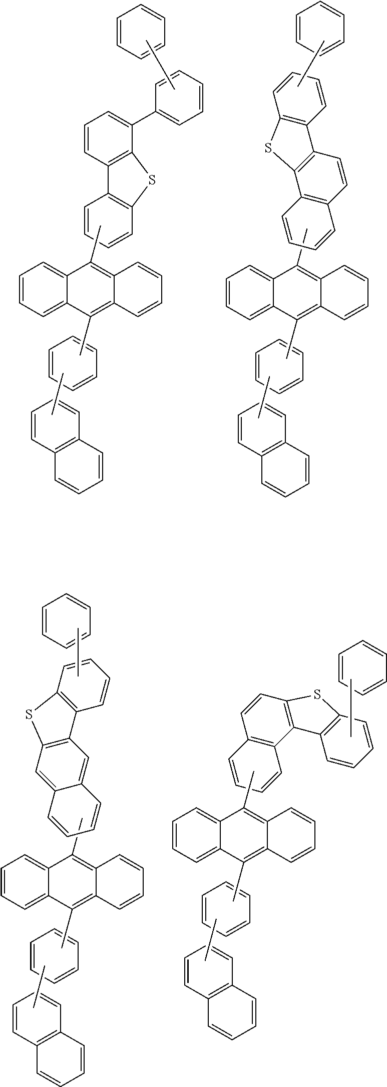

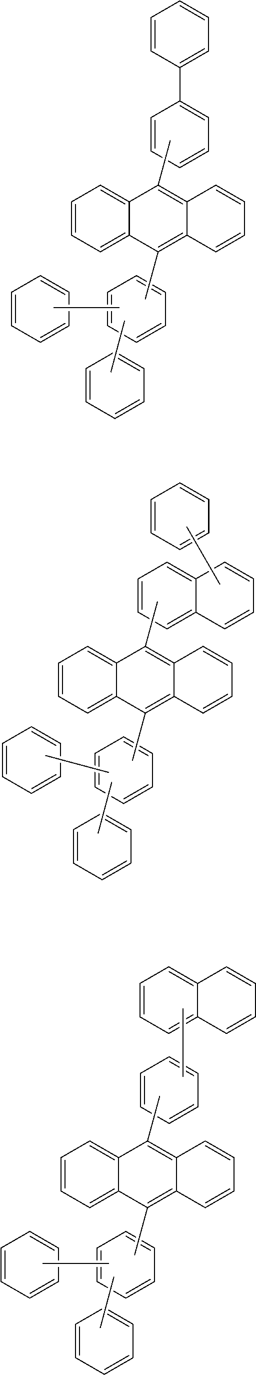

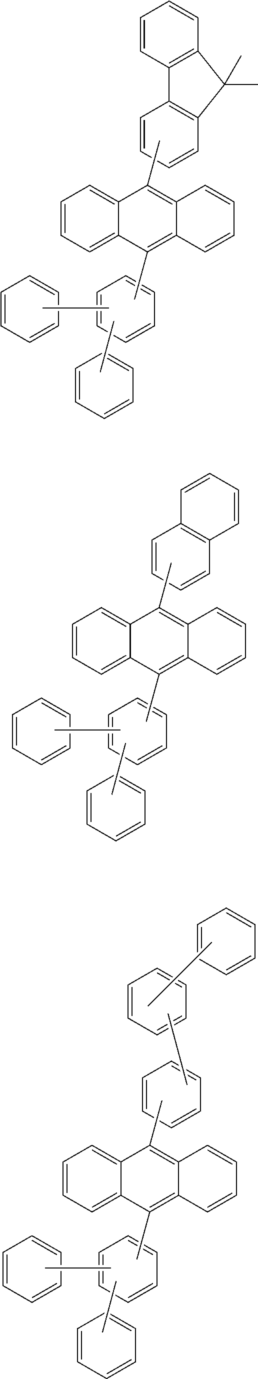

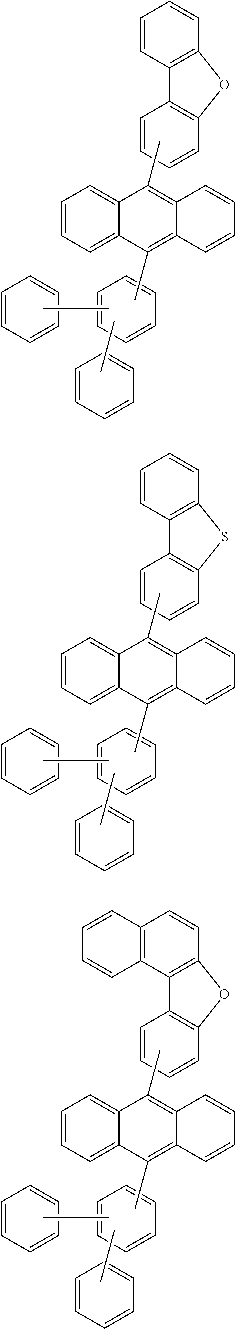

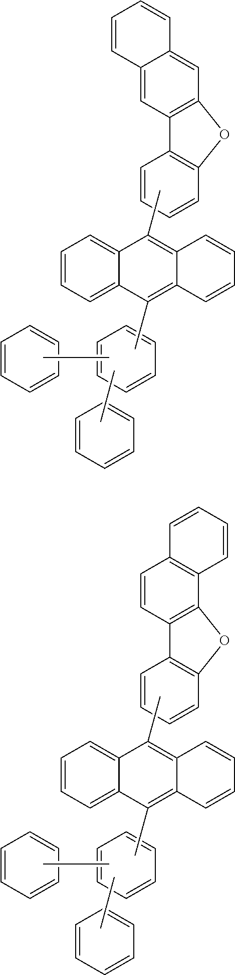

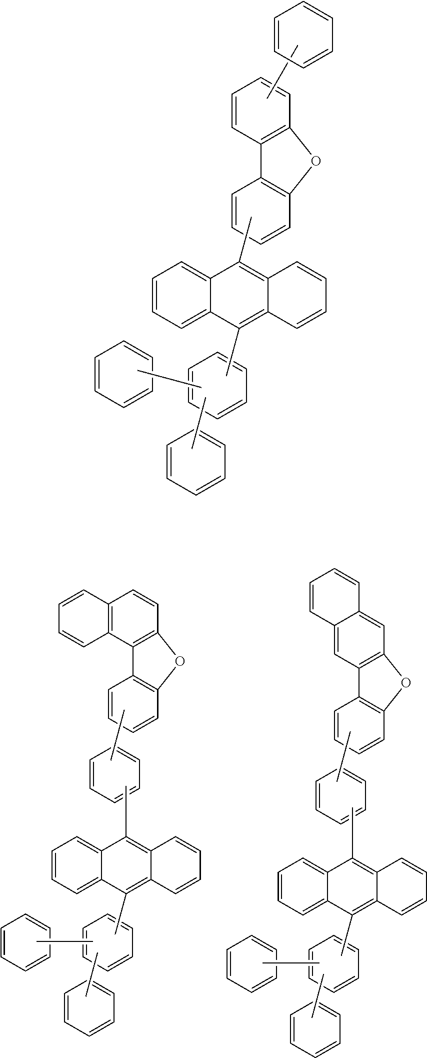

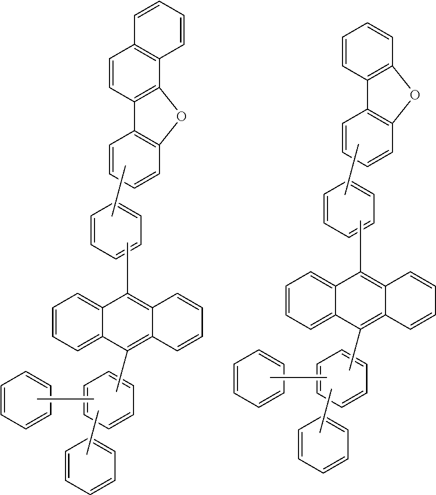

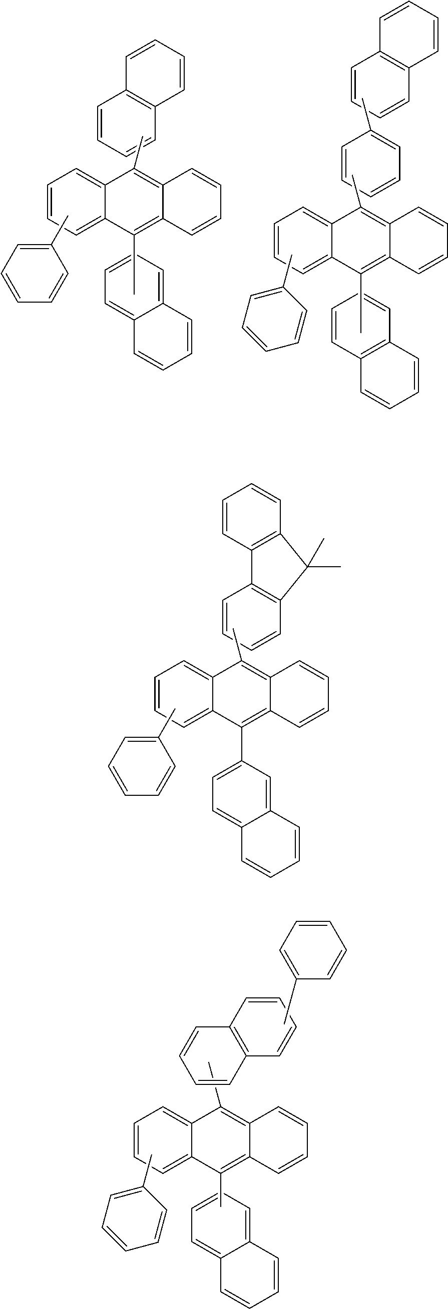

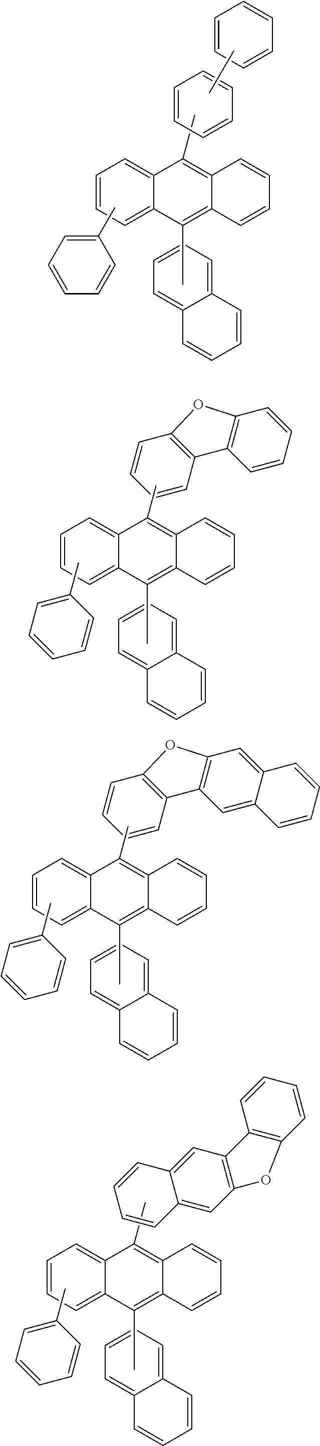

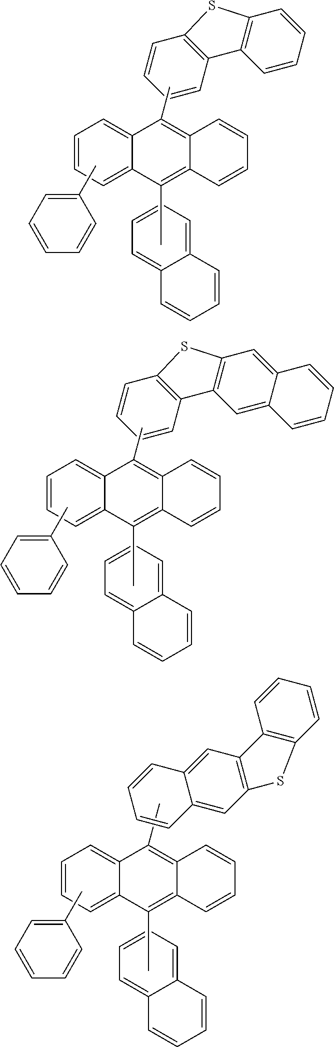

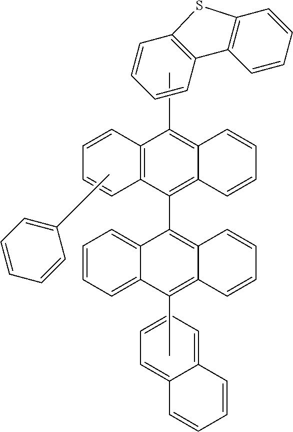

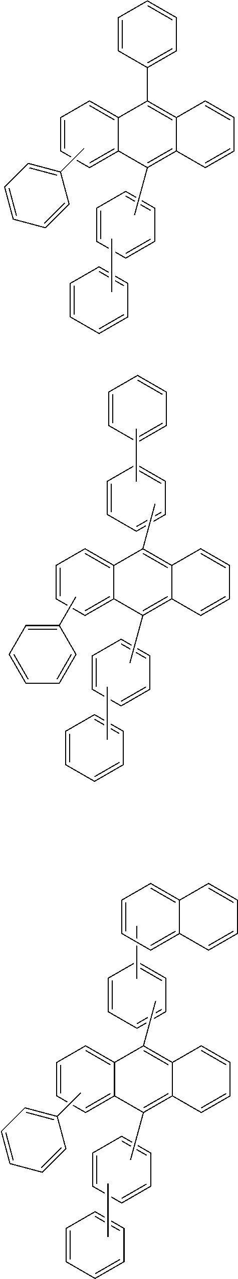

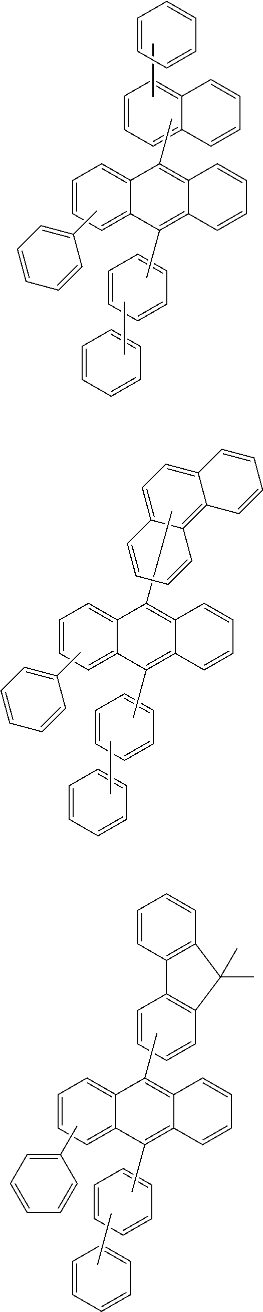

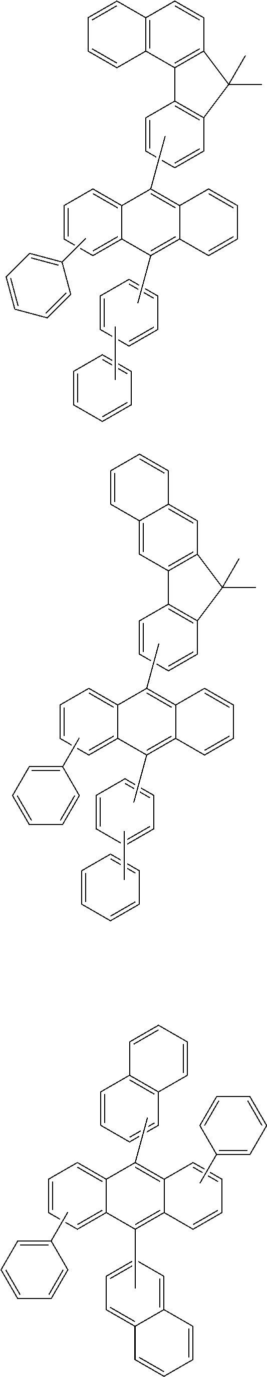

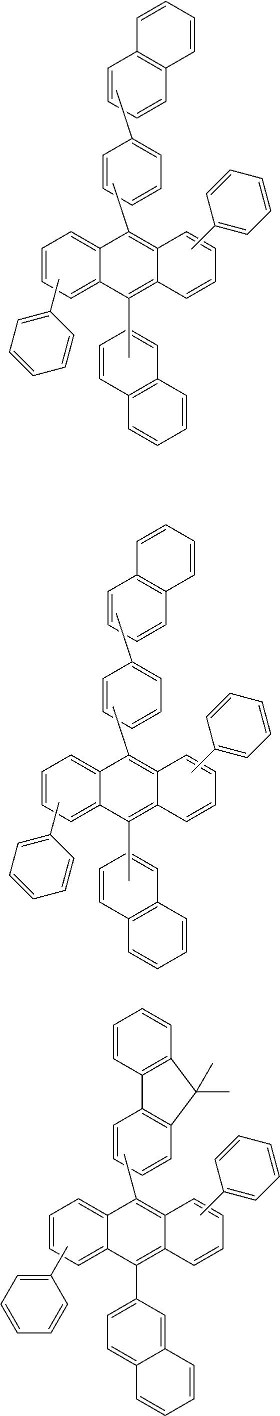

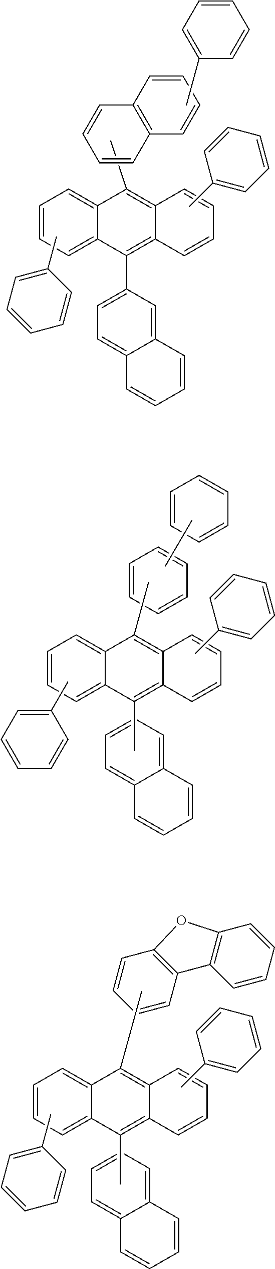

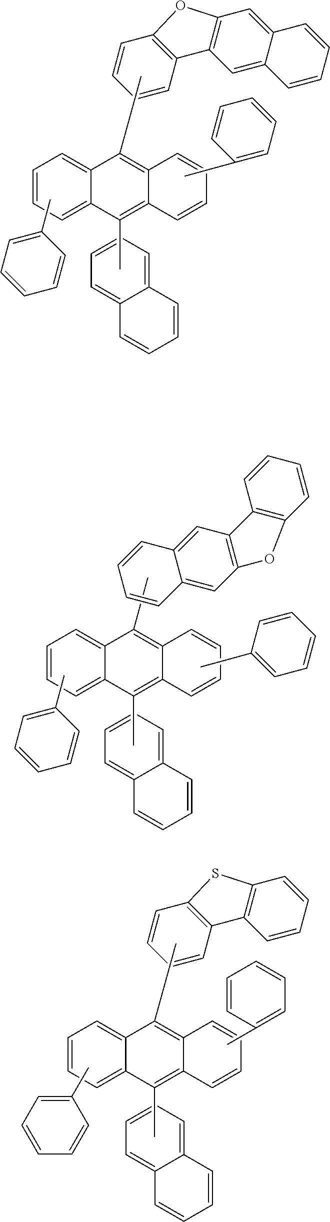

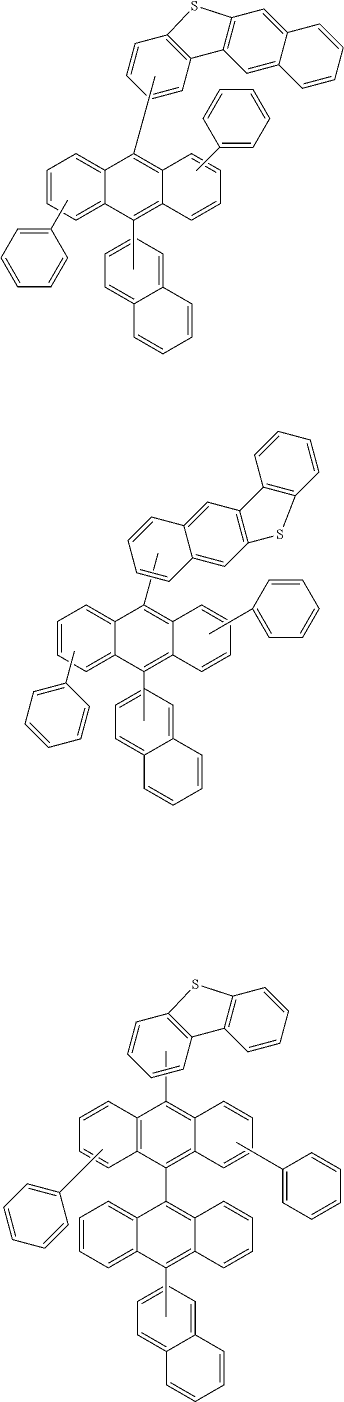

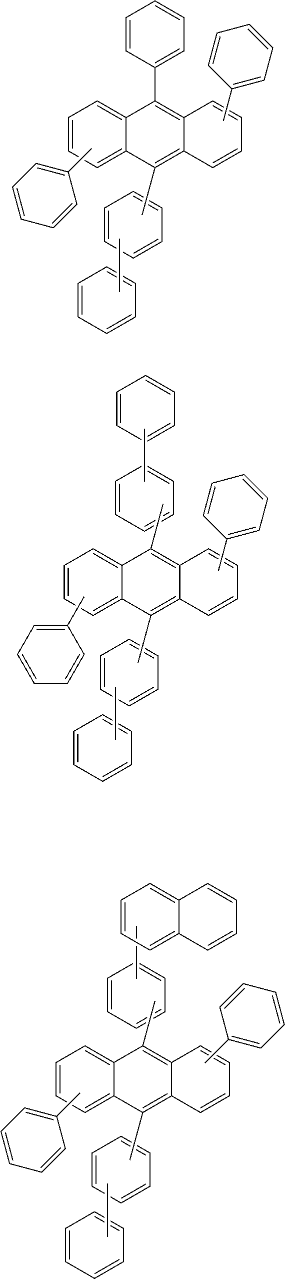

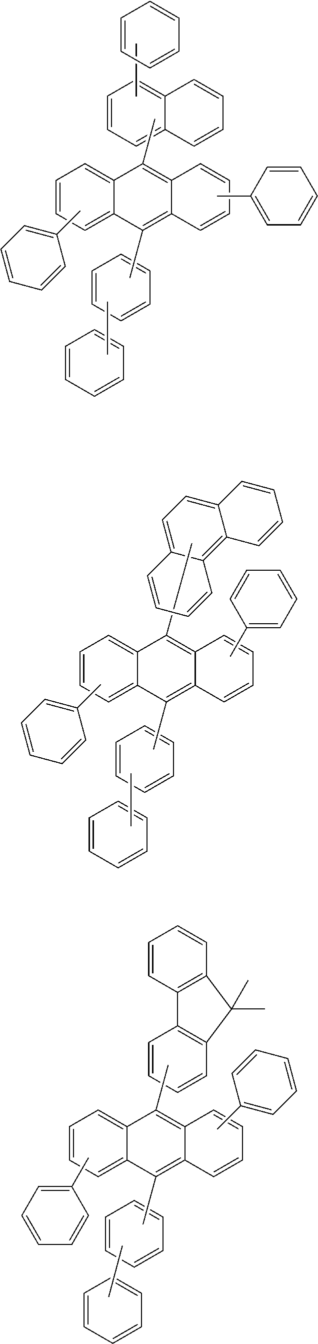

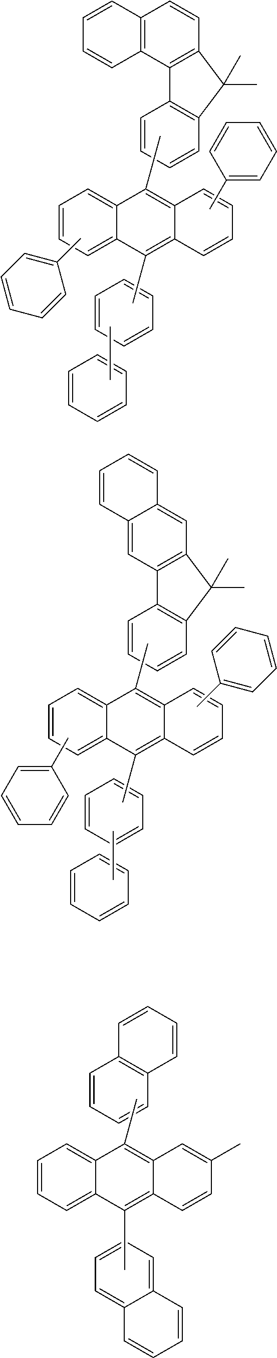

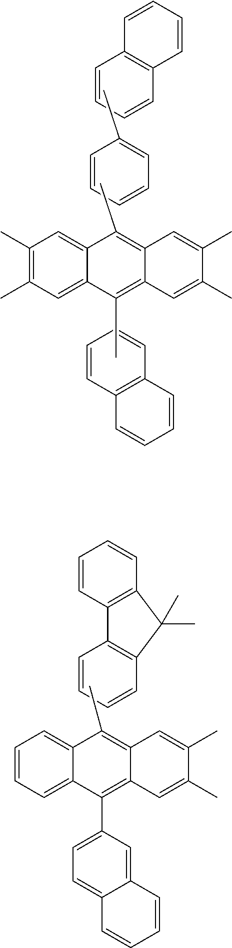

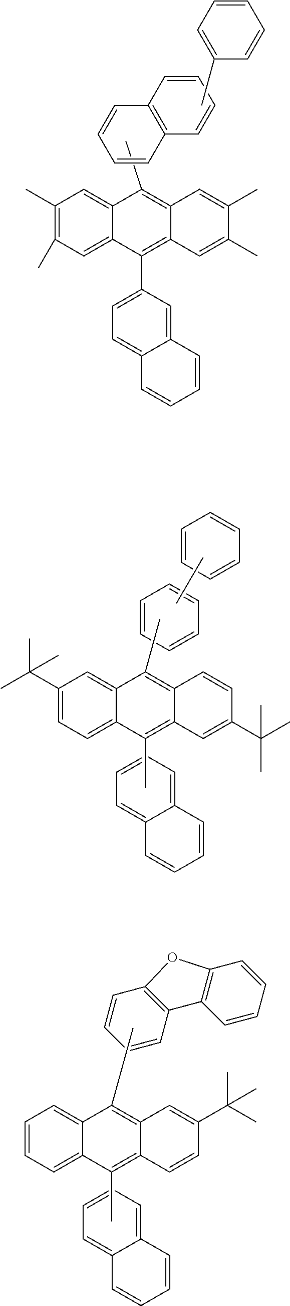

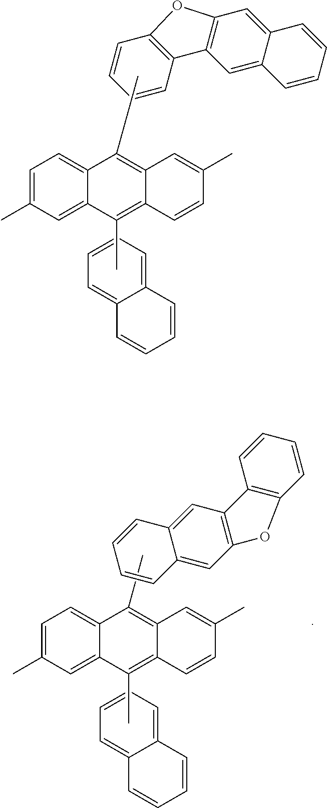

As a preferred technical solution of the present application, a method of the deuterated reaction includes the following steps:
in the presence of a catalyst, placing Compound A in D₂O and a solvent, and conducting the deuterated reaction to obtain the deuterated mixture;
preferably, the solvent is selected from any one or a combination of at least two of benzene, toluene, ethyl acetate or C₆D₆, further preferably C₆D₆.

It is to be noted that C₆D₆ is a product obtained after hydrogen atoms on benzene are all substituted with deuterium atoms.

Optionally, the reaction is conducted in the presence of the catalyst. The catalyst is selected from any one or a combination of at least two of an inorganic salt of a metal, an organic salt of a metal or a complex of a metal. The metals in the inorganic salt of the metal, the organic salt of the metal or the complex of the metal are independently selected from palladium, platinum, rhodium, ruthenium, iridium, iron, copper, cobalt or nickel. The catalyst is further preferably PdCl₂.

Optionally, the reaction is conducted in a hydrogen atmosphere.

Optionally, a volume ratio of D₂O to C₆D₆ is 1: (4-6), which may be, for example, 1:4, 1:4.2, 1:4.4, 1:4.6, 1:4.8, 1:5, 1:5.2, 1:5.4, 1:5.6, 1:5.8 or 1:6, etc.

Optionally, the deuterated reaction is conducted at a temperature of 60-200° C., which may be, for example, 60° C., 70° C., 80° C., 90° C., 100° C., 110° C., 120° C., 130° C., 140° C., 150° C., 160° C., 170° C., 180° C., 190° C. or 200° C., etc.

Optionally, the deuterated reaction lasts for 1-80 h, which may be, for example, 1 h, 2 h, 4 h, 6 h, 10 h, 12 h, 24 h, 30 h, 40 h, 50 h, 60 h, 70 h or 80 h, etc.

Optionally, a pressure of the deuterated reaction is 0.01-2 MPa, which may be, for example, 0.01 MPa, 0.05 MPa, 0.1 MPa, 0.2 MPa, 0.4 MPa, 0.6 MPa, 0.8 MPa, 1 MPa, 1.2 MPa, 1.4 MPa, 1.6 MPa, 1.8 MPa or 2 Mpa, etc.

Optionally, a deuteration rate of the deuterated mixture is 15% to 99% (which may be, for example, 15%, 20%, 28%, 38%, 50%, 53%, 60%, 69%, 70%, 80%, 90% or 99%), further preferably 28% to 70%, and further preferably 38% to 54%.

In the present application, the deuteration rate refers to a number percentage content that the number of deuterium atoms (D) accounts for in a sum of the number of deuterium atoms and the number of hydrogen atoms (H) in a composition or a compound, that is, deuteration rate=y/(x+y)*100%, wherein y is the number of deuterium atoms in the composition or the compound, and x is the number of hydrogen atoms in the composition or the compound. Assuming that what are in the composition or the compound are all H without D, the composition or the compound has a deuteration rate of 0%. If all H in the composition or the compound is substituted with D, the composition or the compound has a deuteration rate of 100%.

It is to be noted that the deuterated reaction in the present application is further conducted in the presence of activated carbon. Moreover, after the deuterated reaction in the present application is finished, steps of post treatment are further included. A post treatment method includes cooling, filtration, layer separation and drying.

As a preferred technical solution of the present application, the deuterated composition further includes Compound B having a structure represented by Formula II:

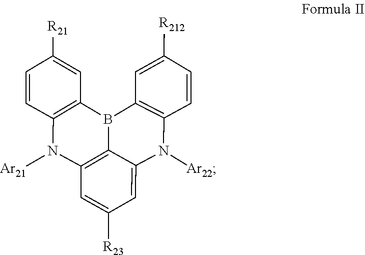

wherein Ar₂₁ and Ar₂₂ are each independently selected from any one of substituted or unsubstituted C6 to C20 aryl (which may be, for example, C6, C8, C10, C12, C14, C16, C18 or C20, etc.), or substituted or unsubstituted C3 to C20 heteroaryl (which may be, for example, C3, C6, C8, C10, C12, C14, C16, C18 or C20, etc.);
R₂₁, R₂₂ and R₂₃ are each independently selected from any one of hydrogen, C1 to C12 linear or branched alkyl (which may be, for example, C1, C2, C4, C6, C8, C10 or C12, etc.), or C6 to C12 cycloalkyl (which may be, for example, C6, C7, C8, C9, C10, C11 or C12, etc.); and
substituted substituents in A₂₁ and Ar₂₂ are each independently selected from C1 to C5 linear or branched alkyl (which may be, for example, methyl, ethyl, propyl, n-butyl, isobutyl or tert-butyl, etc.), or C6 to C12 aryl (which may be, for example, phenyl, tolyl or naphthyl, etc.).

It is to be noted that in Compound B having the structure represented by Formula II, Ar₂₁, Ar₂₂, R₂₁, R₂₂ and R₂₃ are each independently selected from C1 to C6 alkyl (which may be, for example, methyl, ethyl, propyl, n-butyl, isobutyl or tert-butyl), C1 to C6 alkoxy (which may be, for example, methoxy or ethoxy), phenyl, biphenyl, naphthyl, phenanthryl, anthryl, fluorenyl, benzofluorenyl, dibenzofluorenyl, triphenylenyl, fluoranthenyl, pyrenyl, perylenyl, spriofluorenyl, indenofluorenyl or hydrogenated benzanthryl.

As a preferred technical solution of the present application, Ar₂₁ and Ar₂₂ are each independently selected from any one of

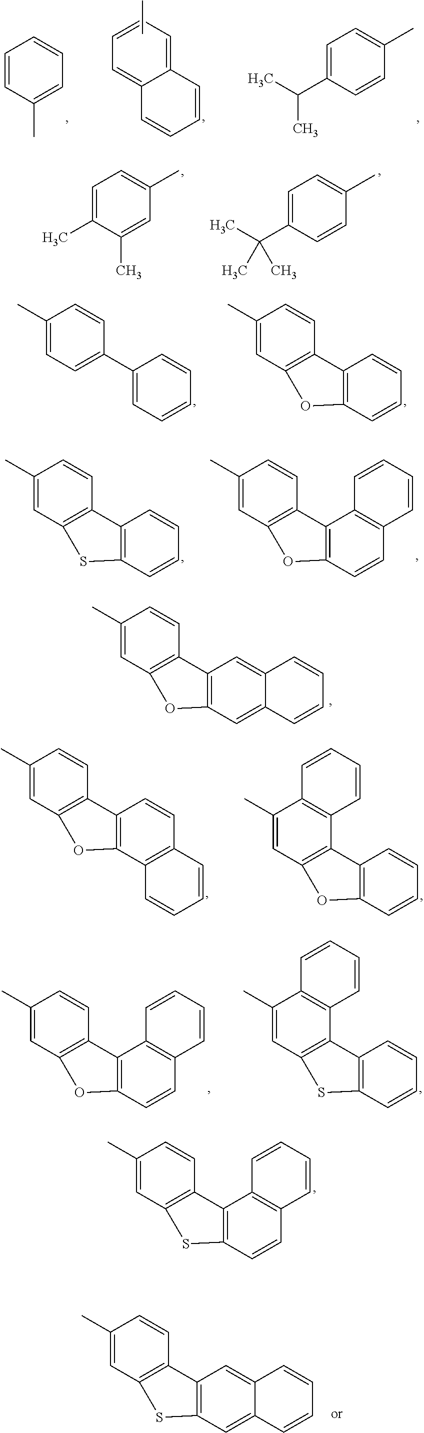

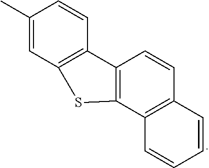 [SMILES: Cc1ccc2c(c1)sc1c3ccccc3ccc21]

Optionally, R₁, R₂ and R₃ are each independently selected from any one of hydrogen, methyl, ethyl, propyl, n-butyl, isobutyl, sec-butyl, tert-butyl, cyclohexyl or adamantyl.

Optionally, Compound B is selected from any one of the following compounds:

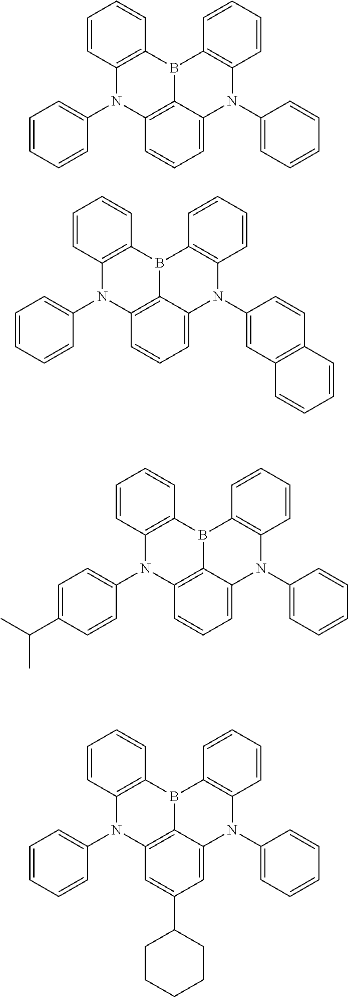 [SMILES: c1ccc(N2c3ccccc3B3c4ccccc4N(c4ccccc4)c4cccc2c43)cc1 ; c1ccc(N2c3ccccc3B3c4ccccc4N(c4ccc5ccccc5c4)c4cccc2c43)cc1 ; CC(C)c1ccc(N2c3ccccc3B3c4ccccc4N(c4ccccc4)c4cccc2c43)cc1 ; c1ccc(N2c3ccccc3B3c4ccccc4N(c4ccccc4)c4cc(C5CCCCC5)cc2c43)cc1]

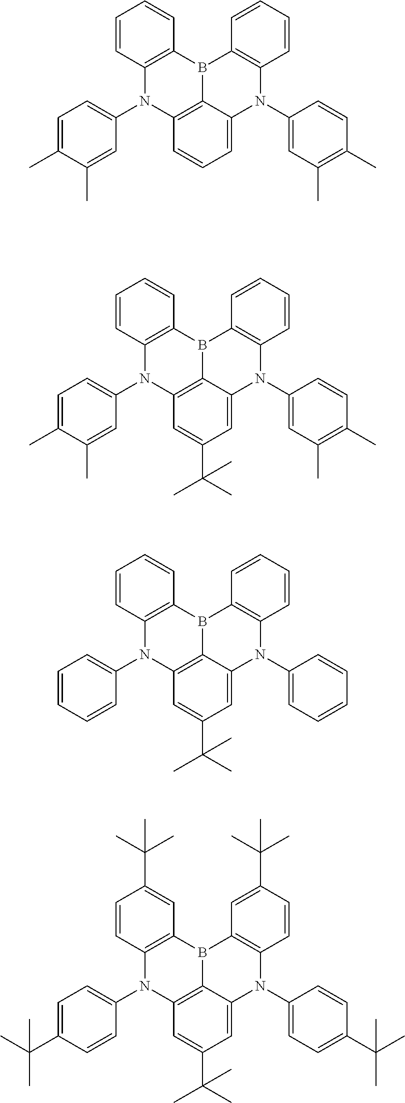

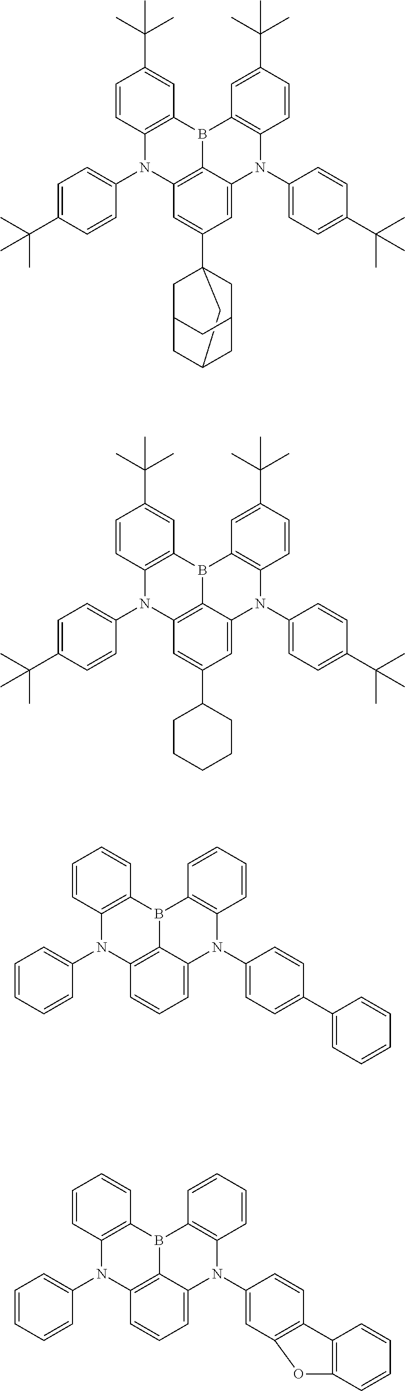

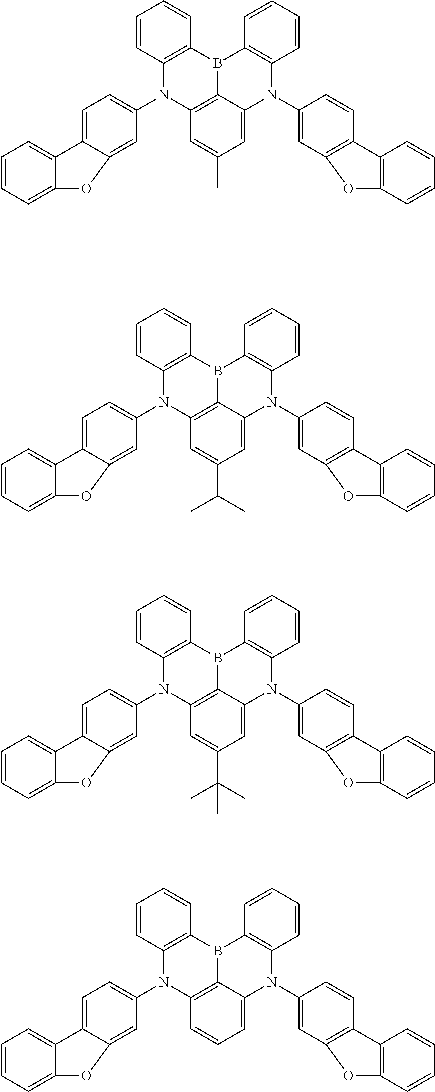 [SMILES: Cc1cc2c3c(c1)N(c1ccc4c(c1)oc1ccccc14)c1ccccc1B3c1ccccc1N2c1ccc2c(c1)oc1ccccc12 ; CC(C)c1cc2c3c(c1)N(c1ccc4c(c1)oc1ccccc14)c1ccccc1B3c1ccccc1N2c1ccc2c(c1)oc1ccccc12 ; CC(C)(C)c1cc2c3c(c1)N(c1ccc4c(c1)oc1ccccc14)c1ccccc1B3c1ccccc1N2c1ccc2c(c1)oc1ccccc12 ; c1ccc2c(c1)B1c3ccccc3N(c3ccc4c(c3)oc3ccccc34)c3cccc(c31)N2c1ccc2c(c1)oc1ccccc12]

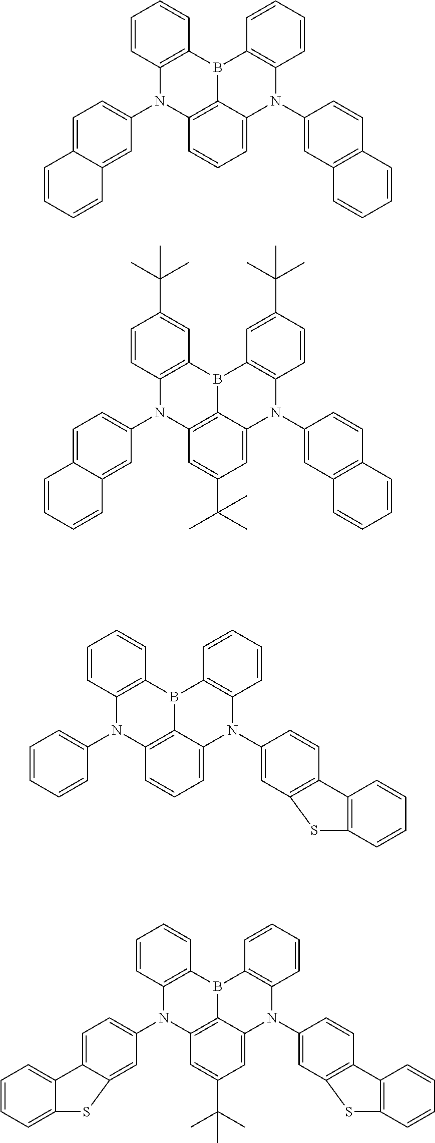 [SMILES: c1ccc2c(c1)B1c3ccccc3N(c3ccc4ccccc4c3)c3cccc(c31)N2c1ccc2ccccc2c1 ; CC(C)(C)c1ccc2c(c1)B1c3cc(C(C)(C)C)ccc3N(c3ccc4ccccc4c3)c3cc(C(C)(C)C)cc(c31)N2c1ccc2ccccc2c1 ; c1ccc(N2c3ccccc3B3c4ccccc4N(c4ccc5c(c4)sc4ccccc45)c4cccc2c43)cc1 ; CC(C)(C)c1cc2c3c(c1)N(c1ccc4c(c1)sc1ccccc14)c1ccccc1B3c1ccccc1N2c1ccc2c(c1)sc1ccccc12]

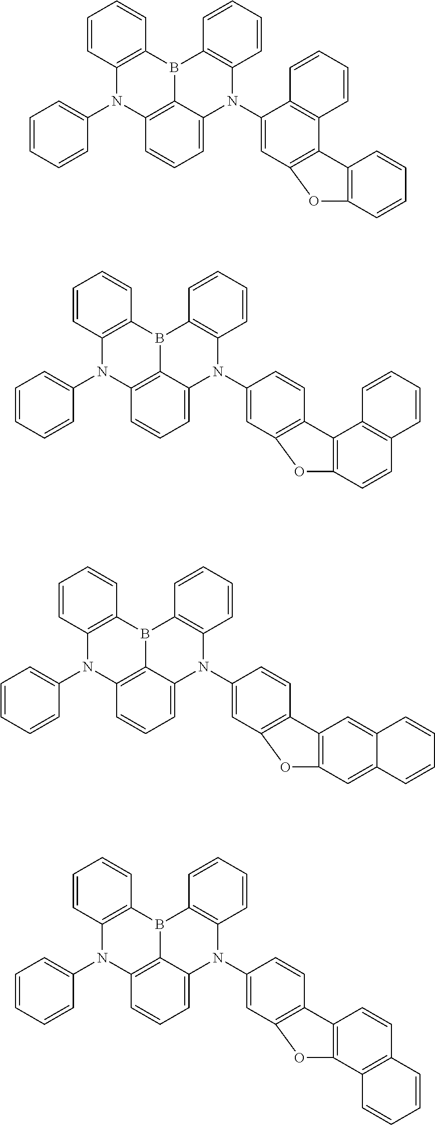 [SMILES: c1ccc(N2c3ccccc3B3c4ccccc4N(c4cc5oc6ccccc6c5c5ccccc45)c4cccc2c43)cc1 ; c1ccc(N2c3ccccc3B3c4ccccc4N(c4ccc5c(c4)oc4ccc6ccccc6c45)c4cccc2c43)cc1 ; c1ccc(N2c3ccccc3B3c4ccccc4N(c4ccc5c(c4)oc4cc6ccccc6cc45)c4cccc2c43)cc1 ; c1ccc(N2c3ccccc3B3c4ccccc4N(c4ccc5c(c4)oc4c6ccccc6ccc54)c4cccc2c43)cc1]

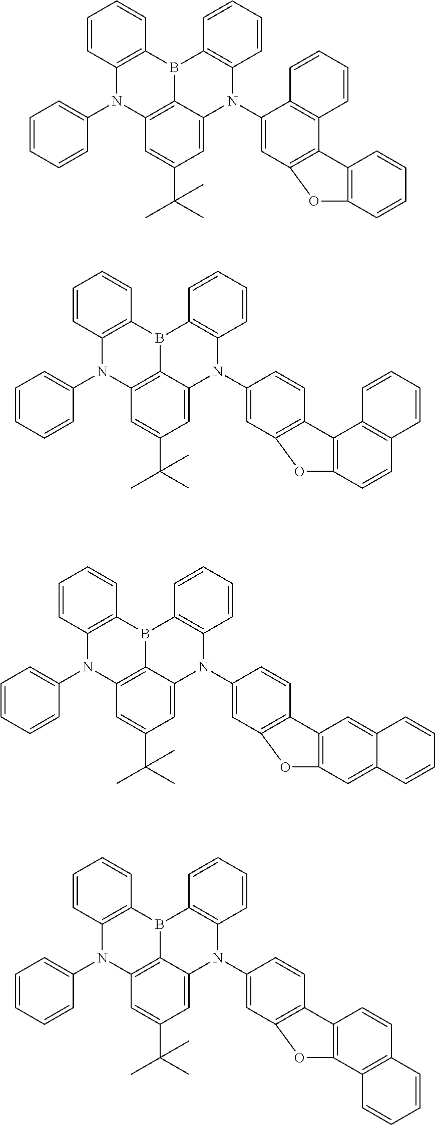 [SMILES: CC(C)(C)c1cc2c3c(c1)N(c1cc4oc5ccccc5c4c4ccccc14)c1ccccc1B3c1ccccc1N2c1ccccc1 ; CC(C)(C)c1cc2c3c(c1)N(c1ccc4c(c1)oc1ccc5ccccc5c14)c1ccccc1B3c1ccccc1N2c1ccccc1 ; CC(C)(C)c1cc2c3c(c1)N(c1ccc4c(c1)oc1cc5ccccc5cc14)c1ccccc1B3c1ccccc1N2c1ccccc1 ; CC(C)(C)c1cc2c3c(c1)N(c1ccc4c(c1)oc1c5ccccc5ccc41)c1ccccc1B3c1ccccc1N2c1ccccc1]

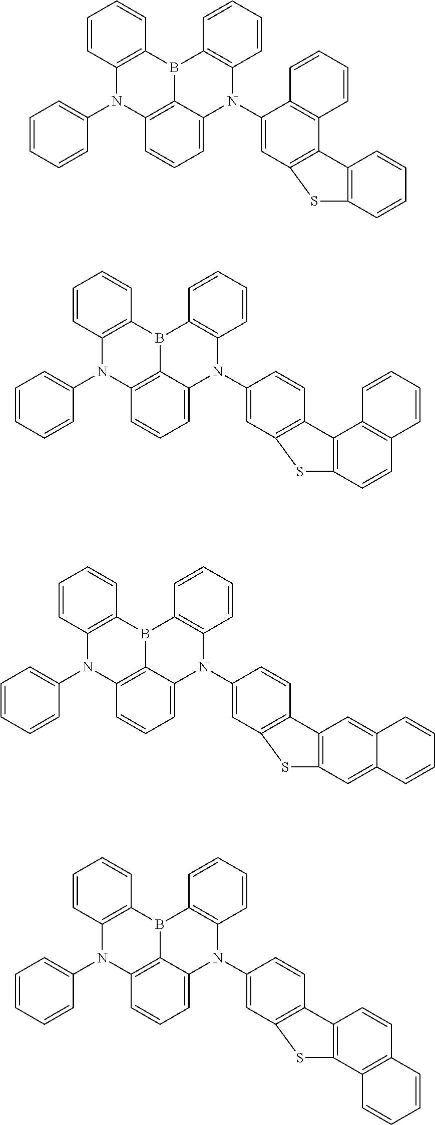 [SMILES: c1ccc(N2c3ccccc3B3c4ccccc4N(c4cc5sc6ccccc6c5c5ccccc45)c4cccc2c43)cc1 ; c1ccc(N2c3ccccc3B3c4ccccc4N(c4ccc5c(c4)sc4ccc6ccccc6c45)c4cccc2c43)cc1 ; c1ccc(N2c3ccccc3B3c4ccccc4N(c4ccc5c(c4)sc4cc6ccccc6cc45)c4cccc2c43)cc1 ; c1ccc(N2c3ccccc3B3c4ccccc4N(c4ccc5c(c4)sc4c6ccccc6ccc54)c4cccc2c43)cc1]

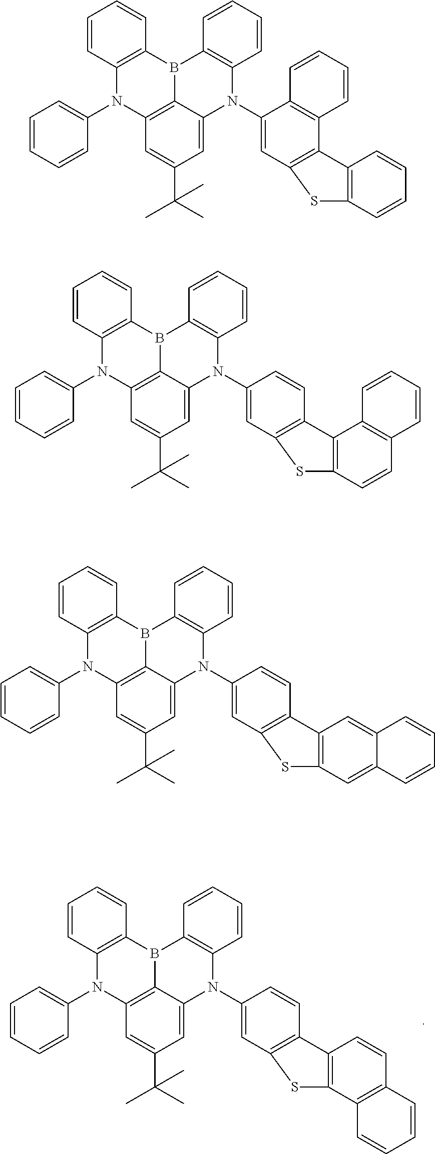 [SMILES: CC(C)(C)c1cc2c3c(c1)N(c1cc4sc5ccccc5c4c4ccccc14)c1ccccc1B3c1ccccc1N2c1ccccc1 ; CC(C)(C)c1cc2c3c(c1)N(c1ccc4c(c1)sc1ccc5ccccc5c14)c1ccccc1B3c1ccccc1N2c1ccccc1 ; CC(C)(C)c1cc2c3c(c1)N(c1ccc4c(c1)sc1cc5ccccc5cc14)c1ccccc1B3c1ccccc1N2c1ccccc1 ; CC(C)(C)c1cc2c3c(c1)N(c1ccc4c(c1)sc1c5ccccc5ccc41)c1ccccc1B3c1ccccc1N2c1ccccc1]

As a preferred technical solution of the present application, the deuterated composition further includes Compound C having a structure represented by Formula III:

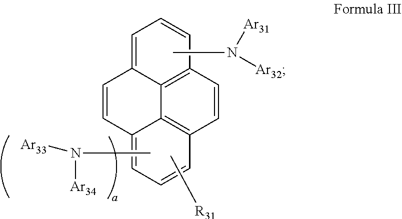

wherein Ar₃₁, Ar₃₂, Ar₃₃ and Ar₃₄ are each independently selected from any one of substituted or unsubstituted C6 to C20 aryl (which may be, for example, C6, C8, C10, C12, C14, C16, C18 or C20, etc.), or substituted or unsubstituted C12 to C40 heteroaryl (which may be, for example, C12, C16, C20, C24, C28, C30, C32, C36 or C40, etc.);
R₃₁ is selected from any one of phenyl, naphthyl or biphenyl;
a is selected from 0 or 1;
substituted substituents in A₃₁, Ar₃₂, Ar₃₃ and Ar₃₄ are each independently selected from C1 to C5 linear or branched alkyl (which may be, for example, methyl, ethyl, propyl, n-butyl, isobutyl or tert-butyl, etc.), or C6 to C12 aryl (which may be, for example, phenyl, etc.);
Optionally, Ar₃₁, Ar₃₂, Ar₃₃ and Ar₃₄ are each independently selected from any one or a combination of at least two of

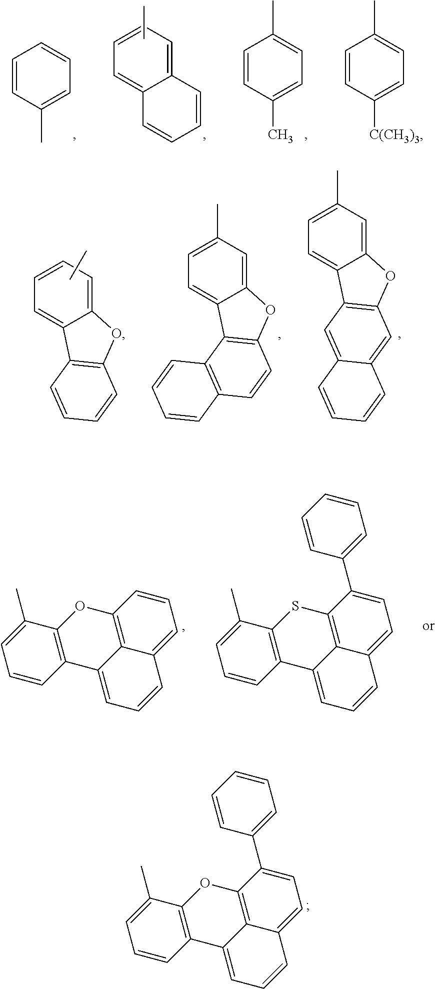

optionally. Compound C is selected from any one of the following compounds:

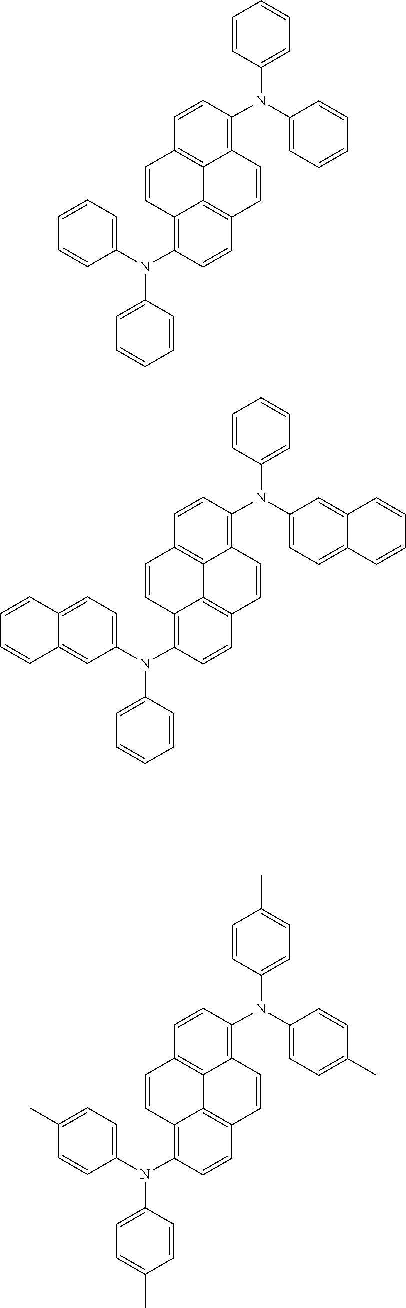

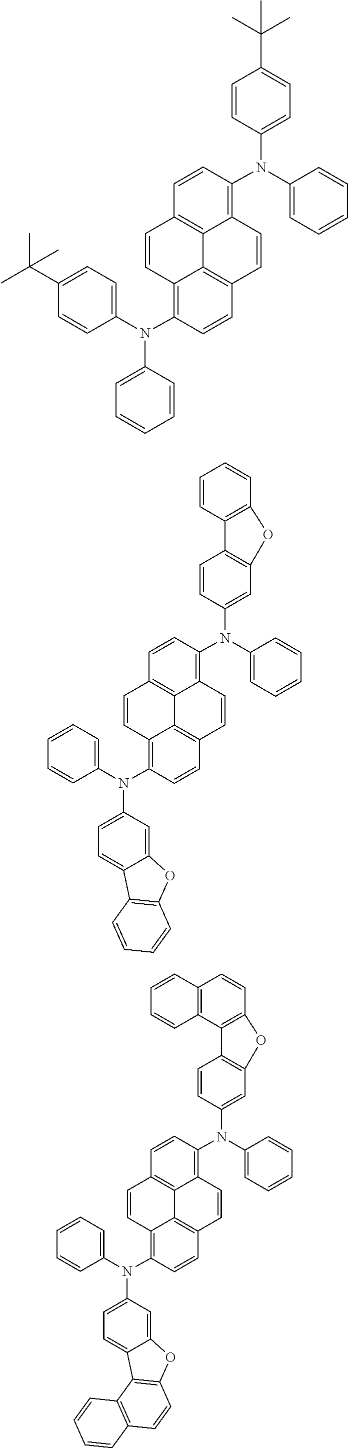

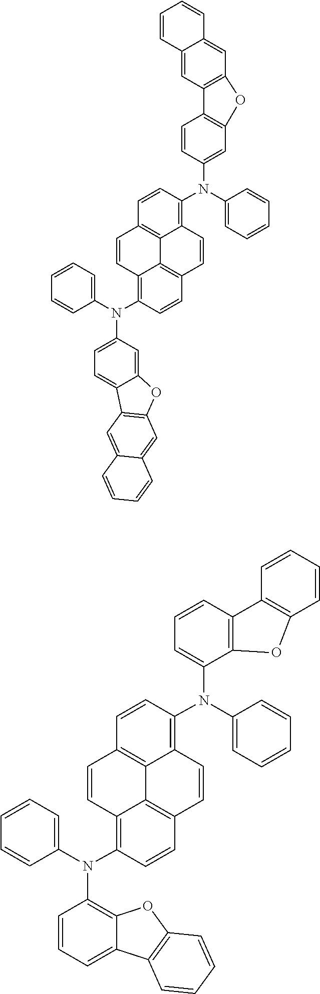 [SMILES: c1ccc(N(c2ccc3c(c2)oc2cc4ccccc4cc23)c2ccc3ccc4c(N(c5ccccc5)c5ccc6c(c5)oc5cc7ccccc7cc56)ccc5ccc2c3c54)cc1 ; c1ccc(N(c2ccc3ccc4c(N(c5ccccc5)c5cccc6c5oc5ccccc56)ccc5ccc2c3c54)c2cccc3c2oc2ccccc23)cc1]

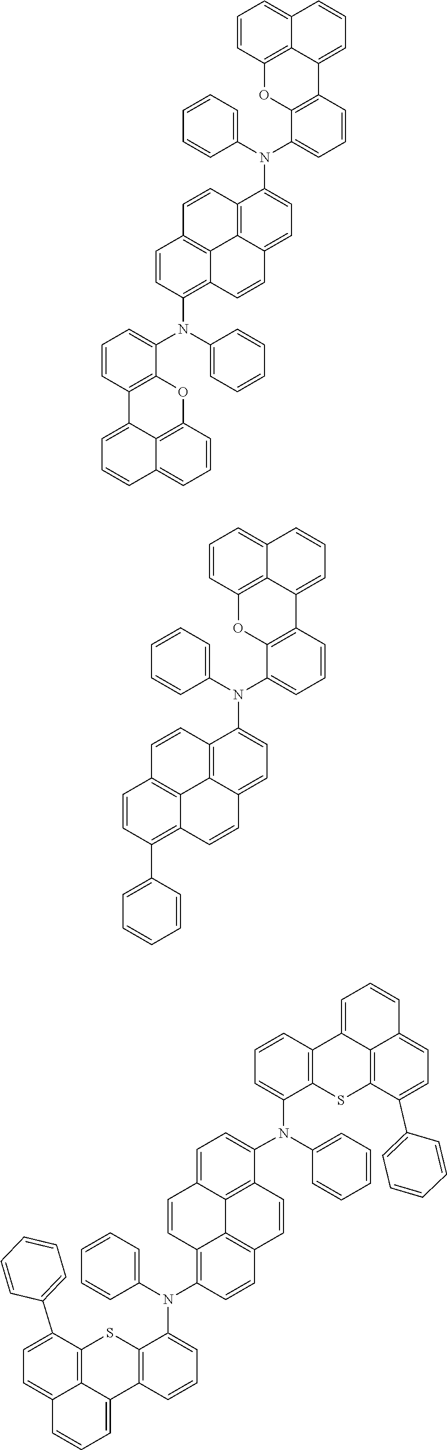 [SMILES: c1ccc(N(c2cccc3c2Oc2cccc4cccc-3c24)c2ccc3ccc4c(N(c5ccccc5)c5cccc6c5Oc5cccc7cccc-6c57)ccc5ccc2c3c54)cc1 ; c1ccc(-c2ccc3ccc4c(N(c5ccccc5)c5cccc6c5Oc5cccc7cccc-6c57)ccc5ccc2c3c54)cc1 ; c1ccc(-c2ccc3cccc4c3c2Sc2c-4cccc2N(c2ccccc2)c2ccc3ccc4c(N(c5ccccc5)c5cccc6c5Sc5c(-c7ccccc7)ccc7cccc-6c57)ccc5ccc2c3c54)cc1]

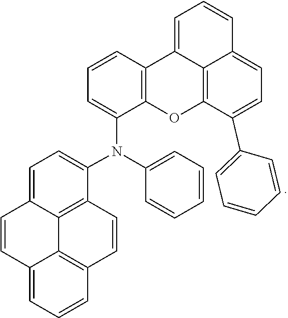 [SMILES: c1ccc(-c2ccc3cccc4c3c2Oc2c-4cccc2N(c2ccccc2)c2ccc3ccc4cccc5ccc2c3c45)cc1]

In a second aspect, the present application provides an organic electroluminescent device including an anode, a cathode and an organic thin-film layer disposed between the anode and the cathode, wherein the organic thin-film layer includes a light-emitting layer; and a material of the organic thin-film layer includes the deuterated composition.

Optionally, the organic layer includes a light-emitting layer.

A material of the light-emitting layer includes a host material, wherein the host material includes the deuterated composition according to the first aspect.

Optionally, the organic layer further includes a hole transport layer (including at least one of a hole injection layer, a hole transport layer or an electron blocking layer) and an electronic layer (including at least one of a hole blocking layer, an electron transport layer or an electron injection layer).

As a preferred technical solution of the present application, the material of the light-emitting layer further includes a doped material:
wherein the doped material includes a compound having a structure represented by Formula BDI:

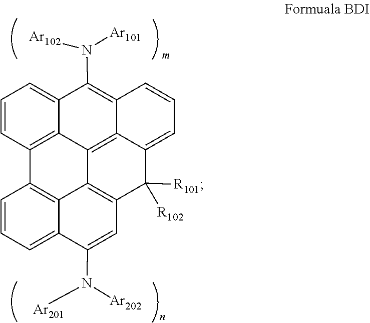

wherein Ar₁₀₁, Ar₁₀₂, Ar₂₀₁ and Ar₂₀₂ are each independently selected from any one of substituted or unsubstituted C6 to C40 (which may be, for example, C6, C10, C12, C15, C18, C24, C30, C36 or C40, etc.) aryl, or substituted or unsubstituted C12 to C20 (which may be, for example, C12, C13, C14, C15, C16, C17, C18, C19 or C20) heteroaryl;
R₁₀₁ and R₁₀₂ are each independently selected from any one of substituted or unsubstituted C1 to C12 (which may be, for example, C1, C2, C3, C4, C5, C6, C7, C8, C9, C10, C11 or C12) alkyl, substituted or unsubstituted C6 to C40 (which may be, for example, C6, C10, C12, C15, C18, C24, C30, C36 or C40, etc.) aryl, or substituted or unsubstituted C12 to C20 (which may be, for example, C12, C13, C14, C15, C16, C17, C18, C19 or C20) heteroaryl;
R₁₀₁ and R₁₀₂ can be joined to form a ring by a single bond;
m and n are each independently selected from 0 or 1, and are not 0 at the same time; and
substituted substituents in Ar₁₀₁, Ar₁₀₂, Ar₂₀₁, Ar₂₀₂, R₁₀₁ and R₁₀₂ are each independently selected from any one or a combination of at least two of -D, —F, —CN, C1 to C12 (which may be, for example, C1, C2, C3, C4, C5, C6, C7, C8, C9, C10, C11 or C12) alkyl, C1 to C6 (which may be, for example, C1, C2, C3, C4, C5 or C6) alkoxy, C2 to C8 (which may be, for example, C2, C3, C4, C5, C6, C7 or C8) alkenyl, C6 to C15 (which may be, for example, C6, C7, C8, C10, C12 or C15, etc.) aryl, or C12 to C20 (which may be, for example, C12, C15, C18 or C20, etc.) heteroaryl.

As a preferred technical solution of the present application, the C6 to C40 aryl is selected from any one of phenyl, biphenyl, terphenyl, naphthyl, anthryl, phenanthryl, fluorenyl, benzofluorenyl, 9,10-diphenylanthryl, dibenzofluorenyl, naphthofluorenyl, pyrenyl, perylenyl, spirofluorenyl, triphenylenyl, fluoranthenyl, hydrogenated benzanthryl, indenofluorenyl, benzindenofluorenyl, dibenzindenofluorenyl, naphthofluorenyl or benzonaphthofluorenyl, preferably any one of phenyl, naphthyl, biphenyl, terphenyl, fluoranthenyl, fluorenyl, 9,10-diphenylanthryl or benzofluorenyl. Optionally, the C12 to C20 heteroaryl is selected from any one of dibenzofuranyl, dibenzothienyl, naphthobenzofuranyl, naphthobenzothienyl, dinaphthofuranyl or dinaphthothienyl.

Optionally, the C1 to C12 alkyl is selected from any one of methyl, ethyl, propyl, butyl, adamantyl, 1-methylcyclohexyl, 1-methylcyclopentyl, cyclopentyl or cyclohexyl.

Optionally, the C1 to C6 alkoxy is selected from any one of methoxy, ethoxy, propoxy, butoxy or

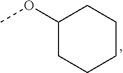

wherein the dashed line represents a linkage site.

Optionally, the C6 to C15 aryl is selected from any one of phenyl, naphthyl or biphenyl.

As a preferred technical solution of the present application, Ar₁₀₁ and Ar₁₀₂ are each independently selected from any one of the following substituted or unsubstituted groups: phenyl, naphthyl, biphenyl, terphenyl, fluoranthenyl, fluorenyl, 9,10-diphenylanthryl, benzofluorenyl, dibenzofuranyl, dibenzothienyl, naphthobenzofuranyl or naphthobenzothienyl:
the substituted substituents are selected from any one or a combination of at least two of -D (a deuterium atom), —F, —CN, phenyl, biphenyl, dibenzofuranyl, methyl, deuterated methyl, adamantyl, tert-butyl, 1-methylcyclopentyl, cyclohexyl, cyclopentyl, methoxy,

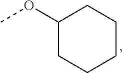

naphthyl, dibenzothienyl or naphthobenzothienyl, wherein the dashed line represents a linkage site;

optionally, Ar₂₀₁ and Ar₂₀₂ are each independently selected from any one of the following substituted or unsubstituted groups: phenyl, naphthyl, dibenzofuranyl or biphenyl;
the substituted substituents are selected from any one or a combination of at least two of methyl, methoxy, phenyl or dibenzofuranyl;
optionally, R₁₀₁ and R₁₀₂ are each independently selected from any one of methyl, ethyl, propyl or phenyl;
optionally, R₁₀₁ is the same as R₁₀₂.

As a preferred technical solution of the present application, the compound having the structure represented by Formula BDI is selected from any one of the following compounds:

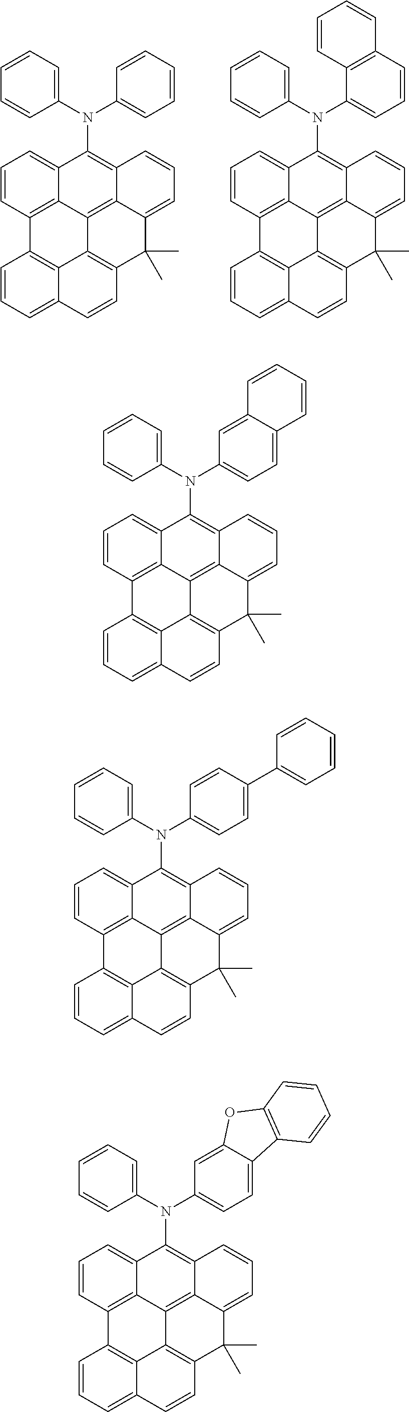 [SMILES: CC1(C)c2cccc3c(N(c4ccccc4)c4ccccc4)c4cccc5c6cccc7ccc1c(c76)c(c23)c45 ; CC1(C)c2cccc3c(N(c4ccccc4)c4cccc5ccccc45)c4cccc5c6cccc7ccc1c(c76)c(c23)c45 ; CC1(C)c2cccc3c(N(c4ccccc4)c4ccc5ccccc5c4)c4cccc5c6cccc7ccc1c(c76)c(c23)c45 ; CC1(C)c2cccc3c(N(c4ccccc4)c4ccc(-c5ccccc5)cc4)c4cccc5c6cccc7ccc1c(c76)c(c23)c45 ; CC1(C)c2cccc3c(N(c4ccccc4)c4ccc5c(c4)oc4ccccc45)c4cccc5c6cccc7ccc1c(c76)c(c23)c45]

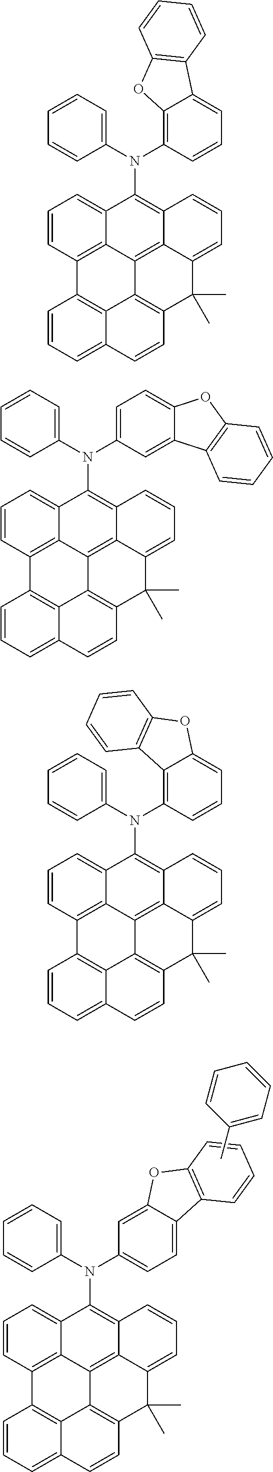

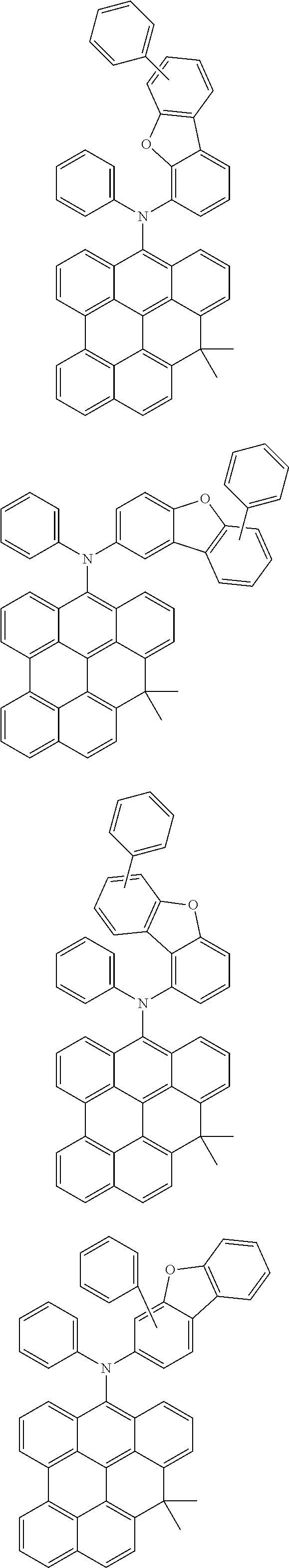

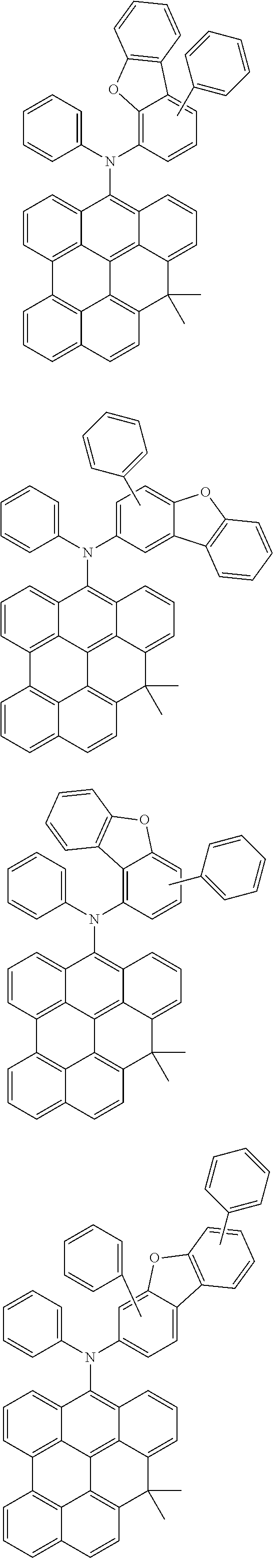

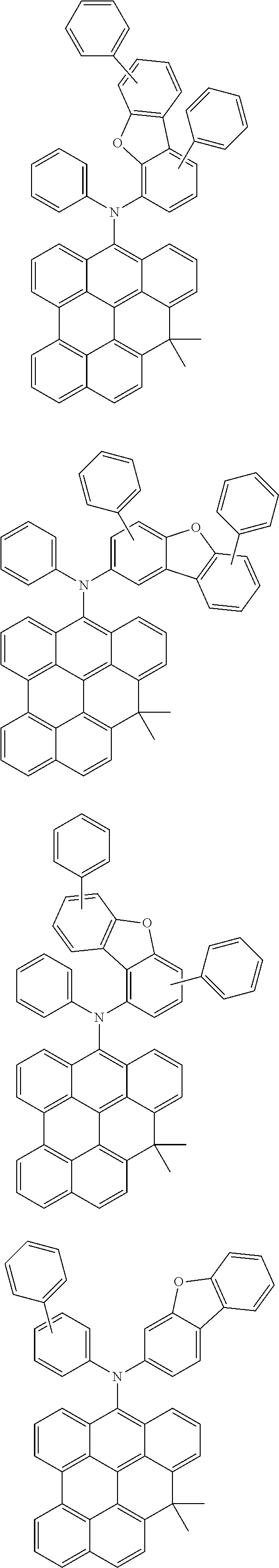

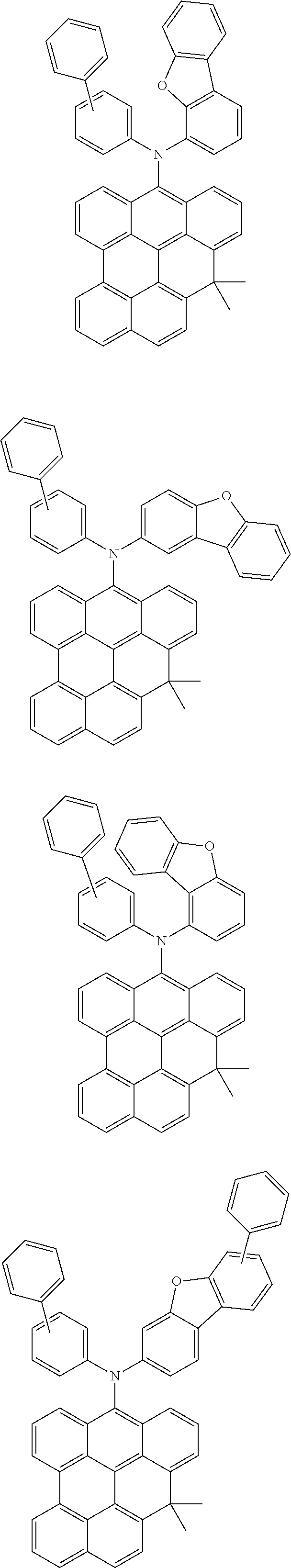

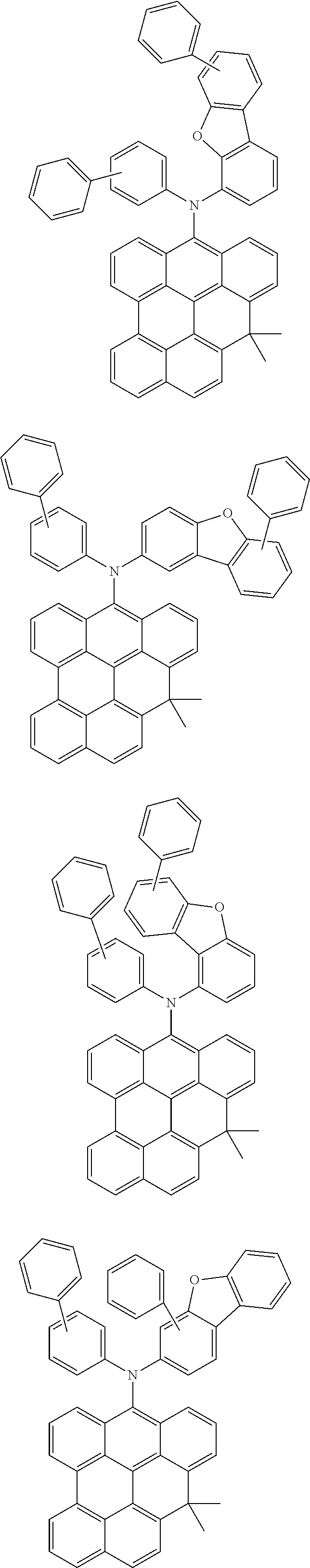

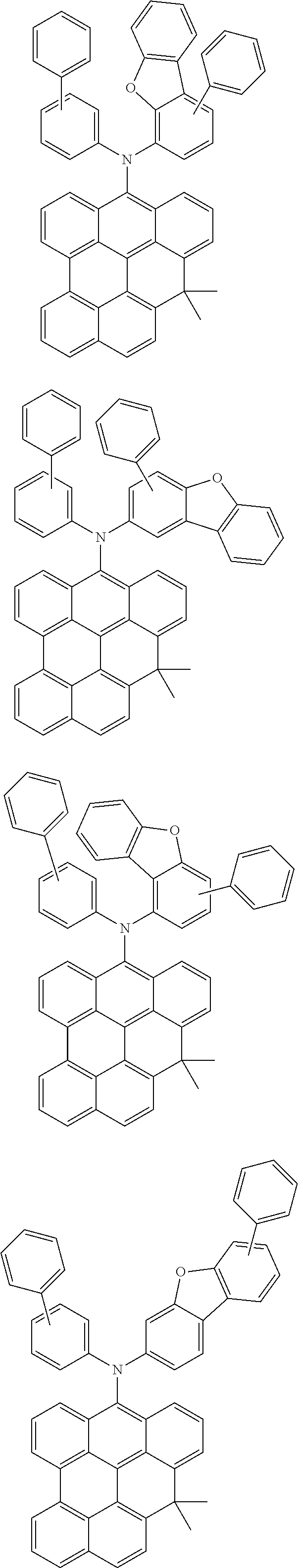

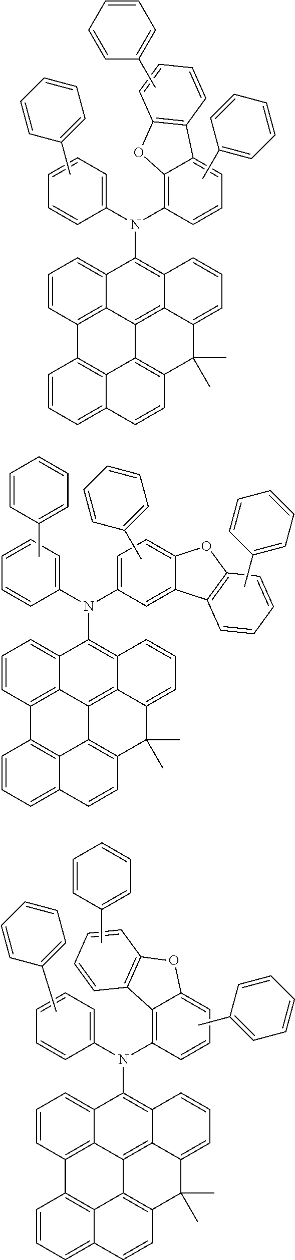

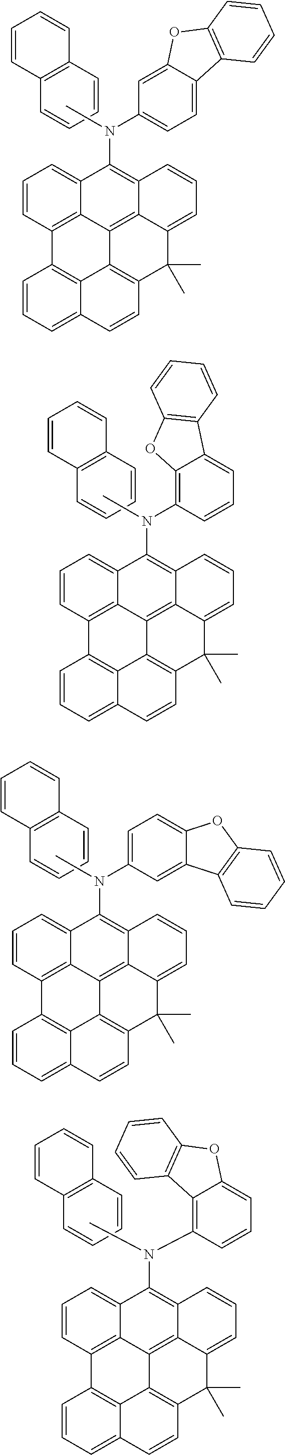

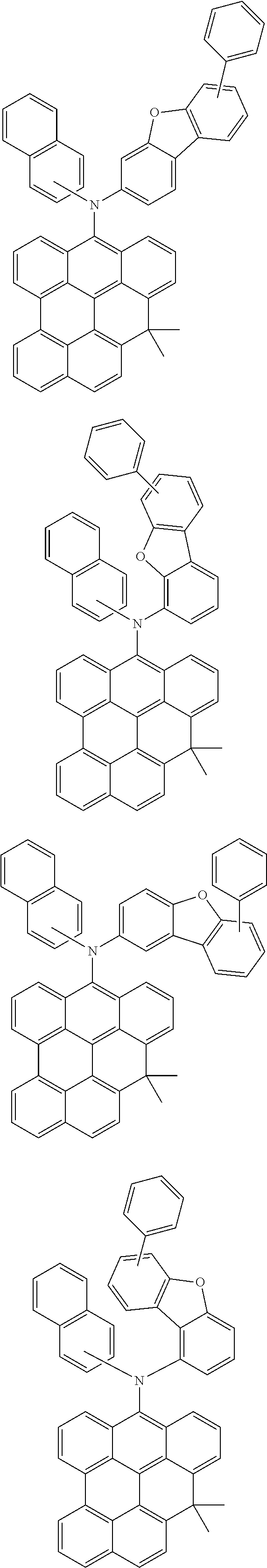

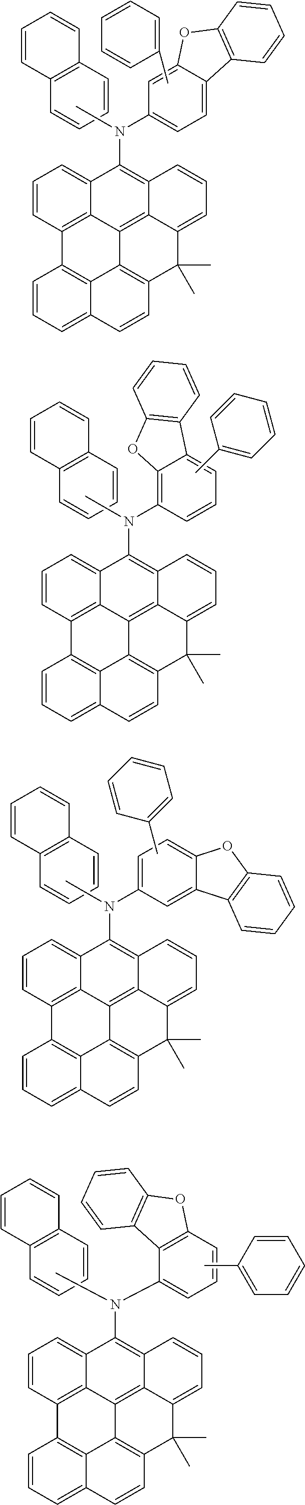

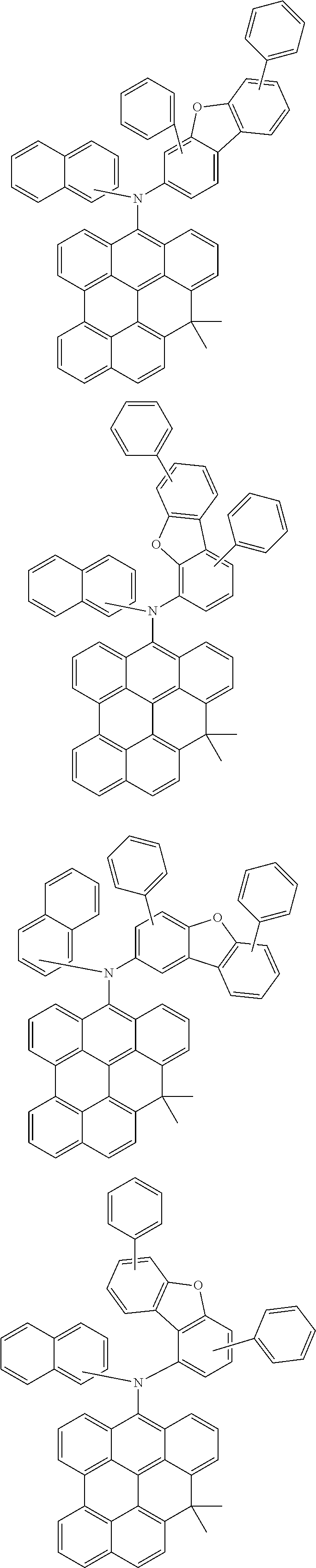

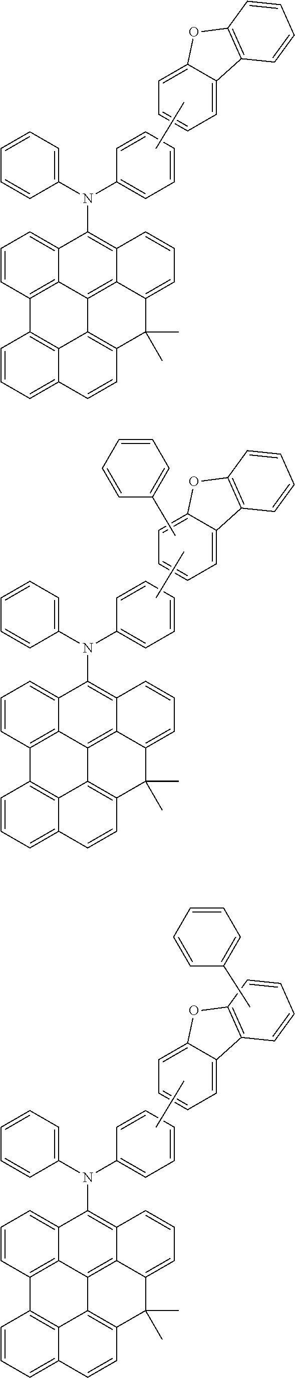

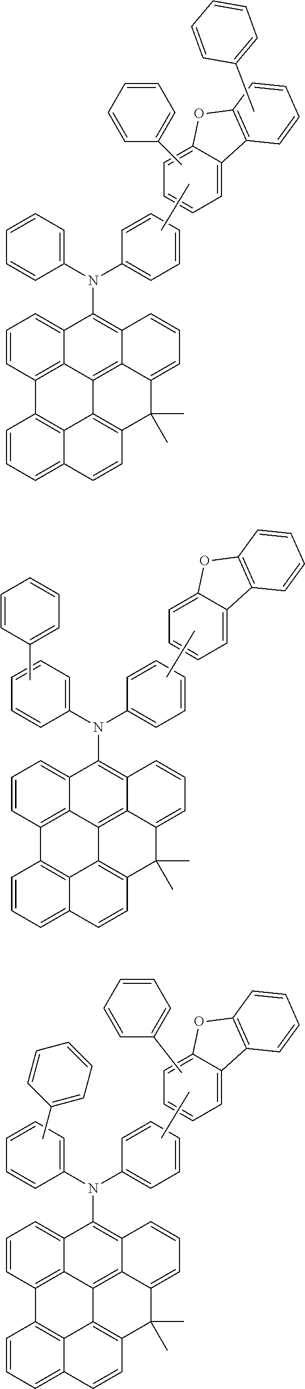

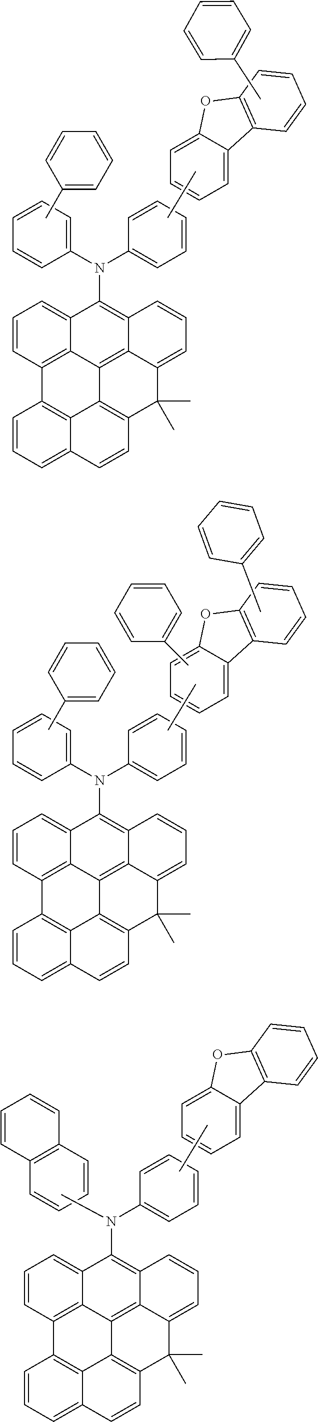

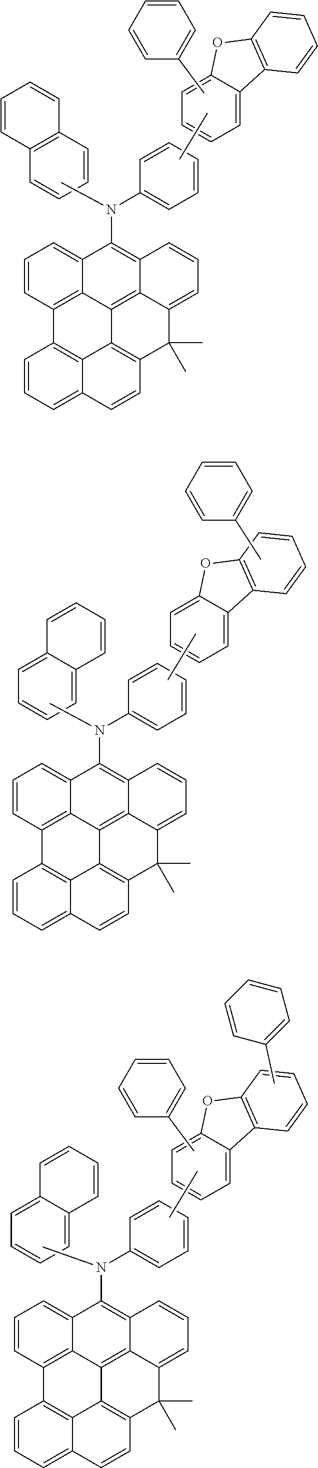

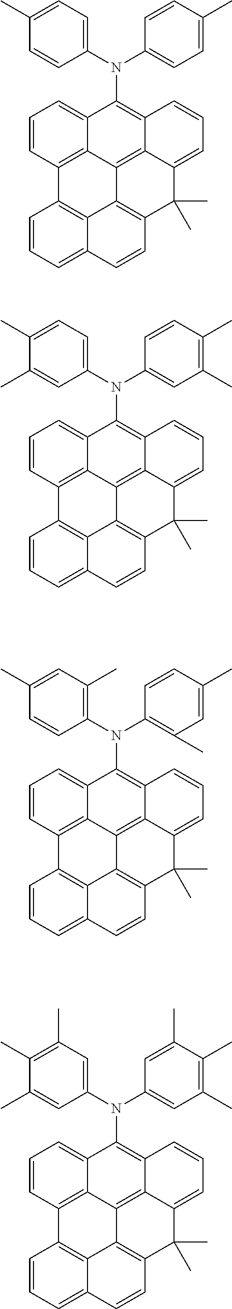

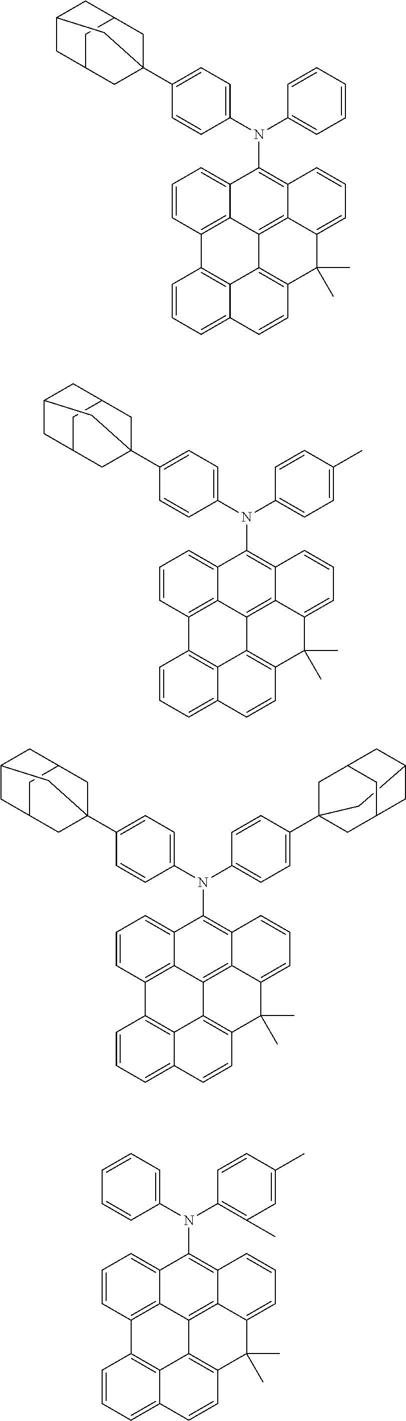 [SMILES: CC1(C)c2cccc3c(N(c4ccccc4)c4ccc(C56CC7CC(CC(C7)C5)C6)cc4)c4cccc5c6cccc7ccc1c(c76)c(c23)c45 ; Cc1ccc(N(c2ccc(C34CC5CC(CC(C5)C3)C4)cc2)c2c3cccc4c3c3c5c(ccc6cccc(c7cccc2c73)c65)C4(C)C)cc1 ; CC1(C)c2cccc3c(N(c4ccc(C56CC7CC(CC(C7)C5)C6)cc4)c4ccc(C56CC7CC(CC(C7)C5)C6)cc4)c4cccc5c6cccc7ccc1c(c76)c(c23)c45 ; Cc1ccc(N(c2ccccc2)c2c3cccc4c3c3c5c(ccc6cccc(c7cccc2c73)c65)C4(C)C)c(C)c1]

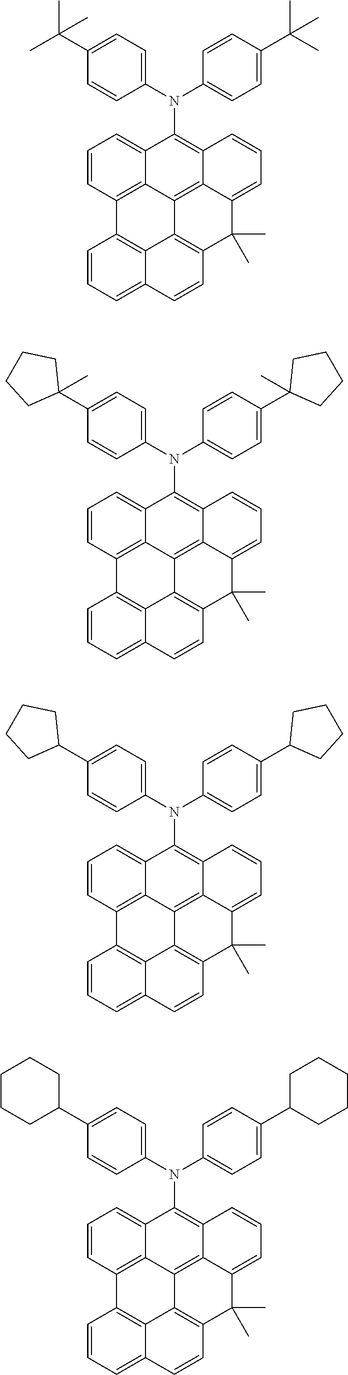 [SMILES: CC(C)(C)c1ccc(N(c2ccc(C(C)(C)C)cc2)c2c3cccc4c3c3c5c(ccc6cccc(c7cccc2c73)c65)C4(C)C)cc1 ; CC1(c2ccc(N(c3ccc(C4(C)CCCC4)cc3)c3c4cccc5c4c4c6c(ccc7cccc(c8cccc3c84)c76)C5(C)C)cc2)CCCC1 ; CC1(C)c2cccc3c(N(c4ccc(C5CCCC5)cc4)c4ccc(C5CCCC5)cc4)c4cccc5c6cccc7ccc1c(c76)c(c23)c45 ; CC1(C)c2cccc3c(N(c4ccc(C5CCCCC5)cc4)c4ccc(C5CCCCC5)cc4)c4cccc5c6cccc7ccc1c(c76)c(c23)c45]

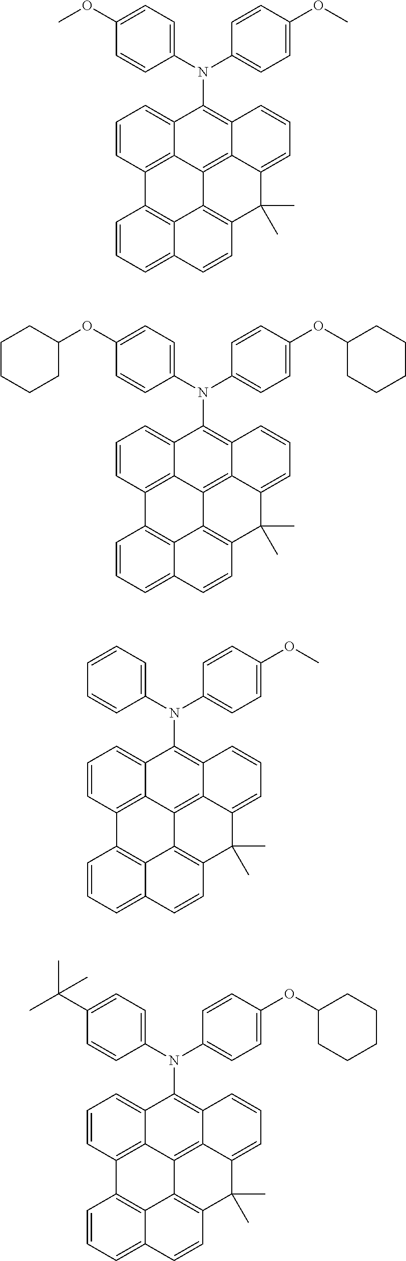

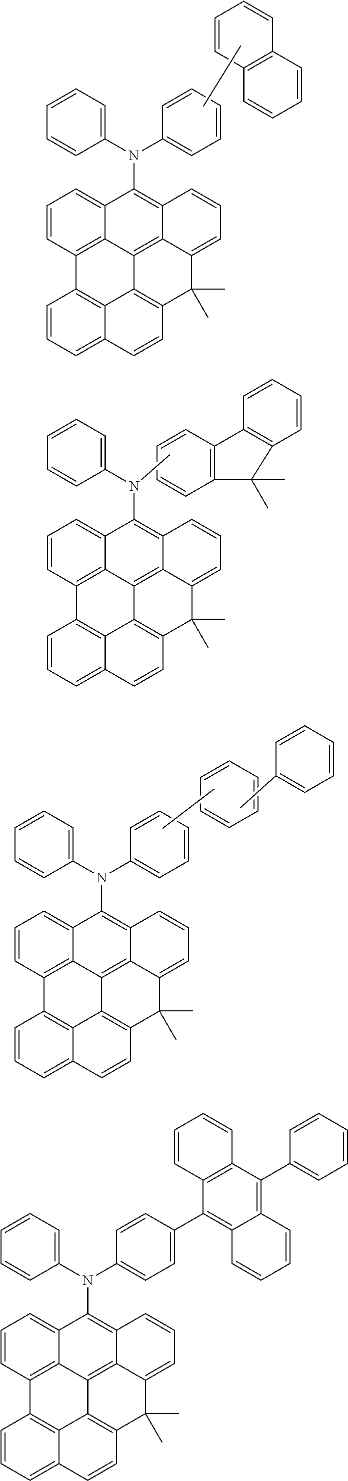

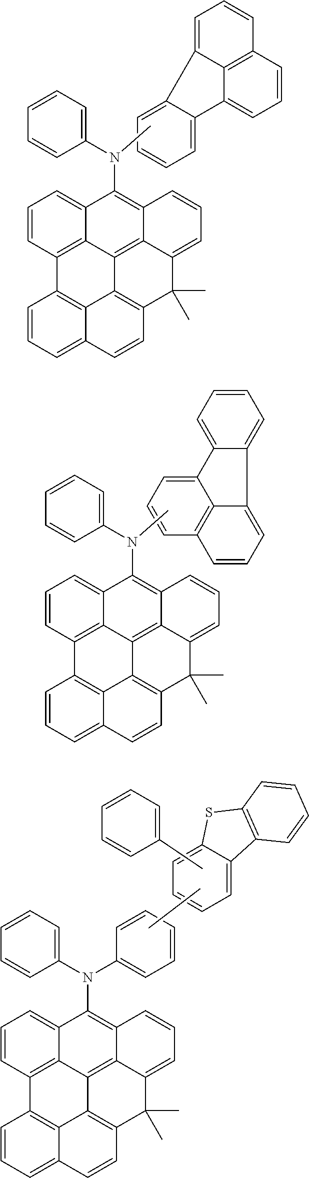

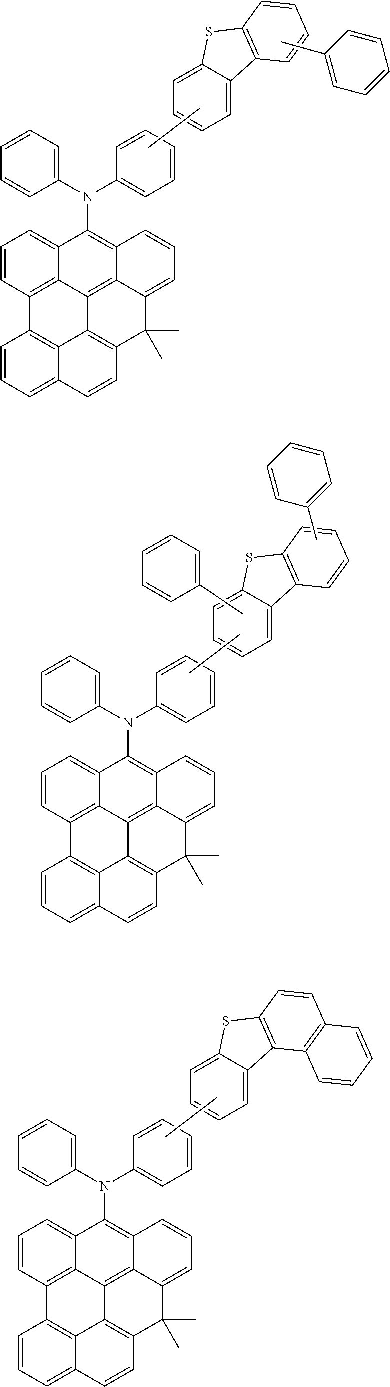

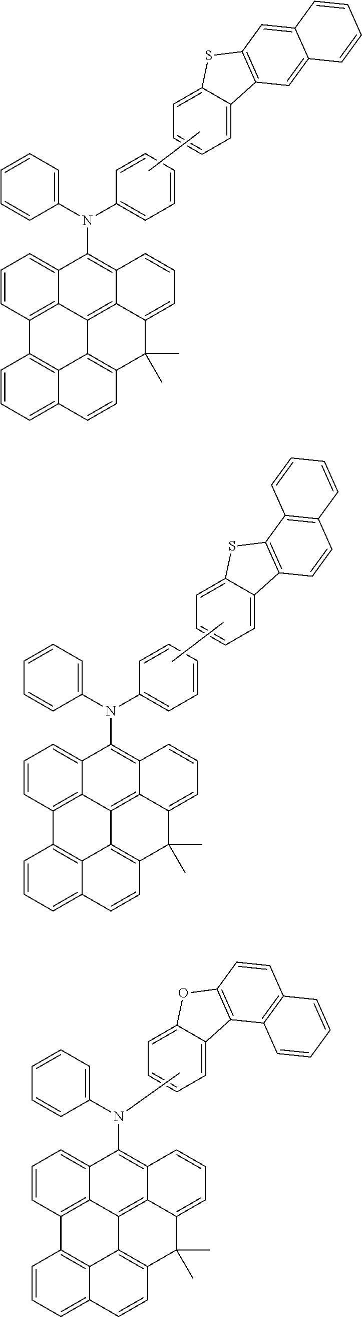

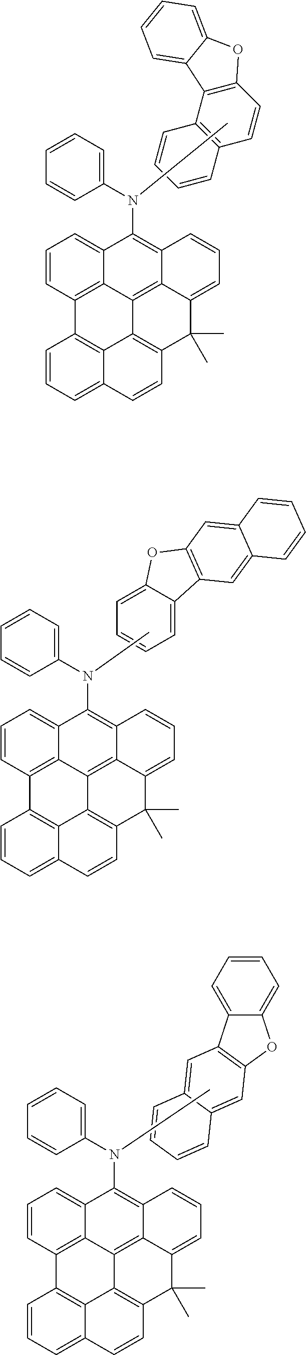

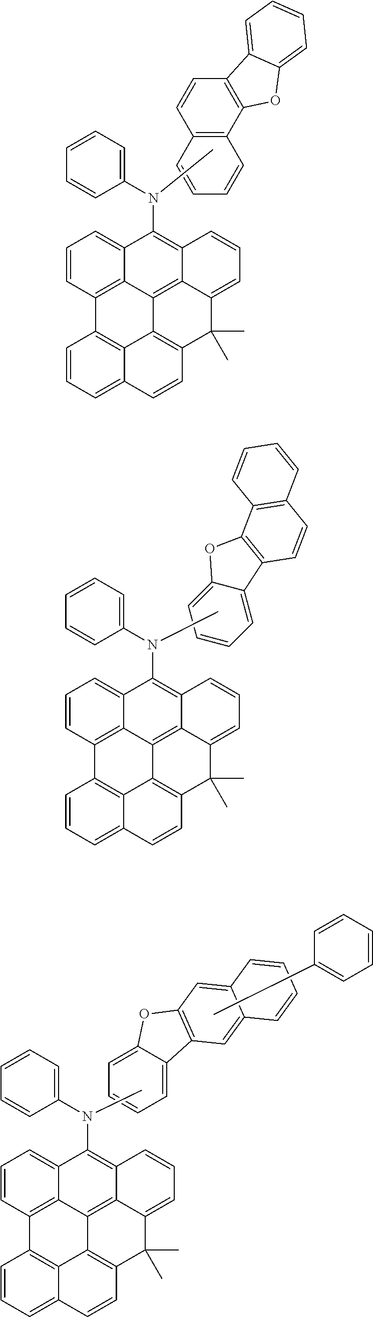

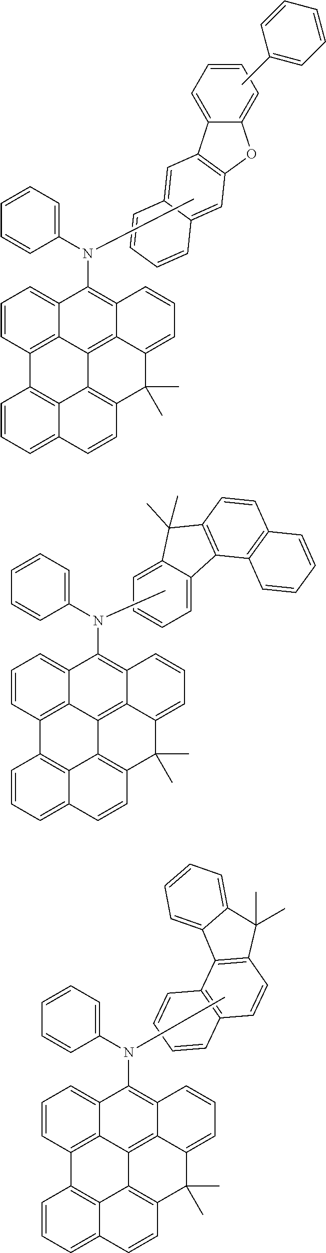

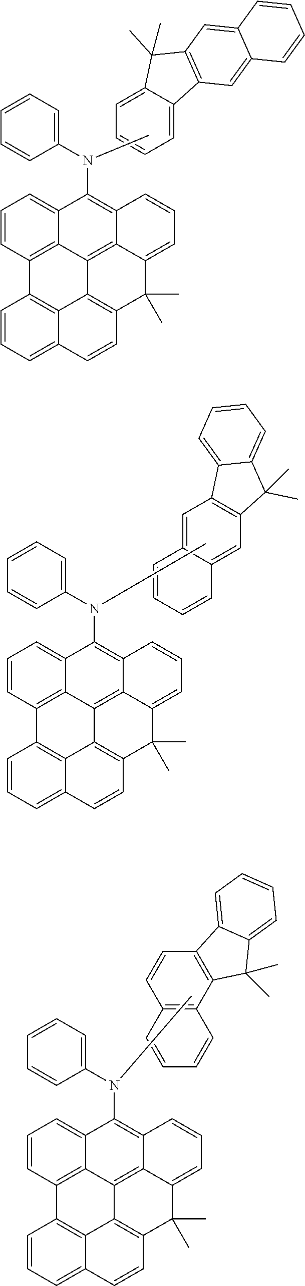

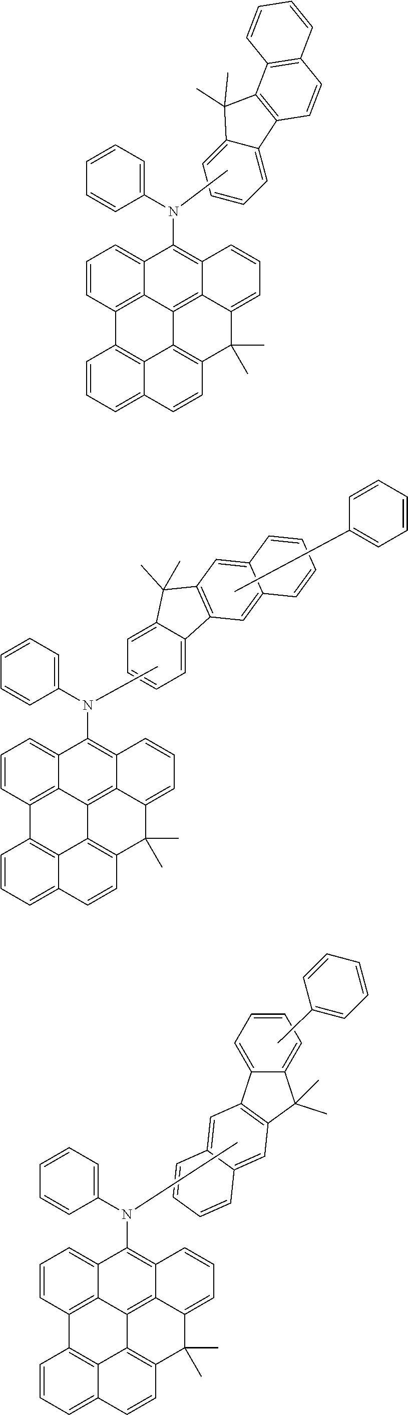

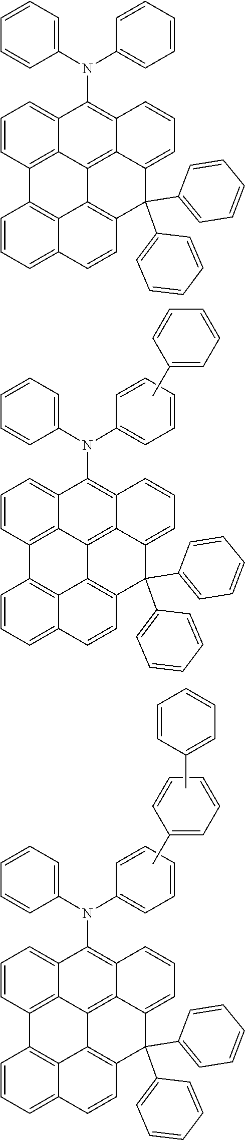

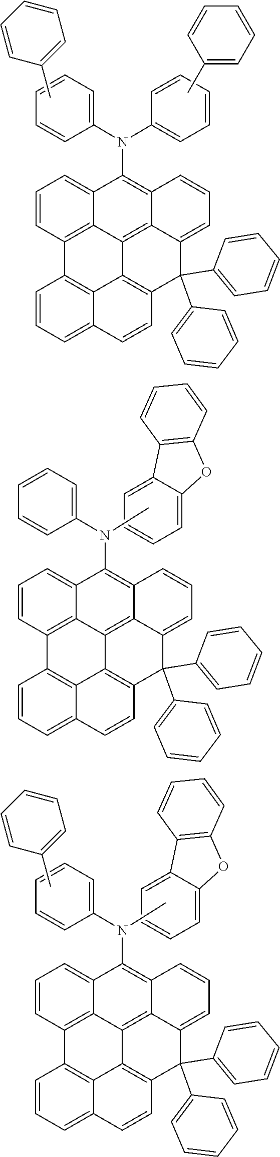

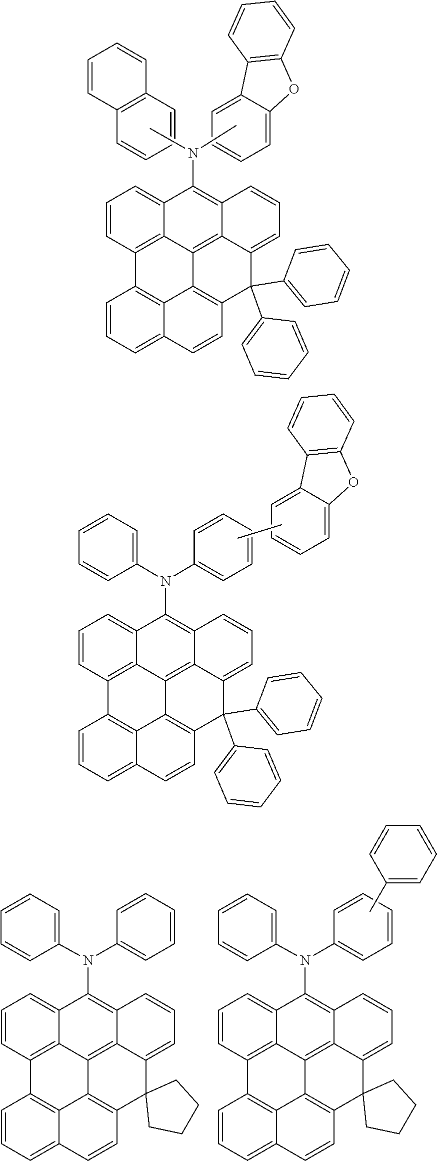

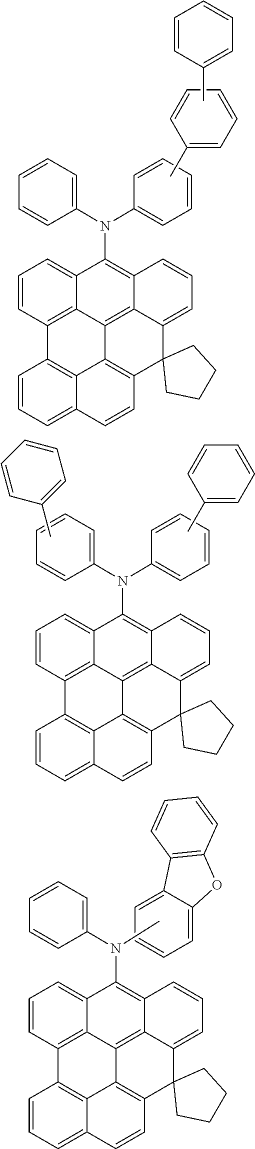

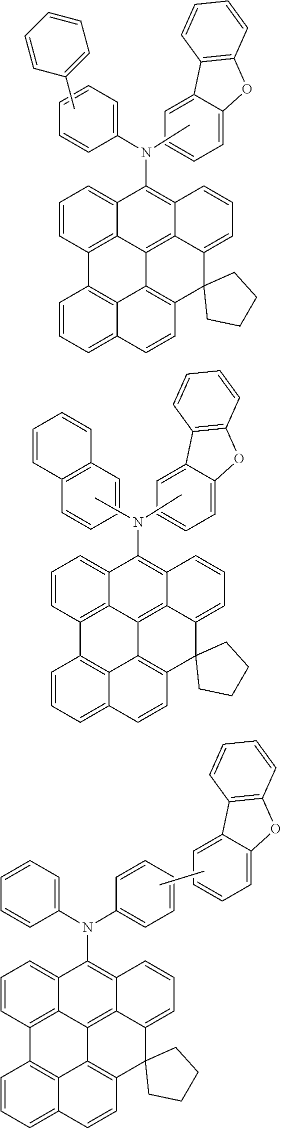

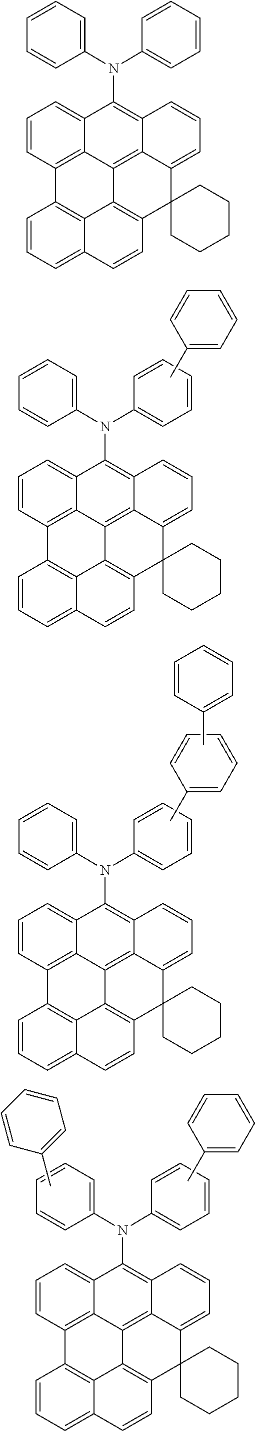

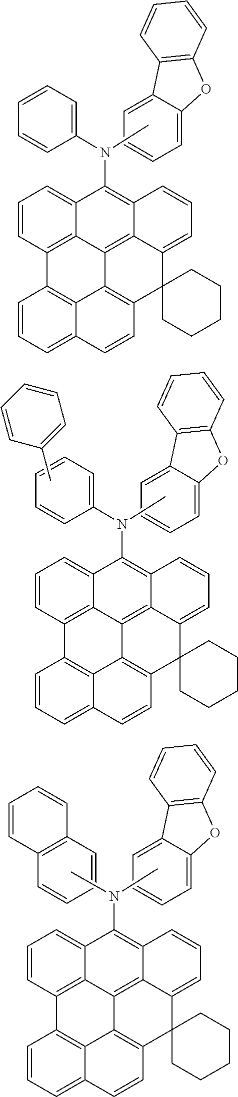

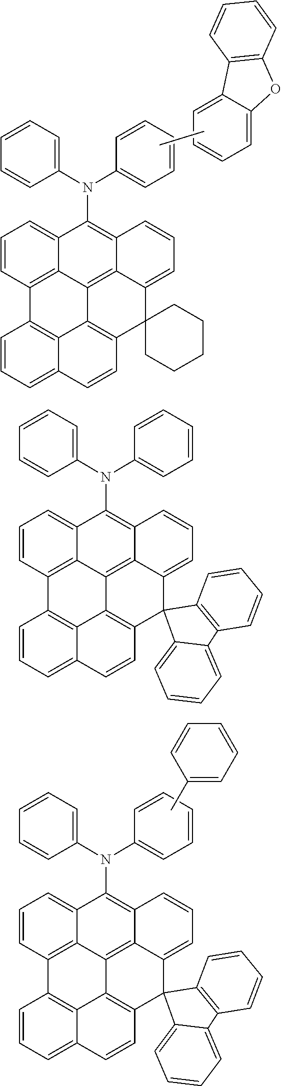

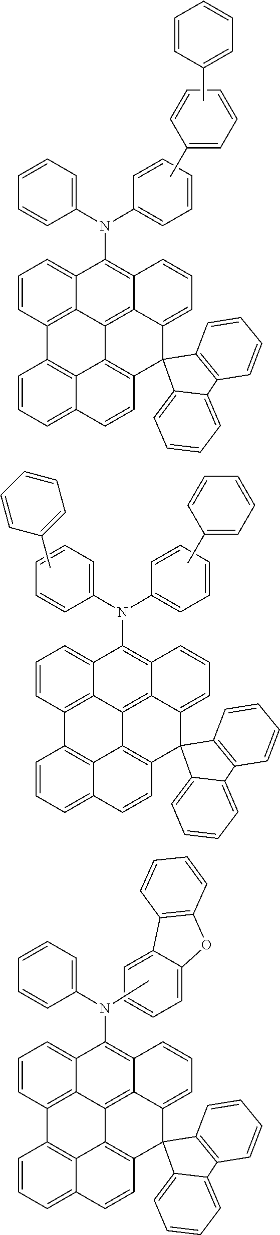

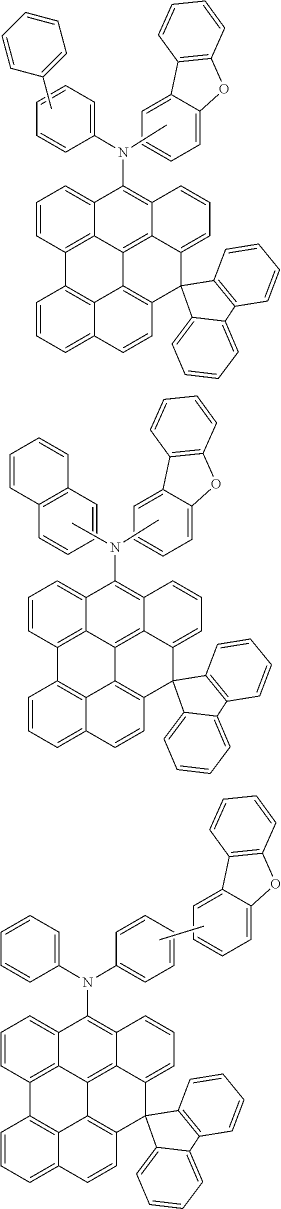

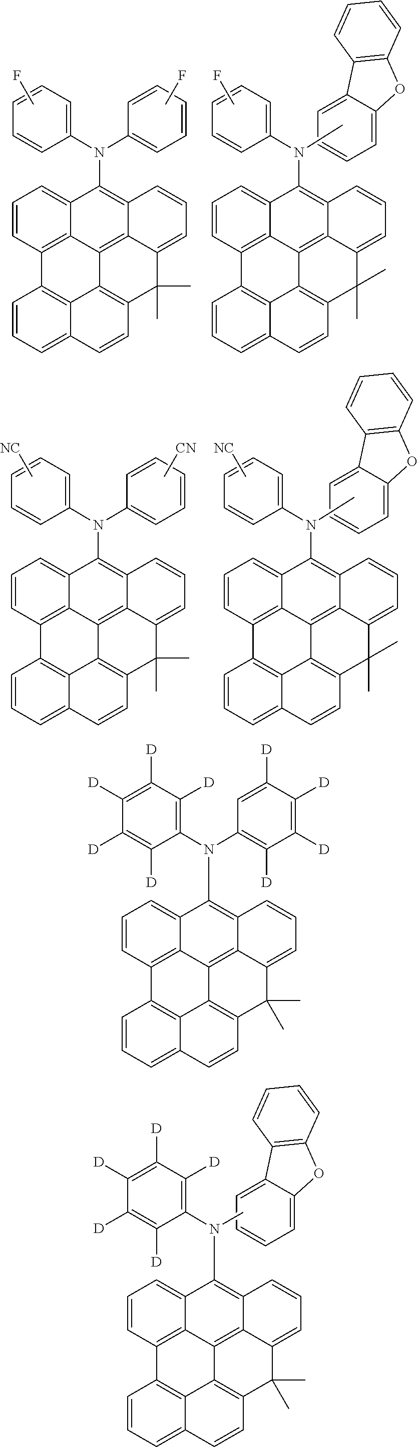

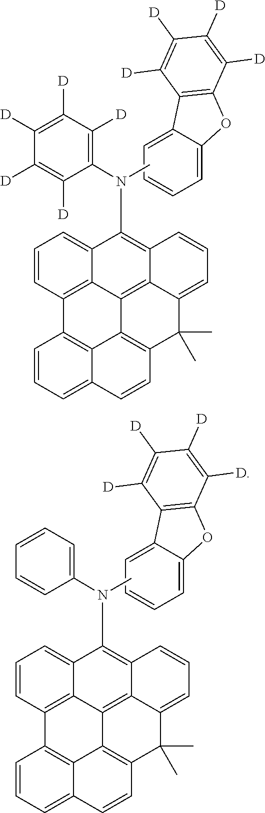

Optionally, the compound having the structure represented by Formula BDI is selected from any one of following compounds:

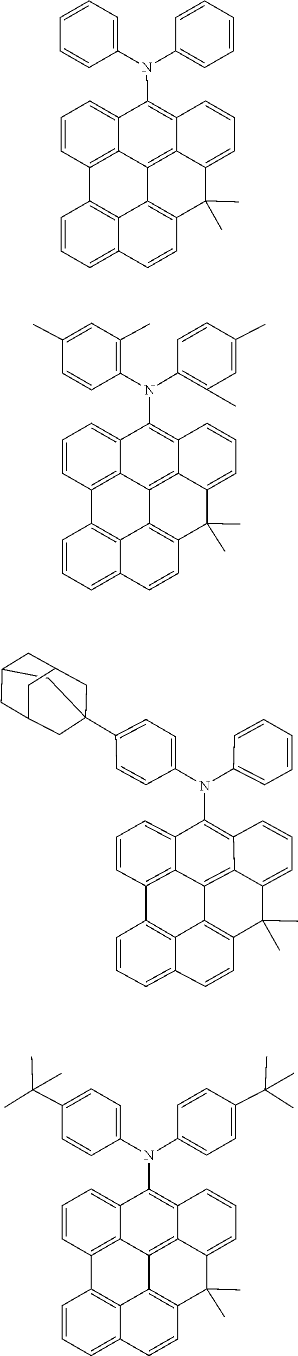

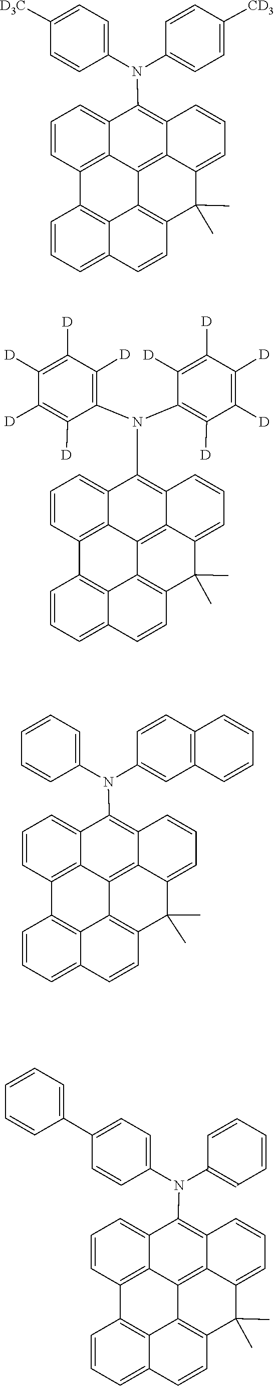 [SMILES: [2H]C([2H])([2H])c1ccc(N(c2ccc(C([2H])([2H])[2H])cc2)c2c3cccc4c3c3c5c(ccc6cccc(c7cccc2c73)c65)C4(C)C)cc1 ; [2H]c1c([2H])c([2H])c(N(c2c([2H])c([2H])c([2H])c([2H])c2[2H])c2c3cccc4c3c3c5c(ccc6cccc(c7cccc2c73)c65)C4(C)C)c([2H])c1[2H] ; CC1(C)c2cccc3c(N(c4ccccc4)c4ccc5ccccc5c4)c4cccc5c6cccc7ccc1c(c76)c(c23)c45 ; CC1(C)c2cccc3c(N(c4ccccc4)c4ccc(-c5ccccc5)cc4)c4cccc5c6cccc7ccc1c(c76)c(c23)c45]

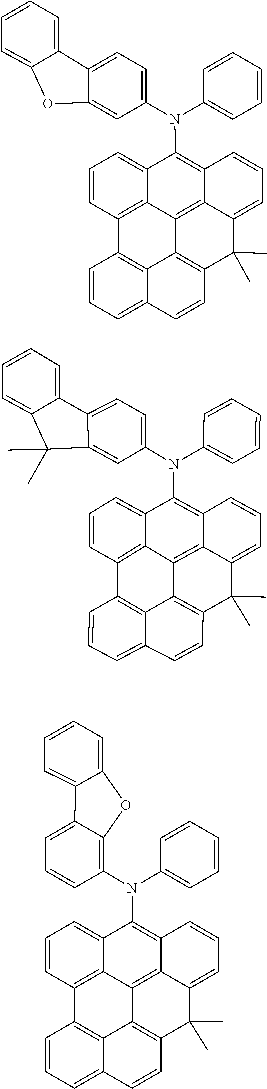 [SMILES: CC1(C)c2cccc3c(N(c4ccccc4)c4ccc5c(c4)oc4ccccc45)c4cccc5c6cccc7ccc1c(c76)c(c23)c45 ; CC1(C)c2ccccc2-c2ccc(N(c3ccccc3)c3c4cccc5c4c4c6c(ccc7cccc(c8cccc3c84)c76)C5(C)C)cc21 ; CC1(C)c2cccc3c(N(c4ccccc4)c4cccc5c4oc4ccccc45)c4cccc5c6cccc7ccc1c(c76)c(c23)c45]

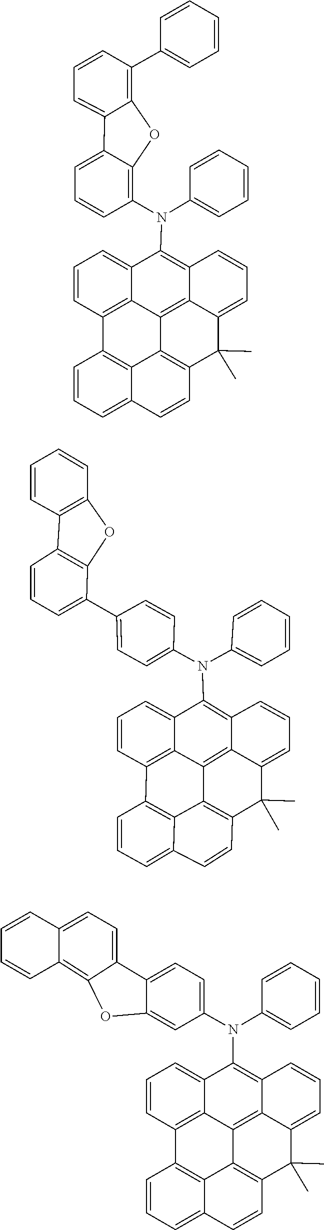 [SMILES: CC1(C)c2cccc3c(N(c4ccccc4)c4cccc5c4oc4c(-c6ccccc6)cccc45)c4cccc5c6cccc7ccc1c(c76)c(c23)c45 ; CC1(C)c2cccc3c(N(c4ccccc4)c4ccc(-c5cccc6c5oc5ccccc56)cc4)c4cccc5c6cccc7ccc1c(c76)c(c23)c45 ; CC1(C)c2cccc3c(N(c4ccccc4)c4ccc5c(c4)oc4c6ccccc6ccc54)c4cccc5c6cccc7ccc1c(c76)c(c23)c45]

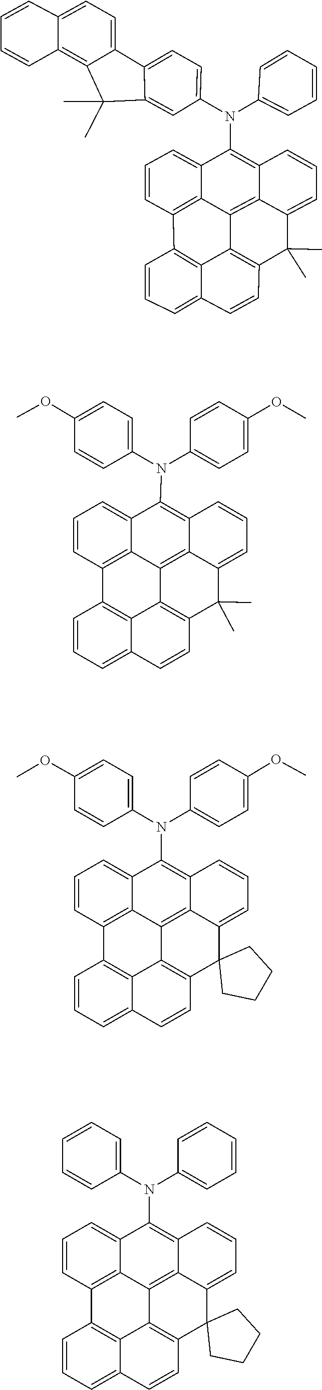

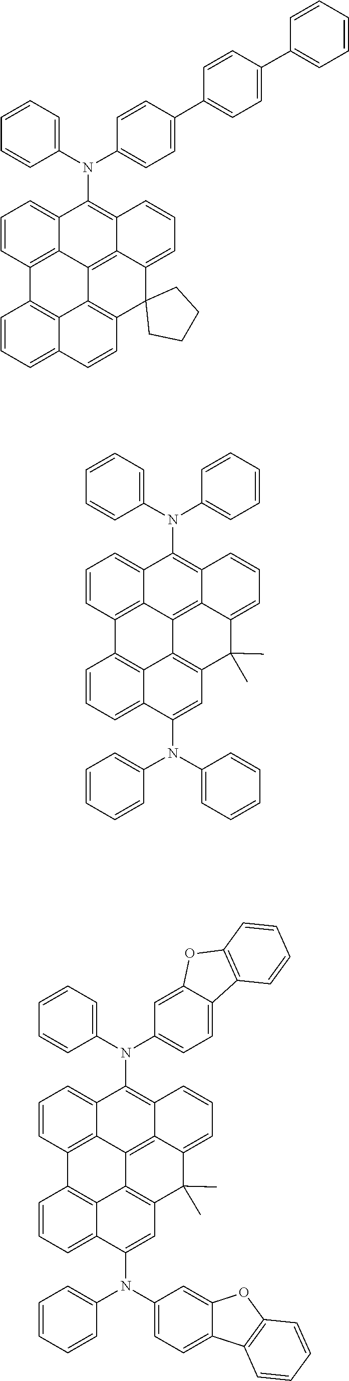 [SMILES: c1ccc(-c2ccc(-c3ccc(N(c4ccccc4)c4c5cccc6c5c5c7c(ccc8cccc(c9cccc4c95)c87)C64CCCC4)cc3)cc2)cc1 ; CC1(C)c2cccc3c(N(c4ccccc4)c4ccccc4)c4cccc5c6cccc7c(N(c8ccccc8)c8ccccc8)cc1c(c76)c(c23)c45 ; CC1(C)c2cccc3c(N(c4ccccc4)c4ccc5c(c4)oc4ccccc45)c4cccc5c6cccc7c(N(c8ccccc8)c8ccc9c(c8)oc8ccccc89)cc1c(c76)c(c23)c45]

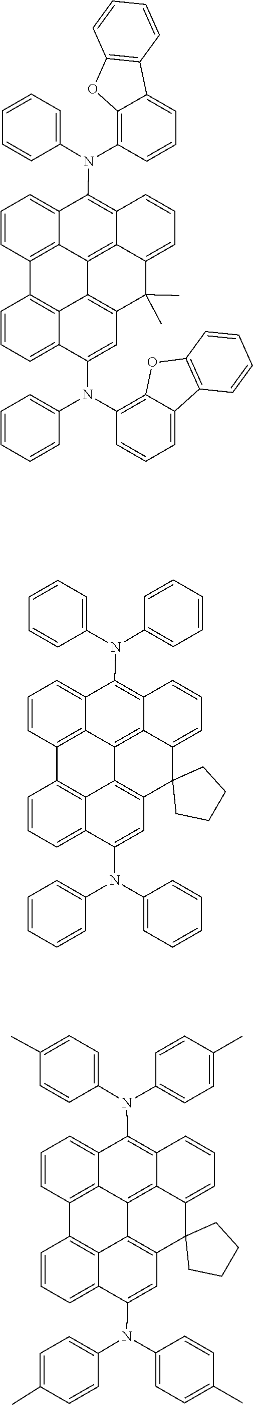

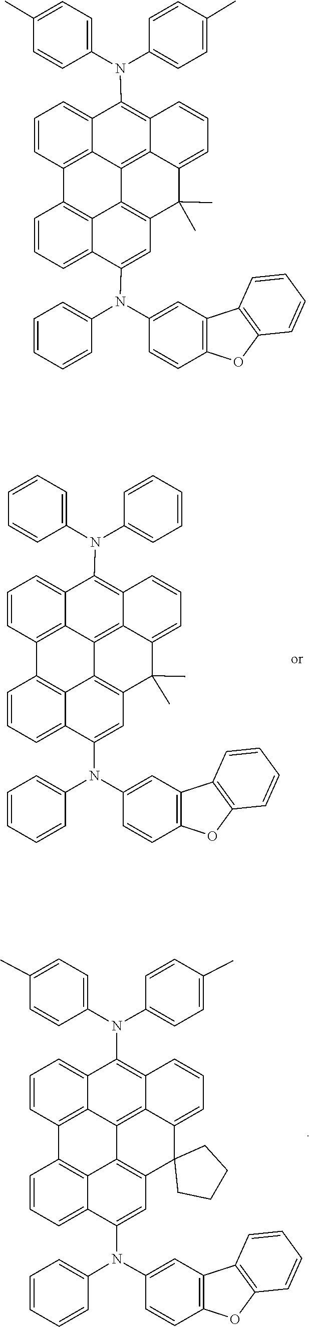 [SMILES: Cc1ccc(N(c2ccc(C)cc2)c2c3cccc4c3c3c5c(cc(N(c6ccccc6)c6ccc7oc8ccccc8c7c6)c6cccc(c7cccc2c73)c65)C4(C)C)cc1 ; CC1(C)c2cccc3c(N(c4ccccc4)c4ccccc4)c4cccc5c6cccc7c(N(c8ccccc8)c8ccc9oc%10ccccc%10c9c8)cc1c(c76)c(c23)c45 ; Cc1ccc(N(c2ccc(C)cc2)c2c3cccc4c3c3c5c(cc(N(c6ccccc6)c6ccc7oc8ccccc8c7c6)c6cccc(c7cccc2c73)c65)C42CCCC2)cc1]

In a third aspect, the present application provides a display device including the organic electroluminescent device according to the second aspect.

Compared with the prior art, the present application has the beneficial effects described below.

In the present application, the anthracene compound substituted with the aryl group is subjected to the deuterated reaction to obtain the deuterated mixture, the obtained deuterated mixture is used as the host material for blue light, and the OLED light-emitting device prepared in this manner has a relatively low driving voltage, relatively high current efficiency and a relatively long lifetime. Moreover, in the present application, the process of the deuterated reaction for preparing the deuterated mixture is simple, the reaction condition is mild, the cumbersome purification process is not needed, and the post treatment is simple so that the deuterated mixture is applicable to prepare the organic electroluminescent device. When the compound represented by BDI is used as the doped material, device performance is more excellent.

Other aspects can be understood after the drawings and the detailed description are read and understood.

### BRIEF DESCRIPTION OF DRAWINGS

The drawings are intended to provide a further understanding of technical solutions herein, constitute part of the specification, and explain the technical solutions herein in conjunction with embodiments of the present application and do not limit the technical solutions herein.

FIG. 1 is a diagram illustrating a relationship between deuteration rates of host materials of light-emitting layers and lifetimes LT90 of organic electroluminescent devices according to Application Examples 1 to 12.

FIG. 2 is a diagram illustrating a relationship between deuteration rates of host materials of light-emitting layers and driving voltages of organic electroluminescent devices provided in Application Examples 1 to 12 and a relationship between the deuteration rates of the host materials of the light-emitting layers and current efficiency of the organic electroluminescent devices according to Application Examples 1 to 12.

FIG. 3 is a mass spectrum of BH3-D03 in a deuterated composition according to Example 5 of the present application.

### DETAILED DESCRIPTION

The technical solutions of the present application are further described hereinafter through examples in conjunction with the drawings. Those skilled in the art are to understand that the examples described herein are used for a better understanding of the present application and are not to be construed as specific limitations to the present application.

### Example 1

This example provides a BH1-D series deuterated composition whose reaction equation is as follows:

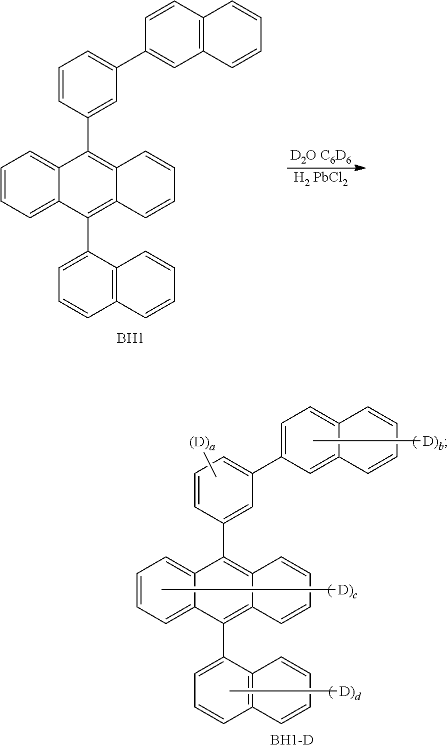

wherein a is selected from integers between 0 and 4, b is selected from integers between 0 and 7, c is selected from integers between 0 and 8, d is selected from integers between 0 and 7, and a+b+c+d≥1.

A method for preparing the above BH1-D series deuterated composition is described below.

At room temperature, BH1 (5.06 g, 0.01 mol), palladium (II) chloride (0.0177 g, 0.0001 mol), activated carbon (0.2 g), D₂O (10 mL) and C₆D₆ (50 mL) were added to a 500 mL autoclave, and hydrogen was introduced into the autoclave until the pressure was 0.02 MPa. Then, after the autoclave was heated to 90° C. and a reaction was conducted for a certain time, the autoclave was cooled to room temperature, the system was filtered, and layers were separated. An organic layer obtained were separated and dried with magnesium sulfate, decolored with a short silica gel column, concentrated to dryness and vacuum dried for 24 h to obtain BH1-D.

Products prepared at different reaction times were weighed. After the products were sublimated, deuteration rates were detected (the deuteration rates were tested through an internal standard method, for example, reference may be made to a method described in a literature “Wu Yurong, Chen Minzhu, Determination of the content of phBr-D with ¹HNMR[J], Journal of Sichuan University: Natural Science Edition, 1997, 34 (6): 2” to perform the test). The reaction times and the values of the deuteration rates of the products are shown in the following Table 1.

TABLE 1

| | Deuterated | Reaction | Product | Deuteration |
|---|---|---|---|---|
| No. | Composition | Time/(h) | Weight/(g) | Rate |
| 1 | BH1-D01 | 1 | 4.88 | 15.11% |
| 2 | BH1-D02 | 2 | 4.96 | 28.27% |
| 3 | BH1-D03 | 3 | 4.99 | 38.66% |
| 4 | BH1-D04 | 4 | 4.98 | 46.00% |
| 5 | BH1-D05 | 6 | 5.01 | 53.77% |
| 6 | BH1-D06 | 8 | 5.06 | 57.26% |
| 7 | BH1-D07 | 10 | 5.06 | 60.11% |
| 8 | BH1-D08 | 15 | 5.11 | 69.36% |
| 9 | BH1-D09 | 20 | 5.13 | 77.68% |
| 10 | BH1-D10 | 30 | 5.12 | 86.19% |
| 11 | BH1-D11 | 40 | 5.16 | 89.36% |
| 12 | BH1-D12 | 80 | 5.11 | 98.22% |

### Example 2

This example provides a BH2-D series deuterated composition whose reaction equation is as follows:

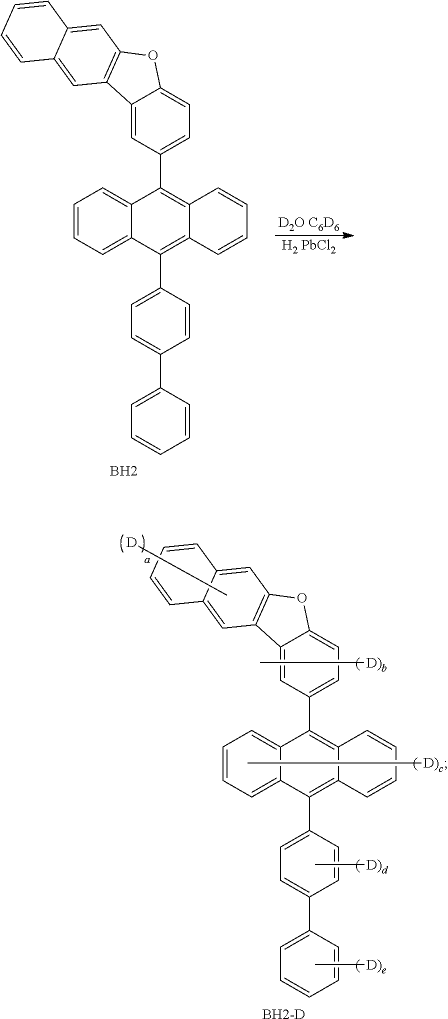

wherein a is selected from integers between 0 and 6, b is selected from integers between 0 and 3, c is selected from integers between 0 and 8, d is selected from integers between 0 and 4, and a+b+c+d+e≥1.

A method for preparing the above BH2-D series deuterated composition is described below.

At room temperature, BH2 (5.46 g, 0.01 mol), palladium (II) chloride (0.0177 g, 0.0001 mol), activated carbon (0.2 g), D₂O (12 mL) and C₆D₆ (50 mL) were added to a 500 mL autoclave, and hydrogen was introduced into the autoclave until the pressure was 0.02 MPa. Then, after the autoclave was heated to 90° C. and a reaction was conducted for a certain time, the autoclave was cooled to room temperature, the system was filtered, and layers were separated. An organic layer obtained after the layers were separated was dried with magnesium sulfate, decolored with a short silica gel column, concentrated to dryness and vacuum dried for 24 h to obtain BH2-D.

Products prepared at different reaction times were weighed. After the products were sublimated, deuteration rates were detected (a test method was the same as above). The reaction times and the values of the deuteration rates of the products are shown in the following Table 2.

TABLE 2

| | Deuterated | Reaction | Product | Deuteration |
|---|---|---|---|---|
| No. | Composition | Time/(h) | Weight/(g) | Rate |
| 1 | BH2-D01 | 2 | 5.23 | 30.11% |
| 2 | BH2-D02 | 8 | 5.32 | 58.31% |
| 3 | BH2-D03 | 20 | 5.33 | 78.16% |
| 4 | BH2-D04 | 40 | 5.35 | 90.21% |
| 5 | BH2-D05 | 80 | 5.35 | 98.21% |

In a process of preparing an OLED device, the deuterated composition provided in the present application needs to be evaporated in an evaporation manner. Therefore, there is a need to ensure that to-be-evaporated components remain relatively stable, and the following evaporation experiment is performed: 2 g BH1-D01, BH1-D08 or BH2-D04 was taken, placed in a crucible of an evaporator and heated to 300° C. at a pressure of 1×10⁻⁵-9×10⁻⁵ Pa, the material in the crucible was sequentially evaporated to glass substrates whose serial numbers were 1 to 9 until the material in the crucible remained about 10%, and a total of nine glass substrates were obtained according to the sequence of the evaporation. Deuteration rates of materials on these nine glass substrates were separately analyzed, as shown in the following Table 3.

TABLE 3

| Glass | | | | | | | | | |
|---|---|---|---|---|---|---|---|---|---|
| Substrate | | | | | | | | | |
| No. | 1 | 2 | 3 | 4 | 5 | 6 | 7 | 8 | 9 |
| BH1-D01 | 15.12 | 15.13 | 15.00 | 15.21 | 15.12 | 15.09 | 15.21 | 15.11 | 15.13 |
| Deuteration | | | | | | | | | |
| Rate/(%) | | | | | | | | | |
| BH1-D08 | 69.33 | 69.34 | 69.41 | 69.30 | 69.29 | 69.39 | 69.42 | 69.38 | 69.38 |
| Deuteration | | | | | | | | | |
| Rate/(%) | | | | | | | | | |
| BH2-D04 | 90.11 | 90.22 | 90.31 | 90.21 | 90.19 | 90.22 | 90.23 | 90.18 | 90.19 |
| Deuteration | | | | | | | | | |
| Rate/(%) | | | | | | | | | |

As can be seen from the contents in Table 3, the deuteration rates of the materials evaporated to the glass substrates in different periods are basically the same, indicating that the components of the deuterated composition provided in the present application can remain to be relatively stable in the evaporation process.

Specific structures of compounds used in the following application examples are shown as follows:

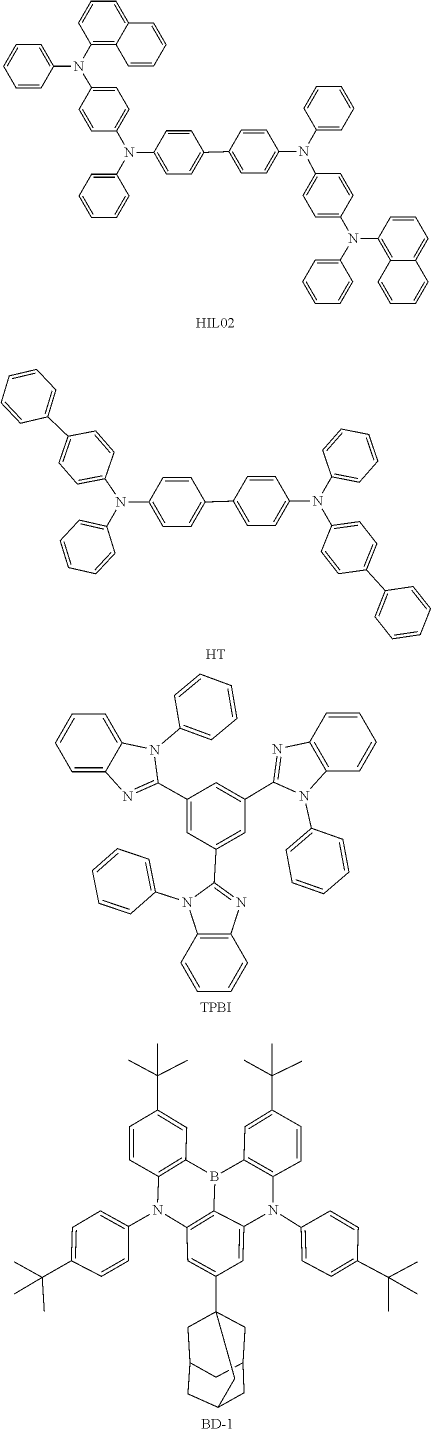

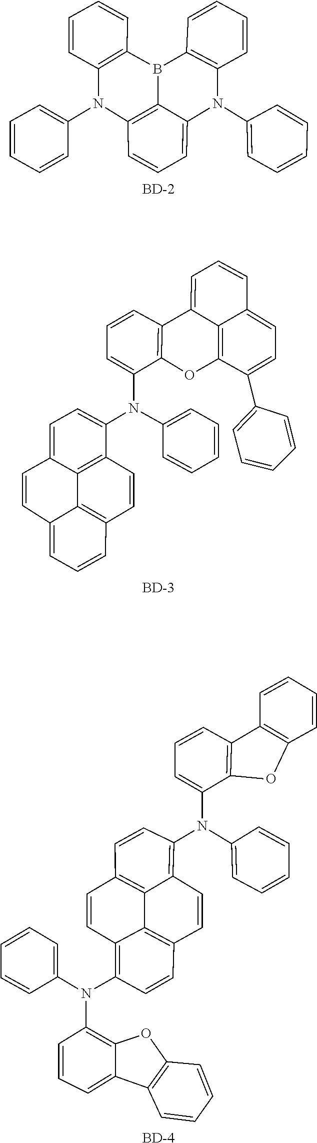

### Application Example 1

This application example provides an organic electroluminescent device. The structure of the organic electroluminescent device is ITO/HIL02 (100 nm)/HT (40 nm) light-emitting layer (30 nm): BD-1 (3%)/TPBI (30 nm)/LiF (0.5 nm)/Al (150 nm).

The method for preparing the above organic electroluminescent device is described below.
(1) A glass substrate coated with the ITO transparent conductive layer (as an anode) was ultrasonically treated in a cleaning agent, flushed in deionized water, ultrasonically degreased in a mixed solvent of acetone and ethanol, dried and cleaned with ultraviolet and ozone. The surface was bombarded with a low-energy cation beam to improve an energy level property of the ITO surface, thereby improving an ability to bind to a hole injection layer.
(2) The above glass substrate was placed in a vacuum chamber, the vacuum chamber was vacuumed to 1×10⁻⁵-1×10⁻⁴ Pa, and HIL02 was vacuum evaporated on the anode as the hole injection layer. The evaporation rate was 0.01 nm/s, and the evaporation film had a thickness of 100 nm.
(3) HT was vacuum evaporated on the hole injection layer as a hole transport layer. The evaporation rate was 0.01 nm/s, and the evaporation film had a thickness of 40 nm.
(4) On the hole transport layer, the light-emitting layer was vacuum evaporated. The evaporation rate was 0.01 nm/s, and the evaporation film had a total thickness of 30 nm. In the light-emitting layer, the host material was BH1-D01, the doped material was BD-1, and the volume ratio of the host material to the doped material in the light-emitting layer was 100:3.
(5) On the organic light-emitting layer, TPBI was vacuum evaporated as an electron transport layer of the organic electroluminescent device. The evaporation rate was 0.01 nm/s, and the evaporation film had a total thickness of 30 nm.
(6) 0.5 nm LiF and 150 nm Al were vacuum evaporated on the electron transport layer as an electron injection layer and a cathode to obtain the organic electroluminescent device.

### Application Examples 2 to 12

Application Examples 2 to 12 provide organic electroluminescent devices, respectively, and differ from Application Example 1 only in that the light-emitting host material BH1-D01 in Application Example 1 was separately replaced with BH1-D02 to BH1-D12, and other structures, materials and preparation methods were all the same as those in Application Example 1.

### Comparative Application Examples 1 to 4

Comparative Application Examples 1 to 4 provide organic electroluminescent devices, respectively, and differ from Application Example 1 only in that the light-emitting host material BH1-D01 in Application Example 1 was separately replaced with BH1, BH1-a, BH1-b and BH1-c, and other structures, materials and preparation methods were all the same as those in Application Example 1.

Structural formulas of BH1, BH1-a, BH1-b, BH1-c, BH2, BH2-a and BH2-b are as follows:

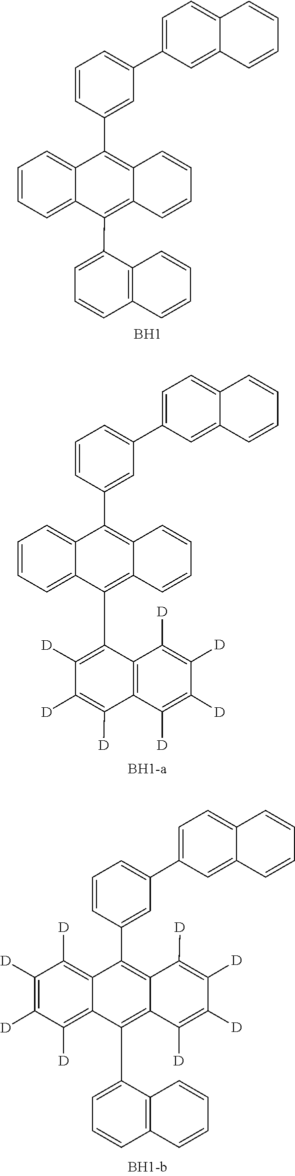

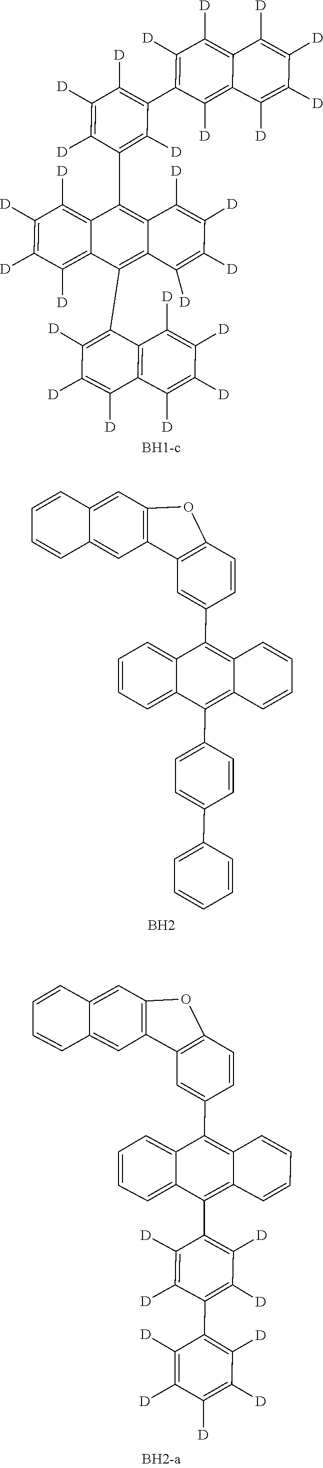

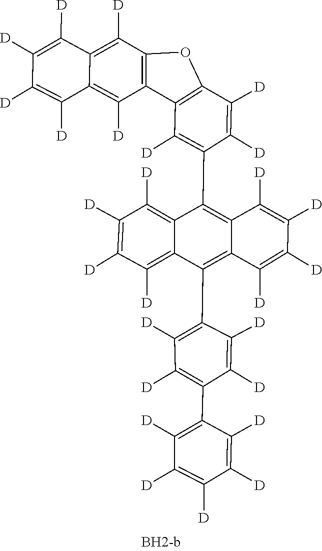 [SMILES: [2H]c1c([2H])c([2H])c(-c2c([2H])c([2H])c(-c3c4c([2H])c([2H])c([2H])c([2H])c4c(-c4c([2H])c([2H])c5oc6c([2H])c7c([2H])c([2H])c([2H])c([2H])c7c([2H])c6c5c4[2H])c4c([2H])c([2H])c([2H])c([2H])c34)c([2H])c2[2H])c([2H])c1[2H]]

Methods for preparing BH1-a and BH2-a are as follows:

### (1) Synthesis of BH1-a

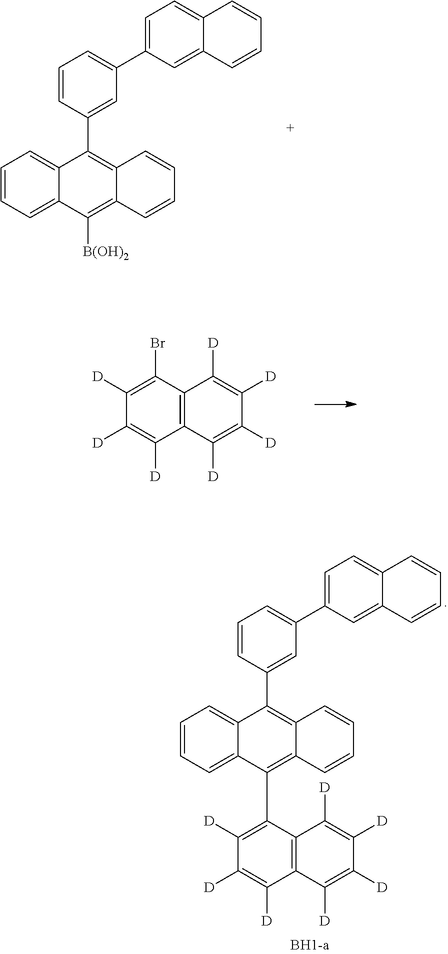 [SMILES: OB(O)c1c2ccccc2c(-c2cccc(-c3ccc4ccccc4c3)c2)c2ccccc12 ; [2H]c1c([2H])c([2H])c2c(Br)c([2H])c([2H])c([2H])c2c1[2H] ; [2H]c1c([2H])c([2H])c2c(-c3c4ccccc4c(-c4cccc(-c5ccc6ccccc6c5)c4)c4ccccc34)c([2H])c([2H])c([2H])c2c1[2H]]

The synthesis method was performed with reference to an existing SUZUKI coupling reaction to obtain the product BH1-a, mass spectrometric detection was performed on BH1-a, and m/z was 513.25.

### (2) Synthesis of BH2-a

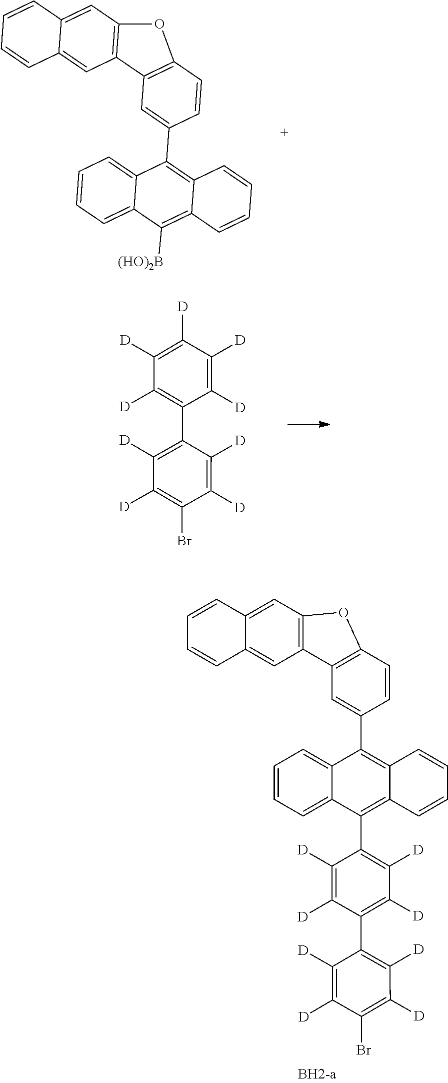 [SMILES: OB(O)c1c2ccccc2c(-c2ccc3oc4cc5ccccc5cc4c3c2)c2ccccc12 ; [2H]c1c([2H])c([2H])c(-c2c([2H])c([2H])c(Br)c([2H])c2[2H])c([2H])c1[2H] ; [2H]c1c([2H])c(-c2c([2H])c([2H])c(-c3c4ccccc4c(-c4ccc5oc6cc7ccccc7cc6c5c4)c4ccccc34)c([2H])c2[2H])c([2H])c([2H])c1Br]

The synthesis method was performed with reference to the existing SUZUKI coupling reaction to obtain the product BH2-a, mass spectrometric detection was performed on BH2-a, and m/z was 555.25.

Deuteration rates of BH1, BH1-a, BH1-b, BH1-c, BH2, BH2-a and BH2-b are shown in the following Table 4. It is to be noted that the deuteration rate data in Table 4 is obtained by means of calculation according to the molecular structures of the products and effects of the purity of the products in actual production processes on the deuteration rates are not considered. If the effects of the purity of the material products on the deuteration rates are considered, actually detected deuteration rates of the products are slightly less than the calculated deuteration rates.

TABLE 4

| | Number of Hydrogen | Number of Deuterium | |
|---|---|---|---|
| Product | Atoms in Single | Atoms in Single | Deuteration |
| Name | Molecule | Molecule | Rate |
| BH1 | 26 | 0 | 0.00% |
| BH1-a | 19 | 7 | 26.92% |
| BH1-b | 18 | 8 | 30.77% |
| BH1-c | 0 | 26 | 100.00% |
| BH2 | 26 | 0 | 0.00% |
| BH2-a | 17 | 9 | 34.62% |
| BH2-b | 0 | 26 | 100.00% |

### Performance Test

An OLED-1000 multi-channel accelerated lifetime & optical property test system for OLED produced by EVERFINE in Hangzhou was used for testing driving voltages, current efficiency and lifetimes LT90 of the organic electroluminescent devices provided above. LT90 refers to a time that brightness needs to reduce to 90% of original brightness in the case where a current density at initial brightness of 2000 nit remains to be constant. The specific test results are shown in Table 5.

TABLE 5

| | Host | | | | | |
|---|---|---|---|---|---|---|
| | Material of | Required | | Current | | Note |
| | Light-emitting | Brightness/ | Driving | Efficiency/ | | (Deuteration |
| | Layer | (cd/m²) | Voltage/V | (cd/A) | LT90/h | Rate) |
| Comparative | BH1 | 1000 | 4.33 | 3.29 | 68 | 0.00% |
| Application | | | | | | |
| Example 1 | | | | | | |
| Comparative | BH1-a | 1000 | 4.26 | 3.58 | 188 | 26.92% |
| Application | | | | | | |
| Example 2 | | | | | | |
| Comparative | BH1-b | 1000 | 4.28 | 3.66 | 190 | 30.77% |
| Application | | | | | | |
| Example 3 | | | | | | |
| Comparative | BH1-c | 1000 | 4.11 | 4.02 | 99 | 100.00% |
| Application | | | | | | |
| Example 4 | | | | | | |
| Application | BH1-D01 | 1000 | 4.02 | 3.55 | 199 | 15.11% |
| Example 1 | | | | | | |
| Application | BH1-D02 | 1000 | 3.99 | 4.12 | 213 | 28.27% |
| Example 2 | | | | | | |
| Application | BH1-D03 | 1000 | 3.16 | 4.18 | 222 | 38.66% |
| Example 3 | | | | | | |
| Application | BH1-D04 | 1000 | 3.11 | 4.26 | 256 | 46.00% |
| Example 4 | | | | | | |
| Application | BH1-D05 | 1000 | 3.12 | 4.26 | 261 | 53.77% |
| Example 5 | | | | | | |
| Application | BH1-D06 | 1000 | 3.33 | 4.11 | 188 | 57.26% |
| Example 6 | | | | | | |
| Application | BH1-D07 | 1000 | 3.33 | 4.16 | 190 | 60.11% |
| Example 7 | | | | | | |
| Application | BH1-D08 | 1000 | 3.29 | 4.09 | 200 | 69.36% |
| Example 8 | | | | | | |
| Application | BH1-D09 | 1000 | 3.33 | 4.00 | 189 | 77.68% |
| Example 9 | | | | | | |
| Application | BH1-D10 | 1000 | 3.42 | 4.10 | 196 | 86.19% |
| Example 10 | | | | | | |
| Application | BH1-D11 | 1000 | 3.39 | 4.02 | 177 | 89.36% |
| Example 11 | | | | | | |
| Application | BH1-D12 | 1000 | 3.37 | 4.02 | 168 | 98.22% |
| Example 12 | | | | | | |

As can be seen from the contents in Comparative Application Examples 1 to 4, as the deuteration rate increases, the voltage gradually decreases, the efficiency gradually increases, and the lifetime first increases and then decreases.

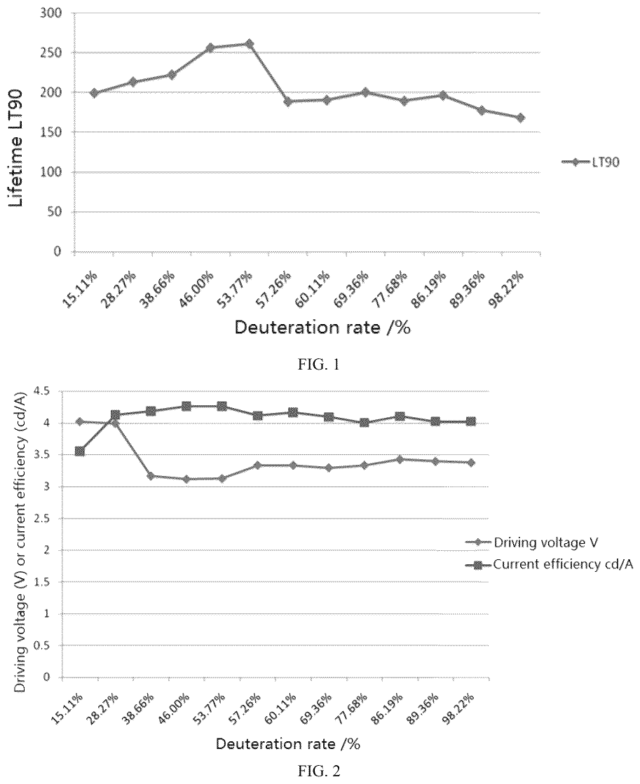

A relationship between the deuteration rates of the host materials of the light-emitting layers and the lifetimes LT90 of the organic electroluminescent devices provided in Application Examples 1 to 12 is shown in FIG. 1. As can be seen from FIG. 1, as the deuteration rate of the host material of the light-emitting layer of the organic electroluminescent device increases, the lifetime LT90 first increases and then decreases.

A relationship between the deuteration rates of the host materials of the light-emitting layers and the driving voltages of the organic electroluminescent devices provided in Application Examples 1 to 12 and a relationship between the deuteration rates of the host materials of the light-emitting layers and current efficiency of the organic electroluminescent devices provided in Application Examples 1 to 12 are shown in FIG. 2. As can be seen from FIG. 2, as the deuteration rate of the host material of the light-emitting layer of the organic electroluminescent device increases, the driving voltage first decreases, then increases and then basically remains constant, and the current efficiency first increases and then basically remains constant.

In the present application, the deuterated composition is a mixture with relatively poor crystal performance and better film formability, and the electroluminescent device prepared in this manner has relatively excellent device performance. The host material used in Application Example 12 has a deuteration rate of 98.22% not significantly different from the deuteration rate of 100% in Comparative Application Example 4 but has relatively good performance. Application Example 2 using the host material with a deuteration rate of 28.27%, Comparative Application Example 2 (the host material has a deuteration rate of 26.92%) and Comparative Application Example 3 (the host material has a deuteration rate of 30.77%) all have relatively good performance. As can be seen from this, when the deuteration rates of the host materials are equivalent, the organic electroluminescent device prepared by the deuterated composition provided in the present application has more excellent performance.

### Application Example 13

This application example provides an organic electroluminescent device and differs from Application Example 1 only in that the host material BH1-D01 of the light-emitting layer in Application Example 1 was replaced with BH1-D05 and the doped material BD-1 was replaced with BD-2, and other structures, materials and preparation methods were all the same as those in Application Example 1.

### Comparative Application Examples 5 and 6

Comparative Application Examples 5 and 6 provide organic electroluminescent devices, respectively, and differ from Application Example 13 only in that the light-emitting host material BH1-D01 in Application Example 13 was replaced with a mixture of BH1-a and BH1-c, and other structures, materials and preparation methods were all the same as those in Application Example 13.

When the light-emitting layers of the organic electroluminescent devices provided in Comparative Application Examples 5 and 6 were prepared, BH1-c and BH1-a were placed in two evaporation sources, respectively, and evaporation rates of BH1-c and BH1-a were controlled so that the mixture of BH1-c and BH1-a that had different deuteration rates was used as the host materials of the light-emitting layers of the organic electroluminescent devices.

The performance of the organic electroluminescent devices provided in Application Example 13 and Comparative Application Examples 5 and 6 was tested. The test method was the same as above. The test results are shown in Table 6.

TABLE 6

| | Host | | | | | |
|---|---|---|---|---|---|---|
| | Material of | Required | | Current | | Note |
| | Light-emitting | Brightness/ | Driving | Efficiency/ | | (Deuteration |
| | Layer | (cd/m²) | Voltage/V | (cd/A) | LT90/h | Rate) |
| Comparative | BH1-a and | 1000 | 4.82 | 3.29 | 66 | 30% |
| Application | BH1-c | | | | | |
| Example 5 | | | | | | |
| Comparative | BH1-a and | 1000 | 4.55 | 3.26 | 65 | 54% |
| Application | BH1-c | | | | | |
| Example 6 | | | | | | |
| Application | BH1-D05 | 1000 | 4.12 | 3.88 | 100 | 53.77% |
| Example 13 | | | | | | |

As can be seen from the contents in Table 6, even if the deuteration rate of the mixture of BH1-c and BH1-a is adjusted to be substantially the same as that of BH1-D05, the performance of the organic electroluminescent device is still relatively poor. As can be seen from this, compared with the simple mixture of the two types of deuterated compounds, the deuterated composition provided in the present application includes more types of deuterated compounds and more components, and the organic electroluminescent device prepared in this manner has more excellent performance.

### Application Examples 14 and 15

Application Examples 14 and 15 provide organic electroluminescent devices, respectively, and differ from Application Example 1 only in that the host material BH1-D01 of the light-emitting layer in Application Example 1 was separately replaced with BH2-D01 and BH2-D02, the doped material BD-1 was replaced with BD-3, and other structures, materials and preparation methods were all the same as those in Application Example 1.

### Comparative Application Examples 7 to 9

Comparative Application Examples 7 to 9 provide organic electroluminescent devices, respectively, and differ from Application Example 14 only in that the light-emitting host material BH2-D01 in Application Example 14 was separately replaced with BH2, BH2-a and BH2-b, and other structures, materials and preparation methods were all the same as those in Application Example 13.

The performance of the organic electroluminescent devices provided in Application Examples 14 and 15 and Comparative Application Examples 7 to 9 was tested. The test method was the same as above. The test results are shown in Table 7.

TABLE 7

| | Host | | | | |
|---|---|---|---|---|---|
| | Material | Required | | Current | Note |
| | of Light- | Bright- | Driving | Effi- | (Deuter- |
| | emitting | ness/ | Voltage/ | ciency/ | ation |
| | Layer | (cd/m²) | V | (cd/A) | Rate) |
| Comparative | BH2 | 1000 | 4.21 | 3.00 | 0.00% |
| Application | | | | | |
| Example 7 | | | | | |
| Comparative | BH2-a | 1000 | 4.16 | 3.07 | 34.62% |
| Application | | | | | |
| Example 8 | | | | | |
| Comparative | BH2-b | 1000 | 4.11 | 3.33 | 100.00% |
| Application | | | | | |
| Example 9 | | | | | |
| Application | BH2- | 1000 | 3.56 | 3.88 | 30.11% |
| Example 14 | D01 | | | | |
| Application | BH2- | 1000 | 3.26 | 3.99 | 58.31% |
| Example 15 | D02 | | | | |

### Application Example 16

Application Example 16 provides an organic electroluminescent device and differs from Application Example 1 only in that the host material BH1-D01 of the light-emitting layer in Application Example 1 was replaced with BH2-D01 and the doped material BD-1 was replaced with BD-4, and other structures, materials and preparation methods were all the same as those in Application Example 1.

The performance of the organic electroluminescent device provided in Application Example 16 was tested. The test method was the same as above. The test results are shown in Table 8.

TABLE 8

| | Host | | | | |
|---|---|---|---|---|---|
| | Material | Required | | Current | Note |
| | of Light- | Bright- | Driving | Effi- | (Deuter- |
| | emitting | ness/ | Voltage/ | ciency/ | ation |
| | Layer | (cd/m²) | V | (cd/A) | Rate) |
| Application | BH2- | 1000 | 3.76 | 4.16 | 30.11% |
| Example | D01 | | | | |
| 16 | | | | | |

As can be seen from the above contents, in the present application, an anthracene compound substituted with an aryl group is subjected to a deuterated reaction to obtain a deuterated mixture, the obtained deuterated mixture is used as a host material for blue light, and an OLED light-emitting device prepared in this manner has a relatively low driving voltage, relatively high current efficiency and a relatively long lifetime. Moreover, in the present application, the process of the deuterated reaction for preparing the deuterated mixture is simple, the reaction condition is mild, and the complex purification process is not needed so that the deuterated mixture is applicable to prepare the organic electroluminescent device.

### Example 3

This example provides a BH11-D series deuterated composition. The deuterated composition was prepared from Compound BH11 by means of a deuterated reaction. A preparation method is as follows:

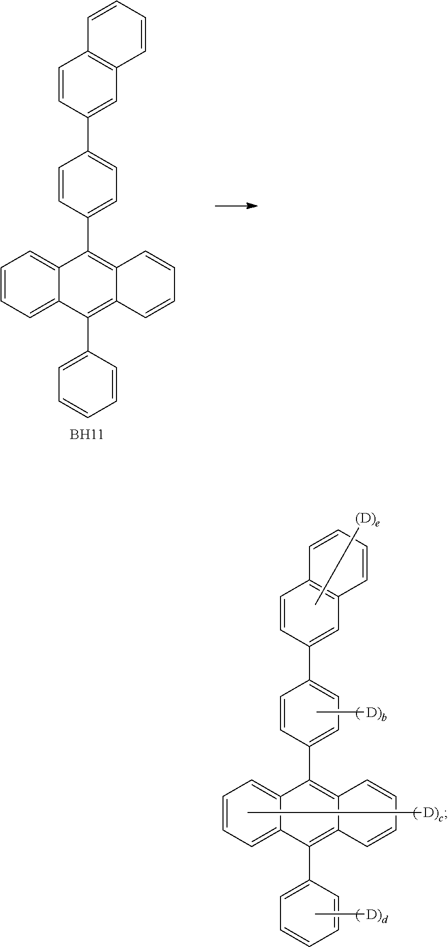

wherein a is selected from integers between 0 and 7, b is selected from integers between 0 and 4, c is selected from integers between 0 and 8, d is selected from integers between 0 and 5, and a+b+c+d≥1.

A specific method for preparing the above BH11-D series deuterated composition is described below.

At room temperature, Compound BH11 (4.56 g, 0.01 mol), palladium (II) chloride (0.0177 g, 0.0001 mol), activated carbon (0.2 g), D₂O (10 mL) and C₆D₆ (50 mL) were added to a 500 mL autoclave, and hydrogen was introduced into the autoclave until the pressure was 0.02 MPa. Then, after the autoclave was heated to 90° C. and a reaction was conducted for a certain time, the autoclave was cooled to room temperature, the system was filtered, and layers were separated. An organic layer obtained after the layers were separated and dried with magnesium sulfate, decolored with a short silica gel column, concentrated to dryness and vacuum dried for 24 h to obtain BH11-D.

Products prepared at different reaction times were weighed. After the products were sublimated, deuteration rates were detected (the deuteration rates were tested through an internal standard method, for example, reference may be made to a method described in a literature “Wu Yurong, Chen Minzhu, Determination of the content of phBr-D with ¹HNMR[J], Journal of Sichuan University: Natural Science Edition, 1997, 34(6): 2” to perform the test). The reaction times and the values of the deuteration rates of the products are shown in the following Table 9.

TABLE 9

| | Deuterated | Reaction | Product | Deuteration |
|---|---|---|---|---|
| No. | Composition | Time/(h) | Weight/(g) | Rate |
| 1 | BH11-D01 | 1 | 4.31 | 15.31% |
| 2 | BH11-D02 | 10 | 4.36 | 58.17% |
| 3 | BH11-D03 | 40 | 4.49 | 88.16% |
| 4 | BH11-D04 | 80 | 4.51 | 97.90% |

### Example 4

This example provides a BH21-D series deuterated composition. The deuterated composition was prepared from Compound BH21 by means of a deuterated reaction. A preparation method is as follows:

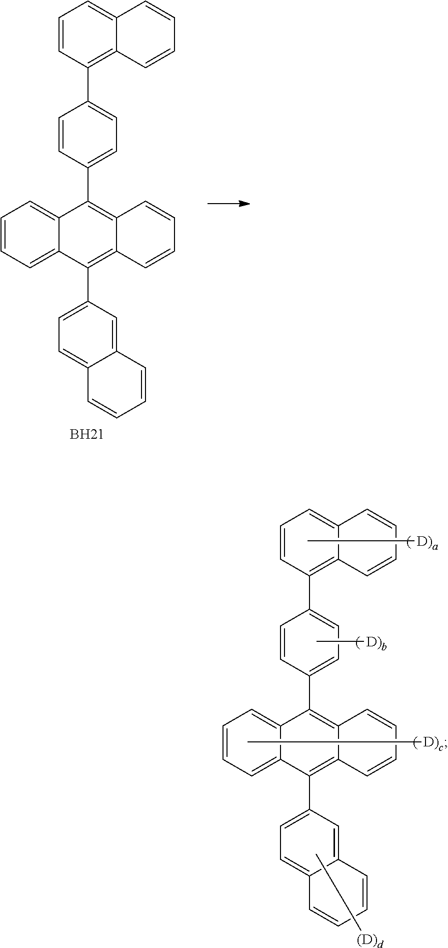

wherein a is selected from integers between 0 and 7, b is selected from integers between 0 and 4, c is selected from integers between 0 and 8, d is selected from integers between 0 and 7, and a+b+c+d≥1.

A specific method for preparing the above BH21-D series deuterated composition is described below.

At room temperature, Compound BH21 (5.06 g, 0.01 mol), palladium (II) chloride (0.0177 g, 0.0001 mol), activated carbon (0.2 g), D₂O (12 mL) and C₆D₆ (50 mL) were added to a 500 mL autoclave, and hydrogen was introduced into the autoclave until the pressure was 0.02 MPa. Then, after the autoclave was heated to 90° C. and a reaction was conducted for a certain time, the autoclave was cooled to room temperature, the system was filtered, and layers were separated. An organic layer obtained after the layers were separated was dried with magnesium sulfate, decolored with a short silica gel column, concentrated to dryness and vacuum dried for 24 h to obtain BH21-D.

Products prepared at different reaction times were weighed. After the products were sublimated, deuteration rates were detected (a test method was the same as above). The reaction times and the values of the deuteration rates of the products are shown in the following Table 10.

TABLE 10

| | Deuterated | Reaction | Product | Deuteration |
|---|---|---|---|---|
| No. | Composition | Time/(h) | Weight/(g) | Rate |
| 1 | BH21-D01 | 1 | 4.91 | 16.12% |
| 2 | BH21-D02 | 10 | 5.00 | 51.32% |
| 3 | BH21-D03 | 40 | 5.13 | 78.26% |
| 4 | BH21-D04 | 80 | 5.15 | 96.99% |

### Example 5

This example provides a BH3-D series deuterated composition. The deuterated composition was prepared from Compound BH3 by means of a deuterated reaction. A preparation method is as follows:

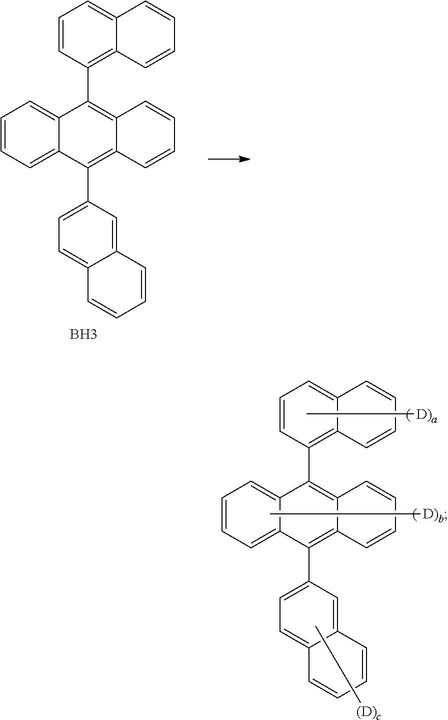

wherein a is selected from integers between 0 and 7, b is selected from integers between 0 and 8, c is selected from integers between 0 and 7, and a+b+c≥1.

For the above method for preparing the BH3-D series deuterated composition, reference may be made to the method for preparing the BH2-D series deuterated composition, that is, Compound BH2 was replaced with Compound BH3 in an equivalent amount of substance.

Products prepared at different reaction times were weighed. After the products were sublimated, deuteration rates were detected (a test method was the same as above). The reaction times and the values of the deuteration rates of the products are shown in the following Table 11.

TABLE 11

| | Deuterated | Reaction | Product | Deuteration |
|---|---|---|---|---|
| No. | Composition | Time/(h) | Weight/(g) | Rate |
| 1 | BH3-D01 | 1 | 4.13 | 18.26% |
| 2 | BH3-D02 | 10 | 4.21 | 46.19% |
| 3 | BH3-D03 | 40 | 4.31 | 79.06% |
| 4 | BH3-D04 | 80 | 4.35 | 97.49% |

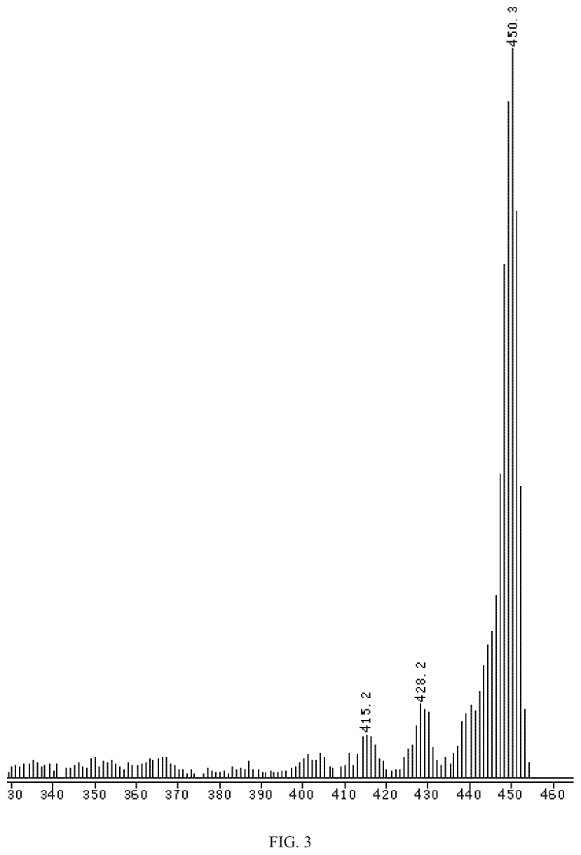

The deuterated composition BH3-D03 was sublimated and subjected to a mass spectrometry test. For details of test results, see FIG. 3. For details of some data and analysis in FIG. 3, see the following Table 12:

TABLE 12

| | How Many D Atoms in Compound | |
|---|---|---|
| m/z | Contained in Product Can Be Indicated | |
| 452 | 22 | a single compound |
| 451 | 21 | not a single compound |
| 450 | 20 | not a single compound |
| 449 | 19 | not a single compound |
| 448 | 18 | not a single compound |
| 447 | 17 | not a single compound |
| 446 | 16 | not a single compound |
| 445 | 15 | not a single compound |
| 444 | 14 | not a single compound |
| 443 | 13 | not a single compound |
| 442 | 12 | not a single compound |
| 441 | 11 | not a single compound |
| 440 | 10 | not a single compound |
| 439 | 9 | not a single compound |
| 438 | 8 | not a single compound |
| 437 | 7 | not a single compound |
| 436 | 6 | not a single compound |

It is to be noted that for ease of recording, the data of m/z in Table 12 are all integers. For example, as can be seen from FIG. 3, the data of m/z is 450.3, which is recorded as an integer 450 in Table 12.

After the deuterated composition BH3-D03 is subjected to the mass spectrometry test, a peak whose m/z is 452 exists, and peaks whose m/z are 453 and 454 are detected, indicating that a compound having 22 deuterium atoms contained in the product is detected. As can be seen from the structure of BH3, after all hydrogen atoms in BH3 are substituted with deuterium atoms, a deuterated substance containing 22 deuterium atoms can be obtained. Therefore, the deuterated substance containing 22 deuterium atoms has a determined structure and is a single compound. A peak whose m/z is 451 indicates that a compound having 21 deuterium atoms contained in the product is detected. As can be seen from the structure of BH3, after only one hydrogen atom in BH3 is not substituted with a deuterium atom and other hydrogen atoms are all substituted with deuterium atoms, a deuterated substance containing 21 deuterium atoms can be obtained. Since the activity of the hydrogen atoms in BH3 is not significantly different, it may be that any one of the hydrogen atoms in BH3 is not be substituted with a deuterium atom. Therefore, the deuterated substance containing 21 deuterium atoms has an uncertain structure and is not a single compound. Similarly, for each of the compounds corresponding to m/z of 436 to 450 in Table 12, a deuterated substance also has an uncertain structure and is not a single compound.

In addition, as can be seen according to FIG. 3, a maximum peak of an abundance of m/z is 450, indicating that the deuterated composition BH3-D03 contains multiple types of compounds but a compound with a largest content is a compound having 20 H atoms substituted with D atoms. It is to be noted that the compound having 20 H atoms substituted with D atoms is not a single compound but a mixture obtained after a hydrogen atom at a not particular position is substituted with a deuterium atom.

### Example 6

This example provides a BH4-D series deuterated composition. The deuterated composition was prepared from Compound BH4 by means of a deuterated reaction. A preparation method is as follows:

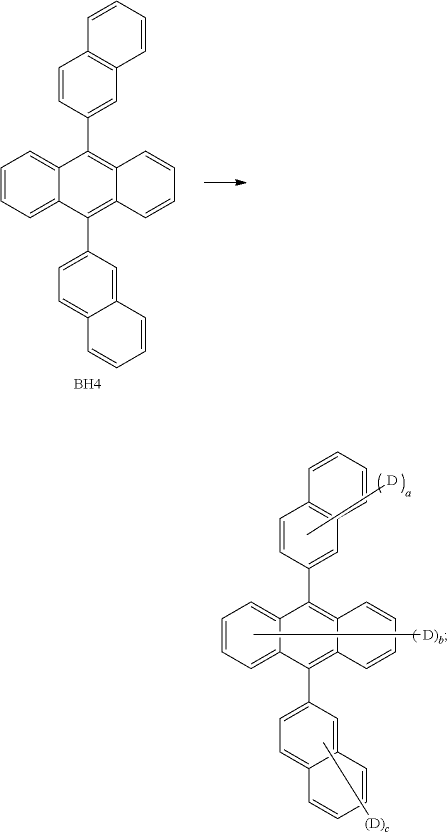

wherein a is selected from integers between 0 and 7, b is selected from integers between 0 and 8, c is selected from integers between 0 and 7, and a+b+c≥1.

For the above method for preparing the BH4-D series deuterated composition, reference may be made to the method for preparing the BH2-D series deuterated composition, that is, Compound BH2 was replaced with Compound BH4 in an equivalent amount of substance.

Products prepared at different reaction times were weighed. After the products were sublimated, deuteration rates were detected (a test method was the same as above). The reaction times and the values of the deuteration rates of the products are shown in the following Table 13.

TABLE 13

| | Deuterated | Reaction | Product | Deuteration |
|---|---|---|---|---|
| No. | Composition | Time/(h) | Weight/(g) | Rate |
| 1 | BH4-D01 | 1 | 4.02 | 17.21% |
| 2 | BH4-D02 | 10 | 4.11 | 47.13% |
| 3 | BH4-D03 | 40 | 4.39 | 78.67% |
| 4 | BH4-D04 | 80 | 4.38 | 98.19% |

### Example 7

This example provides a BH5-D series deuterated composition. The deuterated composition was prepared from Compound BH5 by means of a deuterated reaction. A preparation method is as follows:

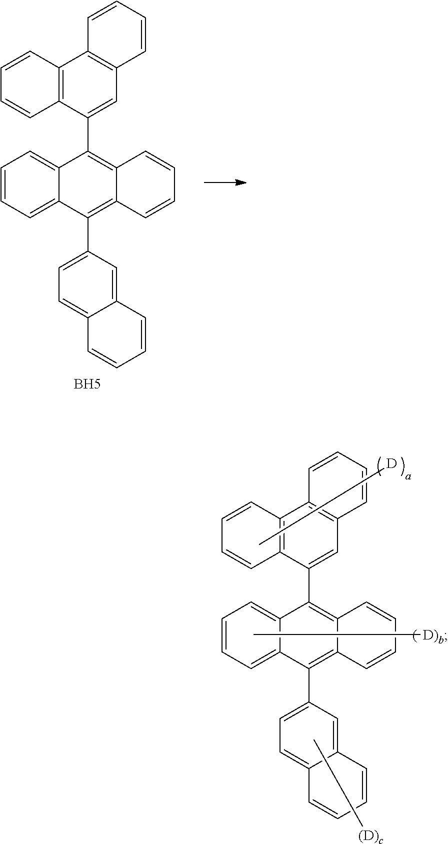

wherein a is selected from integers between 0 and 9, b is selected from integers between 0 and 8, c is selected from integers between 0 and 7, and a+b+c≥1.

For the above method for preparing the BH5-D series deuterated composition, reference may be made to the method for preparing the BH2-D series deuterated composition, that is, Compound BH2 was replaced with Compound BH5 in an equivalent amount of substance.

Products prepared at different reaction times were weighed. After the products were sublimated, deuteration rates were detected (a test method was the same as above). The reaction times and the values of the deuteration rates of the products are shown in the following Table 14.

TABLE 14

| | Deuterated | Reaction | Product | Deuteration |
|---|---|---|---|---|
| No. | Composition | Time/(h) | Weight/(g) | Rate |
| 1 | BH5-D01 | 1 | 4.68 | 17.99% |
| 2 | BH5-D02 | 10 | 4.72 | 58.79% |
| 3 | BH5-D03 | 40 | 4.76 | 81.21% |
| 4 | BH5-D04 | 80 | 4.89 | 97.91% |

### Example 8

This example provides a BH34-D series deuterated composition. The deuterated composition was prepared from a mixture of Compounds BH3 and BH4 by means of a deuterated reaction. A preparation method is as follows:

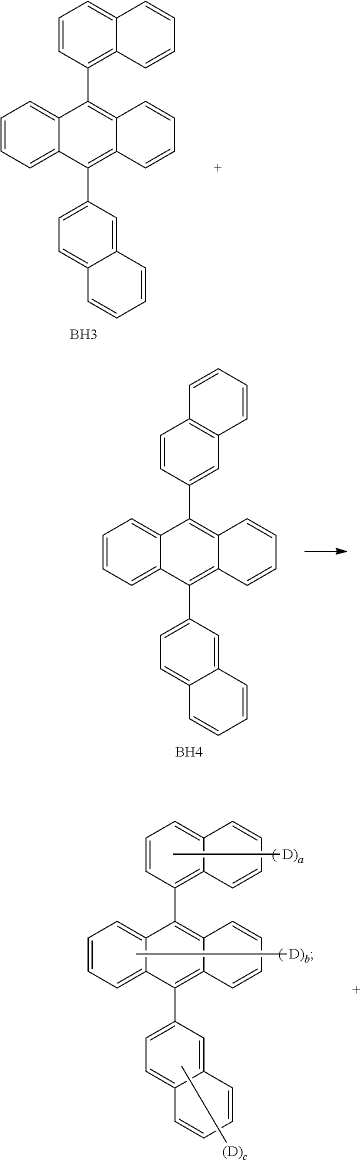

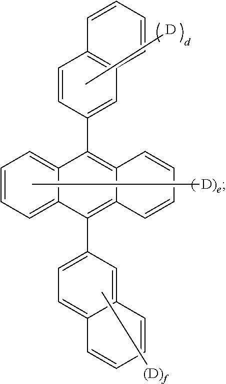

wherein a and d are selected from integers between 0 and 7, b and e are selected from integers between 0 and 8, c and f are selected from integers between 0 and 7, and a+b+c+d+e+f≥2.

A specific method for preparing the above BH34-D series deuterated composition is described below.

At room temperature, BH3 (2.15 g, 0.005 mol), BH4 (2.15 g, 0.005 mol), palladium (II) chloride (0.0177 g, 0.0001 mol), activated carbon (0.2 g), D₂O (12 mL) and C₆D₆ (50 mL) were added to a 500 mL autoclave, and hydrogen was introduced into the autoclave until the pressure was 0.02 MPa. Then, after the autoclave was heated to 90° C. and a reaction was conducted for a certain time, the autoclave was cooled to room temperature, the system was filtered, and layers were separated. An organic layer obtained after the layers were separated was dried with magnesium sulfate, decolored with a short silica gel column, concentrated to dryness and vacuum dried for 24 h to obtain BH34-D.

Products prepared at different reaction times were weighed. After the products were sublimated, deuteration rates were detected (a test method was the same as above). The reaction times and the values of the deuteration rates of the products are shown in the following Table 15.

TABLE 15

| | Deuterated | Reaction | Product | Deuteration |
|---|---|---|---|---|
| No. | Composition | Time/(h) | Weight/(g) | Rate |
| 1 | BH34-D01 | 1 | 4.15 | 18.45% |
| 2 | BH34-D02 | 10 | 4.21 | 55.46% |
| 3 | BH34-D03 | 40 | 4.38 | 79.96% |
| 4 | BH34-D04 | 80 | 4.41 | 98.79% |

In a process of preparing an OLED device, the deuterated composition provided in the present application needs to be evaporated in an evaporation manner. Therefore, there is a need to ensure that to-be-evaporated components remain relatively stable, and the following evaporation experiment is performed: 2 g BH11-D03 was taken, placed in a crucible of an evaporator and heated to 300° C. at a pressure of 1×10⁻⁵-9×10⁻⁵ Pa, the material in the crucible was sequentially evaporated to glass substrates whose serial numbers were 1 to 5 until the material in the crucible remained about 10%, and a total of five glass substrates were obtained according to the sequence of the evaporation.

In addition, referring to the above method, BH3-D02 or BH34-D01 was measured, and a total of ten glass substrates were obtained.

Deuteration rates of materials on these fifteen glass substrates were separately analyzed, as shown in the following Table 16.

TABLE 16

| Glass Substrate No. | 1 | 2 | 3 | 4 | 5 |
|---|---|---|---|---|---|
| BH11-D03 | 88.10 | 87.10 | 87.60 | 88.26 | 87.11 |
| Deuteration Rate/(%) | | | | | |
| BH3-D02 | 46.11 | 46.18 | 47.12 | 47.11 | 46.29 |
| Deuteration Rate/(%) | | | | | |
| BH34-D01 | 18.49 | 17.91 | 18.02 | 19.01 | 18.00 |
| Deuteration Rate/(%) | | | | | |

As can be seen from the contents in Table 16, the deuteration rates of the materials evaporated to the glass substrates in different periods are basically the same, indicating that the components of the deuterated composition provided in the present application can remain to be relatively stable in the evaporation process.

### Comparative Example 1

This comparative example provides BH-D. BH-D was prepared with reference to a preparation method provided in Chinese Patent CN102428158A:

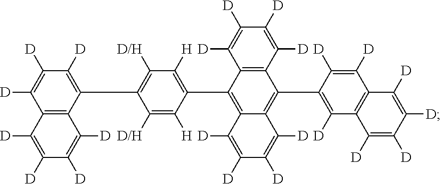

wherein “D/H” indicates an approximately equal probability of H or D at the atomic position.

A deuteration rate of BH-D is between (22/26)*100% and (24/26)*100%, that is, between 84.6% and 92.3%. Further, as can be seen according to description in CN102428158A, in BH-D, uncertain substitution of a D atom only exists at a particular position of a benzene ring, and D atoms definitely exist in other groups except the benzene ring.

Further, it can be seen that a determined structure of BH-D is one of the following compounds or a mixture of the two compounds:

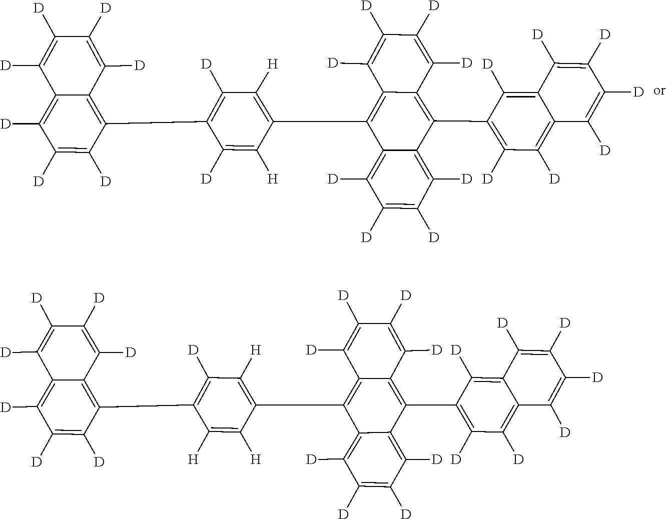 [SMILES: [2H]c1cc(-c2c3c([2H])c([2H])c([2H])c([2H])c3c(-c3c([2H])c([2H])c4c([2H])c([2H])c([2H])c([2H])c4c3[2H])c3c([2H])c([2H])c([2H])c([2H])c23)cc([2H])c1-c1c([2H])c([2H])c([2H])c2c([2H])c([2H])c([2H])c([2H])c12 ; [2H]c1cc(-c2c3c([2H])c([2H])c([2H])c([2H])c3c(-c3c([2H])c([2H])c4c([2H])c([2H])c([2H])c([2H])c4c3[2H])c3c([2H])c([2H])c([2H])c([2H])c23)ccc1-c1c([2H])c([2H])c([2H])c2c([2H])c([2H])c([2H])c([2H])c12]

Other compounds for which specific synthesis steps are not listed may be prepared according to common knowledge in the art in combination with the preceding examples.

Other specific structures used in the following application examples are shown as follows:

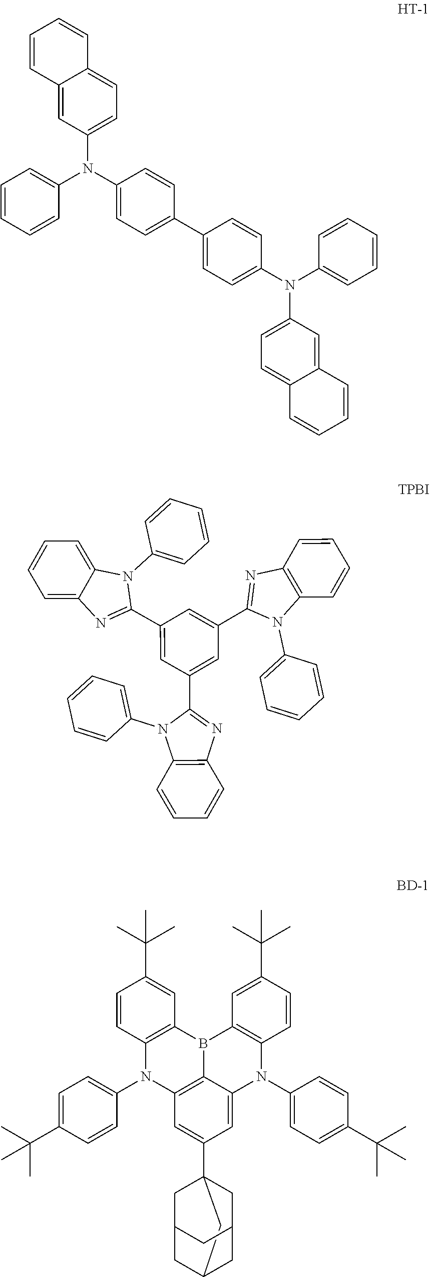 [SMILES: c1ccc(N(c2ccc(-c3ccc(N(c4ccccc4)c4ccc5ccccc5c4)cc3)cc2)c2ccc3ccccc3c2)cc1 ; c1ccc(-n2c(-c3cc(-c4nc5ccccc5n4-c4ccccc4)cc(-c4nc5ccccc5n4-c4ccccc4)c3)nc3ccccc32)cc1 ; CC(C)(C)c1ccc(N2c3ccc(C(C)(C)C)cc3B3c4cc(C(C)(C)C)ccc4N(c4ccc(C(C)(C)C)cc4)c4cc(C56CC7CC(CC(C7)C5)C6)cc2c43)cc1]

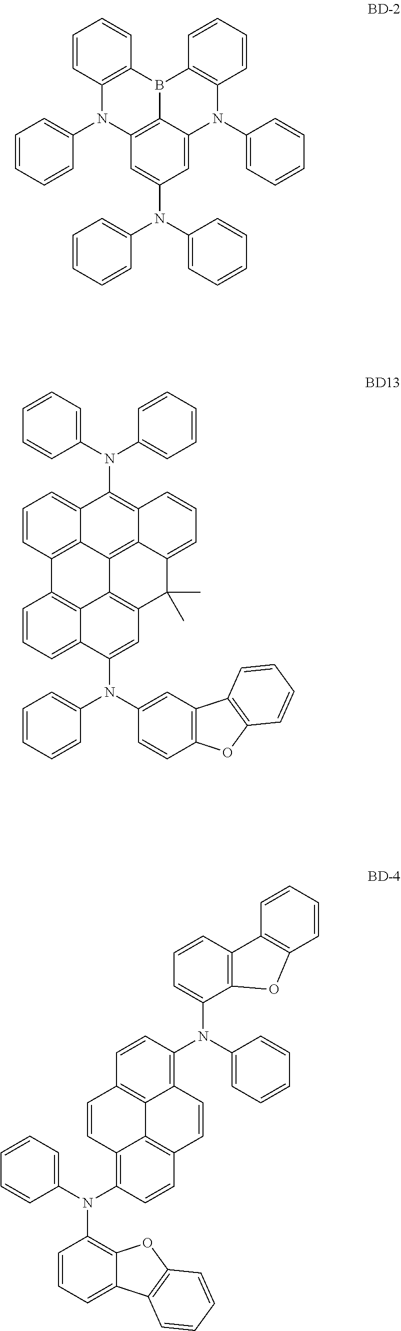 [SMILES: c1ccc(N(c2ccccc2)c2cc3c4c(c2)N(c2ccccc2)c2ccccc2B4c2ccccc2N3c2ccccc2)cc1 ; CC1(C)c2cccc3c(N(c4ccccc4)c4ccccc4)c4cccc5c6cccc7c(N(c8ccccc8)c8ccc9oc%10ccccc%10c9c8)cc1c(c76)c(c23)c45 ; c1ccc(N(c2ccc3ccc4c(N(c5ccccc5)c5cccc6c5oc5ccccc56)ccc5ccc2c3c54)c2cccc3c2oc2ccccc23)cc1]

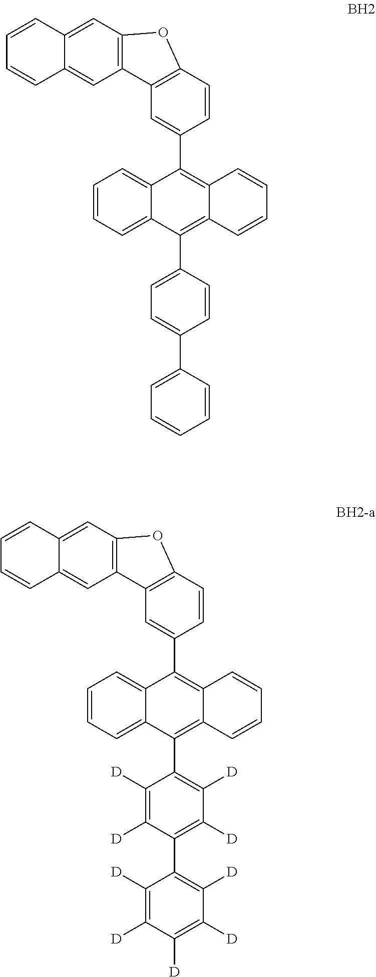 [SMILES: c1ccc(-c2ccc(-c3c4ccccc4c(-c4ccc5oc6cc7ccccc7cc6c5c4)c4ccccc34)cc2)cc1 ; [2H]c1c([2H])c([2H])c(-c2c([2H])c([2H])c(-c3c4ccccc4c(-c4ccc5oc6cc7ccccc7cc6c5c4)c4ccccc34)c([2H])c2[2H])c([2H])c1[2H]]

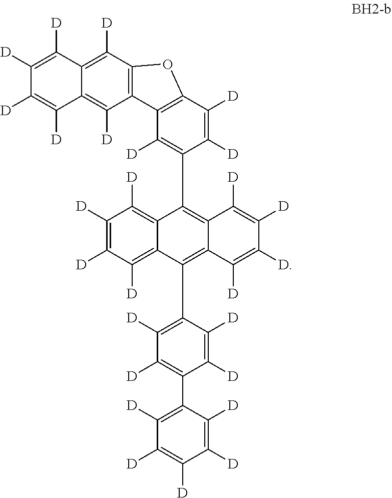

Deuteration rates of BH2, BH2-a and BH2-b are shown in the following Table 17. It is to be noted that the deuteration rate data in Table 17 is obtained by means of calculation according to the molecular structures of the products and effects of the purity of the products in actual production processes on the deuteration rates are not considered. If the effects of the purity of the material products on the deuteration rates are considered, actually detected deuteration rates of the products are slightly less than the calculated deuteration rates.

TABLE 17

| | Number of Hydrogen | Number of | |
|---|---|---|---|
| Product | Atoms in Single | Deuterium Atoms in | Deuteration |
| Name | Molecule | Single Molecule | Rate |
| BH2 | 26 | 0 | 0.00% |
| BH2-a | 17 | 9 | 34.62% |
| BH2-b | 0 | 26 | 100.00% |

### Application Example 17

This application example provides an organic electroluminescent device. A structure of the organic electroluminescent device is ITO/HT-1 (40 nm) light-emitting layer (30 nm): BD13 (3%)/TPBI (30 nm)/LiF (0.5 nm)/Al (150 nm).

A method for preparing the above organic electroluminescent device is described below.
(1) A glass substrate coated with the ITO transparent conductive layer (as an anode) was ultrasonically treated in a cleaning agent, flushed in deionized water, ultrasonically degreased in a mixed solvent of acetone and ethanol, dried and cleaned with ultraviolet and ozone. A surface was bombarded with a low-energy cation beam to improve an energy level property of the ITO surface, thereby improving an ability to bind to a hole injection layer.
(2) The above glass substrate was placed in a vacuum chamber, the vacuum chamber was vacuumed to 1×10⁻⁵-1×10⁻⁴ Pa, and HT-1 was vacuum evaporated on the anode as a hole transport layer. The evaporation rate was 0.01 nm/s, and the evaporation film had a thickness of 40 nm.
(3) On the hole transport layer, the light-emitting layer was vacuum evaporated. The evaporation rate was 0.01 nm/s, and the evaporation film had a total thickness of 30 nm. In the light-emitting layer, the host material was BH11-D01, the doped material was BD13, and the volume ratio of the host material to the doped material in the light-emitting layer was 100:3.
(4) On the organic light-emitting layer, TPBI was vacuum evaporated as an electron transport layer of the organic electroluminescent device. The evaporation rate was 0.01 nm/s, and the evaporation film had a total thickness of 30 nm.
(5) 0.5 nm LiF and 150 nm Al were vacuum evaporated on the electron transport layer as an electron injection layer and a cathode to obtain the organic electroluminescent device.

### Application Examples 17 to 30

Application Examples 17 to 30 provide organic electroluminescent devices, respectively, and differ from Application Example 17 only in that the light-emitting host material BH11-D01 in Application Example 17 was replaced (for details, see Table 18), and other structures, materials and preparation methods were all the same as those in Application Example 17.

### Comparative Application Examples 16 to 18

Comparative Application Examples 16 to 18 provide organic electroluminescent devices, respectively, and differ from Application Example 17 only in that the light-emitting host material BH11-D01 in Application Example 17 was replaced (for details, see Table 18), and other structures, materials and preparation methods were all the same as those in Application Example 17.

### Performance Test

An OLED-1000 multi-channel accelerated lifetime & optical property test system for OLED produced by EVERFINE in Hangzhou was used for testing driving voltages, current efficiency and lifetimes LT90 of the organic electroluminescent devices provided above. LT90 refers to a time that brightness needs to reduce to 90% of original brightness in the case where a current density at initial brightness of 2000 nit remains to be constant. The specific test results are shown in Table 18, and the voltages, the current efficiency and LT90 are all relative values:

TABLE 18

| | Host | | | | |
|---|---|---|---|---|---|
| | Material | | | | |
| | of Light- | Required | | | Note |
| | emitting | Brightness/ | Driving | | (Deuteration |
| | Layer | (cd/m²) | Voltage | LT90 | Rate) |
| Comparative Application | BH-D | 1000 | 1.57 | 0.58 | 84.6~92.3% |
| Example 16 | | | | | |
| Comparative Application | BH2-a | 1000 | 1.59 | 0.36 | 34.62% |
| Example 17 | | | | | |
| Comparative Application | BH2-b | 1000 | 1.61 | 0.41 | 100.00% |
| Example 18 | | | | | |
| Application Example 16 | BH11-D01 | 1000 | 1 | 1 | 15.31% |
| Application Example 17 | BH11-D02 | 1000 | 0.78 | 1.86 | 58.17% |
| Application Example 18 | BH11-D03 | 1000 | 0.86 | 1.18 | 88.16% |
| Application Example 19 | BH11-D04 | 1000 | 1.02 | 0.89 | 97.90% |
| Application Example 20 | BH21-D02 | 1000 | 0.80 | 1.27 | 51.32% |
| Application Example 21 | BH21-D03 | 1000 | 0.71 | 1.97 | 78.26% |
| Application Example 22 | BH3-D01 | 1000 | 0.86 | 1.22 | 18.26% |
| Application Example 23 | BH3-D02 | 1000 | 0.51 | 1.97 | 46.19% |
| Application Example 24 | BH3-D03 | 1000 | 0.79 | 1.29 | 79.06% |
| Application Example 25 | BH3-D04 | 1000 | 0.81 | 1.21 | 97.49% |
| Application Example 26 | BH5-D03 | 1000 | 0.88 | 1.76 | 81.21% |
| Application Example 27 | BH34-D01 | 1000 | 0.66 | 1.66 | 18.45% |
| Application Example 28 | BH34-D02 | 1000 | 0.58 | 2.38 | 55.46% |
| Application Example 29 | BH34-D03 | 1000 | 0.33 | 3.81 | 79.96% |
| Application Example 30 | BH34-D04 | 1000 | 0.61 | 1.56 | 98.79% |

As can be seen from the contents in Table 18, in the present application, an anthracene compound substituted with a particular aryl group is subjected to a deuterated reaction to obtain a deuterated mixture, the obtained deuterated mixture is used as a host material of a light-emitting layer, and an OLED light-emitting device prepared in this manner has a relatively low driving voltage and a relatively long lifetime.

As can be seen from Application Examples 16 to 19, Application Examples 22 to 25 and Application Examples 27 to 30, as the deuteration rate of the deuterated composition increases, for the organic electroluminescent device prepared by using the deuterated compound as the host material of the light-emitting layer, the driving voltage first decreases and then increases, and the service life first increases and then decreases.

As can be seen from Application Examples 27 to 30, when the deuterated composition is prepared using two types of compounds represented by Formula I′, the performance of the organic electroluminescent device can be further improved. The device performance can be further improved.

When the deuteration rate of the deuterated composition is between 46.1% and 88.2%, the organic electroluminescent device prepared by using the deuterated compound as the host material of the light-emitting layer has a relatively low driving voltage and a relatively long service life, and the overall performance is more excellent.

As can be seen from comparison between Application Examples 18 and 19, as the deuteration rate of the host material of the light-emitting layer increases (Application Example 19), the lifetime of the organic electroluminescent device decreases. The reason may be as follows: as the deuteration rate increases, more H atoms in BH11 are substituted with deuterium atoms, and in this manner, types of a compound substituted with the particular number of deuterium atoms are reduced, that is, in Application Example 19, types of a compound having a specific and particular structure are reduced, resulting in relatively poor film formability of the material and an effect on the lifetime.

Further explanation is as follows: in the case where H atoms are all substituted with deuterium atoms, only one certain structure exists: the number of compounds each having a determined structure formed after only one H atom is not substituted with a deuterium atom is less than the number of compounds each having a determined structure formed after only two H atoms are not substituted with deuterium atoms: the number of compounds each having the determined structure formed after only two H atoms are not substituted with deuterium atoms is less than the number of compounds each having a determined structure formed after only three H atoms are not substituted with deuterium atoms; . . . . However, after about half of the hydrogen atoms are substituted with deuterium atoms, as the number of H atoms in the molecular structure increases, the number of compounds each having a determined structure in the deuterated composition gradually decreases.

In the present application, the deuterated composition is a mixture of multiple components with relatively poor crystal performance and better film formability, and the electroluminescent device prepared in this manner has relatively excellent device performance.

### Application Example 31

This application example provides an organic electroluminescent device and differs from Application Example 17 only in that the host material BH11-D01 of the light-emitting layer in Application Example 17 was replaced with BH4-D02 and the doped material was BD-13 (for details, see Table 19), and other structures, materials and preparation methods were all the same as those in Application Example 17.

### Application Examples 32 to 34

Application Examples 32 to 34 provide organic electroluminescent devices, respectively, and differ from Application Example 31 only in that the doped materials were different (for details, see Table 19), and other structures, materials and preparation methods were all the same as those in Application Example 28.

The performance of the organic electroluminescent devices provided in Application Examples 32 to 34 was tested. The test method was the same as above. The test results are shown in Table 19.

TABLE 19

| | Doped | Required | Driving | |
|---|---|---|---|---|
| | Material | Brightness/(cd/m²) | Voltage | LT90 |
| Application Example 31 | BD13 | 1000 | 1 | 1 |
| Application Example 32 | BD-1 | 1000 | 1.21 | 0.78 |
| Application Example 33 | BD-2 | 1000 | 1.17 | 0.92 |
| Application Example 34 | BD-4 | 1000 | 1.58 | 0.21 |

As can be seen from the contents in Table 19, the deuterated composition provided in the present application is used as the host material of the light-emitting layer, and the compound (BD13) having a structure represented by Formula BDI is used as the doped material of the light-emitting layer, thereby further reducing the driving voltage of the organic electroluminescent device and improving the service life of the organic electroluminescent device.

In conclusion, in the present application, an anthracene compound substituted with a particular aryl group is subjected to a deuterated reaction to obtain a deuterated mixture, the obtained deuterated mixture is used as a host material of a light-emitting layer, and an OLED light-emitting device prepared in this manner has a relatively low driving voltage and a relatively long lifetime. Moreover, the compound (BD13) having the structure represented by Formula BDI is used as the doped material of the light-emitting layer, thereby further reducing the driving voltage of the organic electroluminescent device and improving the service life of the organic electroluminescent device.

The applicant has stated that although the detailed process flow of the present application is described through the examples described above, the present application is not limited to the detailed process flow described above, which means that the implementation of the present application does not necessarily depend on the detailed process flow described above. It should be apparent to those skilled in the art that any improvements made to the present application, equivalent replacements of raw materials of the product of the present application, additions of adjuvant ingredients, selections of specific manners, etc., all fall within the protection scope and the disclosure scope of the present application.

1. A deuterated composition, comprising a deuterated mixture prepared from Compound A by means of a deuterated reaction;
wherein Compound A has a structure represented by Formula I: 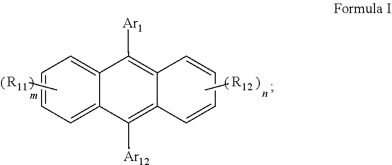
wherein Ar₁₁ and Ar₁₂ are each independently selected from any one of substituted or unsubstituted C6 to C40 aryl, or substituted or unsubstituted C12 to C40 heteroaryl;
R₁₁ and R₁₂ are each independently selected from any one of deuterium, substituted or unsubstituted C1 to C6 linear or branched alkyl, substituted or unsubstituted C6 to C40 aryl, or substituted or unsubstituted C12 to C40 heteroaryl;
substituted substituents in Ar₁₁, Ar₁₂, R₁₁ and R₁₂ are each independently selected from at least one of C1 to C6 linear or branched alkyl, C6 to C20 aryl, or C6 to C20 heteroaryl; and
m and n are each independently selected from integers between 0 and 4.

2. The deuterated composition according to claim 1, wherein Compound A has a structure represented by Formula I′: 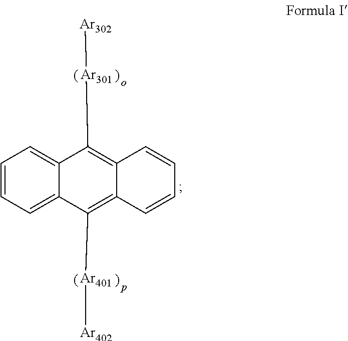
wherein Ar₃₀₁, Ar₃₀₂, Ar₄₀₁ and Ar₄₀₂ are each independently selected from one of phenyl, naphthyl or phenanthryl, and Ar₃₀₁, Ar₃₀₂, Ar₄₀₁ and Ar₄₀₂ are not phenanthryl at the same time; the structure represented by Formula I′ contains only one phenanthryl; and
o and p are each independently selected from 0 or 1.

3. The deuterated composition according to claim 2, wherein Compound A is not

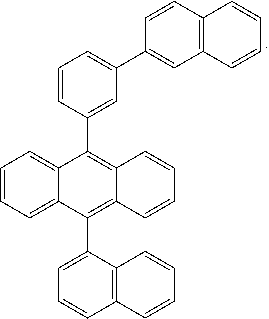 [SMILES: c1cc(-c2ccc3ccccc3c2)cc(-c2c3ccccc3c(-c3cccc4ccccc34)c3ccccc23)c1]

4. The deuterated composition according to claim 2,
wherein the deuterated composition comprises a deuterated mixture prepared from two types of Compound A by means of a deuterated reaction;
preferably, the deuterated mixture comprises at least five types of compounds;
preferably, the deuterated mixture comprises at least six types of compounds;
preferably, the deuterated mixture comprises at least seven types of compounds.

5. The deuterated composition according to claim 2, wherein Compound A is selected from any one of the following compounds:

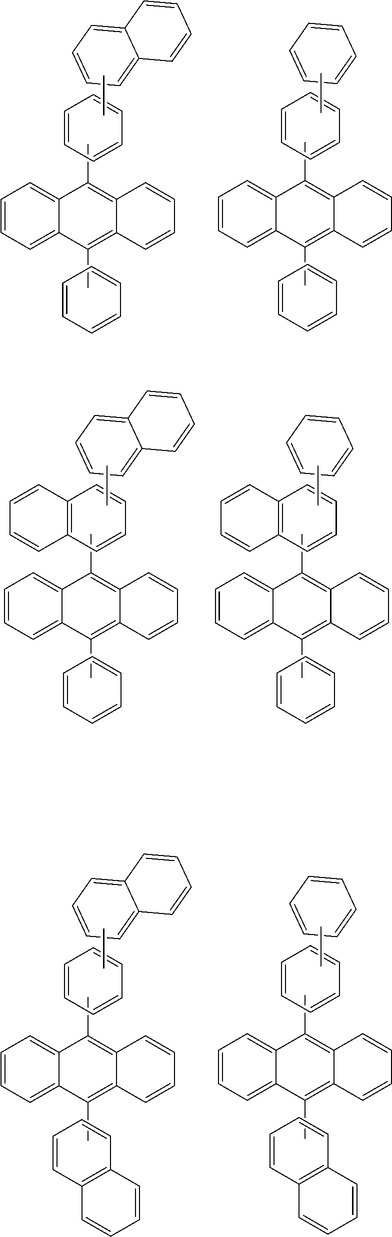

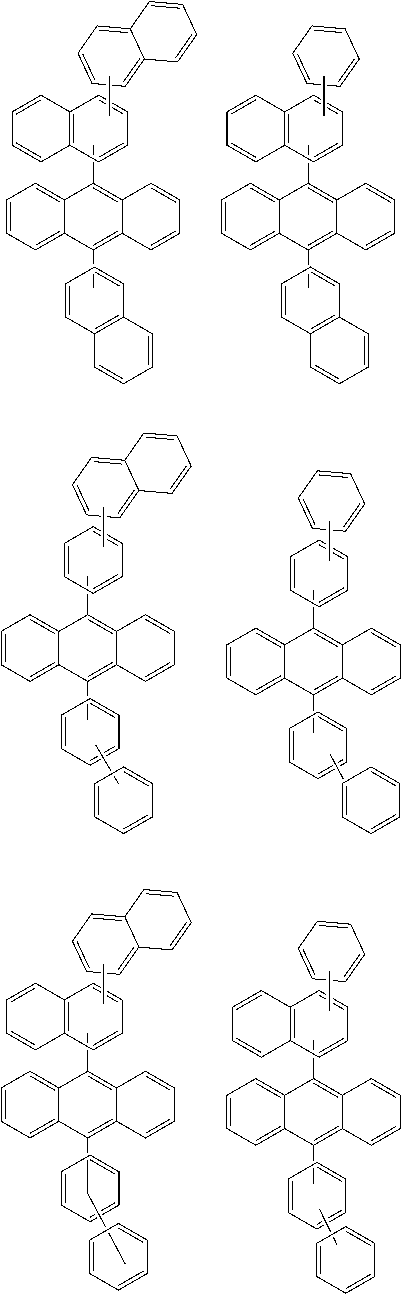

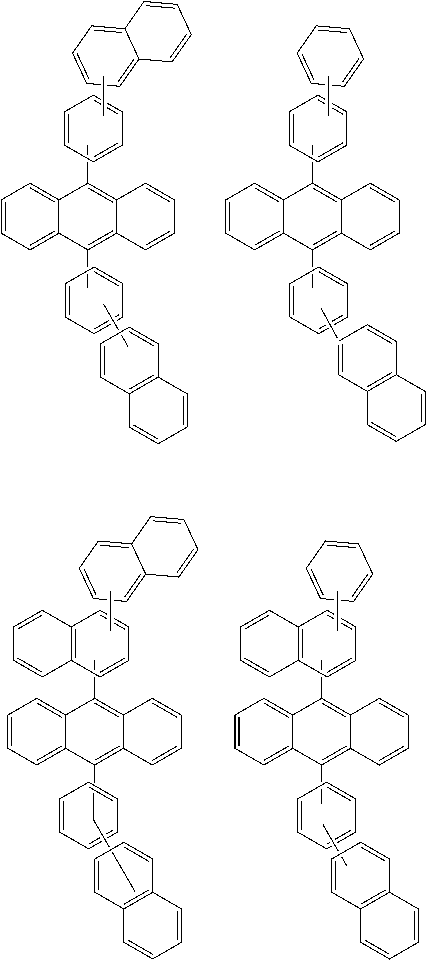

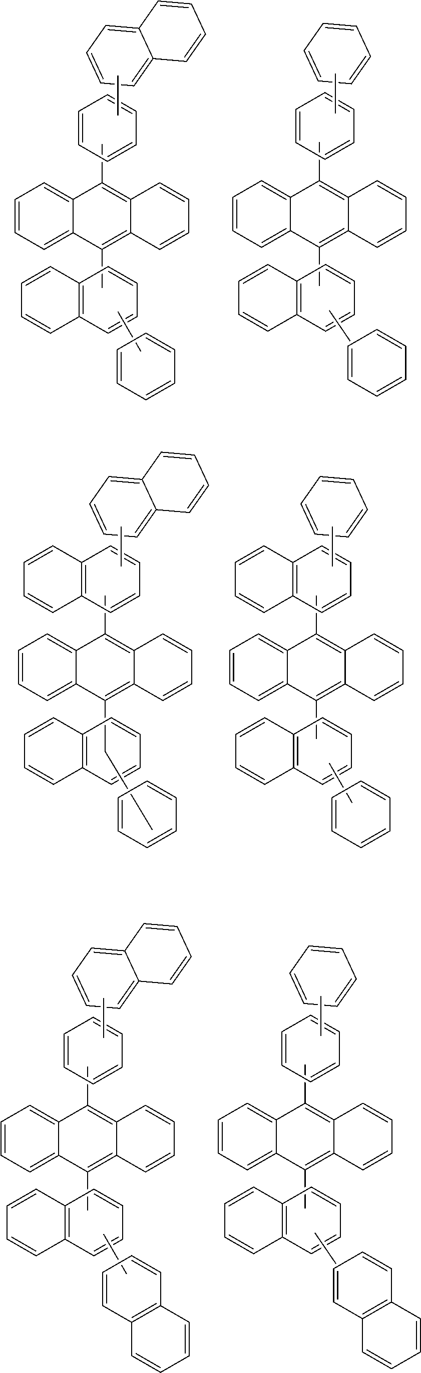

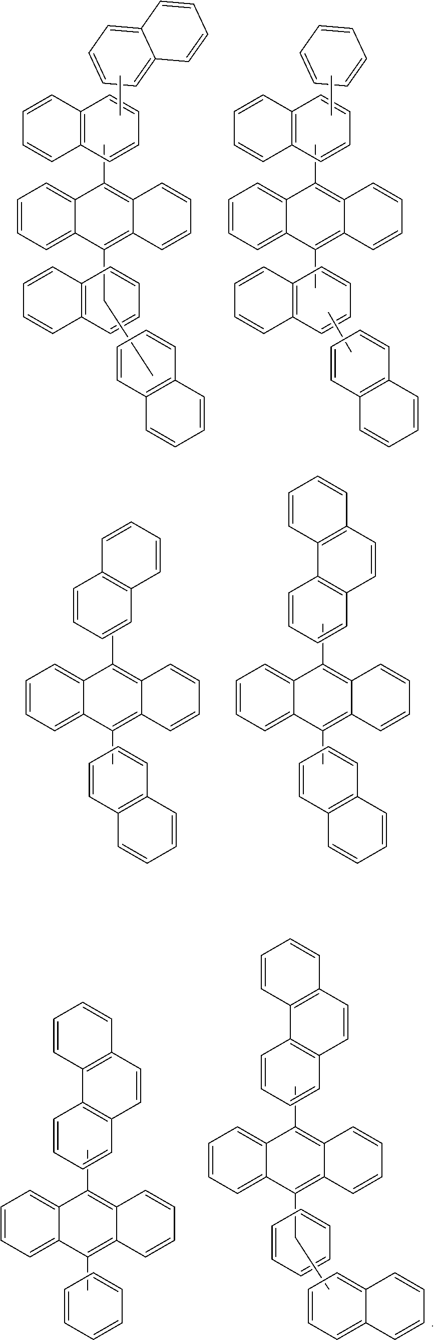

6. The deuterated composition according to claim 1, wherein Ar₁₁, Ar₁₂, R₁₁ and R₁₂ are each independently selected from any one of 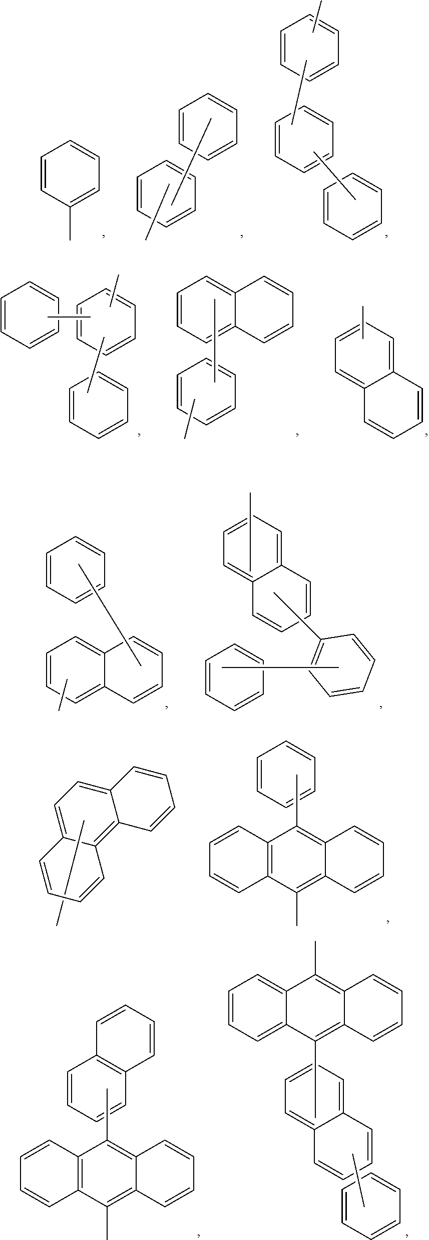 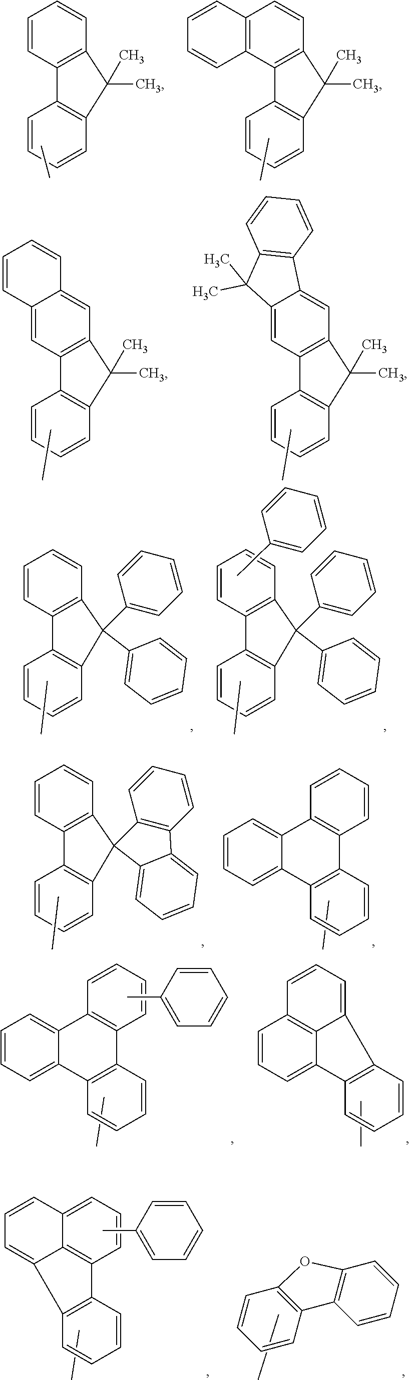 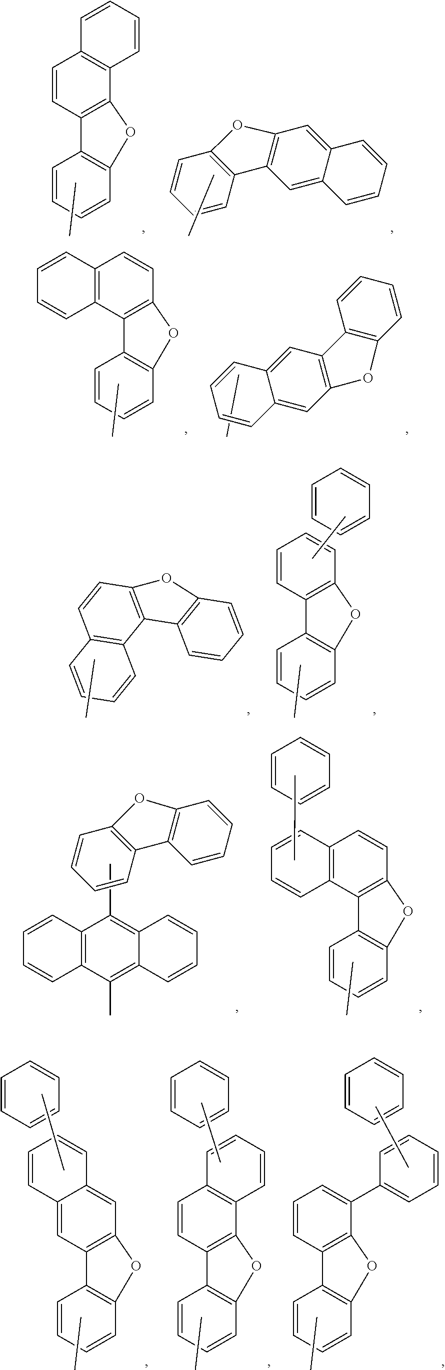 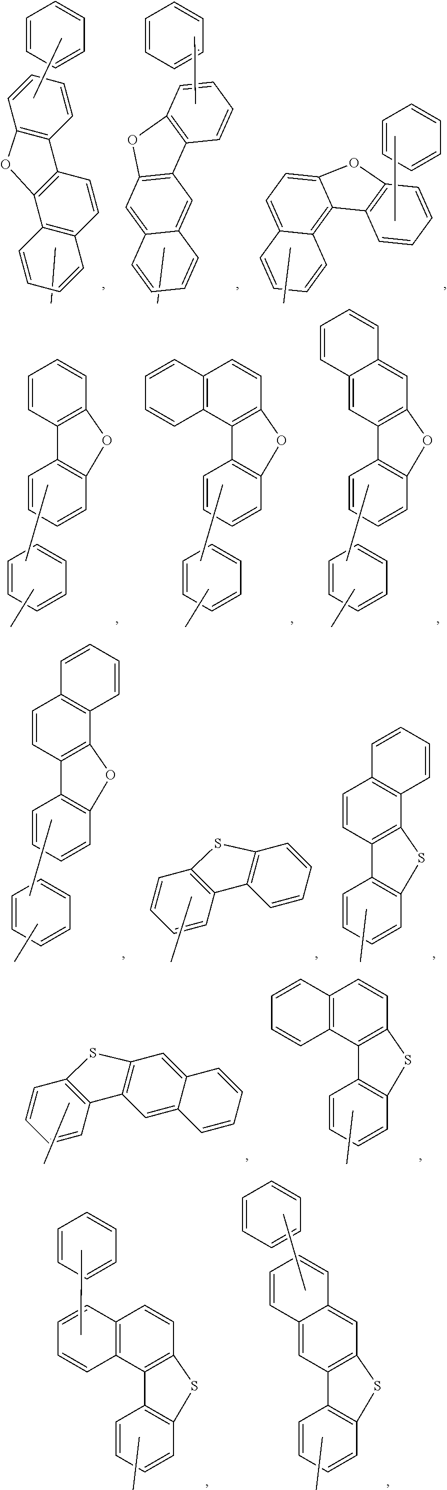 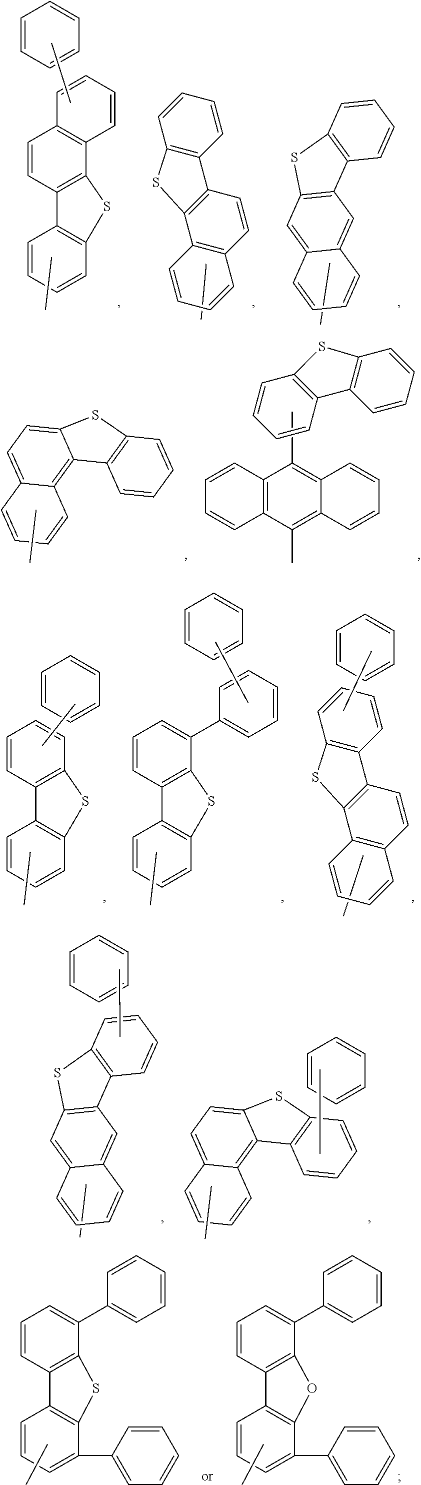
preferably, Ar₁₂ is selected from any one of 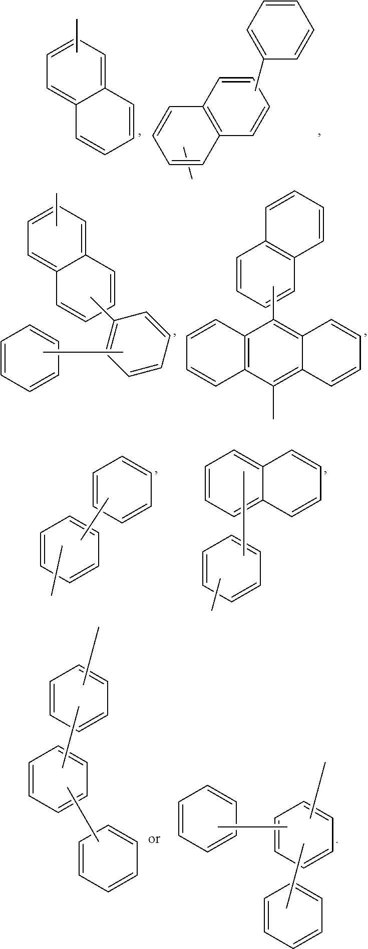
preferably, R₁₁ and R₁₂ are each independently selected from any one of methyl, ethyl, propyl, butyl,

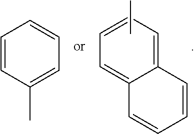

7. The deuterated composition according to claim 1, wherein Compound A is selected from any one of the following compounds:

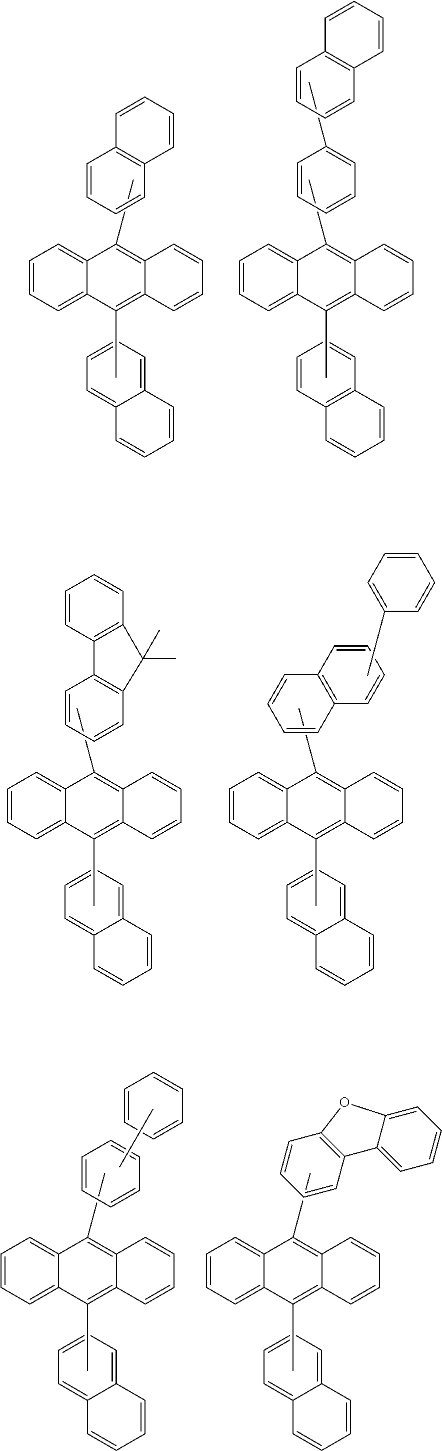

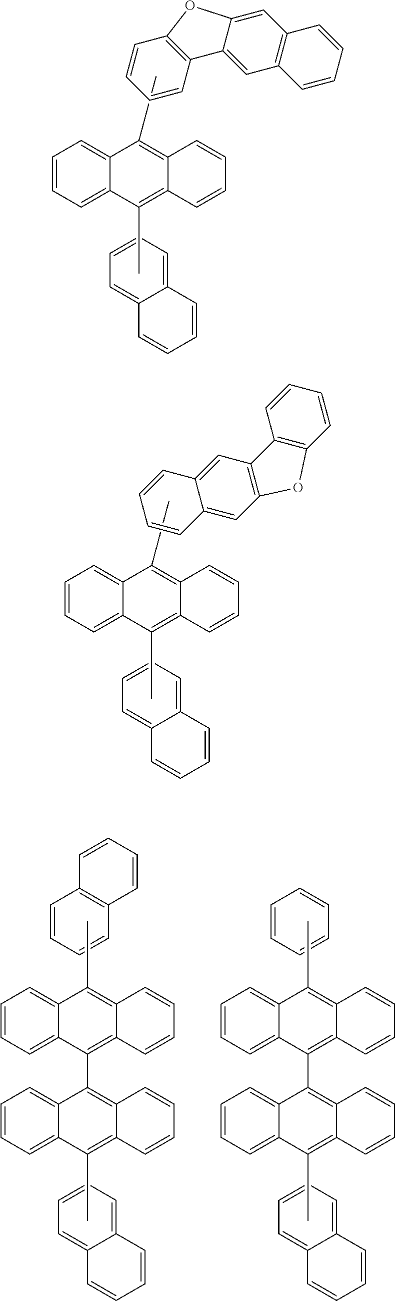

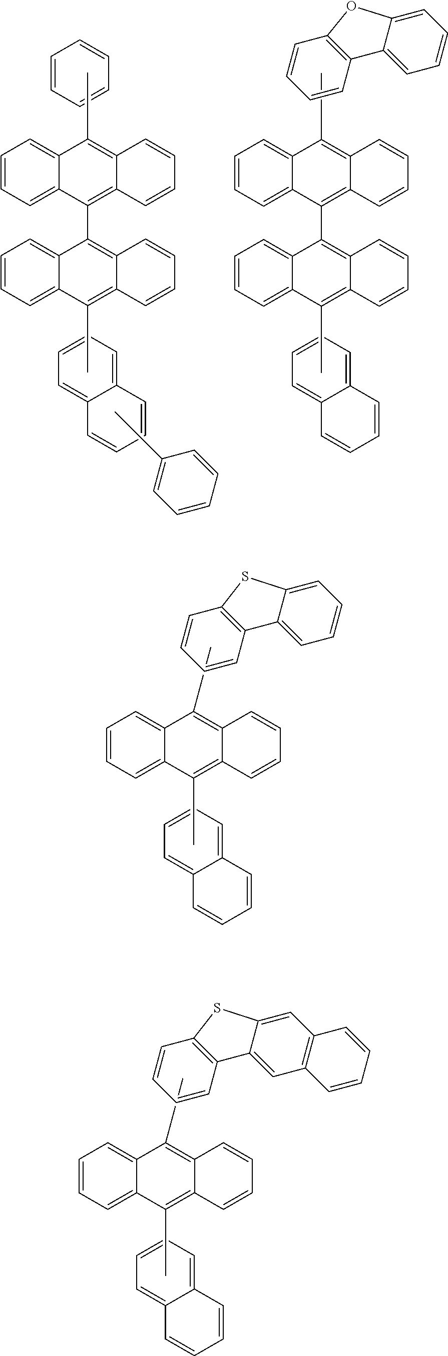

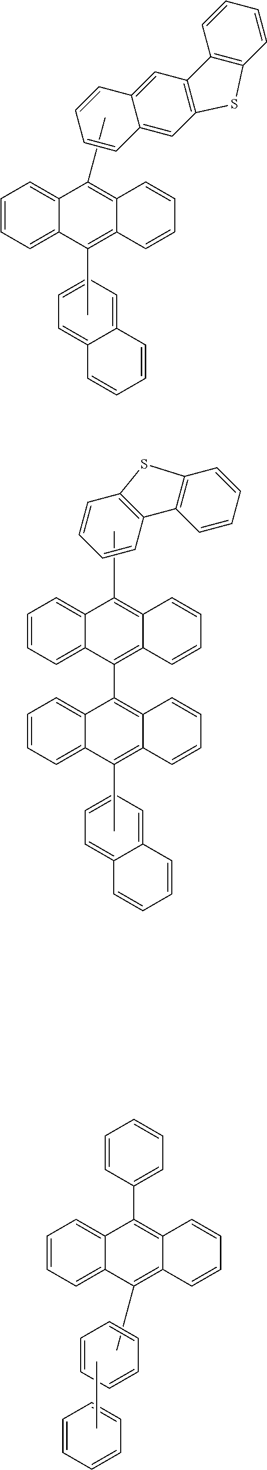

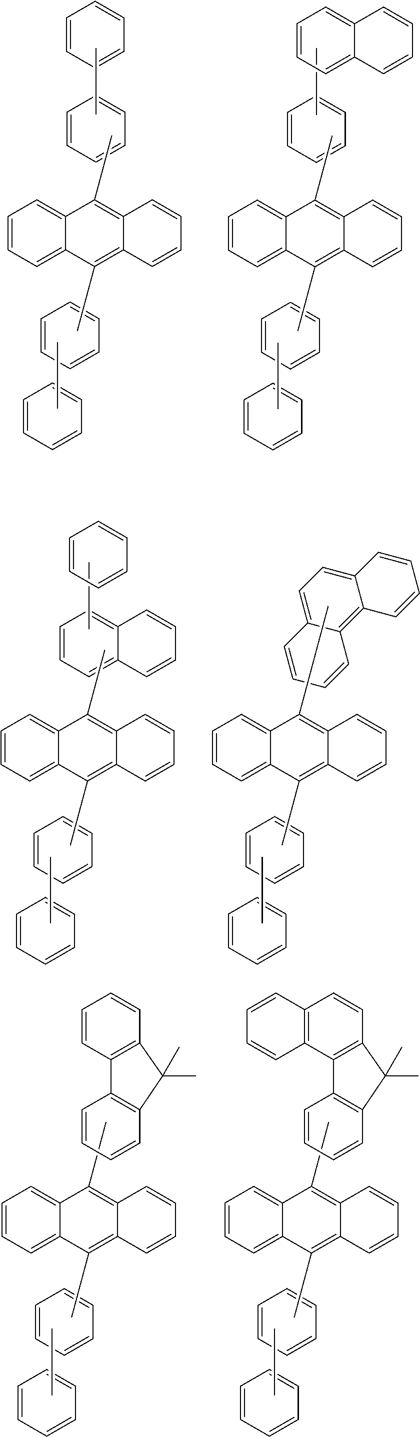

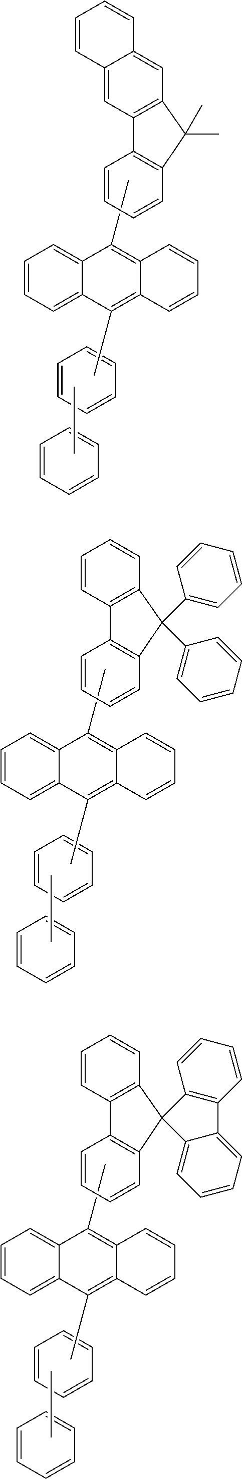

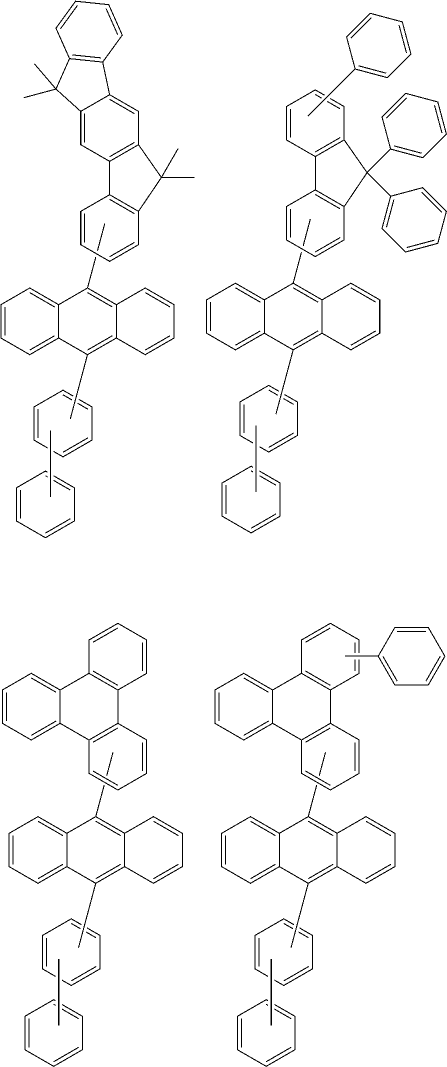

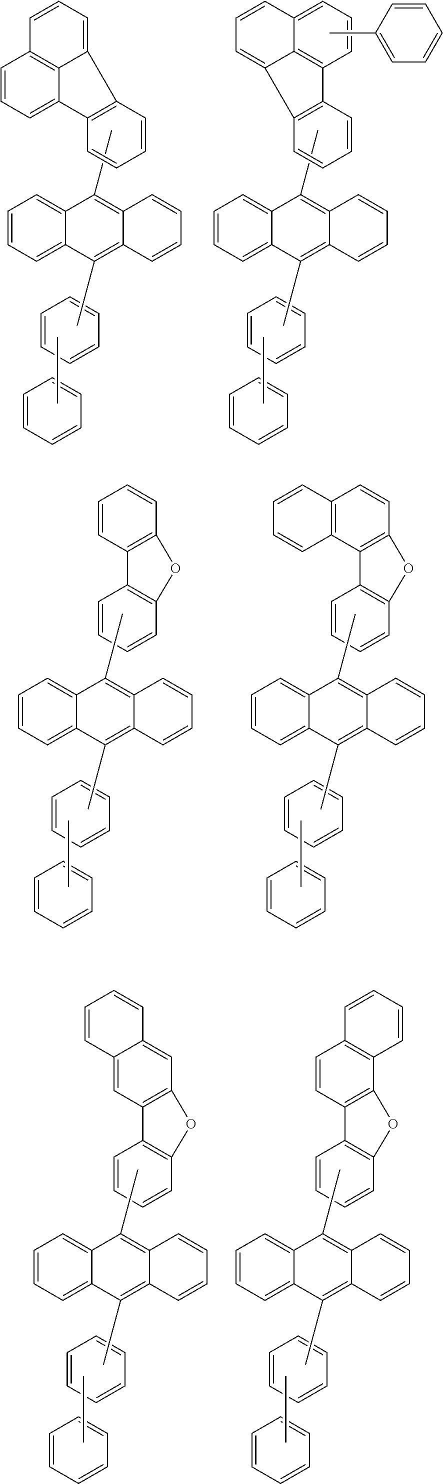

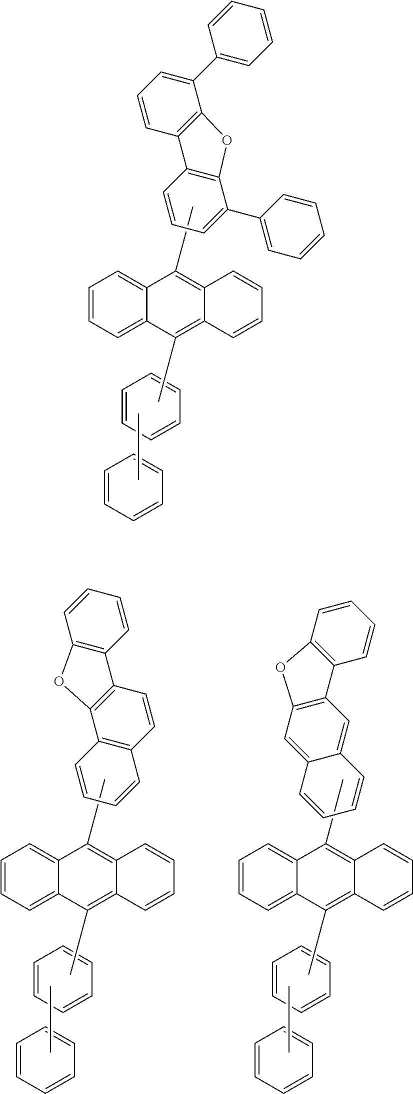

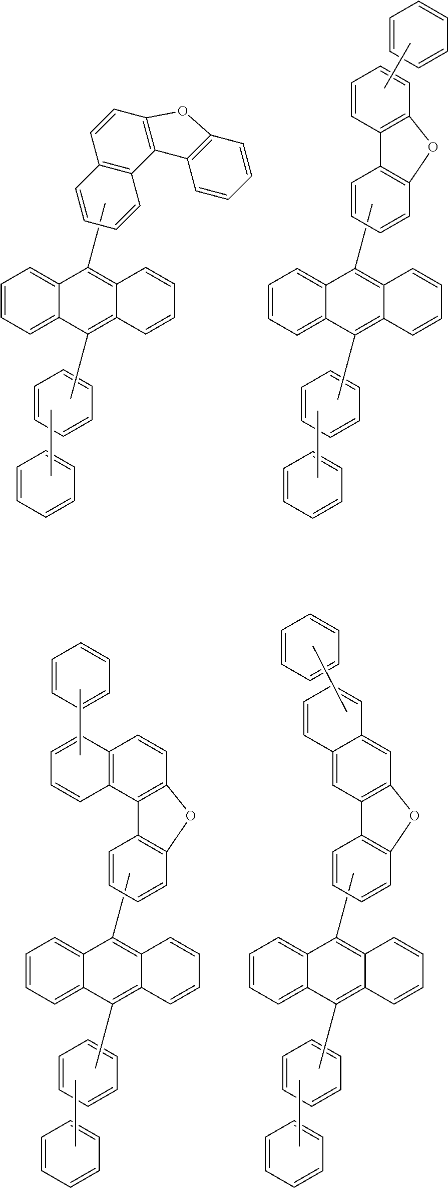

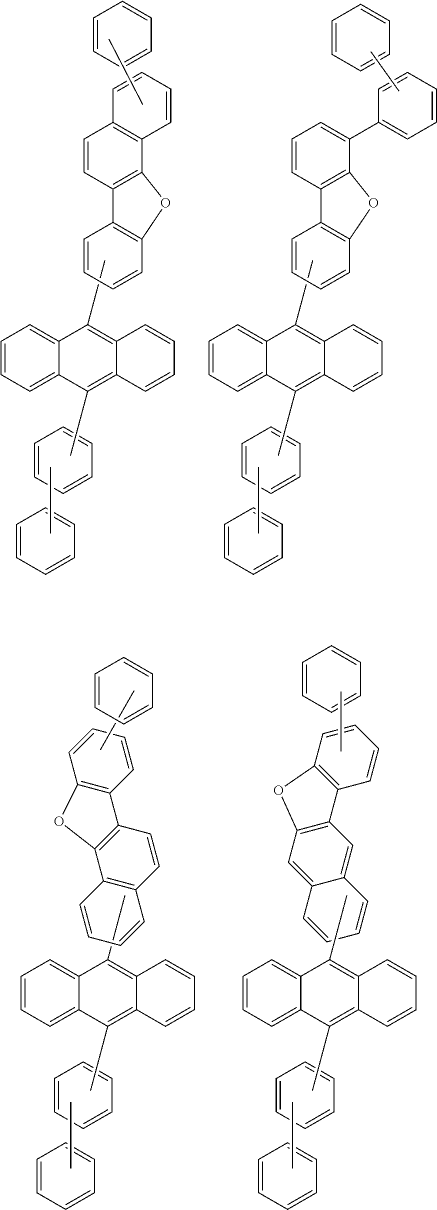

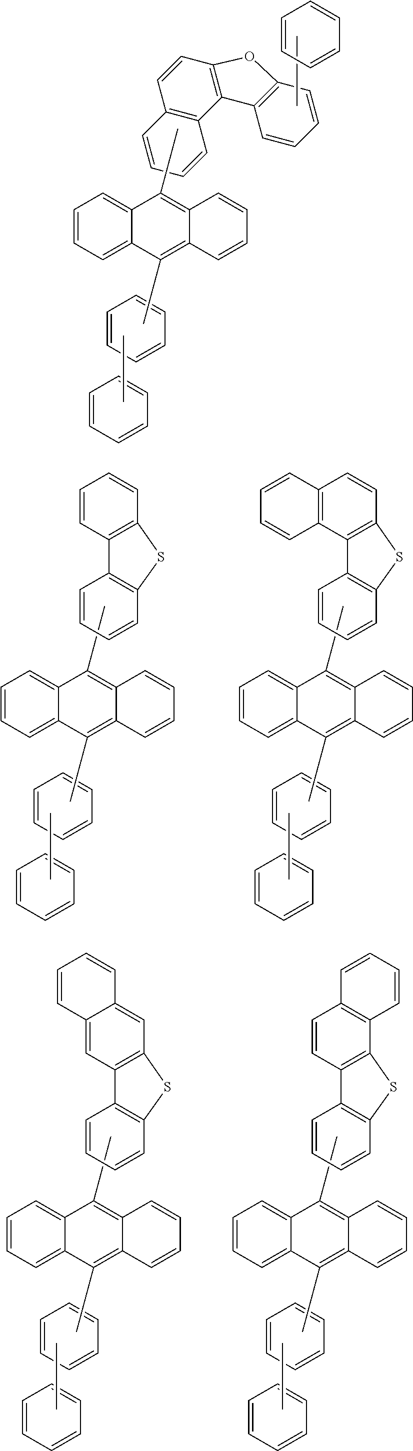

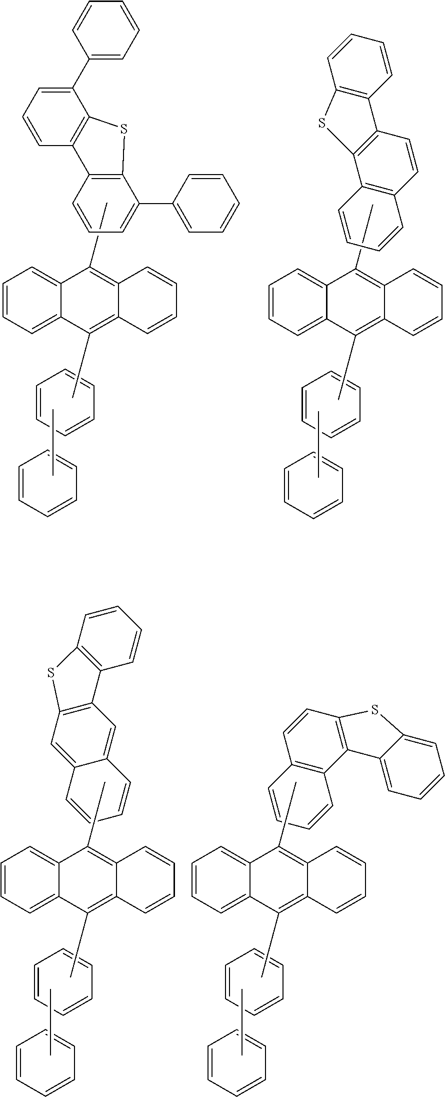

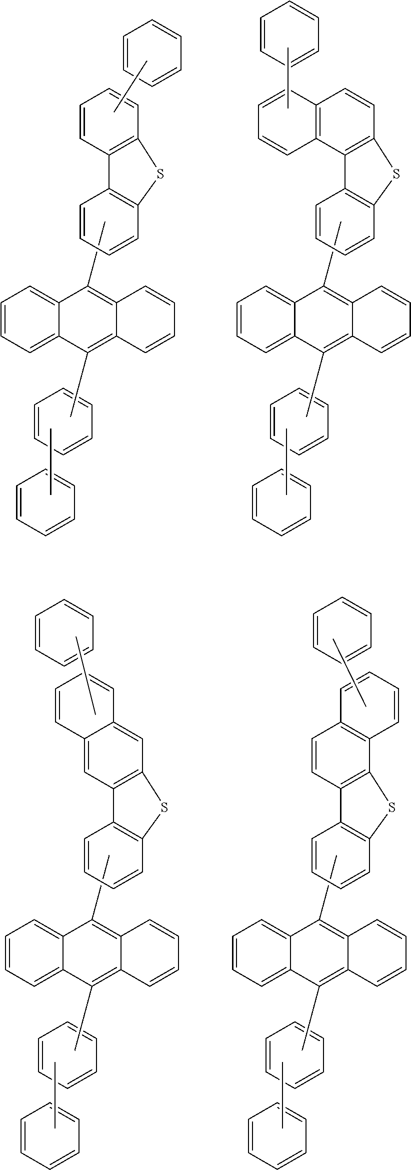

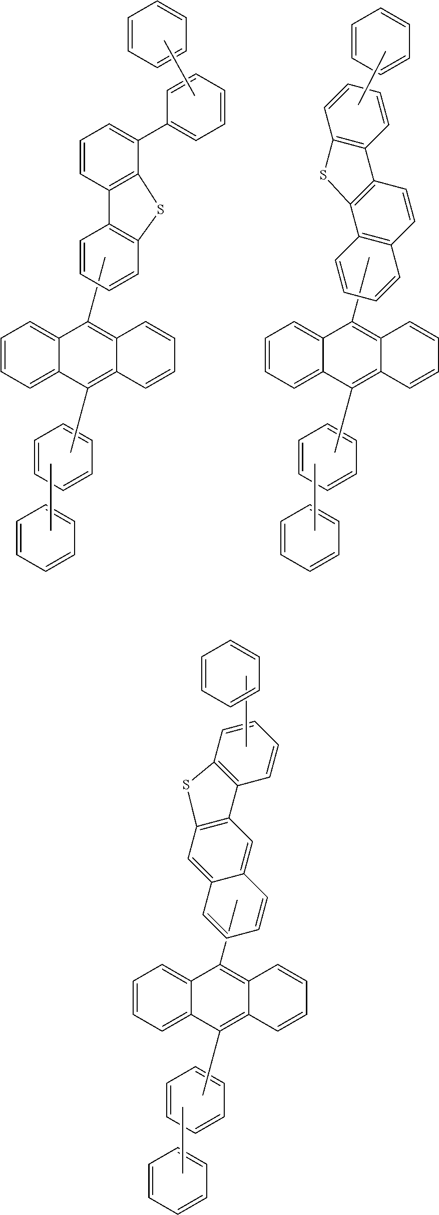

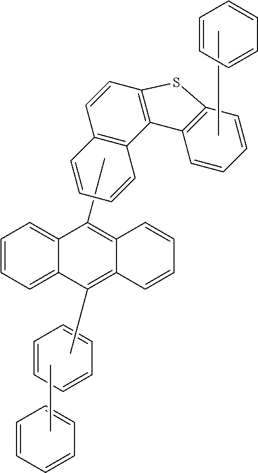

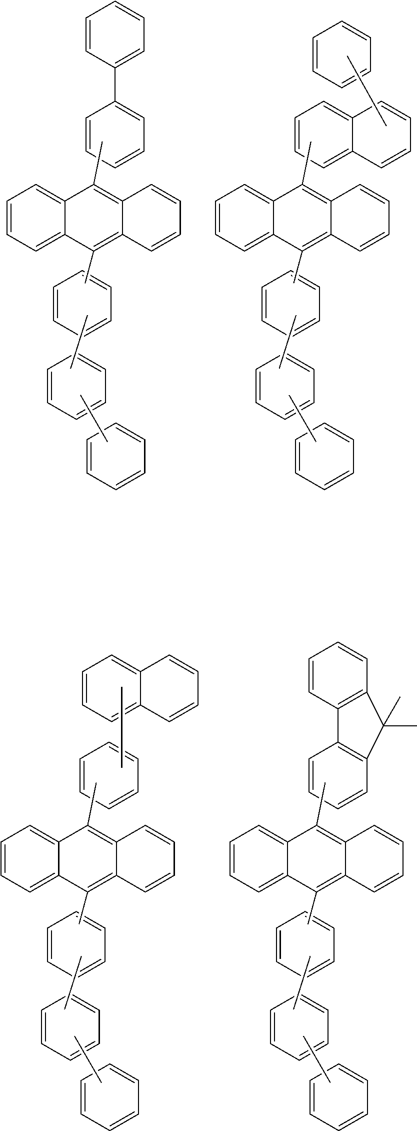

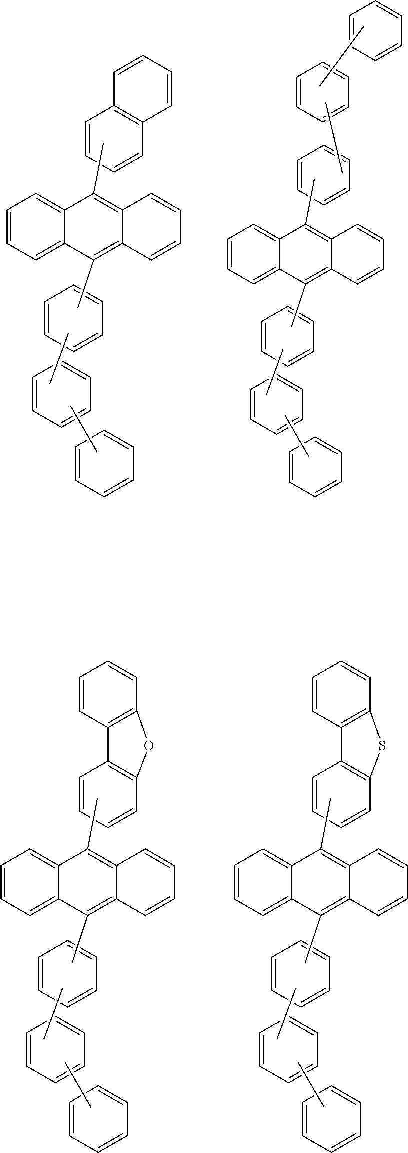

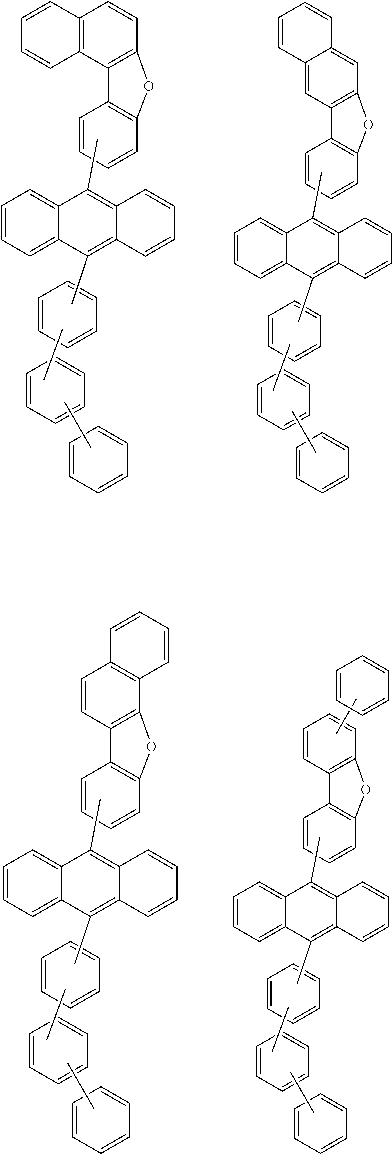

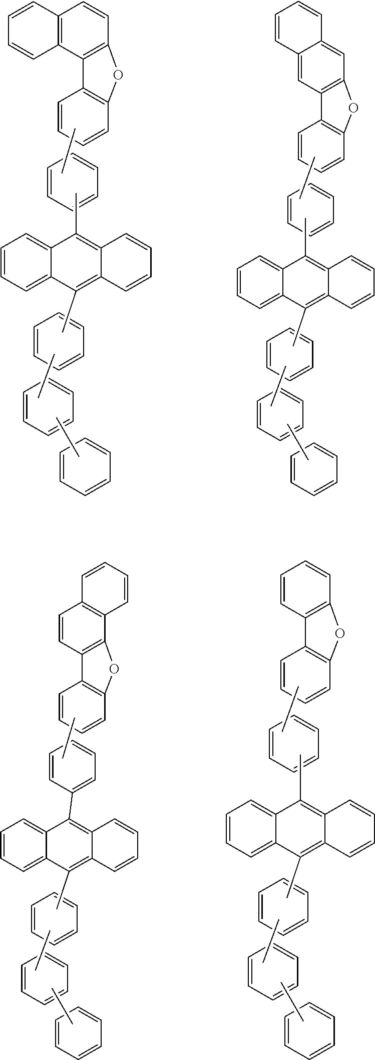

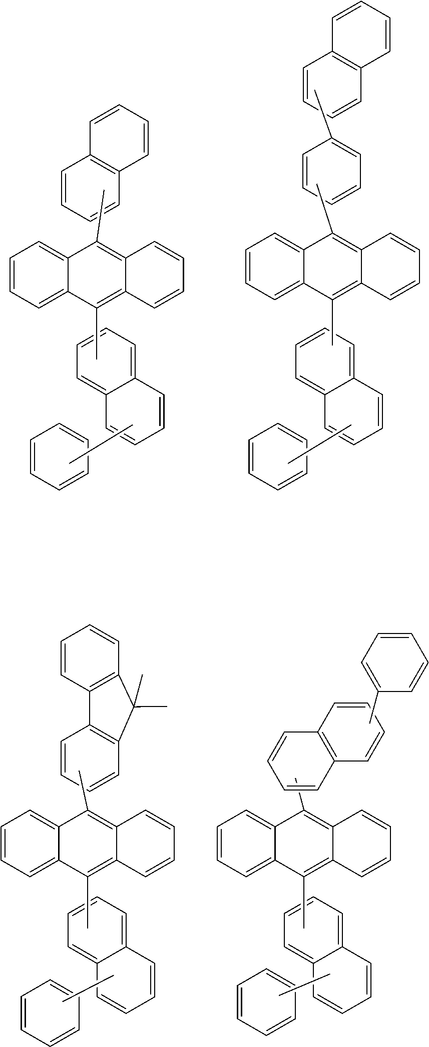

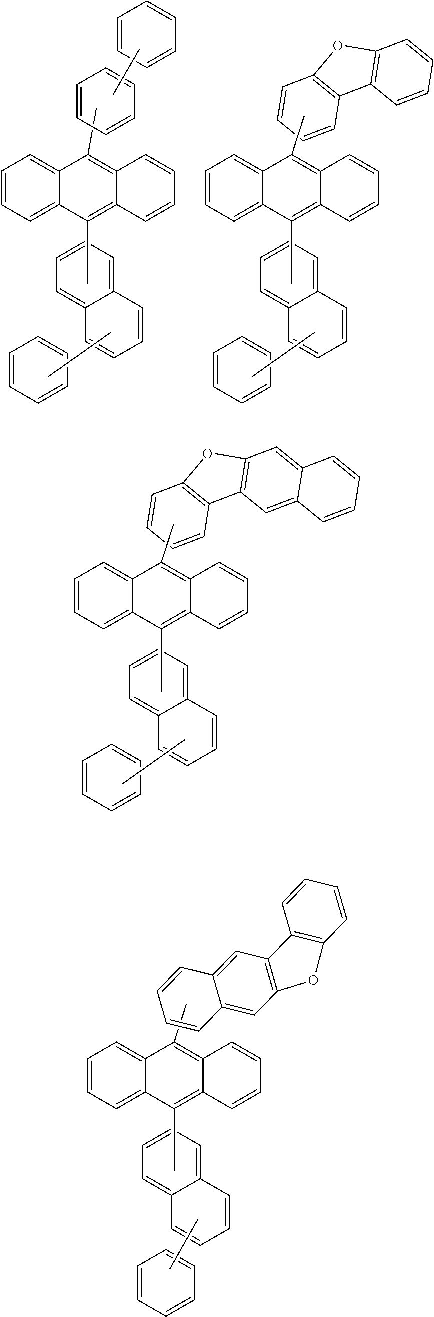

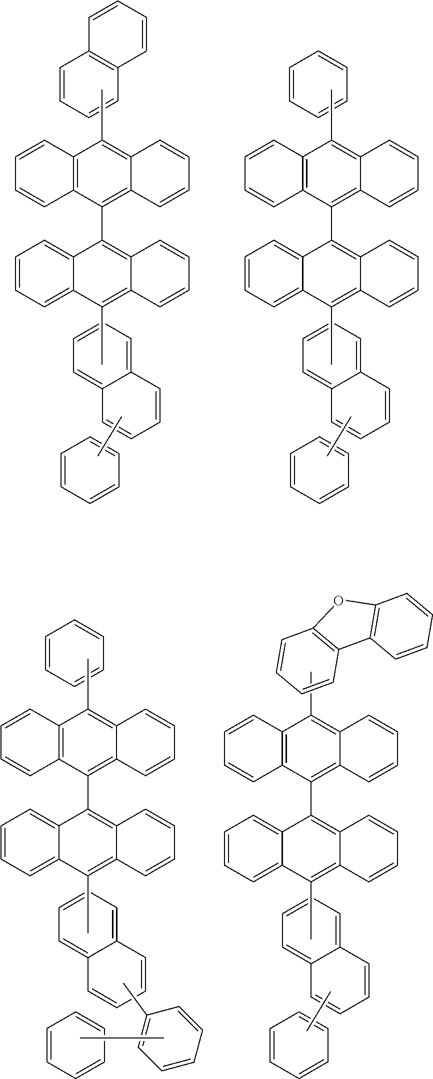

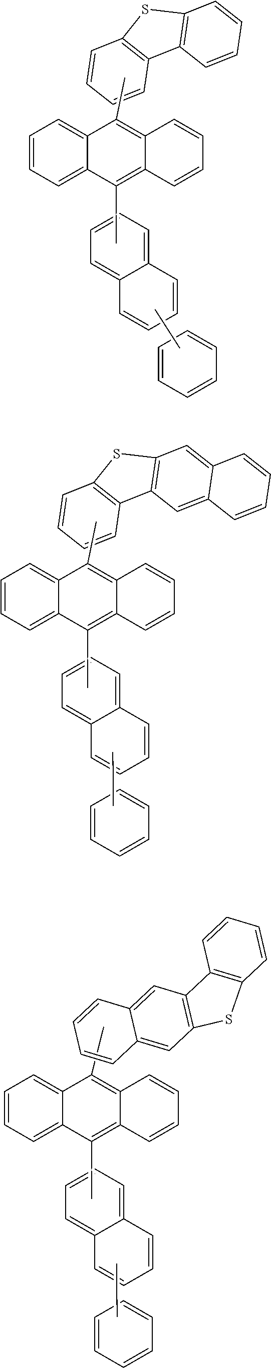

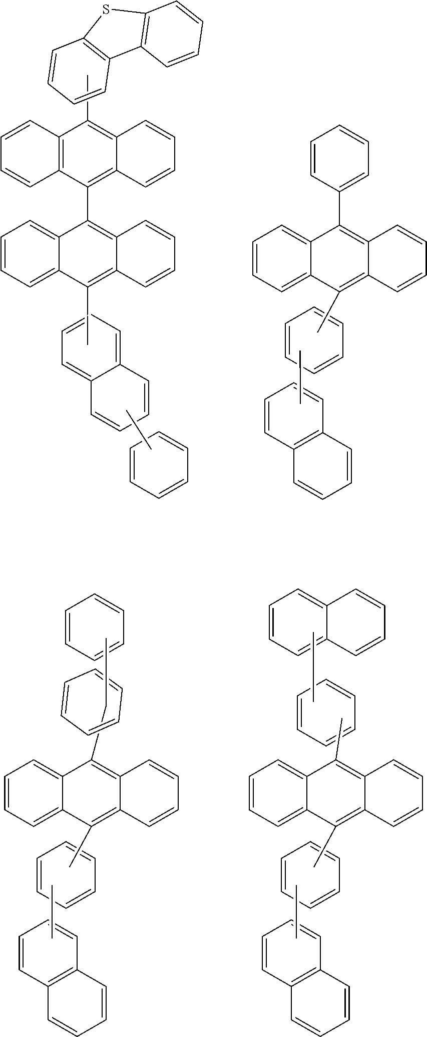

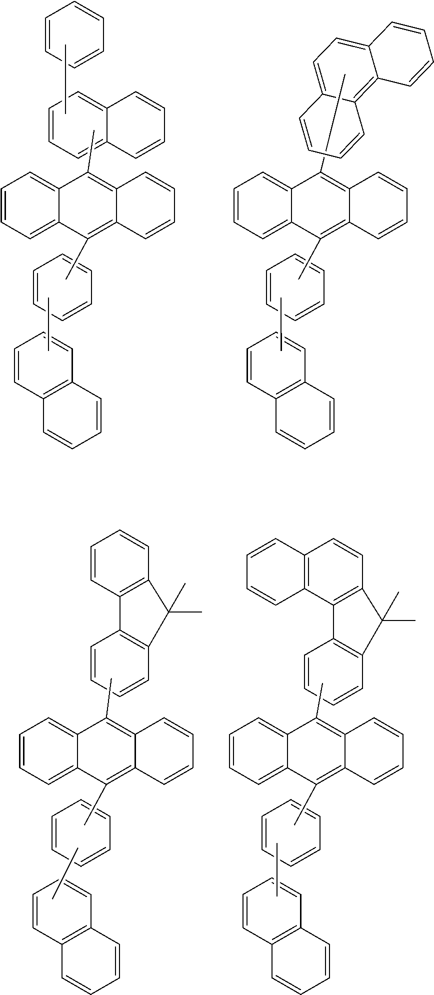

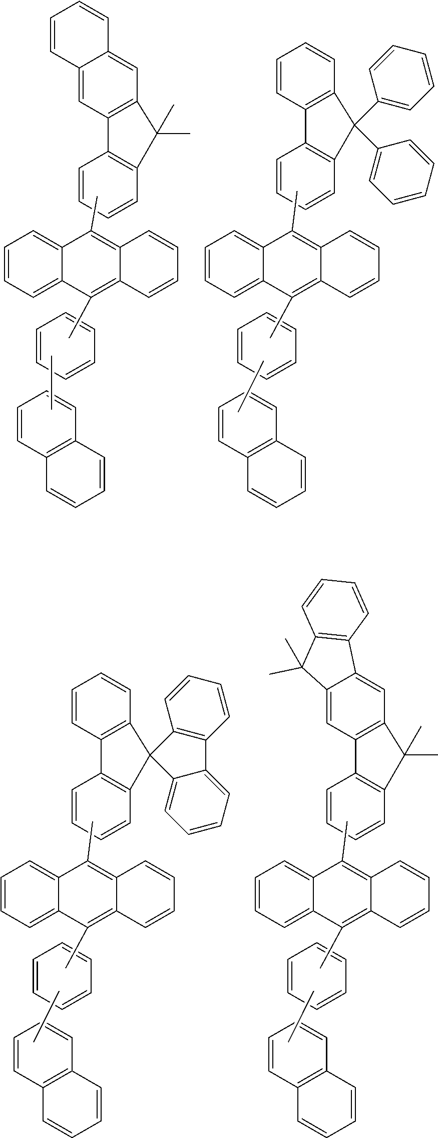

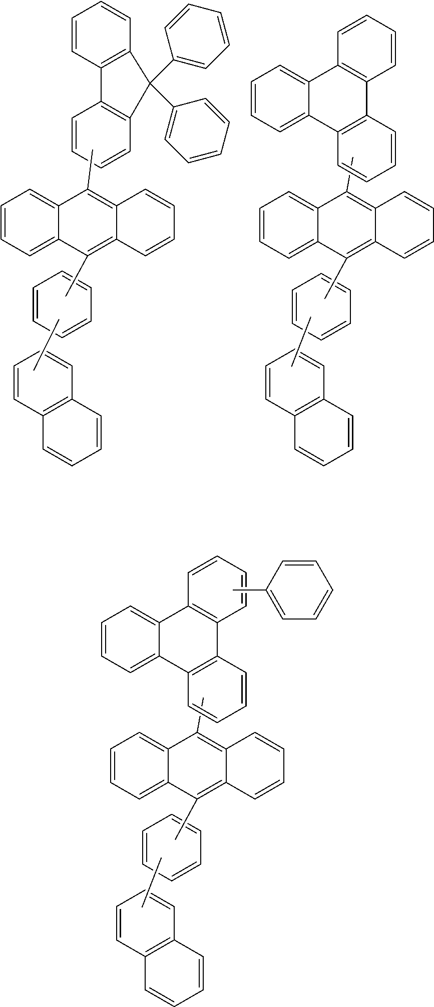

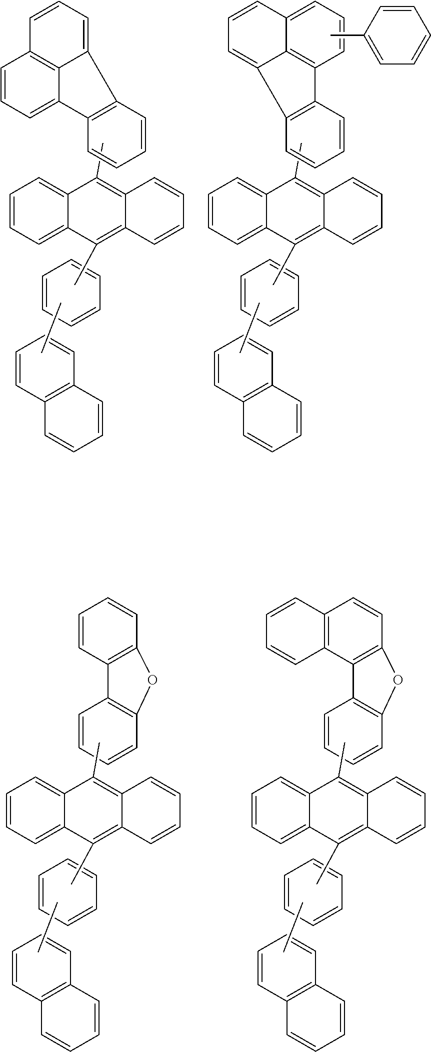

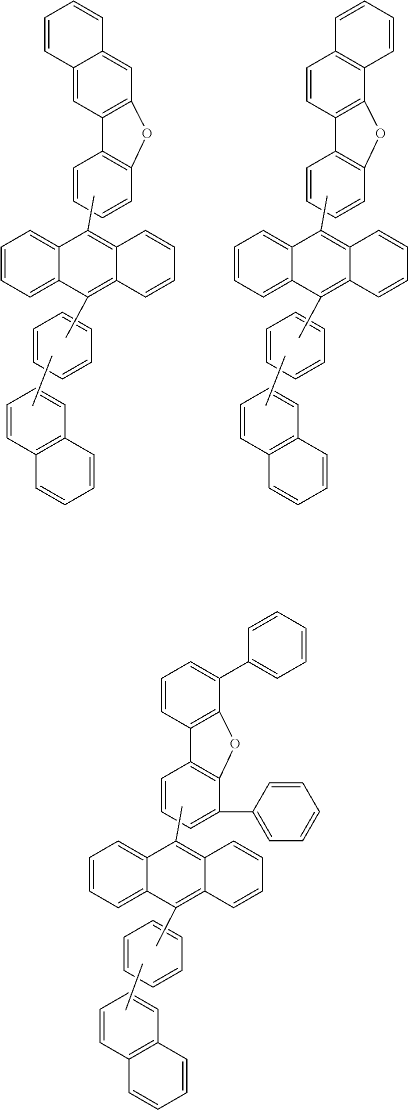

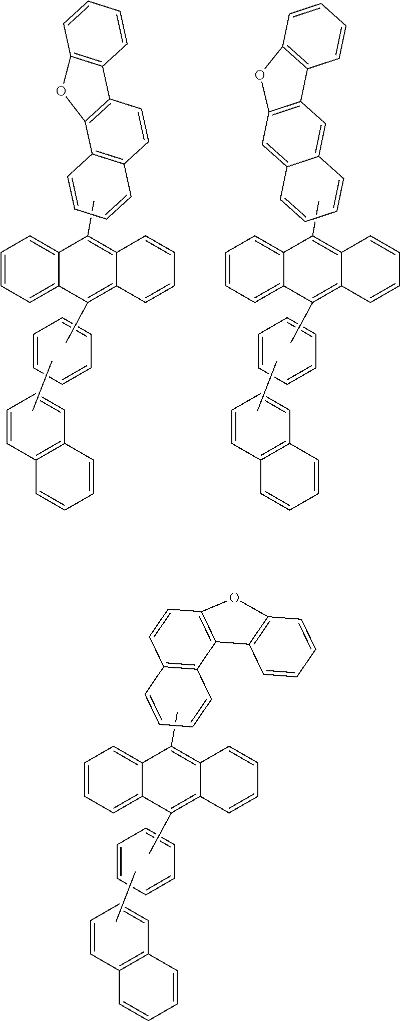

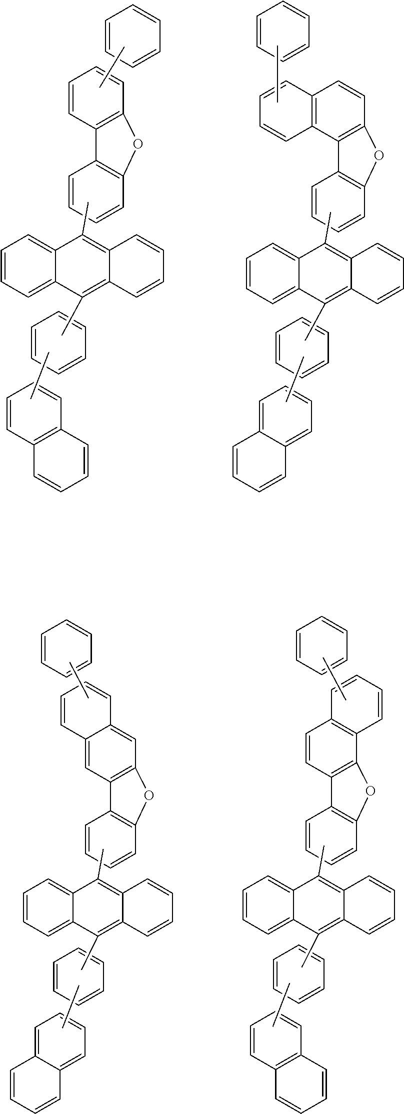

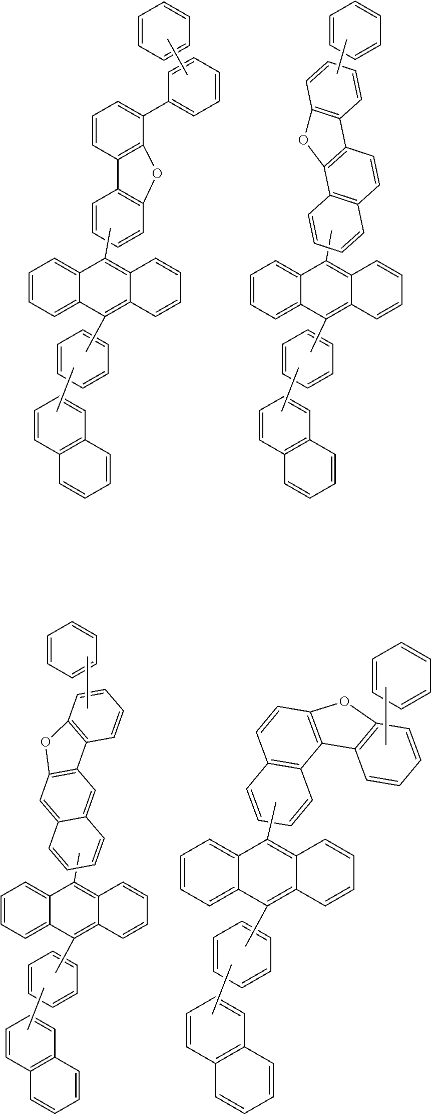

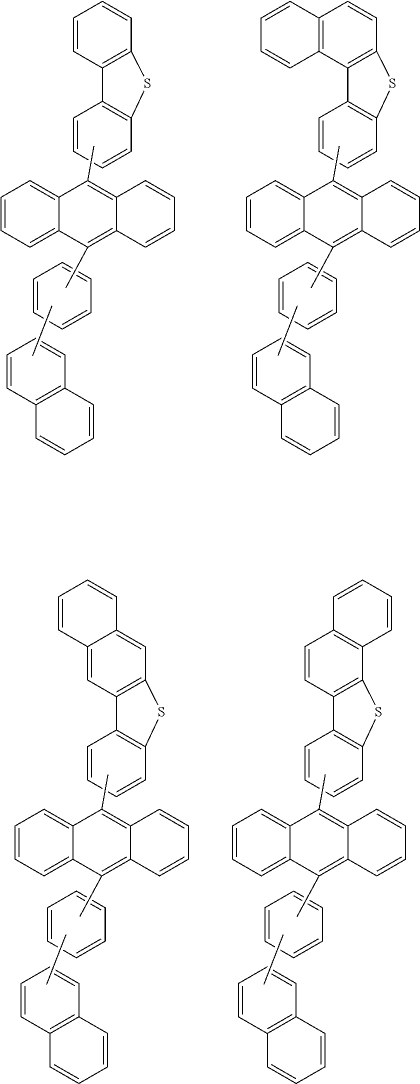

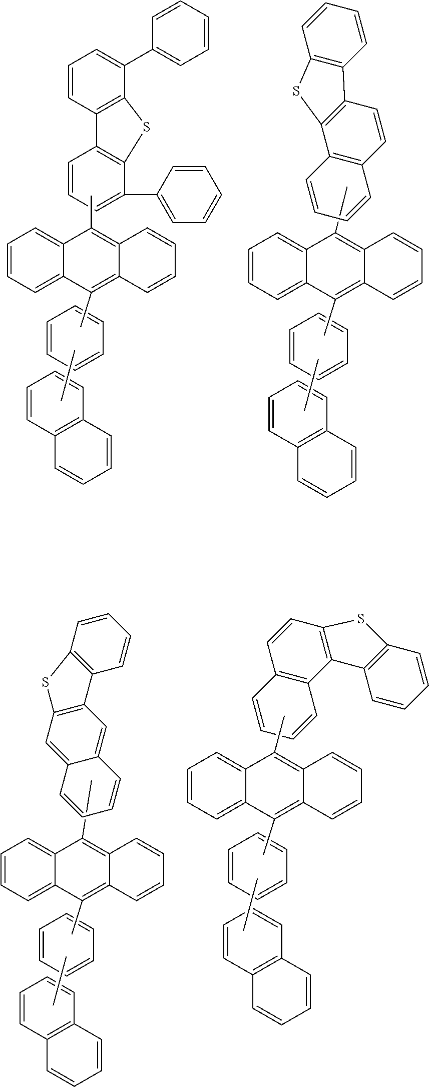

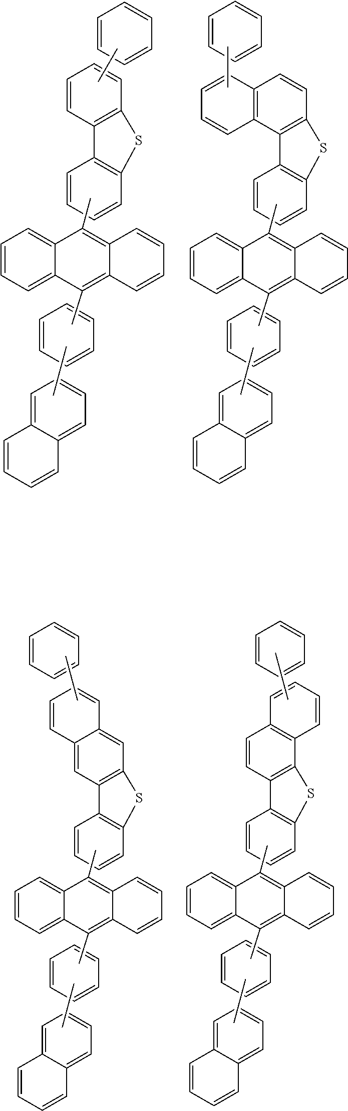

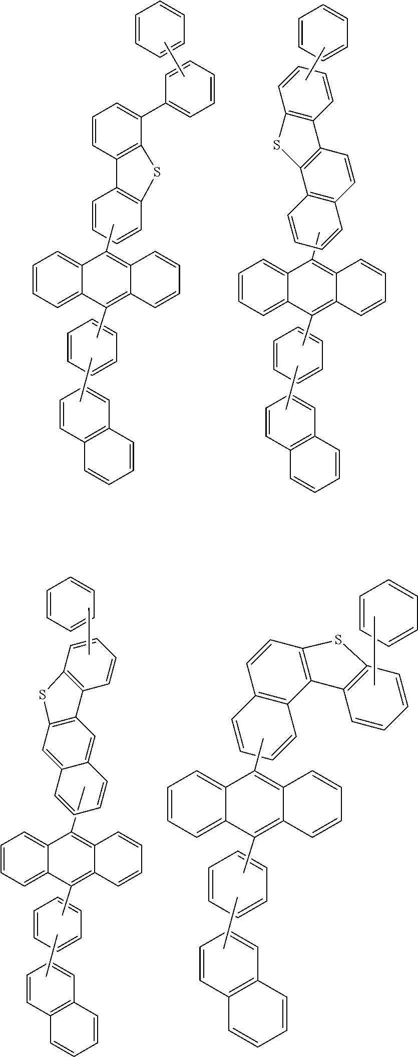

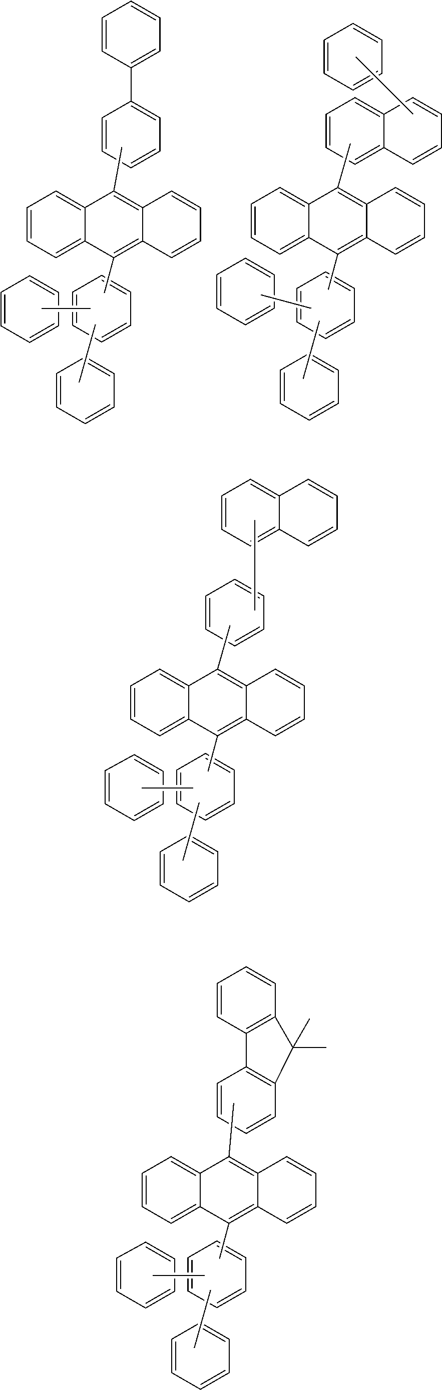

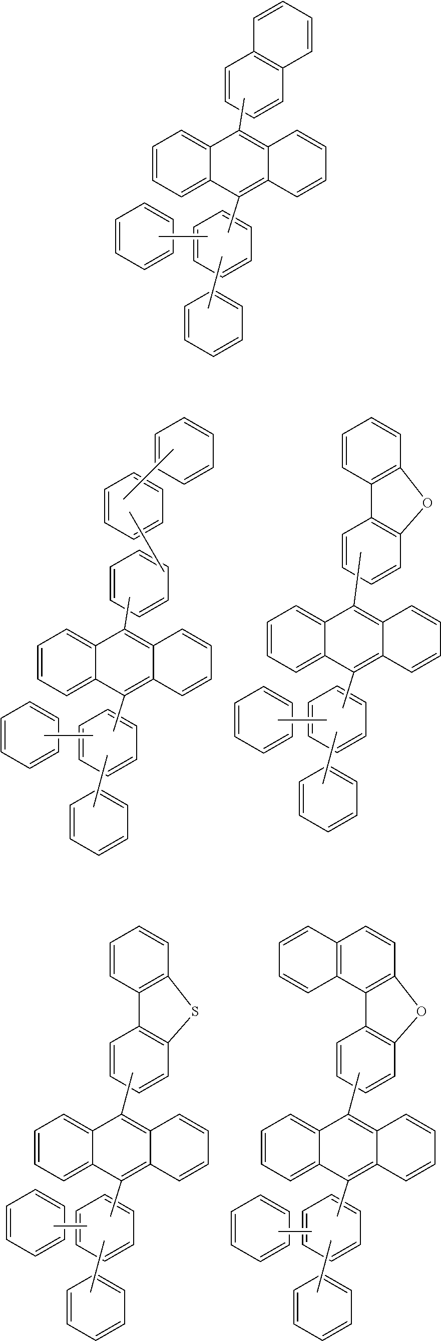

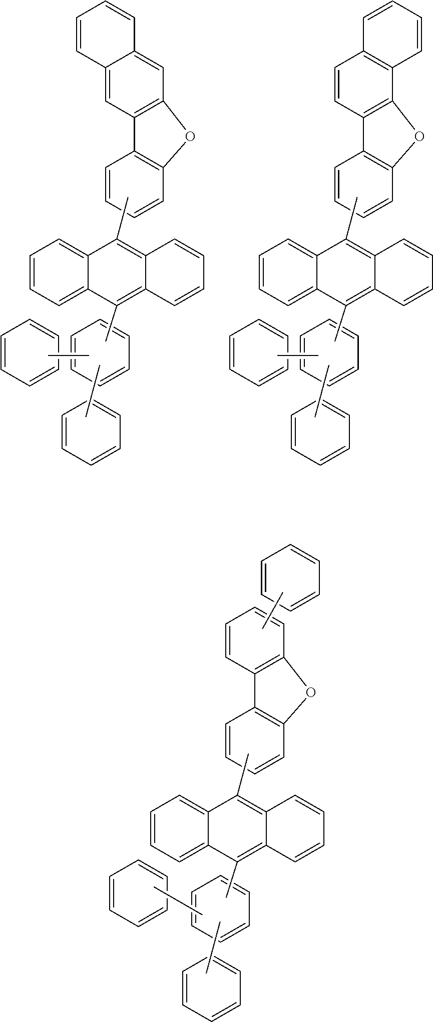

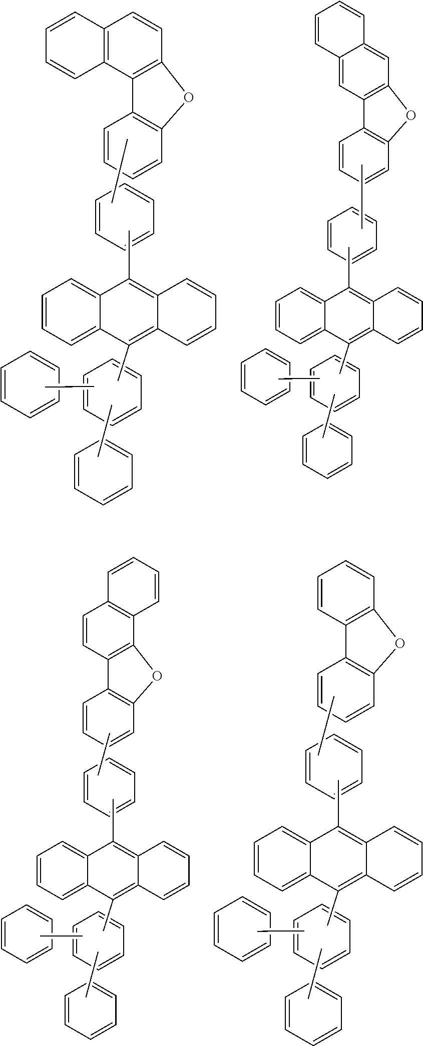

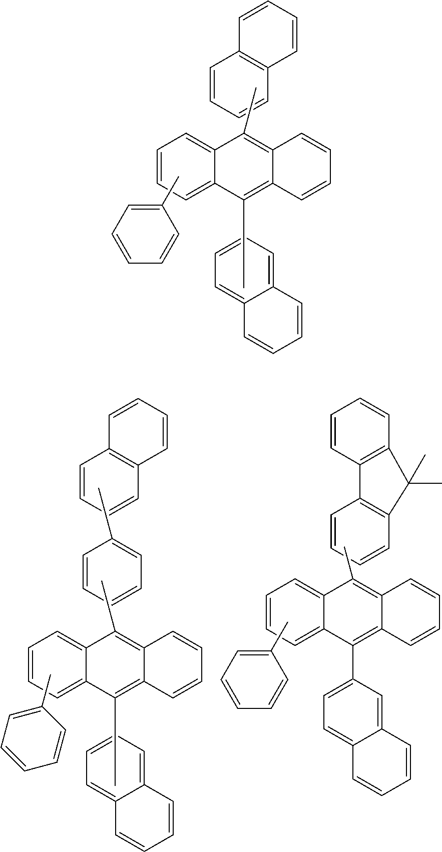

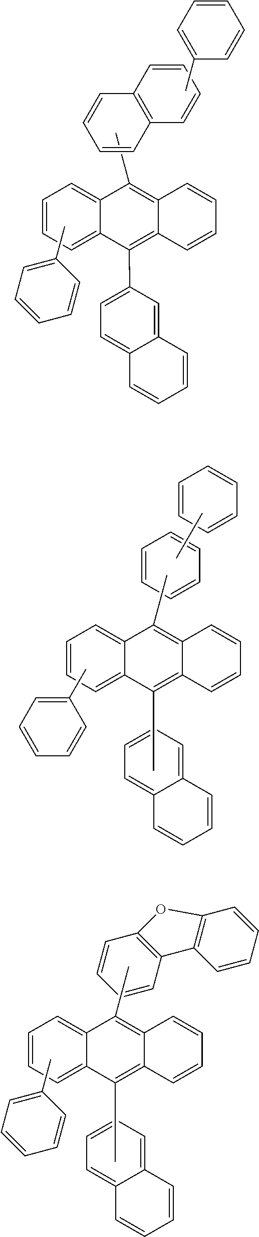

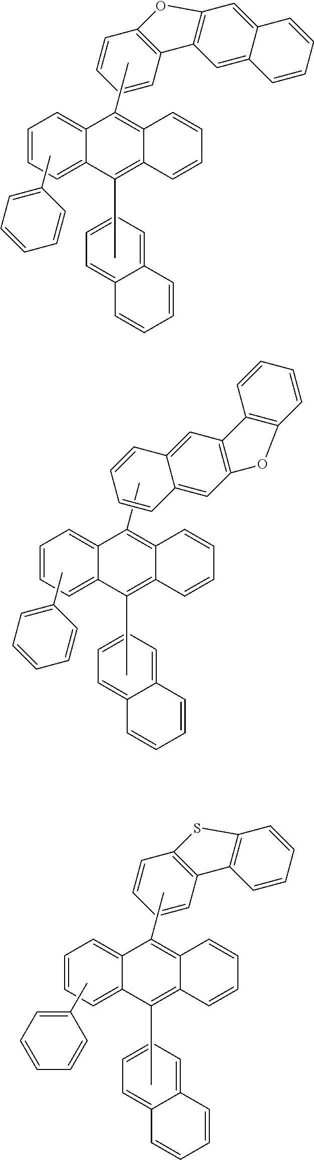

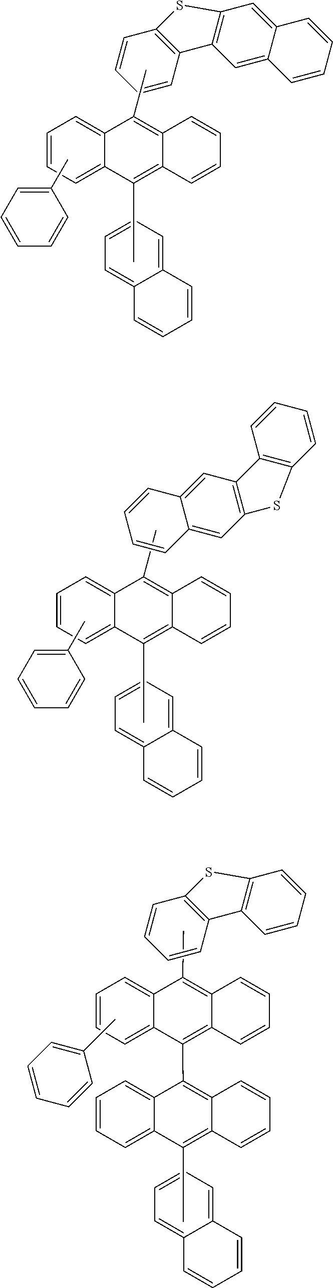

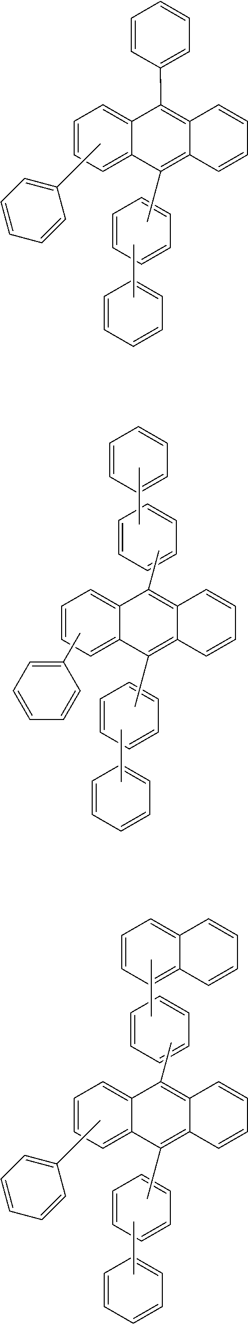

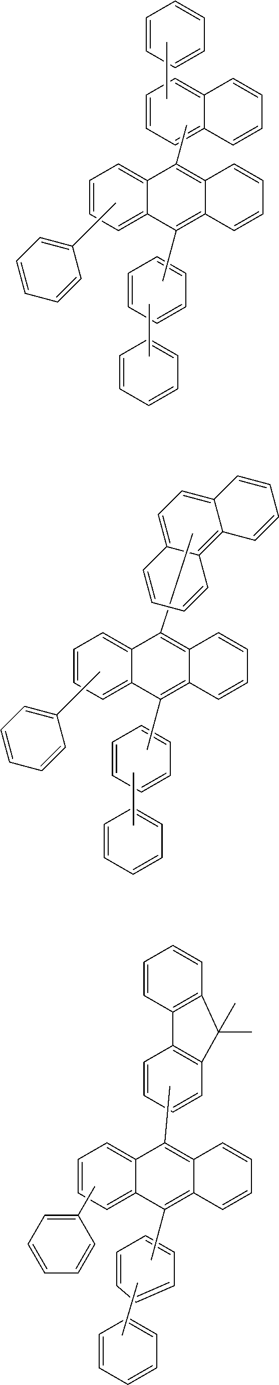

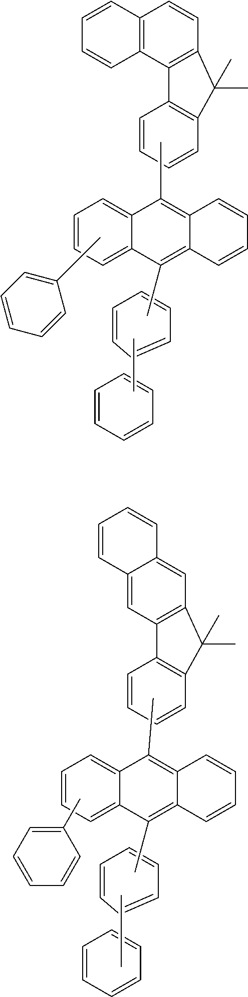

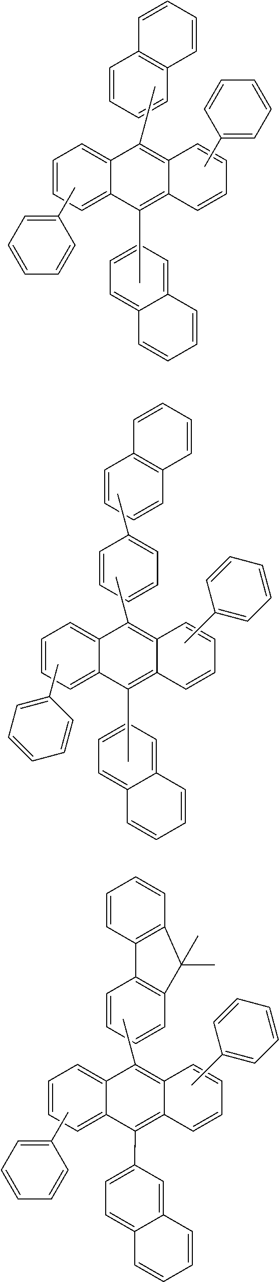

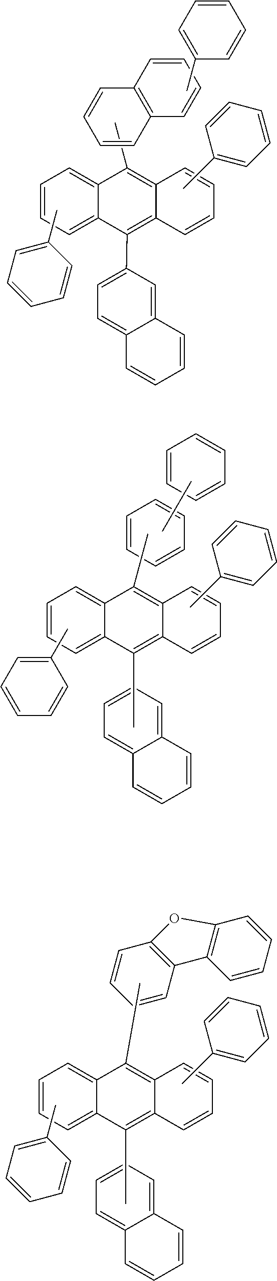

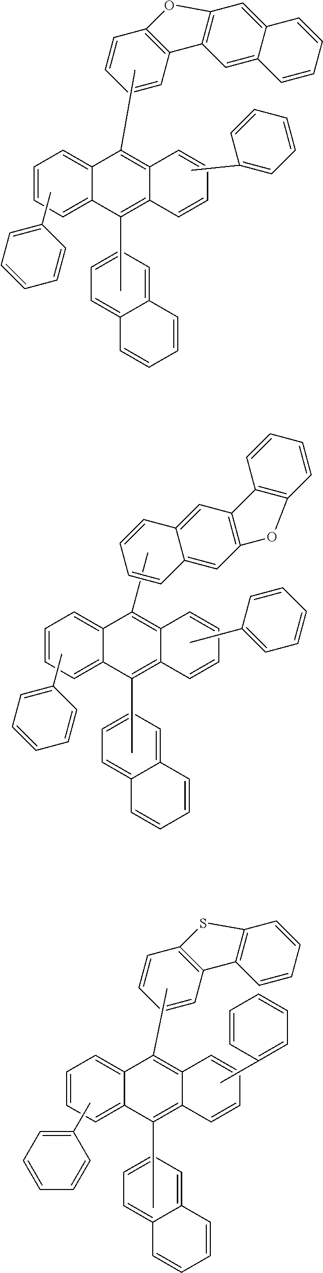

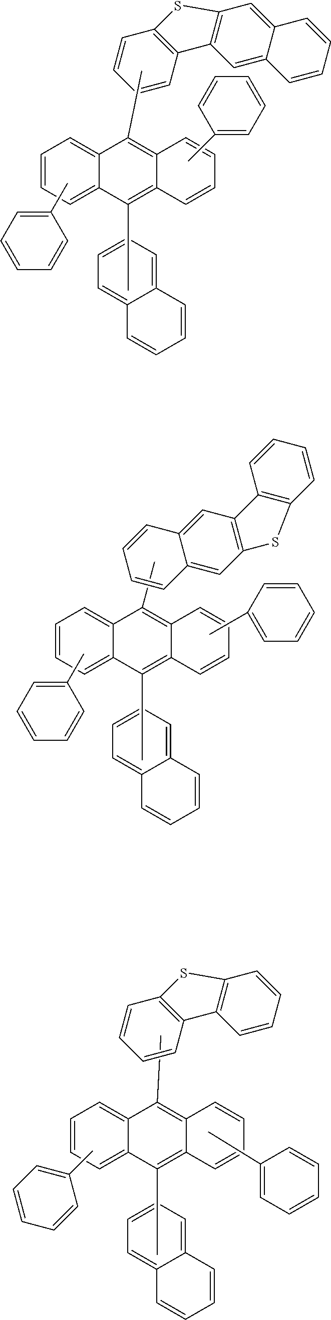

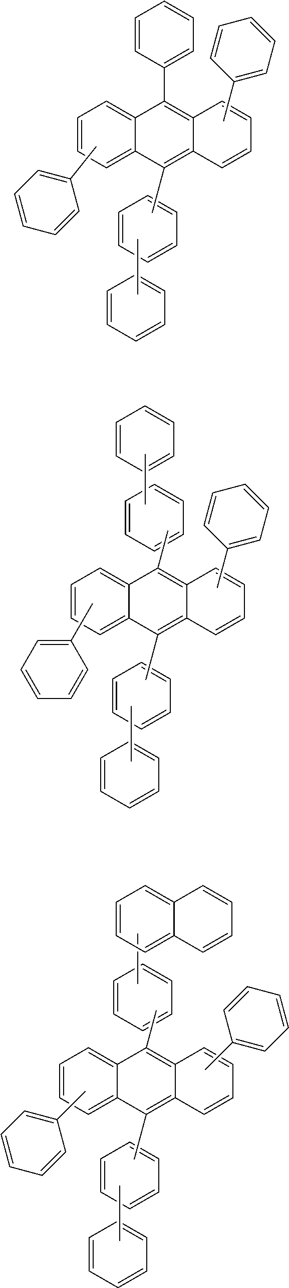

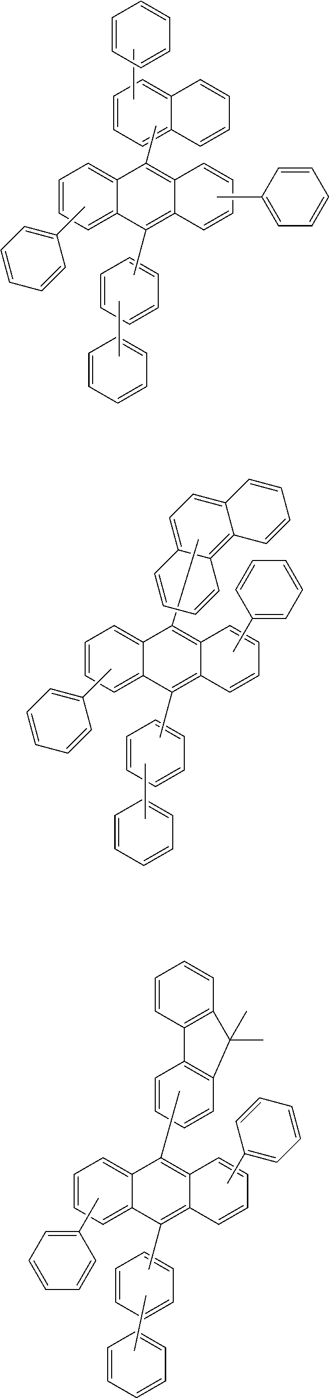

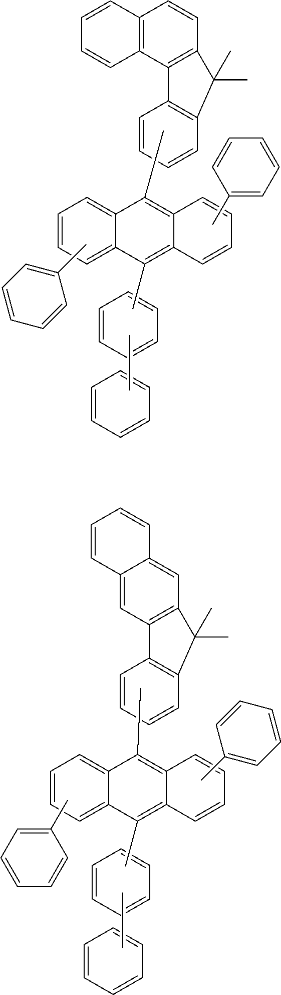

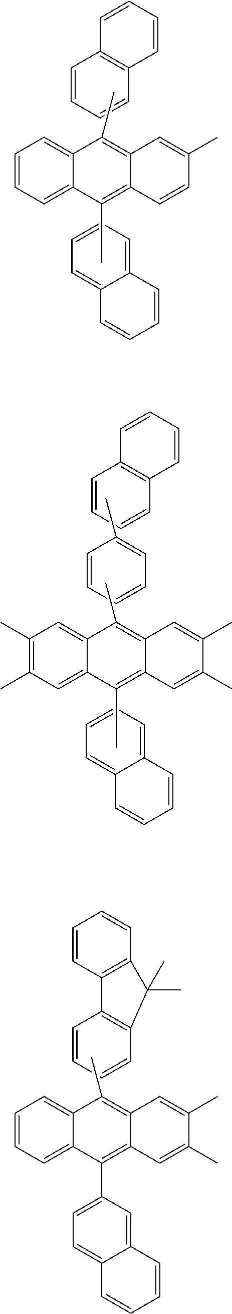

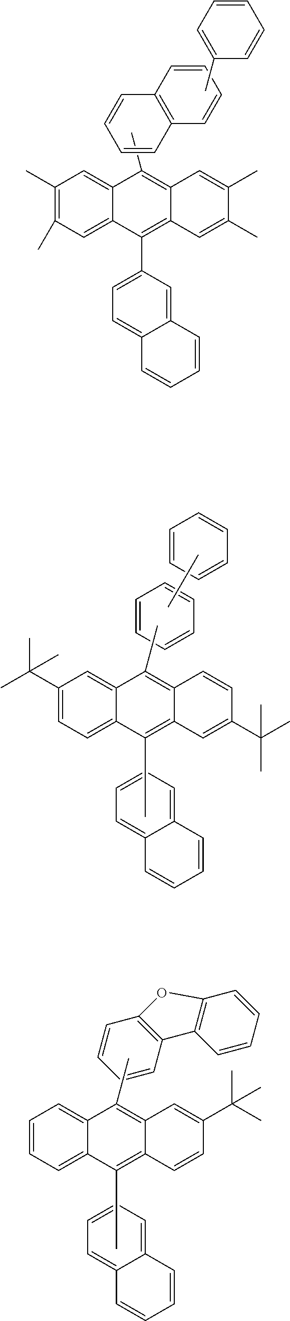

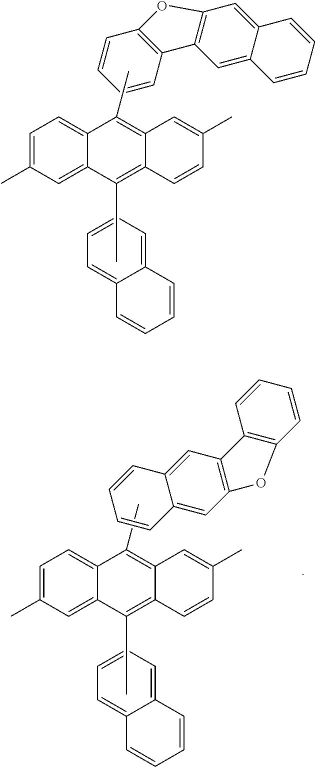

8. The deuterated composition according to claim 1, wherein the deuterated reaction comprises the following steps:
in the presence of a catalyst, placing Compound A in D₂O and a solvent, and conducting the deuterated reaction to obtain the deuterated mixture;
preferably, the solvent is selected from any one or a combination of at least two of benzene, toluene, ethyl acetate or C₆D₆, further preferably C₆D₆;
optionally, the reaction is conducted in the presence of the catalyst;
optionally, the reaction is conducted in a hydrogen atmosphere;
optionally, a volume ratio of D₂O to C₆D₆ is 1: (4-6);
optionally, the deuterated reaction is conducted at a temperature of 60-200° C.;
optionally, the deuterated reaction lasts for 1-80 h;
optionally, a pressure of the deuterated reaction is 0.01-2 MPa; and
optionally, a deuteration rate of the deuterated mixture is 15% to 99%, further preferably 28% to 70%, and further preferably 38% to 54%.

9. The deuterated composition according to claim 1, wherein the deuterated composition further comprises Compound B having a structure represented by Formula II: 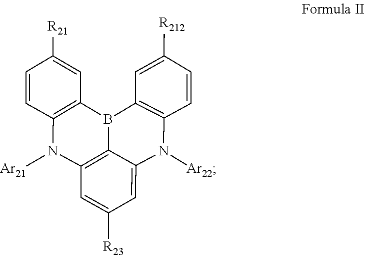
wherein Ar₂₁ and Ar₂₂ are each independently selected from any one of substituted or unsubstituted C6 to C20 aryl, or substituted or unsubstituted C3 to C20 heteroaryl;
R₂₁, R₂₂ and R₂₃ are each independently selected from any one of hydrogen, C1 to C12 linear or branched alkyl, or C6 to C12 cycloalkyl; and
substituted substituents in Ar₂₁ and Ar₂₂ are each independently selected from C1 to C5 linear or branched alkyl, or C6 to C12 aryl.

10. The deuterated composition according to claim 9, wherein Ar₂₁ and Ar₂₂ are each independently selected from any one of 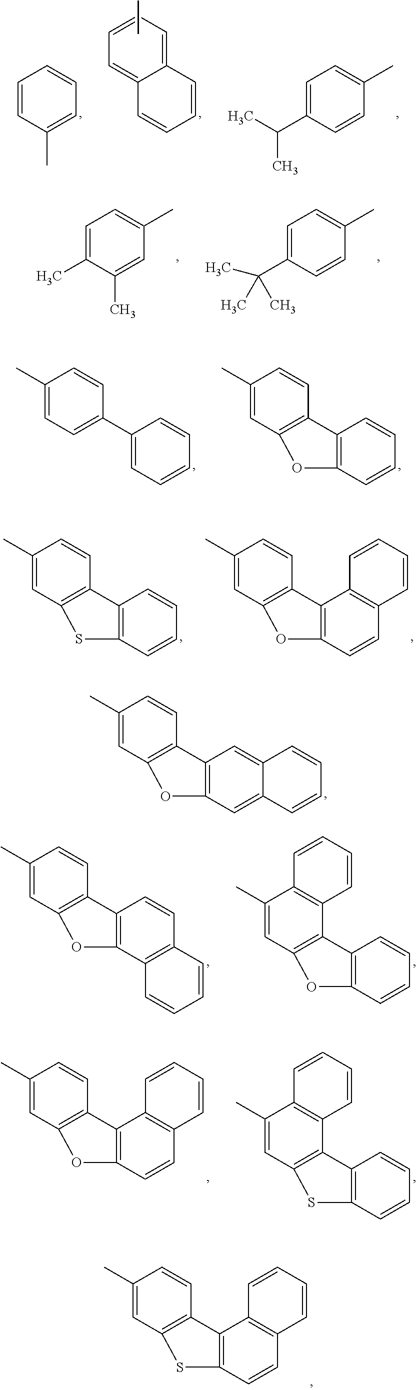 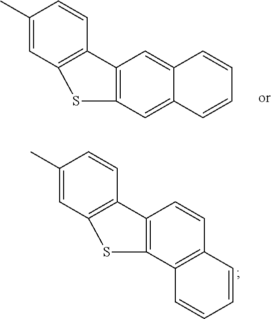 [SMILES: Cc1ccc2c(c1)sc1cc3ccccc3cc12 ; Cc1ccc2c(c1)sc1c3ccccc3ccc21]
optionally, R₁, R₂ and R₃ are each independently selected from any one of hydrogen, methyl, ethyl, propyl, n-butyl, isobutyl, sec-butyl, tert-butyl, cyclohexyl or adamantyl;
optionally, Compound B is selected from any one of the following compounds:

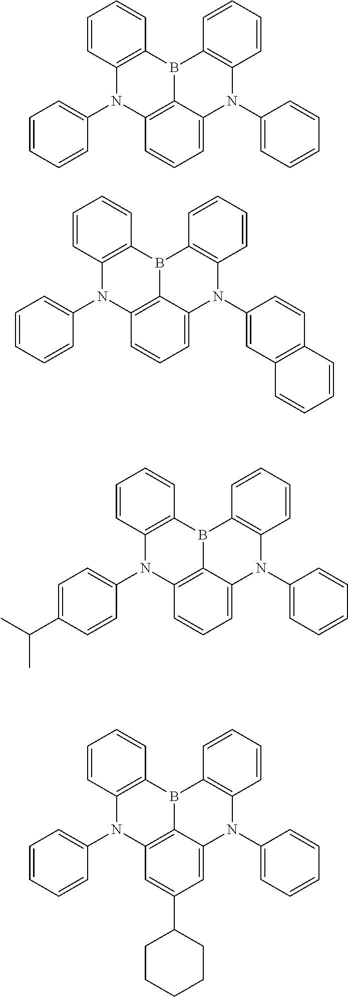 [SMILES: c1ccc(N2c3ccccc3B3c4ccccc4N(c4ccccc4)c4cccc2c43)cc1 ; c1ccc(N2c3ccccc3B3c4ccccc4N(c4ccc5ccccc5c4)c4cccc2c43)cc1 ; CC(C)c1ccc(N2c3ccccc3B3c4ccccc4N(c4ccccc4)c4cccc2c43)cc1 ; c1ccc(N2c3ccccc3B3c4ccccc4N(c4ccccc4)c4cc(C5CCCCC5)cc2c43)cc1]

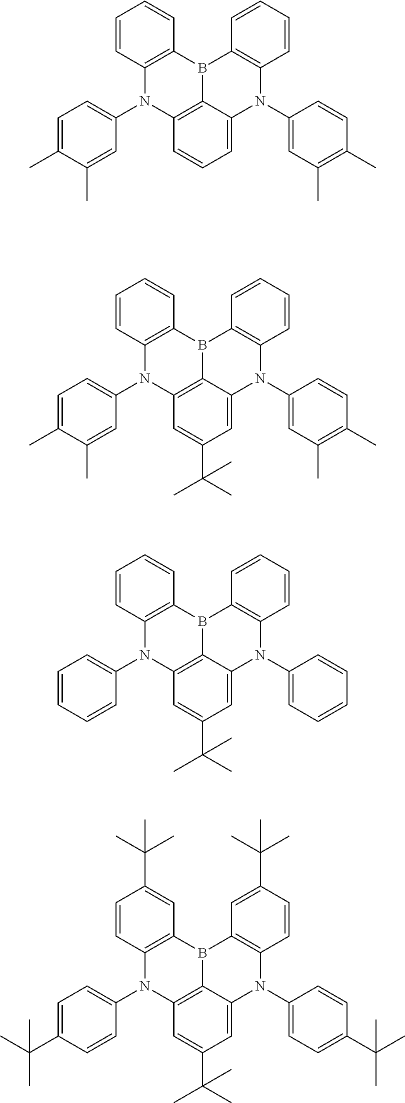

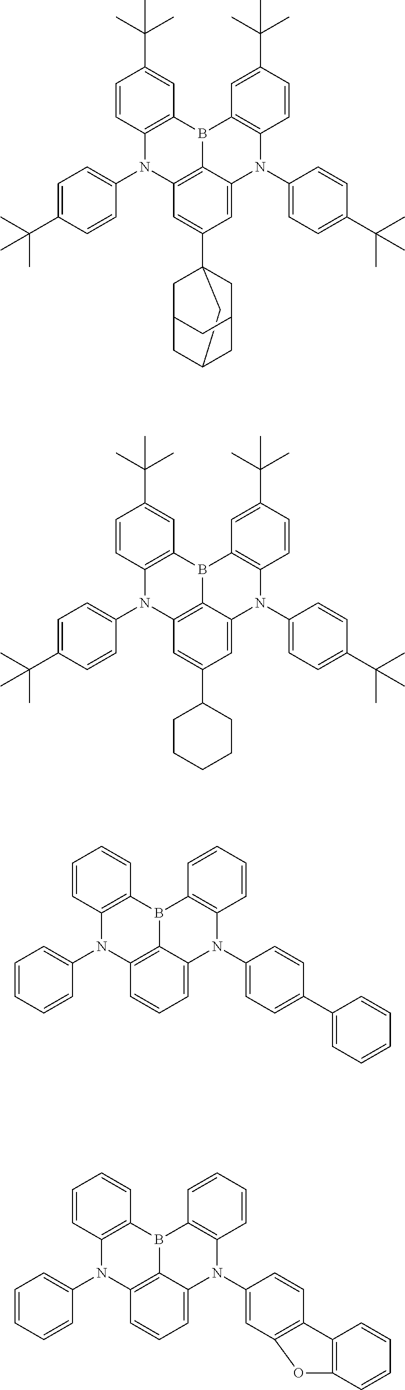

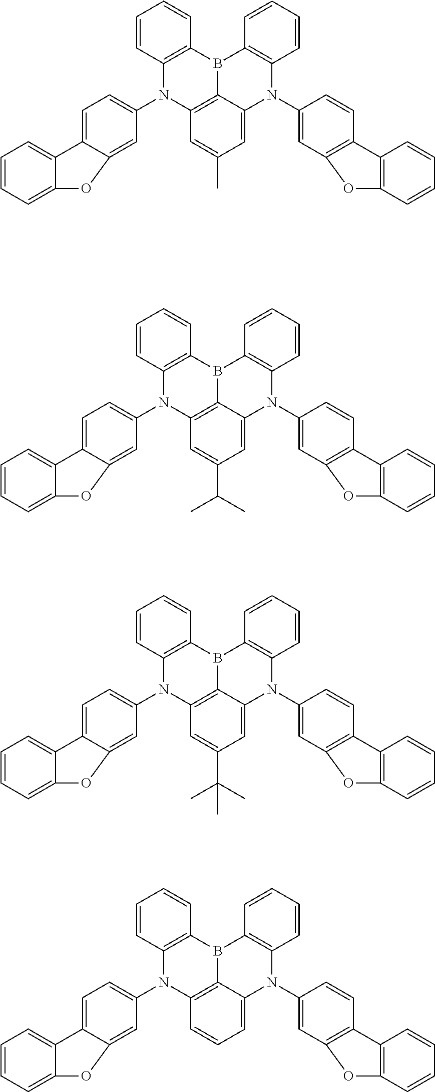 [SMILES: Cc1cc2c3c(c1)N(c1ccc4c(c1)oc1ccccc14)c1ccccc1B3c1ccccc1N2c1ccc2c(c1)oc1ccccc12 ; CC(C)c1cc2c3c(c1)N(c1ccc4c(c1)oc1ccccc14)c1ccccc1B3c1ccccc1N2c1ccc2c(c1)oc1ccccc12 ; CC(C)(C)c1cc2c3c(c1)N(c1ccc4c(c1)oc1ccccc14)c1ccccc1B3c1ccccc1N2c1ccc2c(c1)oc1ccccc12 ; c1ccc2c(c1)B1c3ccccc3N(c3ccc4c(c3)oc3ccccc34)c3cccc(c31)N2c1ccc2c(c1)oc1ccccc12]

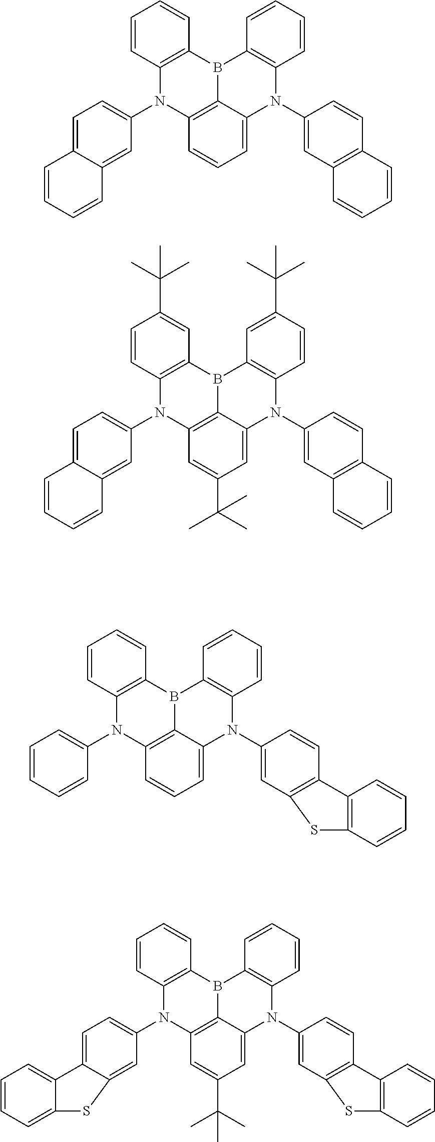 [SMILES: c1ccc2c(c1)B1c3ccccc3N(c3ccc4ccccc4c3)c3cccc(c31)N2c1ccc2ccccc2c1 ; CC(C)(C)c1ccc2c(c1)B1c3cc(C(C)(C)C)ccc3N(c3ccc4ccccc4c3)c3cc(C(C)(C)C)cc(c31)N2c1ccc2ccccc2c1 ; c1ccc(N2c3ccccc3B3c4ccccc4N(c4ccc5c(c4)sc4ccccc45)c4cccc2c43)cc1 ; CC(C)(C)c1cc2c3c(c1)N(c1ccc4c(c1)sc1ccccc14)c1ccccc1B3c1ccccc1N2c1ccc2c(c1)sc1ccccc12]

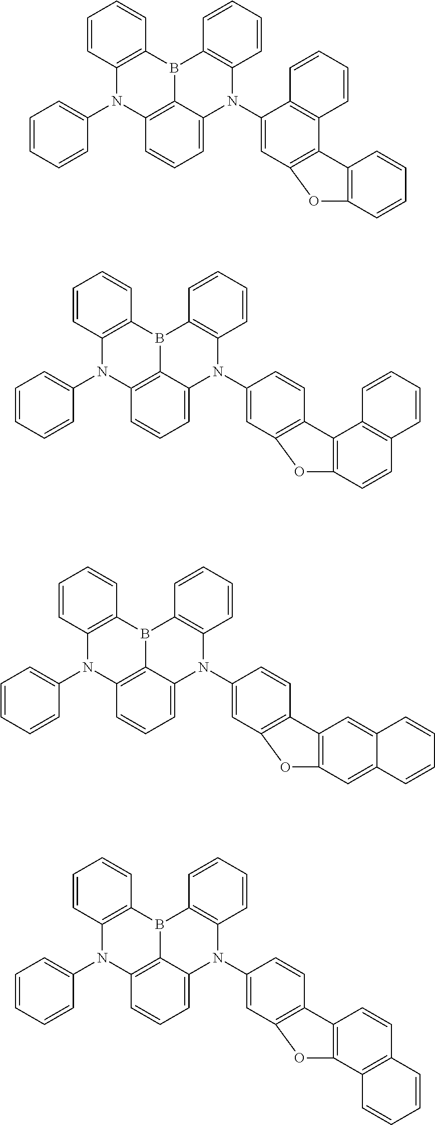 [SMILES: c1ccc(N2c3ccccc3B3c4ccccc4N(c4cc5oc6ccccc6c5c5ccccc45)c4cccc2c43)cc1 ; c1ccc(N2c3ccccc3B3c4ccccc4N(c4ccc5c(c4)oc4ccc6ccccc6c45)c4cccc2c43)cc1 ; c1ccc(N2c3ccccc3B3c4ccccc4N(c4ccc5c(c4)oc4cc6ccccc6cc45)c4cccc2c43)cc1 ; c1ccc(N2c3ccccc3B3c4ccccc4N(c4ccc5c(c4)oc4c6ccccc6ccc54)c4cccc2c43)cc1]

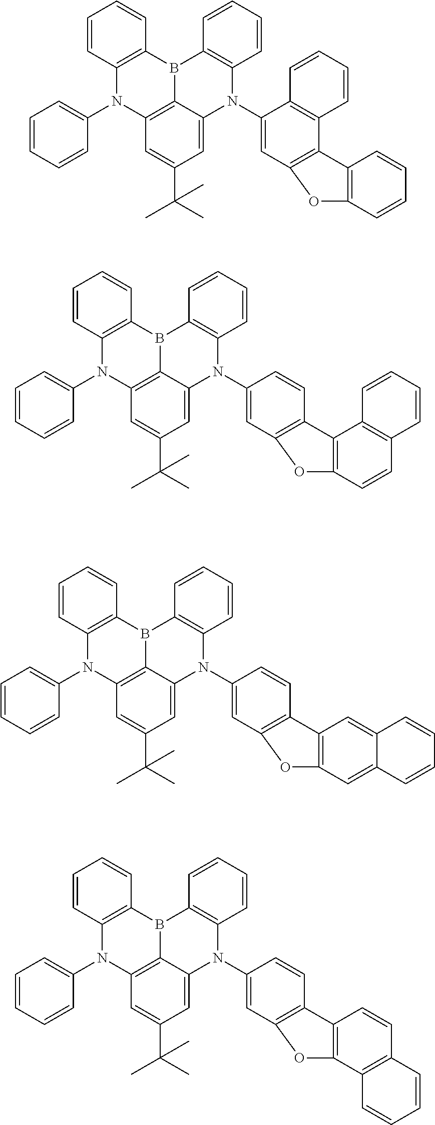 [SMILES: CC(C)(C)c1cc2c3c(c1)N(c1cc4oc5ccccc5c4c4ccccc14)c1ccccc1B3c1ccccc1N2c1ccccc1 ; CC(C)(C)c1cc2c3c(c1)N(c1ccc4c(c1)oc1ccc5ccccc5c14)c1ccccc1B3c1ccccc1N2c1ccccc1 ; CC(C)(C)c1cc2c3c(c1)N(c1ccc4c(c1)oc1cc5ccccc5cc14)c1ccccc1B3c1ccccc1N2c1ccccc1 ; CC(C)(C)c1cc2c3c(c1)N(c1ccc4c(c1)oc1c5ccccc5ccc41)c1ccccc1B3c1ccccc1N2c1ccccc1]

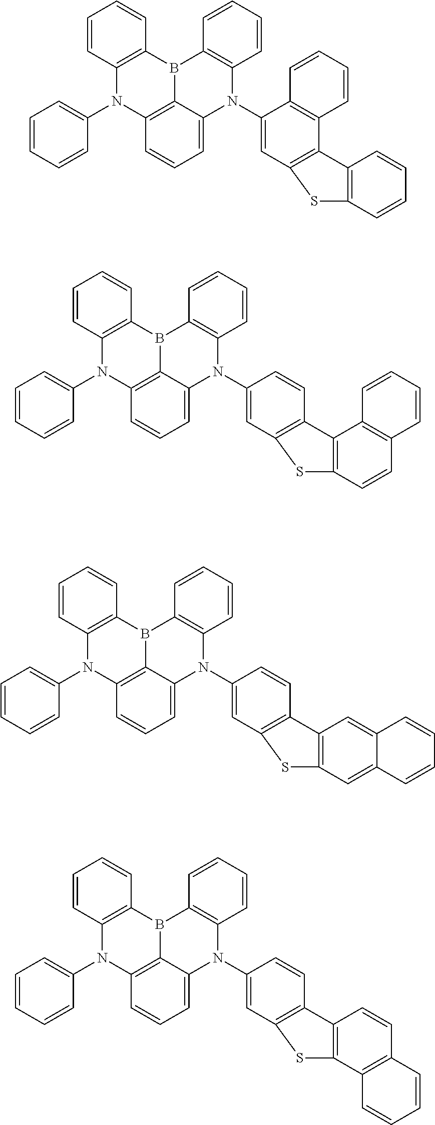 [SMILES: c1ccc(N2c3ccccc3B3c4ccccc4N(c4cc5sc6ccccc6c5c5ccccc45)c4cccc2c43)cc1 ; c1ccc(N2c3ccccc3B3c4ccccc4N(c4ccc5c(c4)sc4ccc6ccccc6c45)c4cccc2c43)cc1 ; c1ccc(N2c3ccccc3B3c4ccccc4N(c4ccc5c(c4)sc4cc6ccccc6cc45)c4cccc2c43)cc1 ; c1ccc(N2c3ccccc3B3c4ccccc4N(c4ccc5c(c4)sc4c6ccccc6ccc54)c4cccc2c43)cc1]

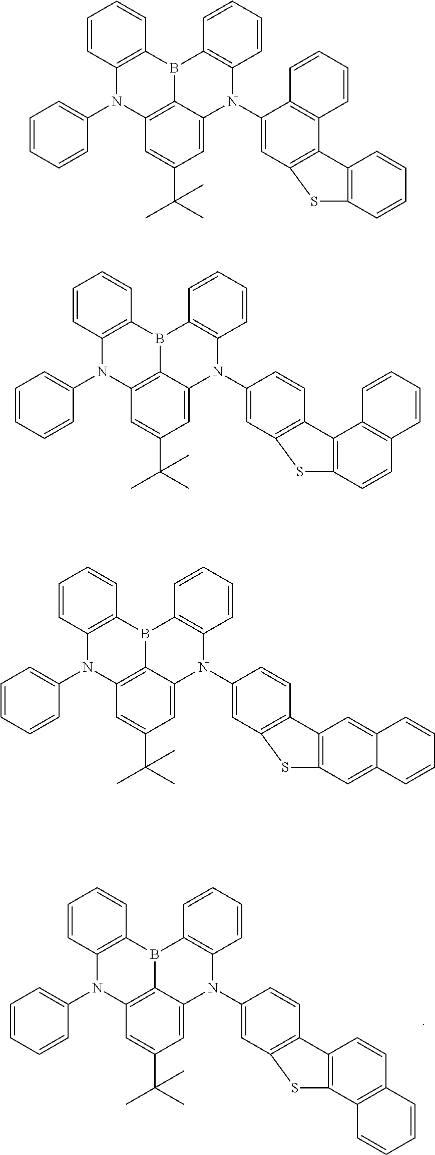 [SMILES: CC(C)(C)c1cc2c3c(c1)N(c1cc4sc5ccccc5c4c4ccccc14)c1ccccc1B3c1ccccc1N2c1ccccc1 ; CC(C)(C)c1cc2c3c(c1)N(c1ccc4c(c1)sc1ccc5ccccc5c14)c1ccccc1B3c1ccccc1N2c1ccccc1 ; CC(C)(C)c1cc2c3c(c1)N(c1ccc4c(c1)sc1cc5ccccc5cc14)c1ccccc1B3c1ccccc1N2c1ccccc1 ; CC(C)(C)c1cc2c3c(c1)N(c1ccc4c(c1)sc1c5ccccc5ccc41)c1ccccc1B3c1ccccc1N2c1ccccc1]

11. The deuterated composition according to claim 1, wherein the deuterated composition further comprises Compound C having a structure represented by Formula III: 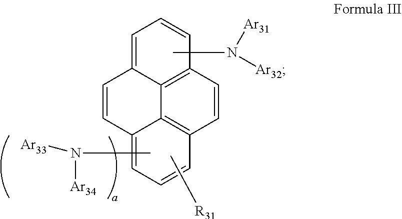
wherein Ar₃₁, Ar₃₂, Ar₃₃ and Ar₃₄ are each independently selected from any one of substituted or unsubstituted C6 to C22 aryl or substituted or unsubstituted C12 to C40 heteroaryl;
R₃₁ is selected from any one of phenyl, naphthyl or biphenyl;
a is selected from 0 or 1;
substituted substituents in Ar₃₁, Ar₃₂, Ar₃₃ and Ar₃₄ are each independently selected from C₁ to C₅ linear or branched alkyl or C6 to C12 aryl;
optionally, Ar₃₁, Ar₃₂, Ar₃₃ and Ar₃₄ are each independently selected from any one or a combination of at least two of 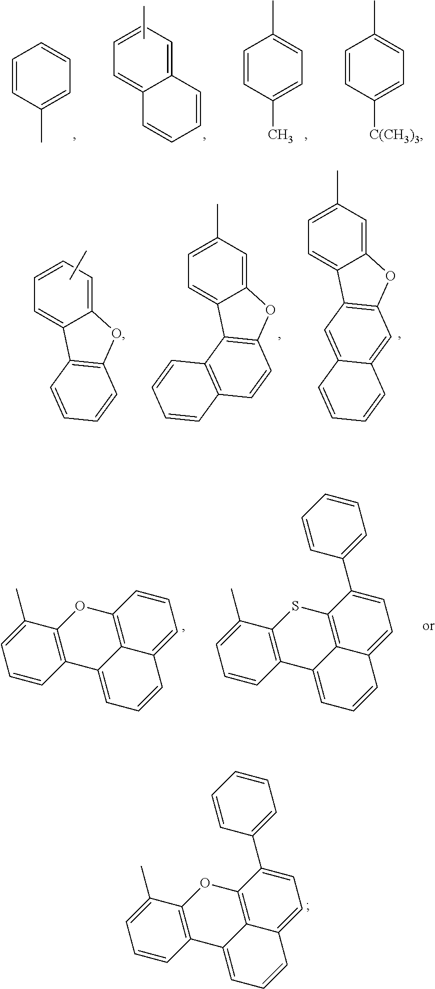
optionally, Compound C is selected from any one of the following compounds:

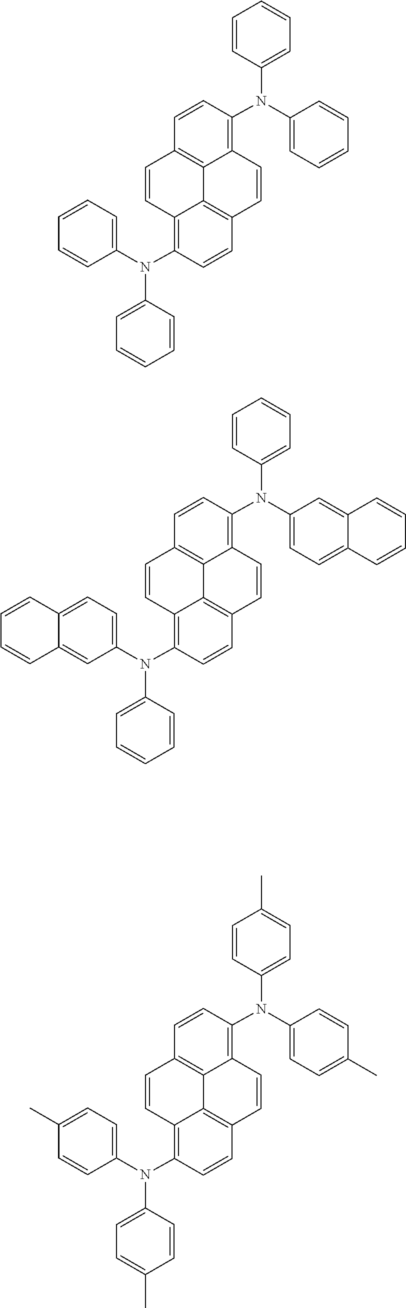

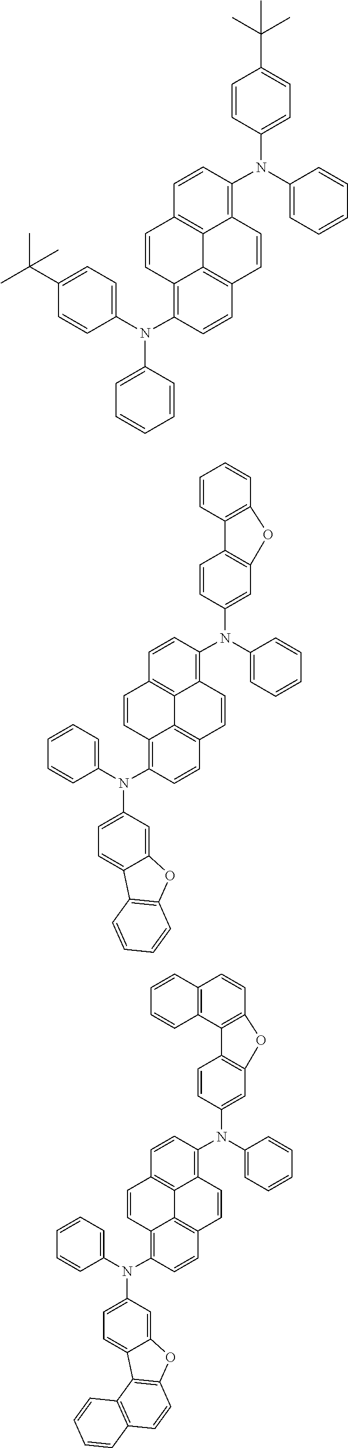

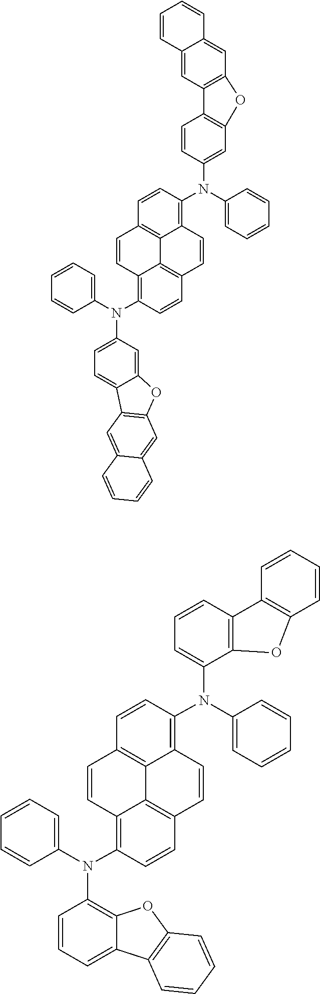 [SMILES: c1ccc(N(c2ccc3c(c2)oc2cc4ccccc4cc23)c2ccc3ccc4c(N(c5ccccc5)c5ccc6c(c5)oc5cc7ccccc7cc56)ccc5ccc2c3c54)cc1 ; c1ccc(N(c2ccc3ccc4c(N(c5ccccc5)c5cccc6c5oc5ccccc56)ccc5ccc2c3c54)c2cccc3c2oc2ccccc23)cc1]

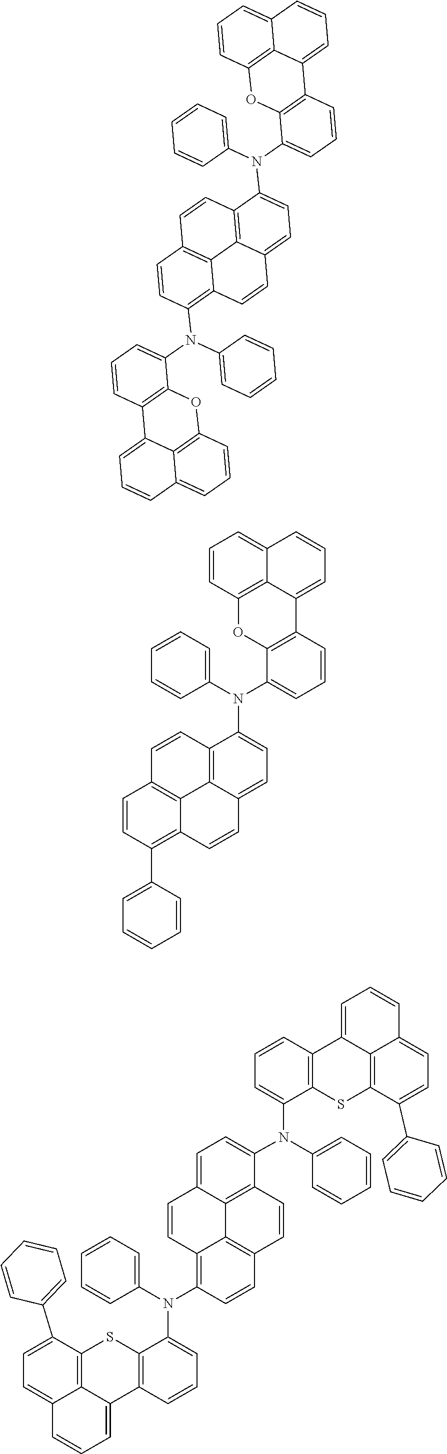 [SMILES: c1ccc(N(c2cccc3c2Oc2cccc4cccc-3c24)c2ccc3ccc4c(N(c5ccccc5)c5cccc6c5Oc5cccc7cccc-6c57)ccc5ccc2c3c54)cc1 ; c1ccc(-c2ccc3ccc4c(N(c5ccccc5)c5cccc6c5Oc5cccc7cccc-6c57)ccc5ccc2c3c54)cc1 ; c1ccc(-c2ccc3cccc4c3c2Sc2c-4cccc2N(c2ccccc2)c2ccc3ccc4c(N(c5ccccc5)c5cccc6c5Sc5c(-c7ccccc7)ccc7cccc-6c57)ccc5ccc2c3c54)cc1]

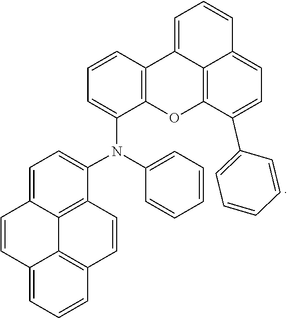 [SMILES: c1ccc(-c2ccc3cccc4c3c2Oc2c-4cccc2N(c2ccccc2)c2ccc3ccc4cccc5ccc2c3c45)cc1]

12. An organic electroluminescent device, comprising an anode, a cathode and an organic thin-film layer disposed between the anode and the cathode, wherein the organic thin-film layer comprises a light-emitting layer; and
a material of the organic thin-film layer comprises the deuterated composition according to claim 1.

13. The organic electroluminescent device according to claim 12, wherein the organic layer comprises a light-emitting layer; and
a material of the light-emitting layer comprises the deuterated composition.

14. The organic electroluminescent device according to claim 12, wherein the material of the light-emitting layer further comprises a doped material;
wherein the doped material comprises a compound having a structure represented by Formula BDI: 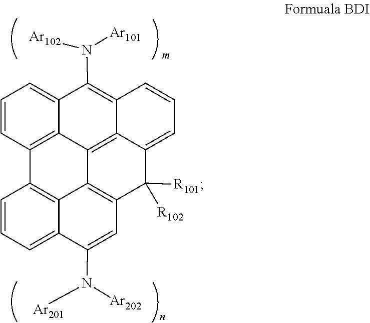
wherein Ar₁₀₁, Ar₁₀₂, Ar₂₀₁ and Ar₂₀₂ are each independently selected from any one of substituted or unsubstituted C6 to C40 aryl or substituted or unsubstituted C12 to C20 heteroaryl;
R₁₀₁ and R₁₀₂ are each independently selected from any one of substituted or unsubstituted C1 to C12 alkyl, substituted or unsubstituted C6 to C40 aryl, or substituted or unsubstituted C12 to C20 heteroaryl;
R₁₀₁ and R₁₀₂ can be joined to form a ring by a single bond;
m and n are each independently selected from 0 or 1 and are not 0 at the same time; and
substituted substituents in Ar₁₀₁, Ar₁₀₂, Ar₂₀₁, Ar₂₀₂, R₁₀₁ and R₁₀₂ are each independently selected from any one or a combination of at least two of -D, —F, —CN, C1 to C12 alkyl, C₁ to C6 alkoxy, C2 to C8 alkenyl, C6 to C15 aryl, or C12 to C20 heteroaryl.

15. The organic electroluminescent device according to claim 14, wherein the C6 to C40 aryl is selected from any one of phenyl, biphenyl, terphenyl, naphthyl, anthryl, phenanthryl, fluorenyl, benzofluorenyl, 9,10-diphenylanthryl, dibenzofluorenyl, naphthofluorenyl, pyrenyl, perylenyl, spirofluorenyl, triphenylenyl, fluoranthenyl, hydrogenated benzanthryl, indenofluorenyl, benzindenofluorenyl, dibenzindenofluorenyl, naphthofluorenyl or benzonaphthofluorenyl, preferably any one of phenyl, naphthyl, biphenyl, terphenyl, fluoranthenyl, fluorenyl, 9,10-diphenylanthryl or benzofluorenyl;
optionally, the C12 to C20 heteroaryl is selected from any one of dibenzofuranyl, dibenzothienyl, naphthobenzofuranyl, naphthobenzothienyl, dinaphthofuranyl or dinaphthothienyl;
optionally, the C1 to C12 alkyl is selected from any one of methyl, ethyl, propyl, butyl, adamantyl, 1-methylcyclohexyl, 1-methylcyclopentyl, cyclopentyl or cyclohexyl;
optionally, the C1 to C6 alkoxy is selected from any one of methoxy, ethoxy, propoxy, butoxy or 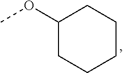
wherein the dashed line represents a linkage site;
optionally, the C6 to C15 aryl is selected from any one of phenyl, naphthyl or biphenyl.

16. The organic electroluminescent device according to claim 14, wherein Ar₁₀₁ and Ar₁₀₂ are each independently selected from any one of the following substituted or unsubstituted groups: phenyl, naphthyl, biphenyl, terphenyl, fluoranthenyl, fluorenyl, 9,10-diphenylanthryl, benzofluorenyl, dibenzofuranyl, dibenzothienyl, naphthobenzofuranyl or naphthobenzothienyl;
the substituted substituents are selected from any one or a combination of at least two of -D, —F, —CN, phenyl, biphenyl, dibenzofuranyl, methyl, deuterated methyl, adamantyl, tert-butyl, 1-methylcyclopentyl, cyclohexyl, cyclopentyl, methoxy, 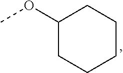
naphthyl, dibenzothienyl or naphthobenzothienyl, wherein the dashed line represents a linkage site;
optionally, Ar₂₀₁ and Ar₂₀₂ are each independently selected from any one of the following substituted or unsubstituted groups: phenyl, naphthyl, dibenzofuranyl or biphenyl;
the substituted substituents are selected from any one or a combination of at least two of methyl, methoxy, phenyl or dibenzofuranyl;
optionally, R₁₀₁ and R₁₀₂ are each independently selected from any one of methyl, ethyl, propyl or phenyl;
optionally, R₁₀₁ is the same as R₁₀₂.

17. The organic electroluminescent device according to claim 14, wherein the compound having the structure represented by Formula BDI is selected from any one of the following compounds:

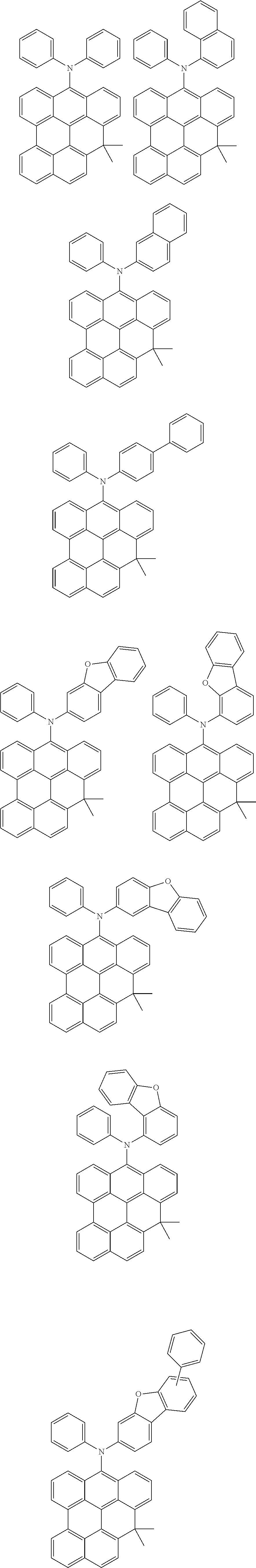

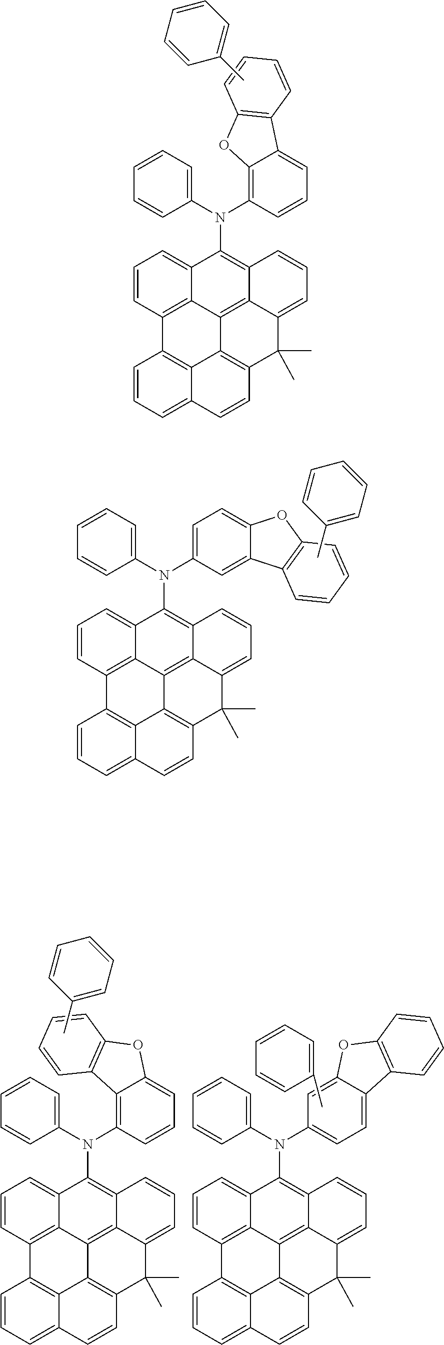

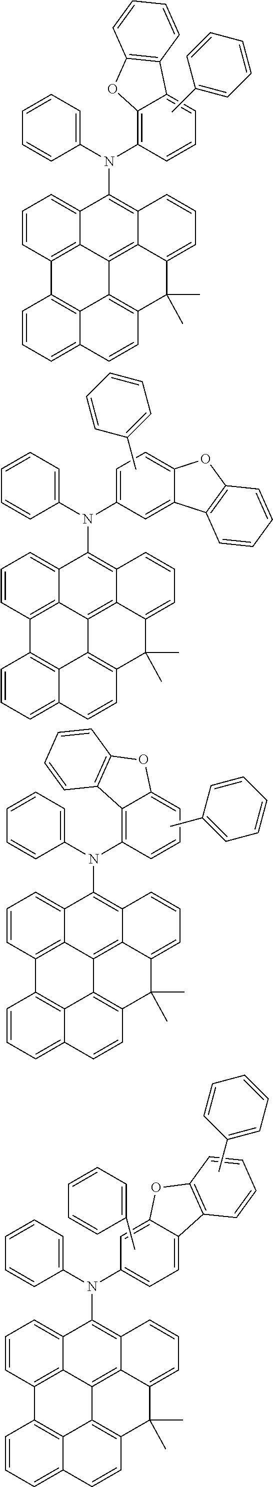

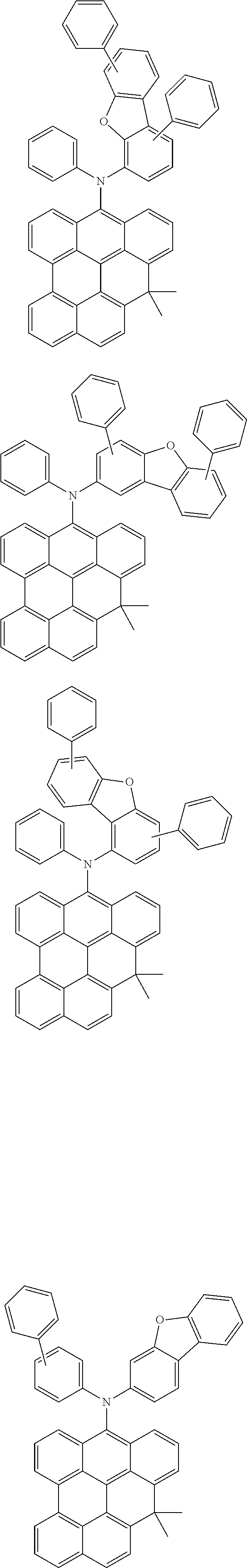

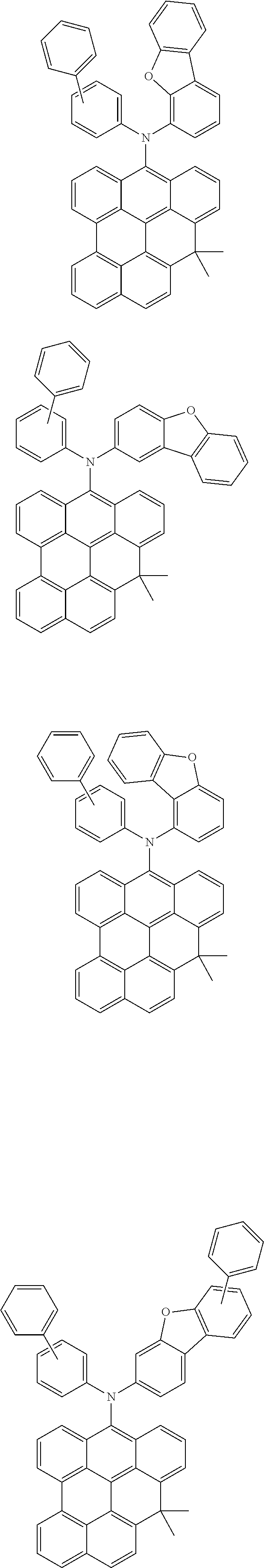

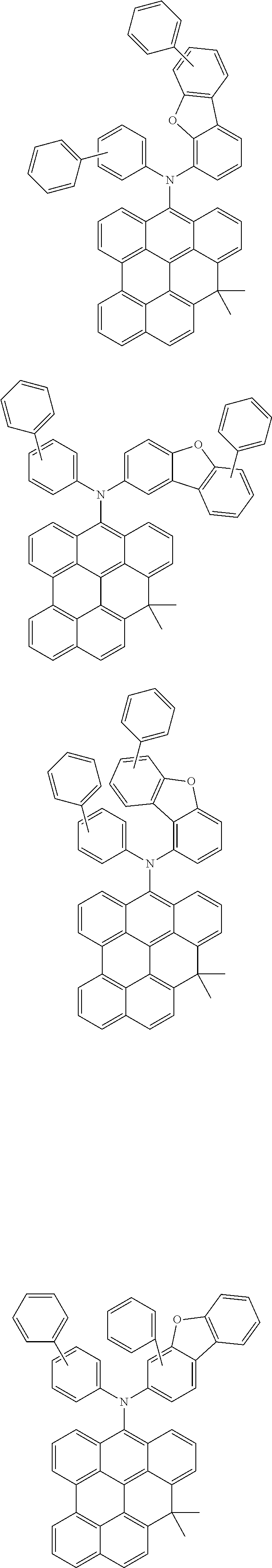

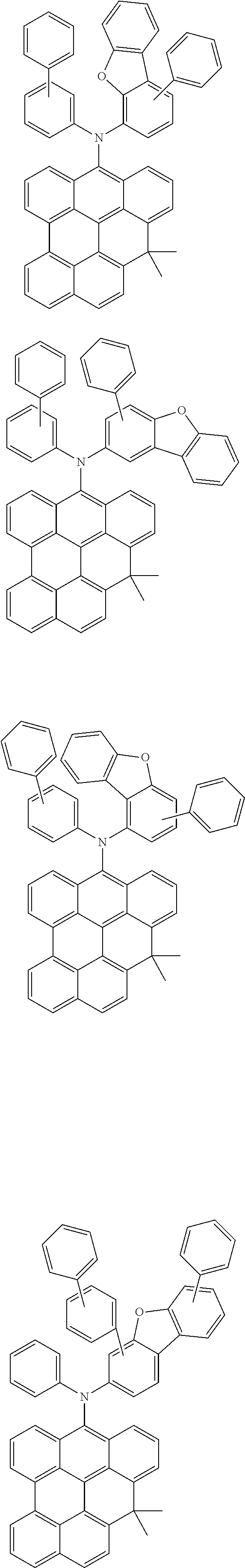

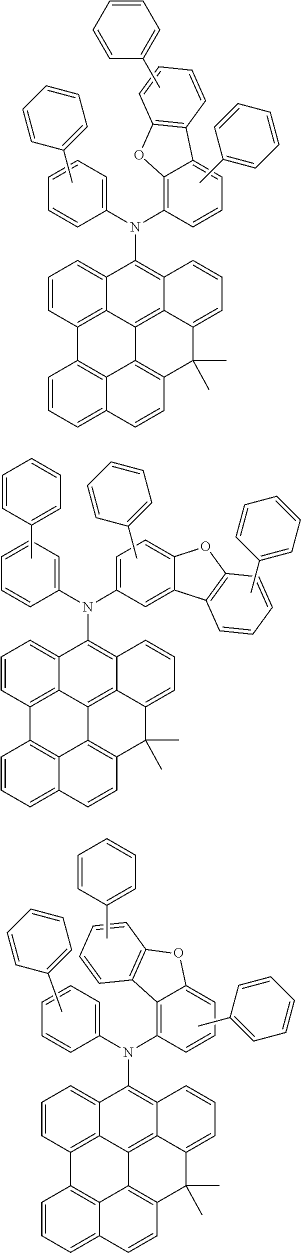

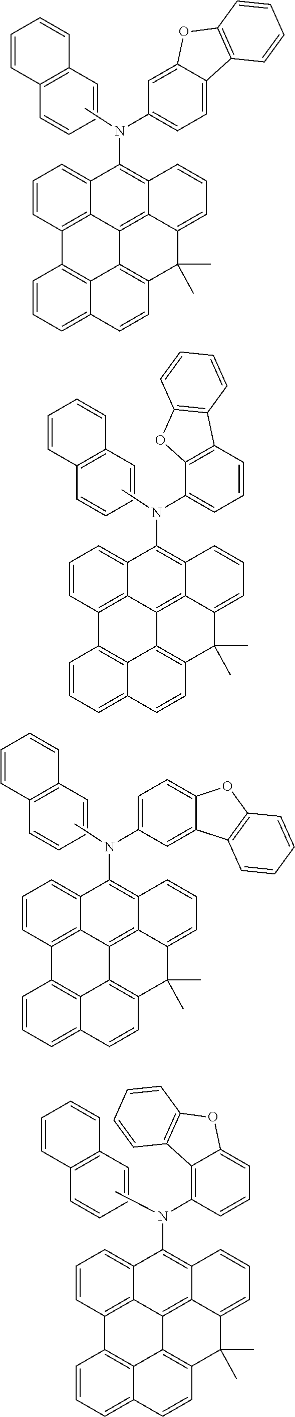

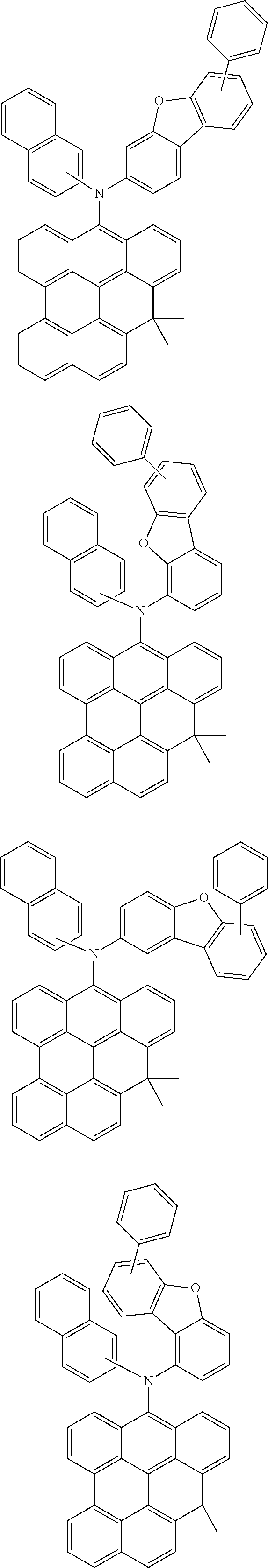

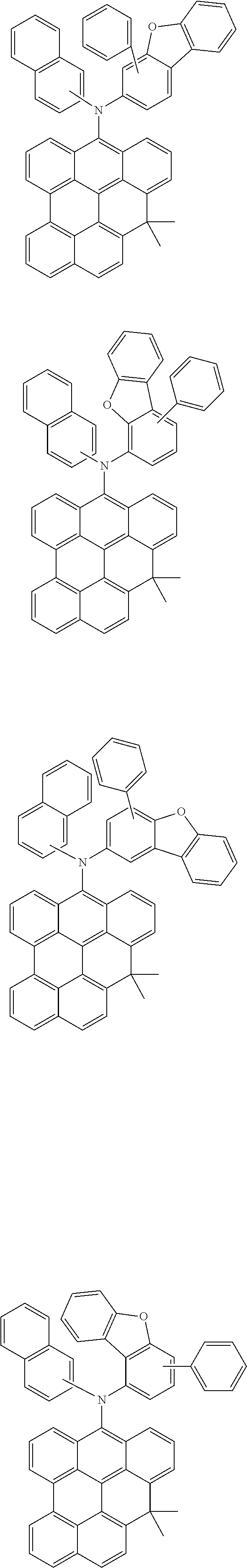

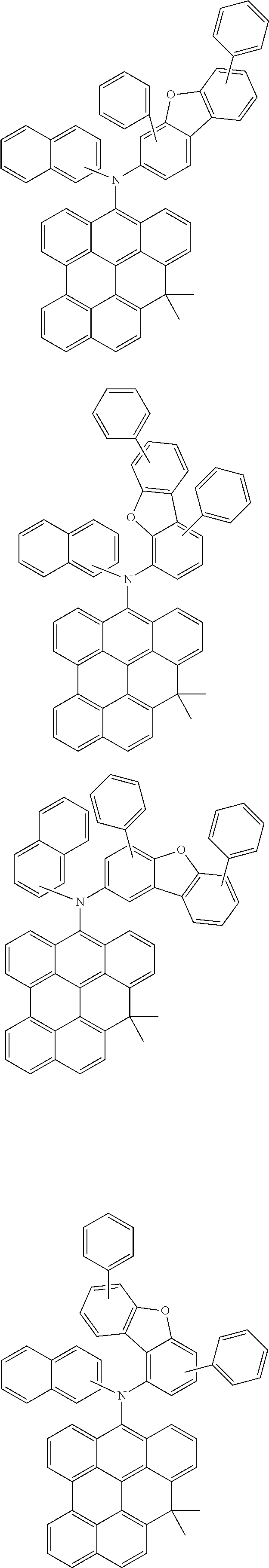

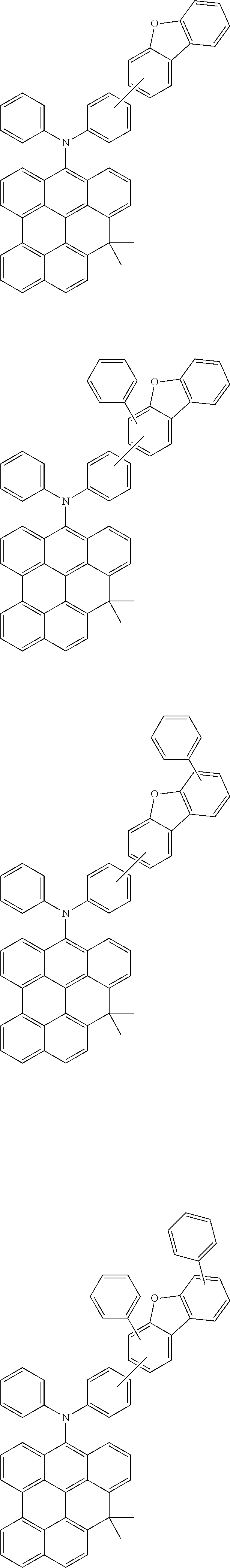

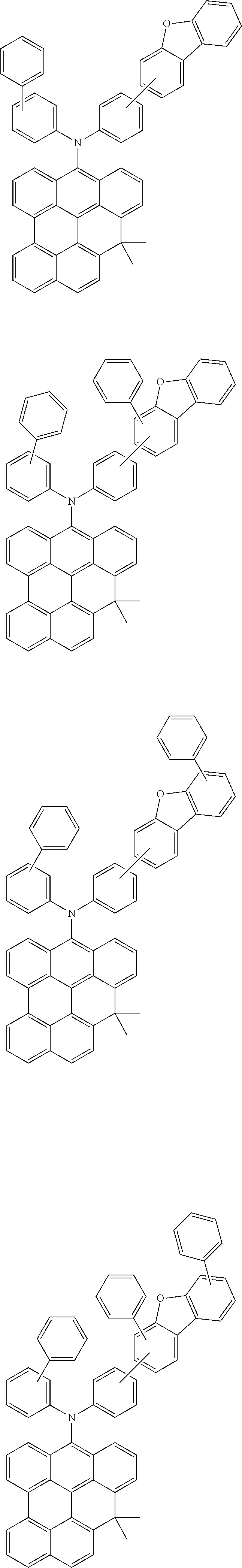

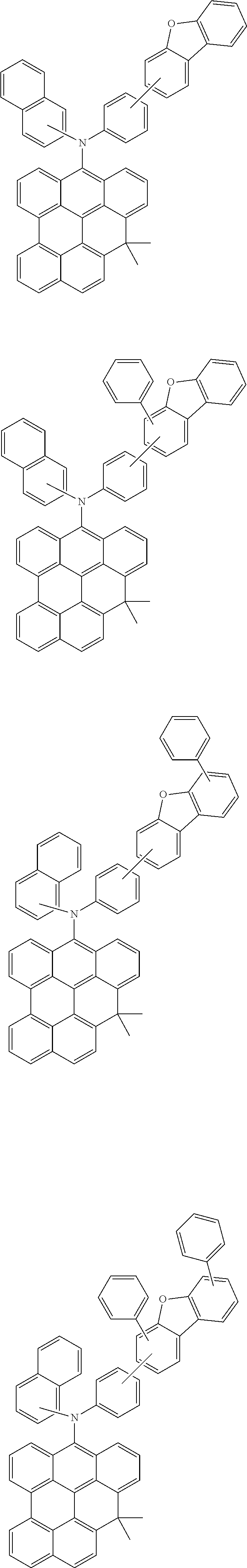

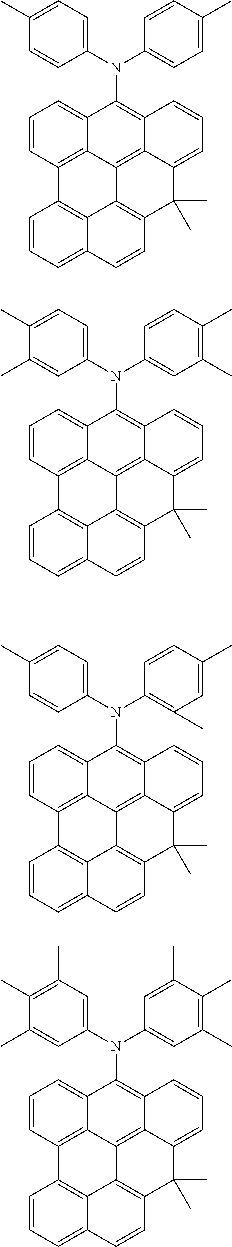

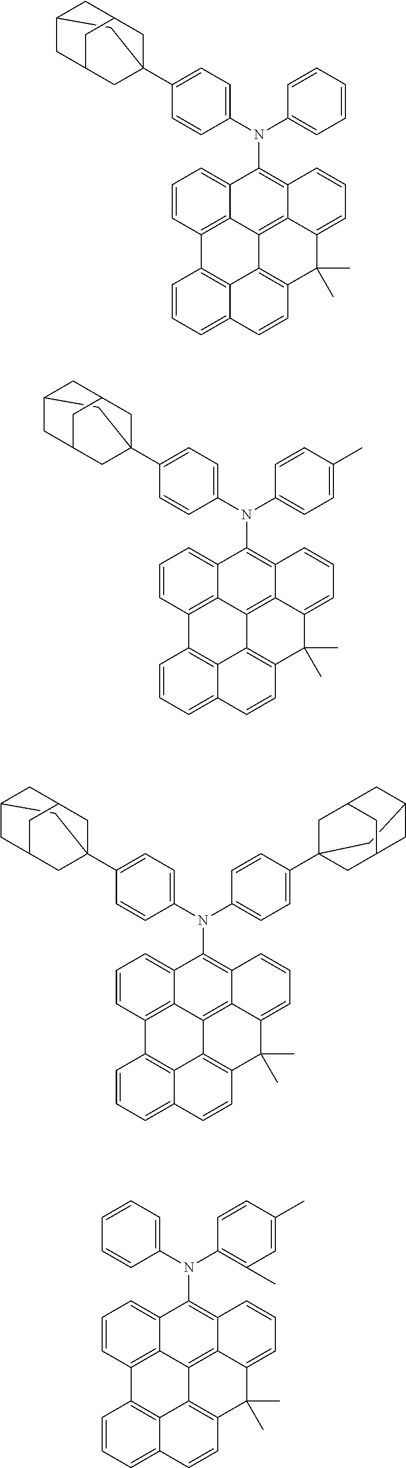 [SMILES: CC1(C)c2cccc3c(N(c4ccccc4)c4ccc(C56CC7CC(CC(C7)C5)C6)cc4)c4cccc5c6cccc7ccc1c(c76)c(c23)c45 ; Cc1ccc(N(c2ccc(C34CC5CC(CC(C5)C3)C4)cc2)c2c3cccc4c3c3c5c(ccc6cccc(c7cccc2c73)c65)C4(C)C)cc1 ; CC1(C)c2cccc3c(N(c4ccc(C56CC7CC(CC(C7)C5)C6)cc4)c4ccc(C56CC7CC(CC(C7)C5)C6)cc4)c4cccc5c6cccc7ccc1c(c76)c(c23)c45 ; Cc1ccc(N(c2ccccc2)c2c3cccc4c3c3c5c(ccc6cccc(c7cccc2c73)c65)C4(C)C)c(C)c1]

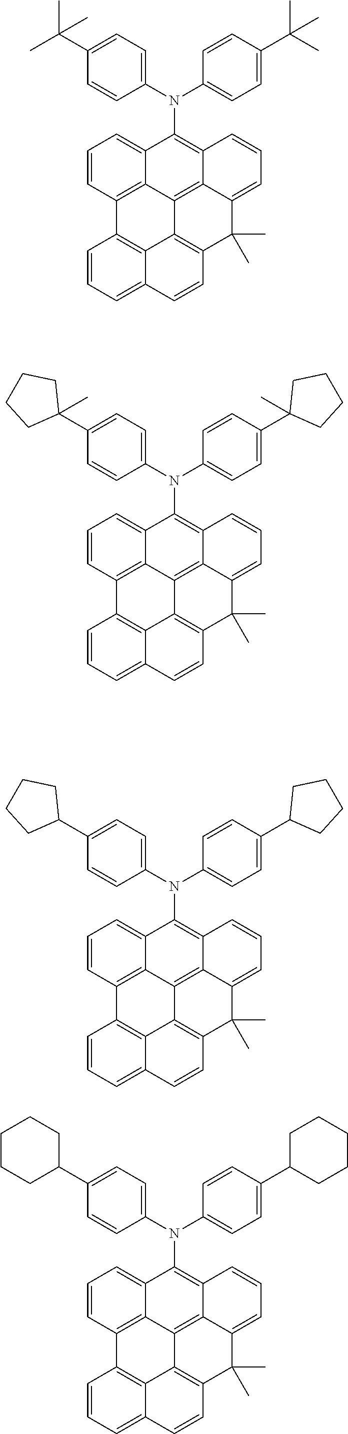 [SMILES: CC(C)(C)c1ccc(N(c2ccc(C(C)(C)C)cc2)c2c3cccc4c3c3c5c(ccc6cccc(c7cccc2c73)c65)C4(C)C)cc1 ; CC1(c2ccc(N(c3ccc(C4(C)CCCC4)cc3)c3c4cccc5c4c4c6c(ccc7cccc(c8cccc3c84)c76)C5(C)C)cc2)CCCC1 ; CC1(C)c2cccc3c(N(c4ccc(C5CCCC5)cc4)c4ccc(C5CCCC5)cc4)c4cccc5c6cccc7ccc1c(c76)c(c23)c45 ; CC1(C)c2cccc3c(N(c4ccc(C5CCCCC5)cc4)c4ccc(C5CCCCC5)cc4)c4cccc5c6cccc7ccc1c(c76)c(c23)c45]

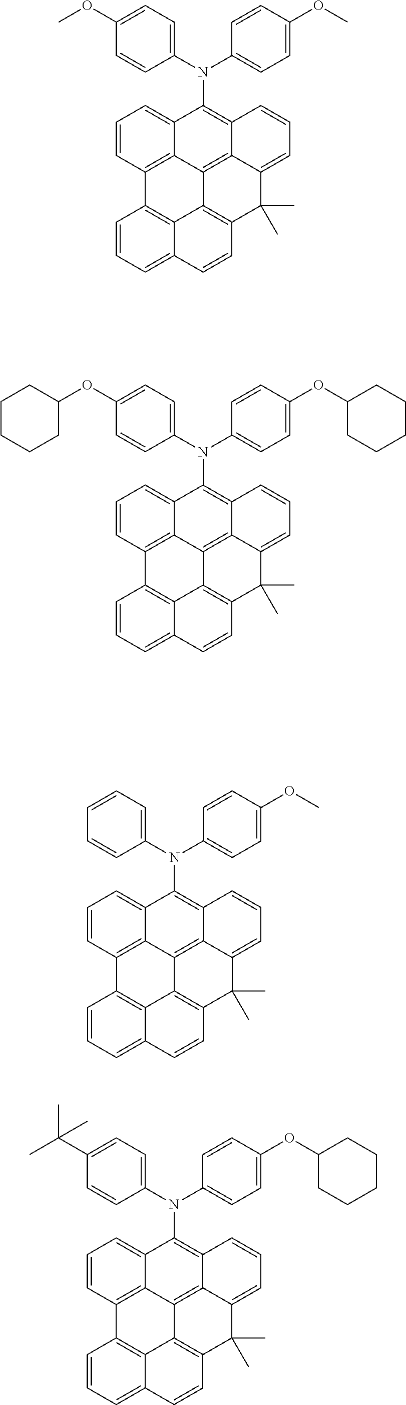

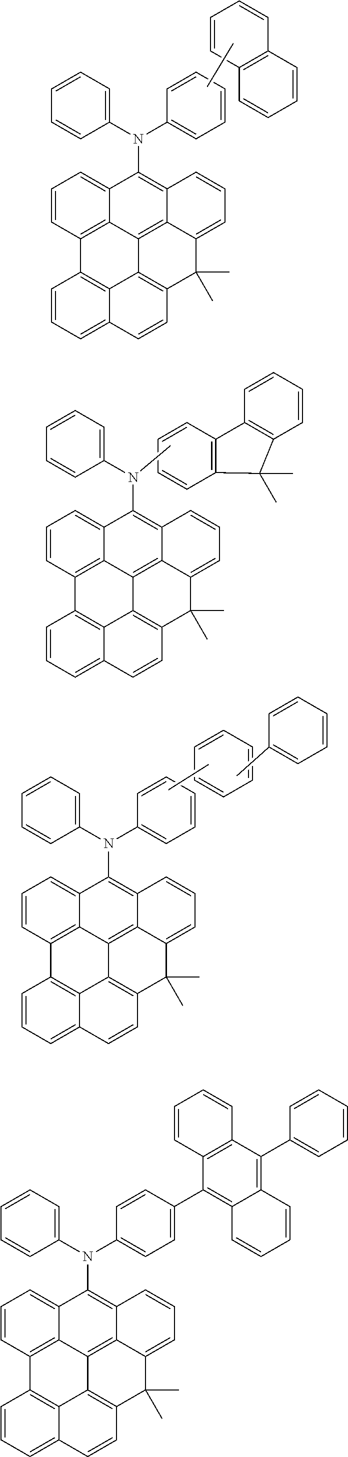

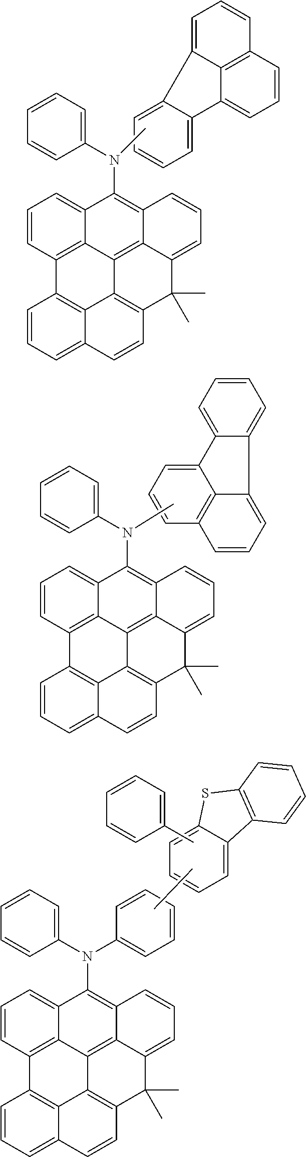

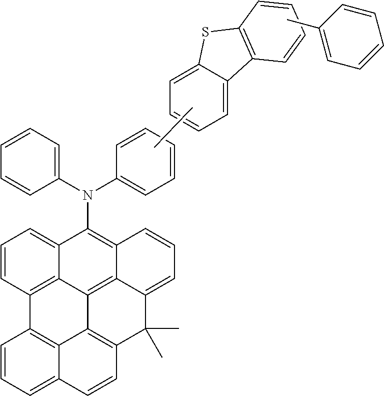

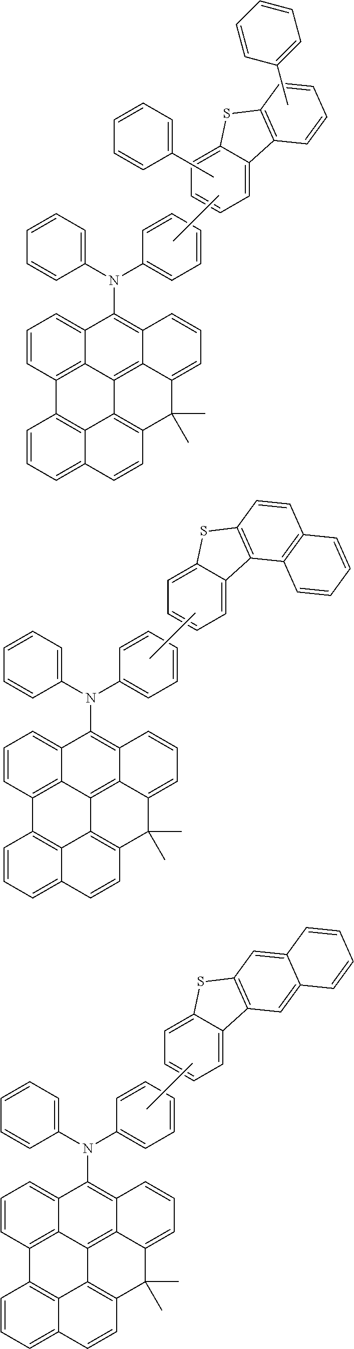

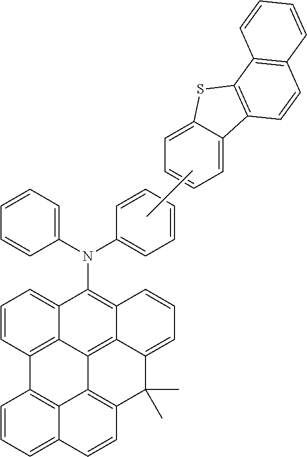

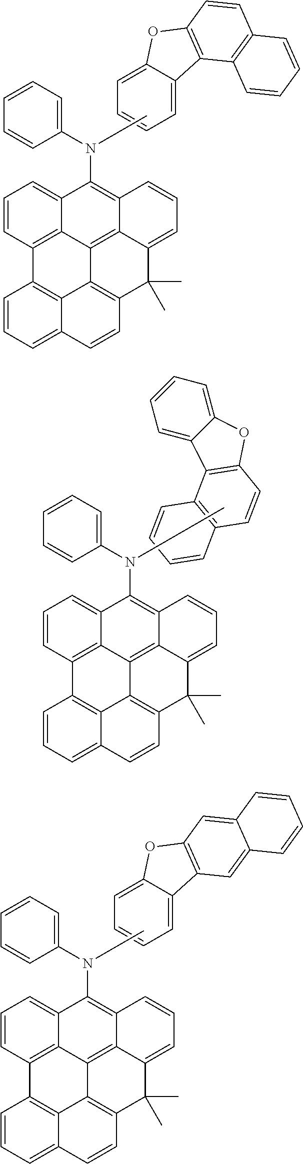

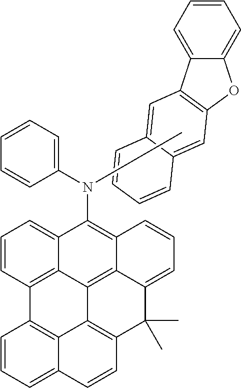

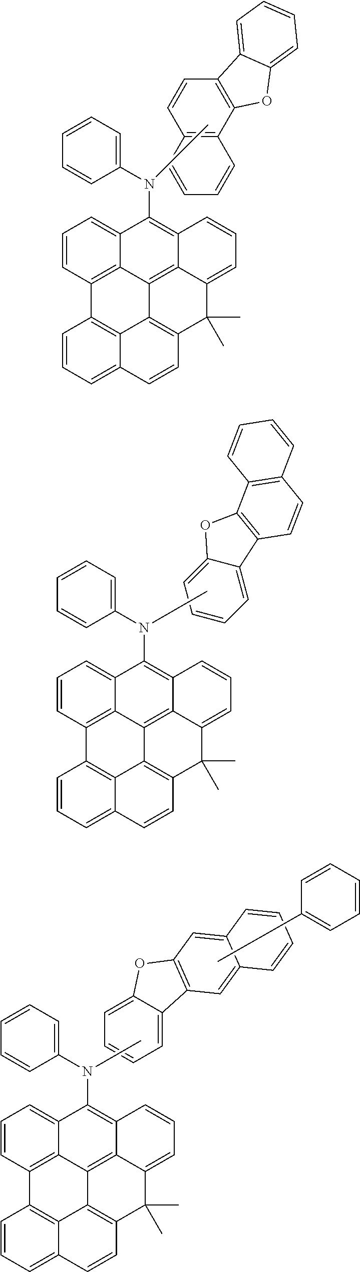

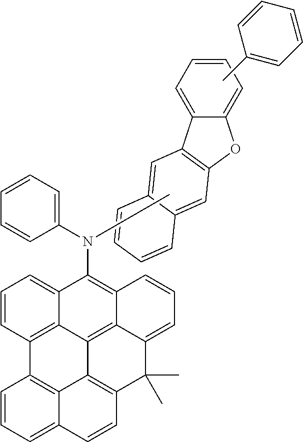

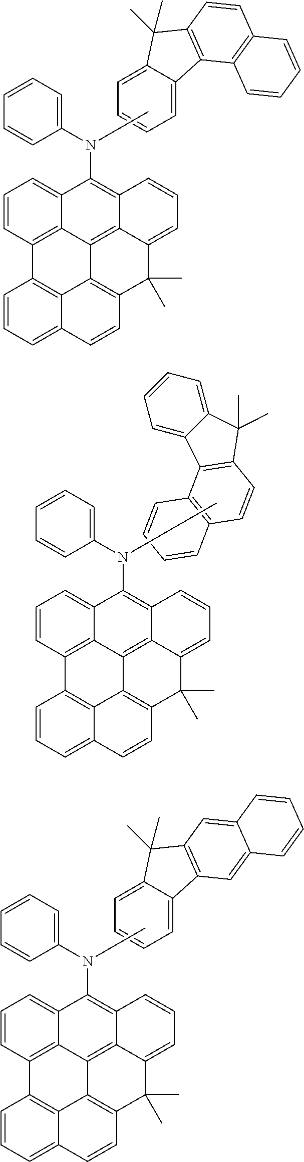

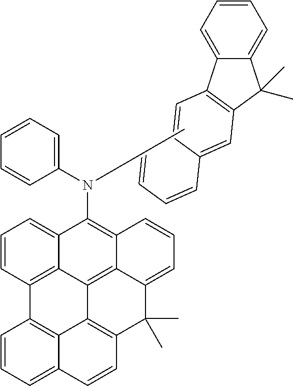

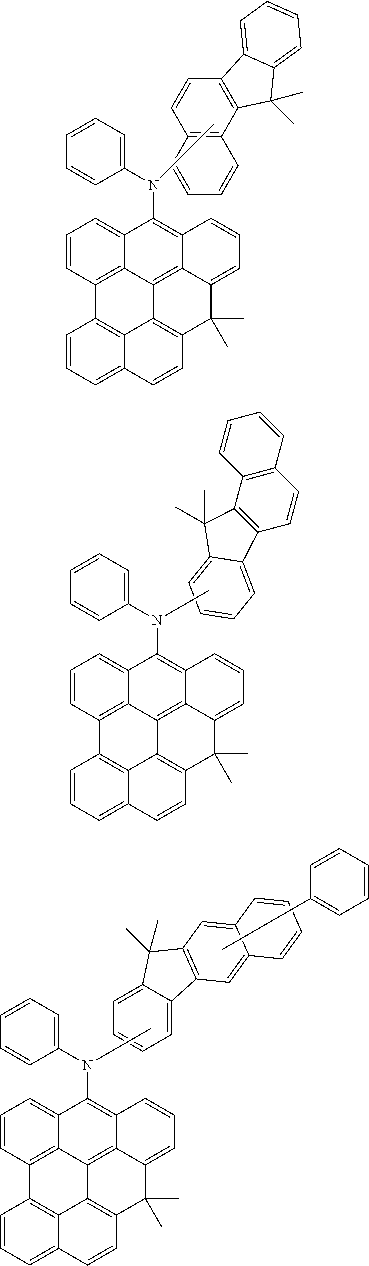

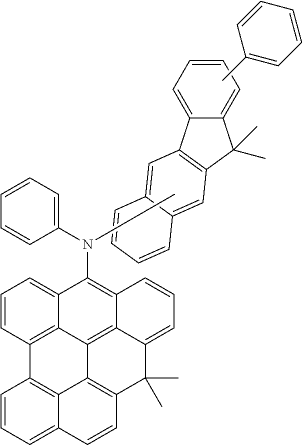

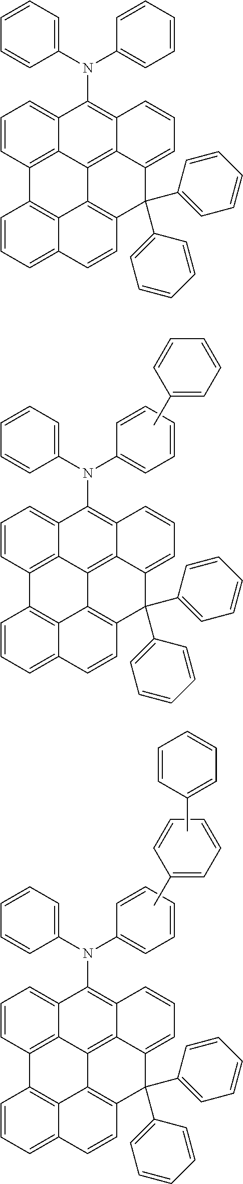

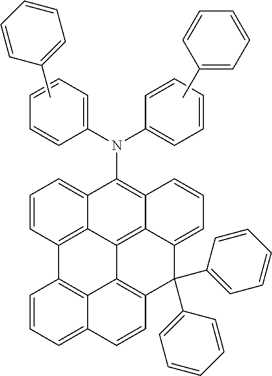

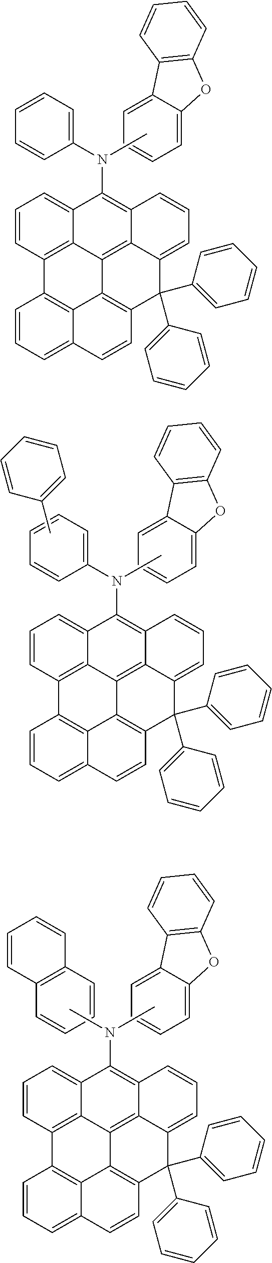

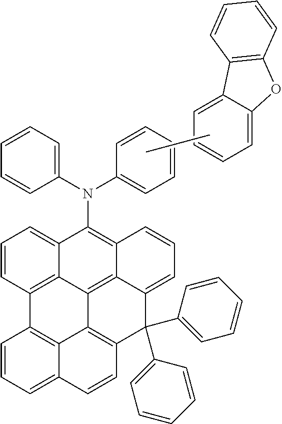

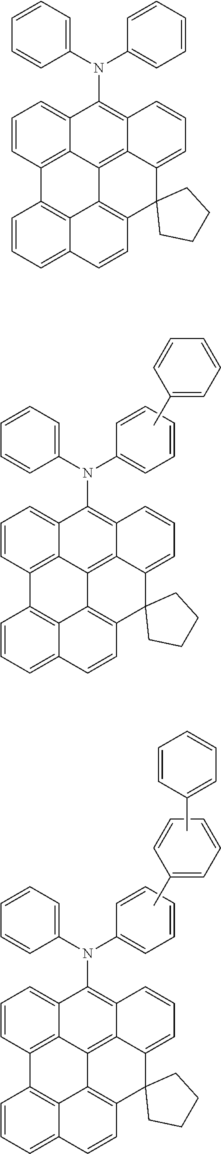

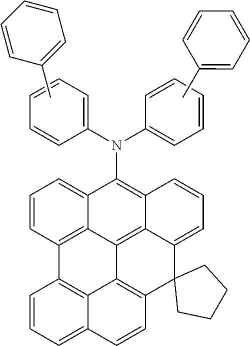

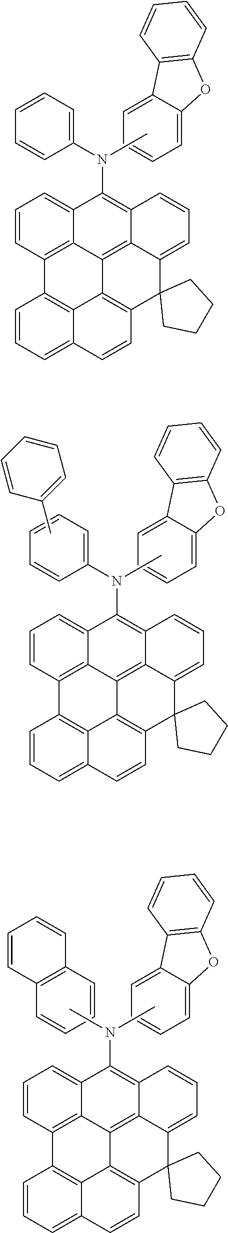

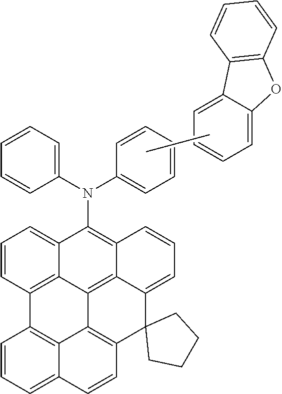

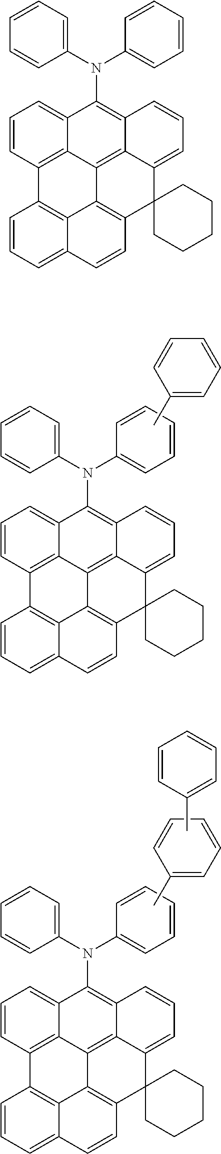

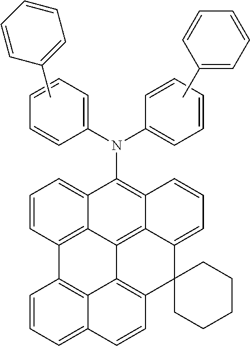

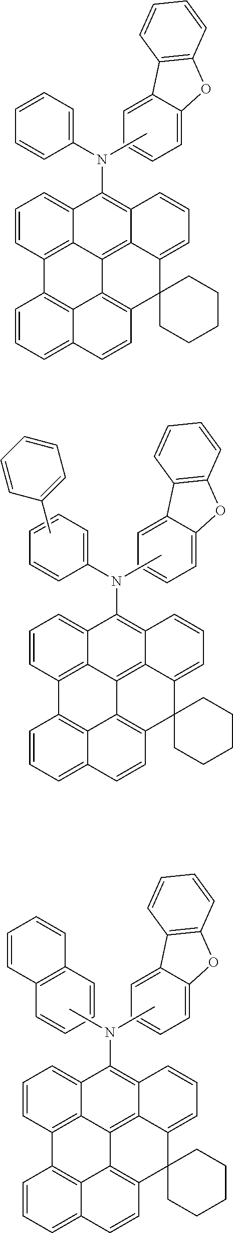

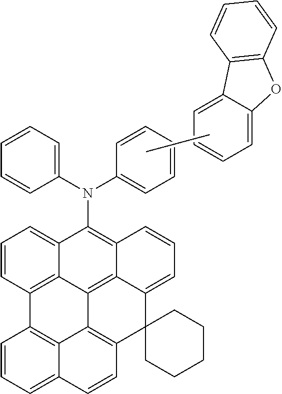

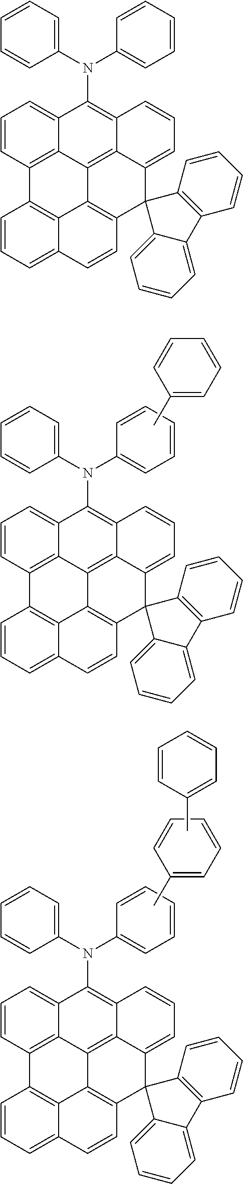

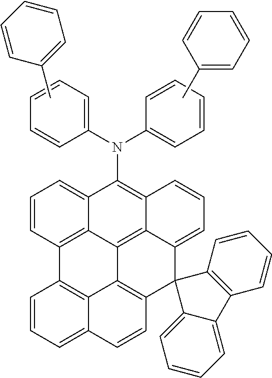

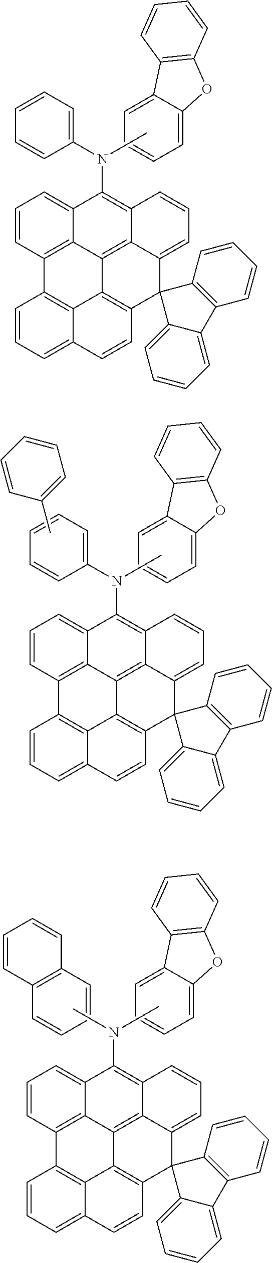

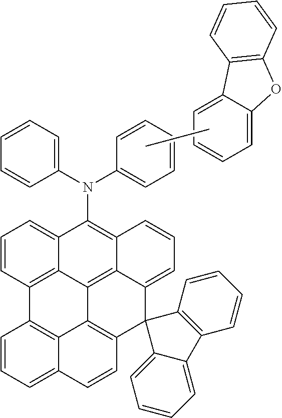

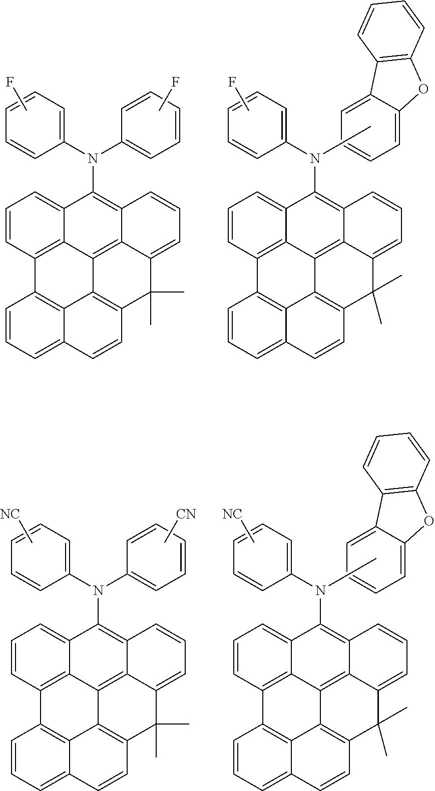

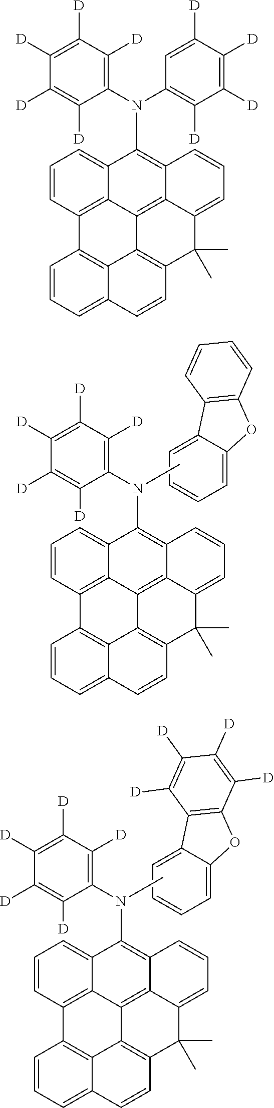

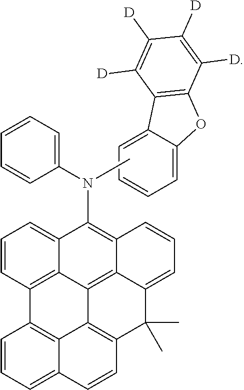

18. A display device, comprising the organic electroluminescent device according to claim 12.
